(19) United States
(12) Patent Application Publication
(10) Pub. No.: US 2025/0179077 A1
(43) Pub. Date: Jun. 5, 2025

(54) QUINAZOLINE COMPOUNDS AND USES THEREOF AS INHIBITORS OF MUTANT KRAS PROTEINS

(71) Applicant: AMGEN INC., Thousand Oaks, CA (US)

(72) Inventors: Brian Alan Lanman, Woodland Hills, CA (US); Ryan Paul Wurz, Newbury Park, CA (US); Wei Zhao, Westford, MA (US); Xiaofen LI, Thousand Oaks, CA (US); Imelda HOT, Thousand Oaks, CA (US); Rene RAHIMOFF, Santa Monica, CA (US); Liping Pettus, Thousand Oaks, CA (US); Ning Chen, Thousand Oaks, CA (US); Fabien EMMETIERE, Thousand Oaks, CA (US); Jeffery Jackson, Thousand Oaks, CA (US); Yunxiao LI, Thousand Oaks, CA (US); Francesco Manoni, Newbury Park, CA (US); Primali Navaratne, Thousand Oaks, CA (US); Andrew Smaligo, Thousand Oaks, CA (US); John Charles Stellwagen, Newbury Park, CA (US); John Allen, Newbury Park, CA (US)

(73) Assignee: AMGEN INC., Thousand Oaks, CA (US)

(21) Appl. No.: 18/838,998

(22) PCT Filed: Feb. 15, 2023
(86) PCT No.: PCT/US2023/062688

§ 371 Date: Aug. 15, 2024

Related U.S. Application Data
(60) Provisional application No. 63/310,914, filed on Feb. 16, 2022.

(51) Int. Cl. C07D 487/04; A61K 31/517; A61K 31/553; C07D 401/14; C07D 498/10; C07D 519/00

(52) U.S. Cl. CPC C07D 487/04; A61K 31/517; A61K 31/553; C07D 401/14; C07D 498/10; C07D 519/00

(57) ABSTRACT

The present disclosure provides compounds useful for the inhibition of KRAS G12D, G12V, G12A, G12S or G12C. The compounds have a general Formula (I): (Formula (I)) wherein the variables of Formula (I) are defined herein. This disclosure also provides pharmaceutical compositions comprising the compounds, uses of the compounds, and compositions for treatment of, for example, cancer.

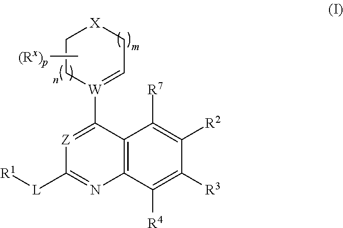

### FIELD

The present disclosure provides compounds having activity as inhibitors of mutant KRAS proteins. This disclosure also provides pharmaceutical compositions comprising the compounds, uses and methods of treating certain disorders, such as cancer, including but not limited to non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

### BACKGROUND

From its identification as one of the first human oncogenes in 1982 (Der et al., 1982), KRAS (the Kirsten rat sarcoma viral oncogene homologue) has been the focus of extensive academic and industrial research, as a key node in the MAPK signal transduction pathway, as a transforming factor in a network of parallel effector pathways (e.g., PI3K/AKT) (Vojtek et al., 1998) and as a potential target for anti-cancer agents (Malumbres et al., 2003). Despite progress in the development of inhibitors of upstream and downstream nodes in the MAPK pathway (e.g., EGFR (Sridhar et al., 2003), BRAF (Holderfield et al., 2014) and MOK (Caunt et al., 2015), the KRAS protein has historically proven resistant to direct inhibition.

KRAS is a G-protein that couples extracellular mitogenic signaling to intracellular, pro-proliferative responses. KRAS serves as an intracellular “on/off” switch. Mitogen stimulation induces the binding of GTP to KRAS, bringing about a conformational change which enables the interaction of KRAS with downstream effector proteins, leading to cellular proliferation. Normally, pro-proliferative signaling is regulated by the action of GTPase-activating proteins (GAPs), which return KRAS to its GDP-bound, non-proliferative state. Mutations in KRAS impair the regulated cycling of KRAS between these GDP- and GTP-bound states, leading to the accumulation of the GTP-bound active state and dysregulated cellular proliferation (Simanshu et al., 2017).

Attempts to develop inhibitors of mutated KRAS proteins have historically been thwarted by the absence of druggable pockets on the surface of the protein (Cox et al., 2014). In 2013, Shokat and colleagues identified covalent inhibitors of a common (O'Bryan, 2019) oncogenic mutant of KRAS, KRAS G12C, which bound to a previously unrecognized allosteric pocket on GDP-KRAS G12C and prevented its subsequent activation (Ostream et al., 2013). This discovery brought about significant new efforts in the KRAS inhibitor research, which have recently culminated in the entry of KRAS inhibitors in human clinical trials.

While some progress has been made on KRAS G12C inhibitors, there is a continued interest and effort to develop inhibitors of KRAS, particularly inhibitors of other KRAS such as KRAS G12D, G12V, G12A or G12S. Thus, there is a need to develop new inhibitors for KRAS G12D, G12V, G12A, G12S or G12C for the treatment of disorders, such as cancer.

### SUMMARY

In one aspect, the present application is directed to compound of formula (I):

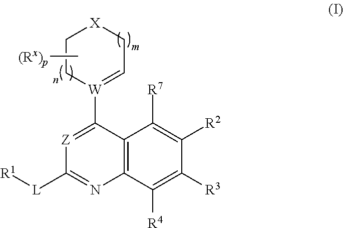

or a pharmaceutically acceptable salt of said compound, wherein;
[symbol] is a single bond or a double bond;
W is C, CH or N;
X is CH₂, O, S, S(O), S(O)(NR^{z}) or S(O)₂;
n is 0, 1, 2, or 3,
m is 0, 1, 2 or 3;
p is 0, 1, 2, 3 or 4;
each R^{x} is hydroxyl, halogen, oxo, cyano, —N(R^{z})₂, C₁₋₄ alkyl, C₁₋₄ alkoxy, C₁₋₄ haloalkyl, C₁₋₄ haloalkoxy, 5-7 membered heteroaryl, -T-R^{y} or two R^{x} taken together with the same carbon or adjacent carbon atoms can form C₃₋₇ cycloalkyl, a 5-7 membered heterocycloalkyl, wherein each C₃₋₇ cycloalkyl or 5-7 membered heterocycloalkyl is further substituted with 0-3 occurrences of R^{y} or two R^{x} taken together can form a bridged ring where the bridge is selected from one of the following: —C₁₋₄ alkylene, —C₁₋₄ alkylene-O—C₁₋₄ alkylene-, —O—, —S— or —C₁₋₄ alkylene-S—C₁₋₄ alkylene- and wherein each C₁₋₄ alkylene is further substituted with 0-2 occurrences of R^{y};
Z is CH, CR′ or N;
R′ is halogen, cyano or C₁₋₄ alkyl;
L is a bond, C₁₋₆ alkylene, C₁₋₆ alkenylene, —O—C₁₋₆ alkylene, —S—C₁₋₆, alkylene, NR^{z}, O or S, wherein each C₁₋₆ alkylene, —O—C₁₋₆ alkylene and —S—C₁₋₆ alkylene chain is substituted with 0-2 occurrences of R²;
R¹ is hydroxyl, aryl, heteroaryl, C₃₋₄ cycloalkyl or heterocycloalkyl optionally substituted with 0-3 occurrences of R⁵;
R² is hydrogen, hydroxyl, halogen, amino, C₁₋₄ alkyl, C₁₋₄ alkoxy, C₁₋₄ haloalkyl, C₂₋₄ alkenyl, C₂₋₄ alkynyl, C₃₋₄ cycloalkyl, cyano, or two R² on the same or adjacent carbon atoms can be taken together to form a C₃₋₇ cycloalkyl;
R³ is aryl or heteroaryl optionally substituted with 0-4 occurrences of R⁶;
R⁴ is hydrogen, hydroxyl, halogen, C₁₋₄ alkyl, C₁₋₄ alkoxy, C₁₋₄ haloalkyl, C₂₋₄ alkenyl, C₂₋₄ alkynyl, C₃₋₈ cycloalkyl or cyano;
each R⁵ is halogen, hydroxyl, oxo, amino, C₁₋₄ alkyl or -T-R^{y};
each R⁶ is halogen, hydroxyl, amino, cyano, C₁₋₄ alkyl, C₂₋₄ alkenyl, C₂₋₄ alknyl, C₁₋₄ haloalkyl, C₃₋₇ cycloalkyl or two R⁶ taken on adjacent carbon atoms can be used to form a C₃₋₇ cycloalkyl, wherein each C₃₋₇ cycloalkyl is further substituted with 0-2 R⁷;
R⁷ is hydrogen, halogen or C₁₋₄ alkyl;
T is C₁₋₄ alkylene, —O—, —S—, —C₁₋₄ alkylene-C(O)— or —NR^{z}—C(O)—;
R^{y} is halogen, hydroxyl, cyano, C₁₋₄ alkoxy or amino; and
R^{z} is hydrogen or C₁₋₄ alkyl.

In a second aspect, provided herein is a pharmaceutical composition comprising a compound of Formula I or a pharmaceutically acceptable salt of said compound and a pharmaceutically acceptable excipient.

In a third aspect, provided herein is a compound of Formula I, or a pharmaceutically acceptable salt of said compound, or the pharmaceutical composition as described herein for use in treating cancer (e.g., NSCLC, colorectal cancer or pancreatic cancer).

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

### DETAILED DESCRIPTION

Provided herein as embodiment 1 is a compound of formula (I):

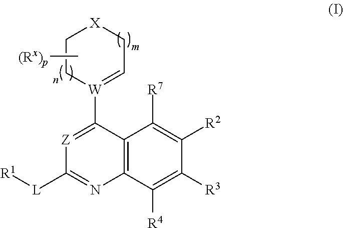

or a pharmaceutically acceptable salt of said compound, wherein;
[symbol] is a single bond or a double bond;
W is C, CH or N;
X is CH₂, O, S, S(O), S(O)(NR^{z}) or S(O)₂;
n is 0, 1, 2, or 3;
m is 0, 1, 2 or 3;
p is 0, 1, 2, 3 or 4;
each R^{x} is hydroxyl, halogen, oxo, cyano, —N(R^{z})₂, C₁₋₄ alkyl, C₁₋₄ alkoxy, C₁₋₄ haloalkyl, C₁₋₄ haloalkoxy, 5-7 membered heteroaryl, -T-R^{y} or two R^{x} taken together with the same carbon or adjacent carbon atoms can form C₃₋₇ cycloalkyl, a 5-7 membered heterocycloalkyl, wherein each C₃₋₇ cycloalkyl or 5-7 membered heterocycloalkyl is further substituted with 0-3 occurrences of R^{y} or two R^{x} taken together can form a bridged ring where the bridge is selected from one of the following: —C₁₋₄ alkylene, —C₁₋₄ alkylene-O—C₁₋₄ alkylene-, —O—, —S— or —C₁₋₄ alkylene-S—C₁₋₄ alkylene- and wherein each C₁₋₄ alkylene is further substituted with 0-2 occurrences of R^{y};
Z is CH, CR′ or N;
R′ is halogen, cyano or C₁₋₄ alkyl;
L is a bond, C₁₋₆ alkylene, C₁₋₆ alkenylene, —O—C₁₋₆ alkylene, —S—C₁₋₆ alkylene, NR^{z}, O or S, wherein each C₁₋₆ alkylene, —O—C₁₋₆ alkylene and —S—C₁₋₆ alkylene chain is substituted with 0-2 occurrences of R²;
R¹ is hydroxyl, aryl, heteroaryl, C₃₋₈ cycloalkyl or heterocycloalkyl optionally substituted with 0-3 occurrences of R⁵;
R² is hydrogen, hydroxyl, halogen, amino, C₁₋₄ alkyl, C₁₋₄ alkoxy, C₁₋₄ haloalkyl, C₂₋₄ alkenyl, C₂₋₄ alkynyl, C₃₋₈ cycloalkyl, cyano, or two R² on the same or adjacent carbon atoms can be taken together to form a C₃₋₇ cycloalkyl;
R³ is aryl or heteroaryl optionally substituted with 0-4 occurrences of R¹;
R⁴ is hydrogen, hydroxyl, halogen, C₁₋₄ alkyl, C₁₋₄ alkoxy, C₁₋₄ haloalkyl, C₂₋₄ alkenyl, C₂₋₄ alkynyl, C₃₋₈ cycloalkyl or cyano;
each R⁵ is halogen, hydroxyl, oxo, amino, C₁₋₄ alkyl or -T-R^{y};
each R⁶ is halogen, hydroxyl, amino, cyano, C₁₋₄ alkyl, C₁₋₄ alkenyl, C₁₋₄ alkynyl, C₁₋₄ haloalkyl, C₃₋₇ cycloalkyl or two R⁶ taken on adjacent carbon atoms can be used to form a C₃₋₇ cycloalkyl, wherein each C₃₋₇ cycloalkyl is further substituted with 0-2 R⁷;
R⁷ is hydrogen, halogen or C₁₋₄ alkyl;
T is C₁₋₄ alkylene, —O—, —S—, —C₁₋₄ alkylene-C(O)— or —NR^{z}—C(O)—;
R^{Y} is halogen, hydroxyl, cyano, C₁₋₄ alkoxy or amino; and
R^{z} is hydrogen or C₁₋₄ alkyl.

Provided herein as embodiment 2 is the compound according to embodiment 1, wherein Z is N.

Provided herein as embodiment 3 is the compound according to any one of embodiments 1-2, wherein L is —O—C₁₋alkylene (e.g., —O-methylene-, —O-ethylene- or —O-n-propylene-) substituted with 0-2 occurrences of R². Provided herein as embodiment 4 is the compound according to embodiment 3, wherein L is —O-methylene substituted with 0 occurrences of R².

Provided herein as embodiment 5 is the compound according to any one of embodiments 1-4, wherein R¹ is heterocycloalkyl optionally substituted with 0-3 occurrences of R⁵. Provided herein as embodiment 6 is the compound according to embodiment 5, wherein R¹ is 7-(hexahydro-1H-pyrrolizine) substituted with 0-3 occurrences of R⁵. Provided herein as embodiment 7 is the compound according to embodiment 6, wherein R¹ is 7-(hexahydro-1H-pyrrolizine) substituted with 0 occurrences of R⁵. Provided herein as embodiment 8 is the compound according to embodiment 6, wherein R¹ is 7-(hexahydro-1H-pyrrolizine) substituted with 1 occurrence of R⁵. Provided herein as embodiment 9 is the compound according to embodiment 8, wherein R⁵ is halogen (e.g., fluorine).

Provided herein as embodiment 10 is the compound according to any one of embodiments 1-4, wherein R¹ is 2-pyrrolidine substituted with 0-3 occurrences of R⁵. Provided herein as embodiment 11 is the compound according to embodiment 10, wherein R¹ is 2-pyrrolidine substituted with 2 occurrences of R⁵. Provided herein as embodiment 12 is the compound according to embodiment 11, wherein one R⁵ is C₁₋₄ alkyl (e.g., methyl) and the other R⁵ is halogen (e.g., fluorine).

Provided herein as embodiment 13 is the compound according to embodiment 3, wherein L is —O-n-propylene substituted with 2 occurrences of R². Provided herein as embodiment 14 is the compound according to embodiment 13, wherein both R² are taken together on the same carbon atom to form a cyclopropyl. Provided herein as embodiment 15 is the compound according to embodiment 14, wherein R¹ is hydroxyl.

Provided herein as embodiment 16 is the compound according to any one of embodiments 1-15, wherein -L-R¹ is

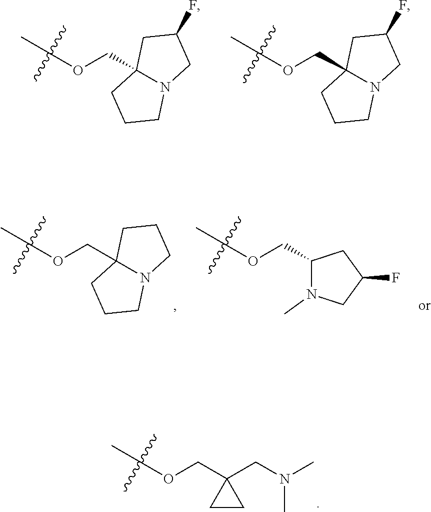

Provided herein as embodiment 17 is the compound according to embodiment 16, wherein -L-R¹ is

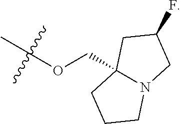 [SMILES: *OC[C@@]12CCCN1C[C@H](F)C2]

Provided herein as embodiment 18 is the compound according to embodiment 16, wherein -L-R¹ is

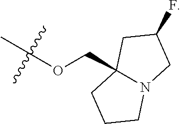 [SMILES: *OC[C@]12CCCN1C[C@H](F)C2]

Provided herein as embodiment 19 is the compound according to embodiment 16, wherein -L-R¹ is

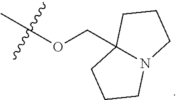 [SMILES: *OCC12CCCN1CCC2]

Provided herein as embodiment 20 is the compound according to embodiment 16, wherein -L-R¹ is

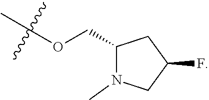 [SMILES: *OC[C@@H]1C[C@@H](F)CN1C]

Provided herein as embodiment 21 is the compound according to embodiment 16, wherein -L-R¹ is

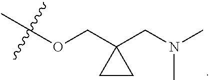 [SMILES: *OCC1(CN(C)C)CC1]

Provided herein as embodiment 22 is the compound according to any one of embodiments 1-21, wherein R³ is aryl (e.g., phenyl or naphthyl) substituted with 0-3 occurrences of R⁶.

Provided herein as embodiment 23 is the compound according to embodiment 22, wherein R³ is naphthyl substituted with 1 occurrence of R⁶. Provided herein as embodiment 24 is the compound according to embodiment 23, wherein R⁶ is hydroxyl.

Provided herein as embodiment 25 is the compound according to embodiment 22, wherein R³ is naphthyl substituted with 2 occurrences of R⁶. Provided herein as embodiment 26 is the compound according to embodiment 25, wherein each occurrence of R⁶ is halogen, hydroxyl, C₂₋₄ alkynyl or C₁₋₄ alkyl. Provided herein as embodiment 27 is the compound according to embodiment 26, wherein each occurrence of R⁶ is fluorine, hydroxyl, 2-ethynyl or ethyl.

Provided herein as embodiment 28 is the compound according to embodiment 22, wherein R³ is naphthyl substituted with 3 occurrences of R⁶. Provided herein as embodiment 29 is the compound according to embodiment 28, wherein each occurrence of R⁶ is hydroxyl, C₂₋₄ alkynyl, C₁₋₄ alkyl, halogen or amino. Provided herein as embodiment 30 is the compound according to embodiment 29, wherein each occurrence of R⁶ is hydroxyl, 2-ethynyl, ethyl, fluorine, chlorine or amino.

Provided herein as embodiment 31 is the compound according to embodiment 22, wherein R³ is phenyl substituted with 3 occurrences of R⁶. Provided herein as embodiment 32 is the compound according to embodiment 31, wherein each occurrence of R⁶ is halogen, hydroxyl, C₁₋₄ haloalkyl, C₃₋₇ cycloalkyl or amino wherein each C₃₋₇ cycloalkyl is further substituted with 0-3 occurrences of R⁷. Provided herein as embodiment 33 is the compound according to embodiment 32, wherein each occurrence of R⁶ is hydroxyl, chloro, cyclopropyl, trifluoromethyl or amino, wherein each cyclopropyl is further substituted with 0 occurrences of R⁷.

Provided herein as embodiment 34 is the compound according to embodiment 31, wherein each occurrence of R⁶ is hydroxyl and the other two R⁶ are on adjacent carbon atoms form a C₃₋₇ cycloalkyl, wherein C₃₋₇ cycloalkyl is further substituted with 0-2 R⁷. Provided herein as embodiment 35 is the compound according to embodiment 34, wherein each occurrence of R⁶ is hydroxyl and the other two R⁶ are on adjacent carbon atoms form a cyclohexyl further substituted with one R⁷. Provided herein as embodiment 36 is the compound according to embodiment 35, wherein R⁷ is C₁₋₄ alkyl (e.g., methyl or ethyl).

Provided herein as embodiment 37 is the compound according to any one of embodiments 1-21, wherein R³ is heteroaryl substituted with 0-3 occurrences of R⁶. Provided herein as embodiment 38 is the compound according to embodiment 37, wherein R³ is 4-(1H-indazolyl) or 7-(1H-indazolyl) substituted with 0-3 occurrences of R⁶.

Provided herein as embodiment 39 is the compound according to embodiment 38, wherein R³ is 4-(1H-indazolyl) substituted with 0-3 occurrences of R⁶. Provided herein as embodiment 40 is the compound according to embodiment 39, wherein R³ is 4-(1H-indazolyl) substituted with 2 occurrences of R⁶. Provided herein as embodiment 41 is the compound according to embodiment 40, wherein each R⁶ is C₁₋₄ alkyl, C₂₋₄ alkenyl or halogen. Provided herein as embodiment 42 is the compound according to embodiment 41, wherein each R⁶ is cyclopropyl, methylcyclopropyl, cis-prop-1-enyl, methyl or chloro, wherein cyclopropyl is further substituted with one occurrence of R⁷. Provided herein as embodiment 43 is the compound according to embodiment 42, wherein R⁷ is C₁₋₄ alkyl (e.g., methyl).

Provided herein as embodiment 44 is the compound according to embodiment 38, wherein R³ is 7-(1H-indazolyl) substituted with 0-3 occurrences of R⁶. Provided herein as embodiment 45 is the compound according to embodiment 44, wherein R³ is 7-(1H-indazolyl) substituted with 2 occurrences of R. Provided herein as embodiment 46 is the compound according to embodiment 45, wherein each R⁶ is C₁₋₄ alkyl or halogen. Provided herein as embodiment 47 is the compound according to embodiment 46, wherein each R⁶ is methyl or chloro.

Provided herein as embodiment 48 is the compound according to any one of embodiments 1-47, wherein R³ is

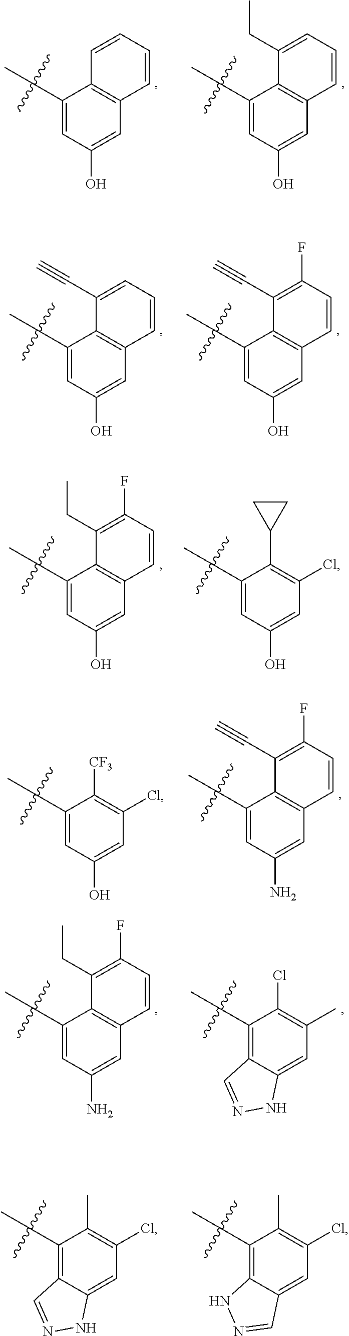

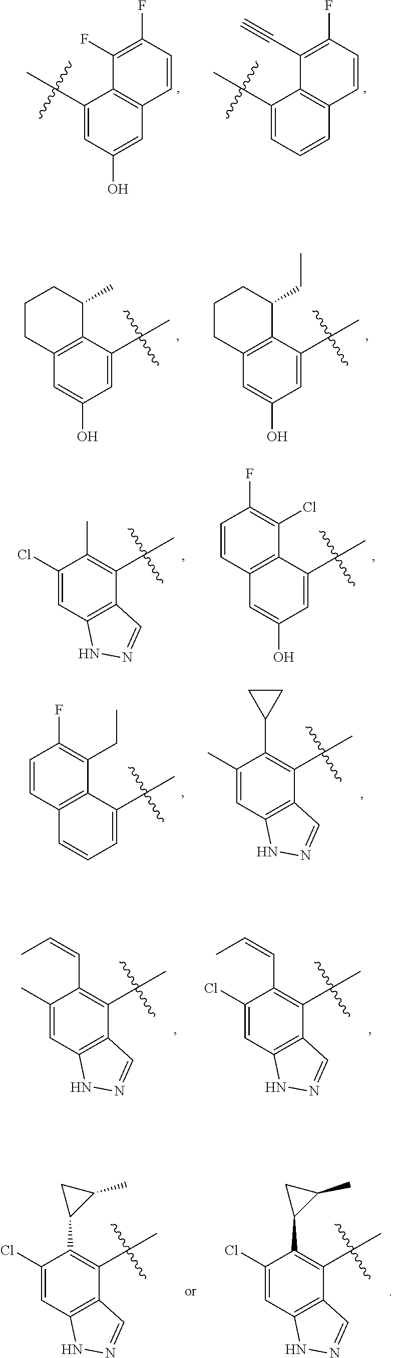

Provided herein as embodiment 49 is the compound according to embodiment 48, wherein R³ is

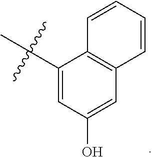 [SMILES: *c1cc(O)cc2ccccc12]

Provided herein as embodiment 50 is the compound according to embodiment 48, wherein R³ is

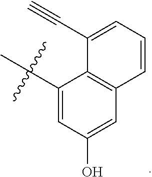 [SMILES: *c1cc(O)cc2cccc(C#C)c12]

Provided herein as embodiment 51 is the compound according to embodiment 48, wherein R³ is

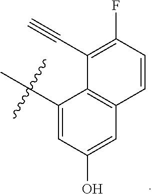 [SMILES: *c1cc(O)cc2ccc(F)c(C#C)c12]

Provided herein as embodiment 52 is the compound according to embodiment 48, wherein R³ is

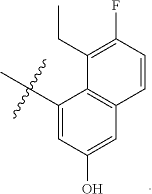 [SMILES: *c1cc(O)cc2ccc(F)c(CC)c12]

Provided herein as embodiment 53 is the compound according to embodiment 48, wherein R³ is

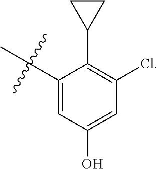 [SMILES: *c1cc(O)cc(Cl)c1C1CC1]

Provided herein as embodiment 54 is the compound according to embodiment 48, wherein R³ is

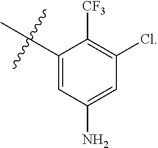 [SMILES: *c1cc(N)cc(Cl)c1C(F)(F)F]

Provided herein as embodiment 55 is the compound according to embodiment 48, wherein R³ is

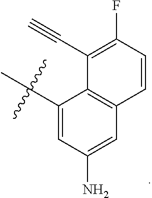 [SMILES: *c1cc(N)cc2ccc(F)c(C#C)c12]

Provided herein as embodiment 56 is the compound according to embodiment 48, wherein R³ is

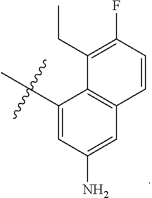 [SMILES: *c1cc(N)cc2ccc(F)c(CC)c12]

Provided herein as embodiment 57 is the compound according to embodiment 48, wherein R³ is

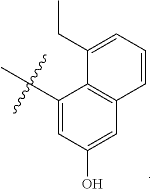 [SMILES: *c1cc(O)cc2cccc(CC)c12]

Provided herein as embodiment 58 is the compound according to embodiment 48, wherein R³ is

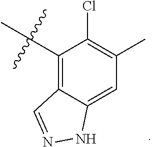 [SMILES: *c1c(Cl)c(C)cc2[nH]ncc12]

Provided herein as embodiment 59 is the compound according to embodiment 48, wherein R³ is

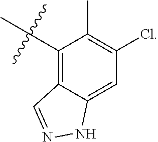 [SMILES: *c1c(C)c(Cl)cc2[nH]ncc12]

Provided herein as embodiment 60 is the compound according to embodiment 48, wherein R³ is

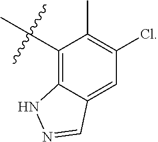 [SMILES: *c1c(C)c(Cl)cc2cn[nH]c12]

Provided herein as embodiment 61 is the compound according to embodiment 48, wherein R³ is

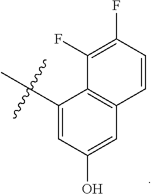 [SMILES: *c1cc(O)cc2ccc(F)c(F)c12]

Provided herein as embodiment 62 is the compound according to embodiment 48, wherein R³ is

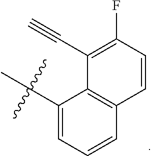 [SMILES: *c1cccc2ccc(F)c(C#C)c12]

Provided herein as embodiment 63 is the compound according to embodiment 48, wherein R³ is

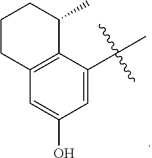 [SMILES: *c1cc(O)cc2c1[C@@H](C)CCC2]

Provided herein as embodiment 64 is the compound according to embodiment 48, wherein R³ is

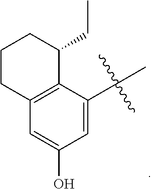 [SMILES: *c1cc(O)cc2c1[C@@H](CC)CCC2]

Provided herein as embodiment 65 is the compound according to embodiment 48, wherein R³ is

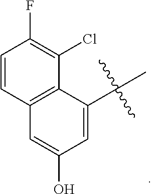 [SMILES: *c1cc(O)cc2ccc(F)c(Cl)c12]

Provided herein as embodiment 66 is the compound according to embodiment 48, wherein R³ is

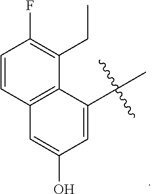 [SMILES: *c1cc(O)cc2ccc(F)c(CC)c12]

Provided herein as embodiment 67 is the compound according to embodiment 48, wherein R³ is

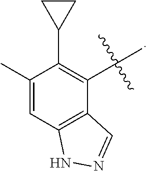

Provided herein as embodiment 68 is the compound according to embodiment 48, wherein R³ is

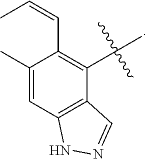

Provided herein as embodiment 69 is the compound according to embodiment 48, wherein R³ is

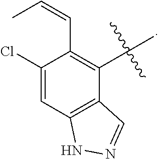

Provided herein as embodiment 70 is the compound according to embodiment 48, wherein R³ is

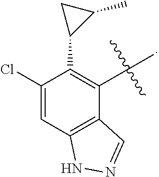

Provided herein as embodiment 71 is the compound according to embodiment 48, wherein R³ is

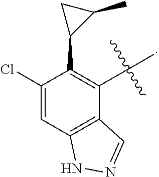

Provided herein as embodiment 72 is the compound according to any one of embodiments 1-71, wherein W is N.

Provided herein as embodiment 73 is the compound according to embodiment 72, wherein X is CH₂.

Provided herein as embodiment 74 is the compound according to embodiment 73, wherein n is 1 and m is 0 or m is 1 and n is 0. Provided herein as embodiment 75 is the compound according to embodiment 74, wherein p is 1.

Provided herein as embodiment 76 is the compound according to embodiment 75, wherein R^{x} is hydroxy or -T-R^{y}. Provided herein as embodiment 77 is the compound according to embodiment 76, wherein R^{x} is hydroxy or —C(O)NH₂.

Provided herein as embodiment 78 is the compound according to embodiment 73, wherein n is 1 and m is 1. Provided herein as embodiment 79 is the compound according to embodiment 78, wherein p is 2.

Provided herein as embodiment 80 is the compound according to embodiment 79, wherein each R is independently hydroxy, C₁₋₄ alkyl, C₁₋₄ haloalkyl or two R^{x} taken together with the same carbon or adjacent carbon atoms can form a 5-7 membered heterocycloalkyl further substituted with 0-3 occurrences of R^{y}. Provided herein as embodiment 81 is the compound according to embodiment 80, wherein each R^{x} is independently hydroxy, methyl, fluoromethyl or difluoromethyl. Provided herein as embodiment 82 is the compound according to embodiment 80, wherein two R^{x} are taken together with the same carbon to form a 1-oxetanyl, 1-azetidinyl, 2-azetidinyl, 2-pyrrolidinyl, cyclobutyl, 1,3-dioxolanyl, 3-tetrahydrothiophenyl, 2-tetrahydrofuranyl or 5-oxazolidinyl further substituted with 0-3 occurrences of R^{y}. Provided herein as embodiment 83 is the compound according to embodiment 82, wherein two R^{x} are taken together with the same carbon to form a 1-oxetanyl further substituted with 0 occurrences of R^{y}. Provided herein as embodiment 84 is the compound according to embodiment 82, wherein two R^{x} are taken together with the same carbon to form a 1-azetidinyl, 2-azetidinyl, 2-pyrrolidinyl, 1,3-dioxolanyl, 2-tetrahydrofuranyl or 5-oxazolidinyl further substituted with 1 occurrence of R^{y}. Provided herein as embodiment 85 is the compound according to embodiment 84, wherein R^{y} is oxo. Provided herein as embodiment 86 is the compound according to embodiment 82, wherein two R^{x} are taken together with the same carbon to form a cyclobutyl further substituted with 1 occurrence of R^{y}. Provided herein as embodiment 87 is the compound according to embodiment 86, wherein R^{y} is hydroxyl. Provided herein as embodiment 88 is the compound according to embodiment 82, wherein two R^{x} are taken together with the same carbon to form a 3-tetrahydrothiophenyl further substituted with 2 occurrences of R^{y}. Provided herein as embodiment 89 is the compound according to embodiment 88, wherein both R^{y} are oxo.

Provided herein as embodiment 90 is the compound according to embodiment 78, wherein p is 3. Provided herein as embodiment 91 is the compound according to embodiment 90, wherein each R^{x} is independently hydroxy, halogen, —N(R)₂, -T-R^{y} or two R^{x} taken together form a bridged ring where the bridge is C₁₋₄ alkylene is further substituted with 0-2 occurrences of R^{y}. Provided herein as embodiment 92 is the compound according to embodiment 91, wherein two R are fluorine and the other R^{x} is —NH₂. Provided herein as embodiment 93 is the compound according to embodiment 91, wherein two R^{x} are fluorine and the other R^{x} is -T-R^{y}. Provided herein as embodiment 94 is the compound according to embodiment 93, wherein two R^{x} are fluorine and the other R^{x} is —NH—C(O)—OMe. Provided herein as embodiment 95 is the compound according to embodiment 91, wherein one R^{x} is hydroxy and two R^{x} taken together form a bridged ring where the bridge is ethylene is further substituted with 0 occurrences of R^{y}.

Provided herein as embodiment 96 is the compound according to embodiment 73, wherein n is 1 and m is 2 or n is 2 and m is 1. Provided herein as embodiment 97 is the compound according to embodiment 96, wherein p is 3.

Provided herein as embodiment 98 is the compound according to embodiment 97, wherein one R^{x} is hydroxyl and the other two R^{x} taken together form a bridged ring where the bridge is —C₁₋₄ alkylene further substituted with 0-2 occurrences of R^{y}. Provided herein as embodiment 99 is the compound according to embodiment 98, wherein one R^{x} is hydroxyl and the other two R^{x} taken together form a bridged ring where the bridge is methylene further substituted with 0 occurrences of R^{y}.

Provided herein as embodiment 100 is the compound according to embodiment 73, wherein

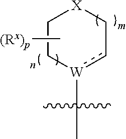

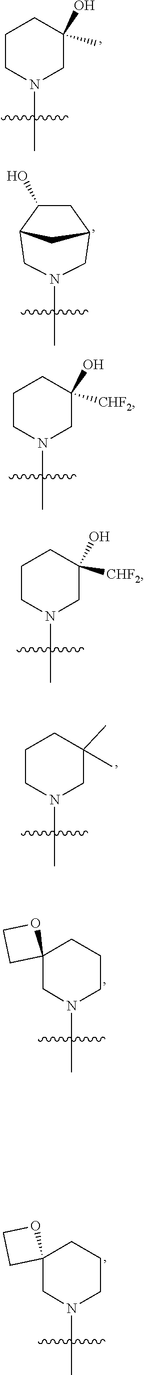

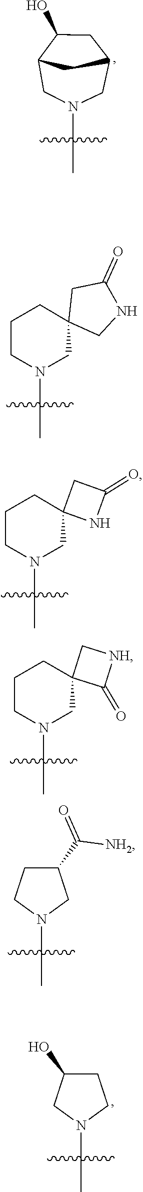

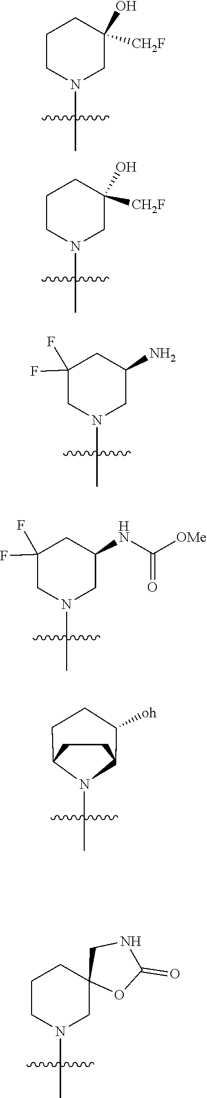

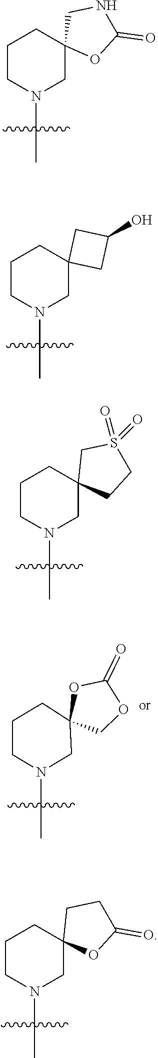

is

Provided herein as embodiment 101 is the compound according to embodiment 100, wherein

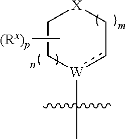

is

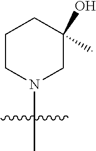

Provided herein as embodiment 102 is the compound according to embodiment 100, wherein

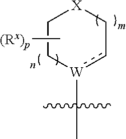

is

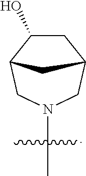

Provided herein as embodiment 103 is the compound according to embodiment 100, wherein

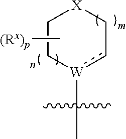

is

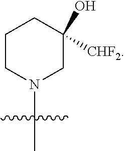 [SMILES: *N1CCC[C@](O)(C(F)F)C1]

Provided herein as embodiment 104 is the compound according to embodiment 100, wherein

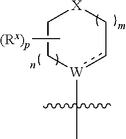

is

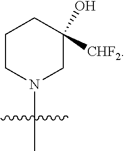 [SMILES: *N1CCC[C@@](O)(C(F)F)C1]

Provided herein as embodiment 105 is the compound according to embodiment 100, wherein

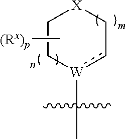

is

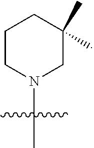

Provided herein as embodiment 106 is the compound according to embodiment 100, wherein

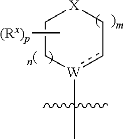

is

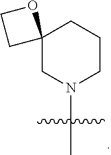 [SMILES: *N1CCC[C@@]2(CCO2)C1]

Provided herein as embodiment 107 is the compound according to embodiment 100, wherein

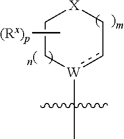

is

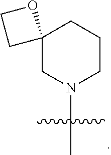 [SMILES: *N1CCC[C@]2(CCO2)C1]

Provided herein as embodiment 108 is the compound according to embodiment 100, wherein

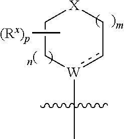

is

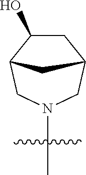 [SMILES: *N1C[C@@H]2C[C@H](C1)[C@@H](O)C2]

Provided herein as embodiment 109 is the compound according to embodiment 100, wherein

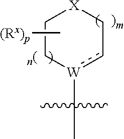

is

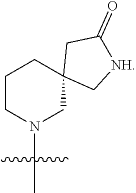 [SMILES: *N1CCC[C@@]2(CNC(=O)C2)C1]

Provided herein as embodiment 110 is the compound according to embodiment 100, wherein

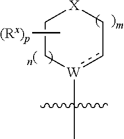

is

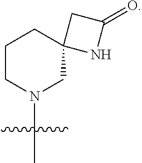 [SMILES: *N1CCC[C@]2(CC(=O)N2)C1]

Provided herein as embodiment 111 is the compound according to embodiment 100, wherein

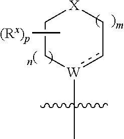

is

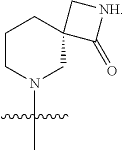 [SMILES: *N1CCC[C@]2(CNC2=O)C1]

Provided herein as embodiment 112 is the compound according to embodiment 100, wherein

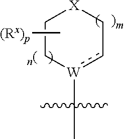

is,

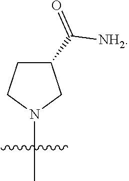 [SMILES: *N1CC[C@H](C(N)=O)C1]

Provided herein as embodiment 113 is the compound according to embodiment 100, wherein

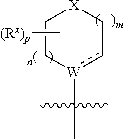

is

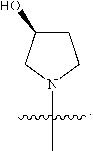 [SMILES: *N1CC[C@H](O)C1]

Provided herein as embodiment 114 is the compound according to embodiment 100, wherein

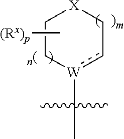

is

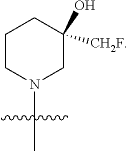

Provided herein as embodiment 115 is the compound according to embodiment 100, wherein

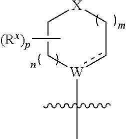

is

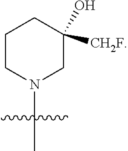

Provided herein as embodiment 116 is the compound according to embodiment 100, wherein

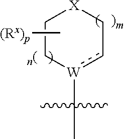

is

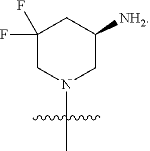 [SMILES: *N1C[C@H](N)CC(F)(F)C1]

Provided herein as embodiment 117 is the compound according to embodiment 100, wherein

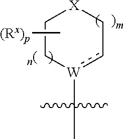

is

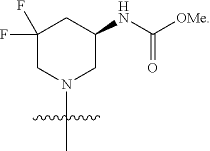 [SMILES: *N1C[C@H](NC(=O)OC)CC(F)(F)C1]

Provided herein as embodiment 118 is the compound according to embodiment 100, wherein

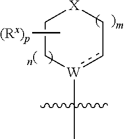

is

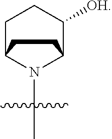

Provided herein as embodiment 119 is the compound according to embodiment 100, wherein

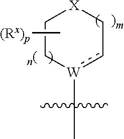

is

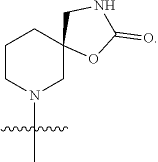 [SMILES: *N1CCC[C@@]2(CNC(=O)O2)C1]

Provided herein as embodiment 120 is the compound according to embodiment 100, wherein

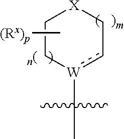

is

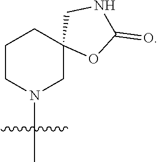 [SMILES: *N1CCC[C@]2(CNC(=O)O2)C1]

Provided herein as embodiment 121 is the compound according to embodiment 100, wherein

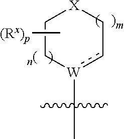

is

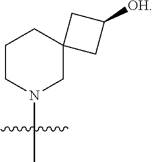

Provided herein as embodiment 122 is the compound according to embodiment 100, wherein

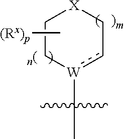

is

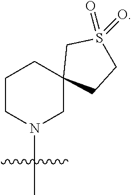 [SMILES: *N1CCC[C@@]2(CCS(=O)(=O)C2)C1]

Provided herein as embodiment 123 is the compound according to embodiment 100, wherein

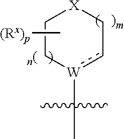

is

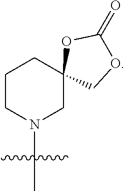 [SMILES: *N1CCC[C@]2(COC(=O)O2)C1]

Provided herein as embodiment 124 is the compound according to embodiment 100, wherein

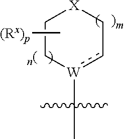

is

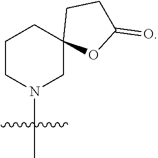 [SMILES: *N1CCC[C@]2(CCC(=O)O2)C1]

Provided herein as embodiment 125 is the compound according to embodiment 72, wherein X is O.

Provided herein as embodiment 126 is the compound according to embodiment 125, wherein n is 1 and m is 1. Provided herein as embodiment 127 is the compound according to embodiment 126, wherein p is 2.

Provided herein as embodiment 128 is the compound according to embodiment 127, wherein two R^{x} are taken together to form a bridged ring where the bridge is —C₁₋₄ alkylene further substituted with 0-2 occurrences of R^{y}. Provided herein as embodiment 129 is the compound according to embodiment 128, wherein two R^{x} are taken together to form a bridged ring where the bridge is ethylene further substituted with 0 occurrences of R^{y}.

Provided herein as embodiment 130 is the compound according to embodiment 125, wherein n is 1 and m is 2 or m is 1 and n is 2. Provided herein as embodiment 131 is the compound according to embodiment 130, wherein p is 0. Provided herein as embodiment 132 is the compound according to embodiment 130, wherein p is 2.

Provided herein as embodiment 133 is the compound according to embodiment 132, wherein each R^{x} is halogen, hydroxyl, C₁₋₄ haloalkyl or C₁₋₄ alkyl or two R^{x} are taken together to form a bridged ring where the bridge is —C₁₋₄ alkylene further substituted with 0-2 occurrences of R^{y}. Provided herein as embodiment 134 is the compound according to embodiment 133, wherein one R^{x} is hydroxyl and the other R^{x} is methyl. Provided herein as embodiment 135 is the compound according to embodiment 134, wherein one R^{x} is hydroxyl and the other R^{x} is fluoromethyl. Provided herein as embodiment 136 is the compound according to embodiment 133, wherein both R^{x} are fluorine. Provided herein as embodiment 137 is the compound according to embodiment 133, wherein two R^{x} are taken together to form a bridged ring where the bridge is methylene further substituted with 0 occurrences of R^{y}.

Provided herein as embodiment 138 is the compound according to embodiment 125, wherein

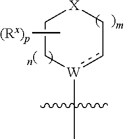

is

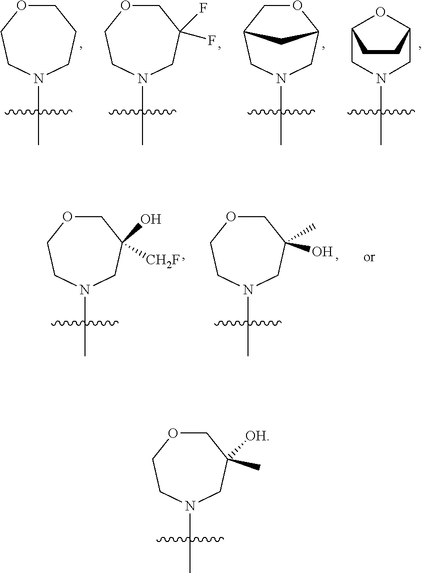

Provided herein as embodiment 139 is the compound according to embodiment 138, wherein

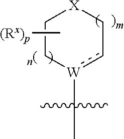

is

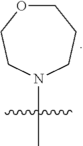 [SMILES: *N1CCCOCC1]

Provided herein as embodiment 140 is the compound according to embodiment 138, wherein

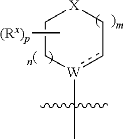

is,

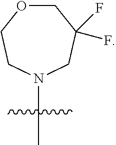 [SMILES: *N1CCOCC(F)(F)C1]

Provided herein as embodiment 141 is the compound according to embodiment 138, wherein

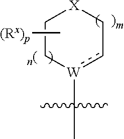

is

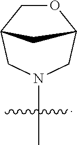

Provided herein as embodiment 142 is the compound according to embodiment 138, wherein

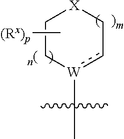

is

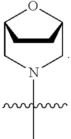

Provided herein as embodiment 143 is the compound according to embodiment 138, wherein

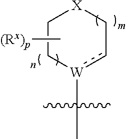

is

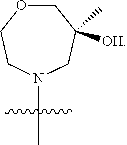 [SMILES: *N1CCOC[C@@](C)(O)C1]

Provided herein as embodiment 144 is the compound according to embodiment 138, wherein

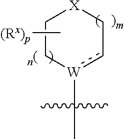

is

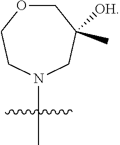 [SMILES: *N1CCOC[C@](C)(O)C1]

Provided herein as embodiment 145 is the compound according to any one of embodiments 1-144, wherein R² is hydrogen. Provided herein as embodiment 146 is the compound according to any one of embodiments 1-144, wherein R² is halogen (e.g., fluorine). Provided herein as embodiment 147 is the compound according to any one of embodiments 1-144, wherein R² is cyano. Provided herein as embodiment 148 is the compound according to ay one of embodiments 1-144, wherein R² is hydroxyl. Provided herein as embodiment 149 is the compound according to any one of embodiments 1-144, wherein R² is amino.

Provided herein as embodiment 150 is the compound according to any one of embodiments 1-149, wherein R⁴ is halogen (e.g., fluorine). Provided herein as embodiment 151 is the compound according to any one of embodiments 1-149, wherein R² is C₁₋₄ alkyl (e.g., methyl).

Provided herein as embodiment 152 is the compound according to any one of embodiments 1-151, wherein R⁷ is hydrogen.

Provided herein as embodiment 153 is the compound according to embodiment 1, wherein is the compound is a compound of formula (II):

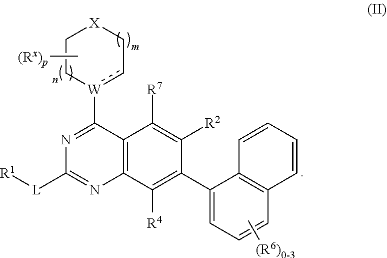

Provided herein as embodiment 154 is the compound according to embodiment 1, wherein is the compound is a compound of formula (III):

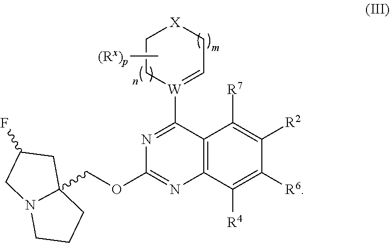

Provided herein as embodiment 155 is the compound according to embodiment 1, wherein is the compound is a compound of formula (IV):

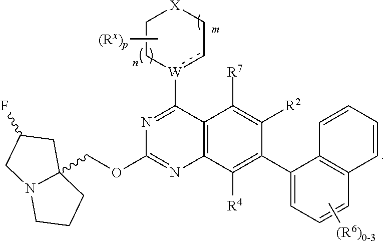

Provided herein as embodiment 156 is the compound according to embodiment 1, wherein the compound is selected from one of the following compounds:
(6S)-4-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol:
(3R)-1-(7-(8-ethynyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol;
(5S)-7-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
(6S)-4-(7-(8-ethyl-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol;
(3R)-1-(7-(8-ethynyl-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol;
(3R)-1-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol;
4-(6,8-difluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)-5-ethyl-6-fluoronaphthalen-2-ol;
(S)-4-(7-(8-ethyl-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol;
(S)-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol;
7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-6-ol;
(R)-1-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol;
(R)-1-(7-(8-ethynyl-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol;
(R)-7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
(6S)-4-(7-(7,8-difluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol;
(S)-4-(7-(7,8-difluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol;
(5S)-7-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
(S)-6-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-1,6-diazaspiro[3.5]nonan-2-one;
(R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol;
5-ethyl-6-fluoro-4-(8-fluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)naphthalen-2-ol;
(3R)-3-(difluoromethyl)-1-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)piperidin-3-ol;
(3R)-1-(7-(8-ethynyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol;
(5R)-7-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
(3R)-1-(7-(3-chloro-2-cyclopropyl-5-hydroxyphenyl)-6,8-difluoro-2-(((2R,7aS)-2-fluorotetrahydro-1H-pyrrolizin-7a(5H)-yl)methoxy)quinazolin-4-yl)-3-methylpiperidin-3-ol;
(1R,5R,6R)-3-(7-(8-ethynyl-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-azabicyclo[3.2.1]octan-6-ol; or
(3R)-1-(6-chloro-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol.
Provided herein as embodiment 157 is the compound according to embodiment 1, wherein the compound is selected from one of the following compounds:
(6S)-4-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol;
(3R)-1-(7-(8-ethynyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol;
(5S)-7-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
(6S)-4-(7-(8-ethyl-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol;
(3R)-1-(7-(8-ethynyl-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol:
(3R)-1-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol;
4-(6,8-difluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)-5-ethyl-6-fluoronaphthalen-2-ol;
(S)-4-(7-(8-ethyl-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol;
(S)-4-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol; or
7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-6-ol.

Provided herein as embodiment 158 is the compound according to embodiment 1, wherein the compound is selected from one of the following compounds:
4-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol (Isomer 1);
(3R)-1-(7-(8-Ethynyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Isomer 2):
7-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
4-(7-(8-Ethyl-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol (Isomer 1).
(3R)-1-(7-(8-Ethynyl-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Isomer 2);
(5S)-7-(7-(8-Ethyl-7-fluoro-3-hydroxynaphthalen-1-yl)-6,8-difluoro-2-(((2S,4R)-4-fluoro-1-methylpyrrolidin-2-yl)methoxy)quinazolin-4-yl)-1-oxa-3,7-diazaspiro[4.5]decan-2-one;
4-(6,8-Difluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)-5-ethyl-6-fluoronaphthalen-2-ol;
4-(7-(8-Chloro-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol (Isomer 1);
(3R)-1-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Isomer 1);
5-Ethyl-6-fluoro-4-(8-fluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)naphthalen-2-ol;
4-(6,8-Difluoro-2-(((2R,7aS)-2-fluorotetrahydro-1H-pyrrolizin-7a(5H)-yl)methoxy)-4-(1-oxa-6-azaspiro[3.5]nonan-6-yl)quinazolin-7-yl)-5-ethyl-6-fluoronaphthalen-2-ol (Isomer 2);
4-(7-(8-Ethyl-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol (Isomer 1);
(S)-7-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
(R)-7-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol (Isomer 1);
5-Chloro-4-(6,8-difluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)-6-fluoronaphthalen-2-ol;
(2S,4s)-6-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-azaspiro[3.5]nonan-2-ol;
7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-6-ol (Isomer 1);
(R)-1-(7-(8-Ethynyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol;
(R)-1-(7-(8-Ethynyl-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol;
7-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
4-(7-(8-Chloro-7-fluoro-3-hydroxynaphthalen-1-yl)-8-fluoro-2-(((2R,7aS)-2-fluorotetrahydro-H-pyrrolizin-7a(5H)-yl)methoxy)quinazolin-4-yl)-6-methyl-1,4-oxazepan-6-ol (Isomer 1);
4-(7-(7,8-Difluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol (Isomer 1);
(5S)-7-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-2-thia-7-azaspiro[4.5]decane 2,2-dioxide; or
4-(7-(7,8-Difluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol (Isomer 1).

Provided herein as embodiment 159 is the compound according to embodiment 1, wherein the compound is selected from one of the following compounds:
4-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol (Isomer 1);
(3R)-1-(7-(8-Ethynyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Isomer 2);
7-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
4-(7-(8-Ethyl-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol (Isomer 1);
(3R)-1-(7-(8-Ethynyl-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Isomer 2);
(5S)-7-(7-(8-Ethyl-7-fluoro-3-hydroxynaphthalen-1-yl)-6,8-difluoro-2-(((2S,4R)-4-fluoro-1-methylpyrrolidin-2-yl)methoxy)quinazolin-4-yl)-1-oxa-3,7-diazaspiro[4.5]decan-2-one;
4-(6,8-Difluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)-5-ethyl-6-fluoronaphthalen-2-ol;
4-(7-(8-Chloro-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol (Isomer 1);
(3R)-1-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Isomer 1); or
5-Ethyl-6-fluoro-4-(8-fluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)naphthalen-2-ol.

The foregoing merely summarizes certain aspects of this disclosure and is not intended, nor should it be construed, as limiting the disclosure in any way.

### Formulation, and Route of Administration

While it may be possible to administer a compound disclosed herein alone in the uses described, the compound administered normally will be present as an active ingredient in a pharmaceutical composition. Thus, in one embodiment, provided herein is a pharmaceutical composition comprising a compound disclosed herein in combination with one or more pharmaceutically acceptable excipients, such as diluents, carriers, adjuvants and the like, and, if desired, other active ingredients. See, e.g., Remington: The Science and Practice of Pharmacy, Volume I and Volume II, twenty-second edition, edited by Loyd V. Allen Jr., Philadelphia, PA, Pharmaceutical Press, 2012; Pharmaceutical Dosage Forms (Vol. 1-3), Liberman et al., Eds., Marcel Dekker. New York, NY, 1992; Handbook of Pharmaceutical Excipients (3rd Ed.), edited by Arthur H. Kibbe, American Pharmaceutical Association, Washington, 2000; Pharmaceutical Formulation: The Science and Technology of Dosage Forms (Drug Discovery), first edition, edited by GD Tovey, Royal Society of Chemistry, 2018. In one embodiment, a pharmaceutical composition comprises a therapeutically effective amount of a compound disclosed herein.

The compound(s) disclosed herein may be administered by any suitable route in the form of a pharmaceutical composition adapted to such a route and in a dose effective for the treatment intended. The compounds and compositions presented herein may, for example, be administered orally, mucosally, topically, transdermally, rectally, pulmonarily, parentally, intranasally, intravascularly, intravenously, intraarterial, intraperitoneally, intrathecally, subcutaneously, sublingually, intramuscularly, intrasternally, vaginally or by infusion techniques, in dosage unit formulations containing conventional pharmaceutically acceptable excipients.

The pharmaceutical composition may be in the form of, for example, a tablet, chewable tablet, minitablet, caplet, pill, bead, hard capsule, soft capsule, gelatin capsule, granule, powder, lozenge, patch, cream, gel, sachet, microneedle array, syrup, flavored syrup, juice, drop, injectable solution, emulsion, microemulsion, ointment, aerosol, aqueous suspension, or oily suspension. The pharmaceutical composition is typically made in the form of a dosage unit containing a particular amount of the active ingredient.

Provided herein as embodiment 160 is a pharmaceutical composition comprising the compound according to any one of embodiments 1-159, or a tautomer thereof, or a pharmaceutically acceptable salt of said compound or said tautomer, and a pharmaceutically acceptable excipient.

Provided herein as embodiment 161 is a compound according to any one of Embodiments 1-159, or a tautomer thereof, or a pharmaceutically acceptable salt of said compound or said tautomer, or the pharmaceutical composition according to embodiment 160 for use as a medicament.

### Methods of Use

As discussed herein (see, section entitled “Definitions”), the compounds described herein are to be understood to include all stereoisomers, tautomers, or pharmaceutically acceptable salts of any of the foregoing or solvates of any of the foregoing. Accordingly, the scope of the methods and uses provided in the instant disclosure is to be understood to encompass also methods and uses employing all such forms.

Besides being useful for human treatment, the compounds provided herein may be useful for veterinary treatment of companion animals, exotic animals and farm animals, including mammals, rodents, and the like. For example, animals including horses, dogs, and cats may be treated with compounds provided herein.

In one embodiment, the disclosure provides methods of using the compounds or pharmaceutical compositions of the present disclosure to treat disease conditions, including but not limited to conditions implicated by KRAS G12D, G12V, G12A, G12S or G12C mutation (e.g., cancer). The cancer types are non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

KRAS G12D mutations occur with the alteration frequencies shown in the table below (TCGA data sets;¹⁻³ For example, the table shows that 32.4% of subjects with pancreatic cancer have a cancer wherein one or more cells express KRAS G12D mutant protein. Accordingly, the compounds provided herein, which bind to KRAS^{G12D} (see Section entitled “Biological Evaluation” below) are useful for treatment of subjects having a cancer, including, but not limited to the cancers listed in the table below.

| | Alteration |
|---|---|
| Cancer Type | Frequency |
| Pancreatic Adenocarcinoma (PAAD) | 32.4 |
| Colon Adenocarcinoma (COAD) | 12.25 |
| Rectal adenocarcinoma (READ) | 8.03 |
| Uterine corpus endometrial carcinoma | 6.04 |
| (UCEC) | |
| Lung Adenocarcinoma (LUAD) | 3.53 |
| Plasma Cell Tumors | 2.92 |
| Stomach Adenocarcinoma (STAD) | 2.27 |
| Bladder urothelial carcinoma (BLCA) | 1.46 |
| Cervical Squamous carcinoma (CESC) | 1.38 |
| Kidney Adenocarcinoma | 1.07 |
| Thymic Cancer | 0.81 |
| Myeloid Leukemia (LAML) | 0.69 |
| Liver Hepatocellular Carcinoma (LIHC) | 0.55 |
| Glioblastoma multiforme (GBM) | 0.51 |
| Skin Cutaneous Melanoma (SKCM) | 0.43 |
| Bladder Cancer | 0.4 |
| Prostate Adenocarcinoma (PRAD) | 0.2 |
| Breast Invasive Carcinoma (BRCA) | 0.1 |

Provided herein as embodiment 162 is a compound according to any one of embodiments 1-159 or a pharmaceutically acceptable salt thereof, or the pharmaceutical composition according to embodiment 159 for use in treating cancer.

Provided herein as Embodiment 163 is a compound according to any one of Embodiments 1-159 or a pharmaceutically acceptable salt thereof, or the pharmaceutical composition according to Embodiment 159 for use in treating cancer, wherein one or more cells express KRAS G12D, G12V, G12A, G12S or G12C mutant protein.

Provided herein as Embodiment 164 is the compound or pharmaceutical composition for use of Embodiment 162 or 163, wherein the cancer is pancreatic cancer, colorectal cancer, non-small cell lung cancer, small bowel cancer, appendiceal cancer, cancer of unknown primary, endometrial cancer, mixed cancer types, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 165 is a use of the compound according to any one of Embodiments 1-159 or a pharmaceutically acceptable salt thereof, or the pharmaceutical composition according to Embodiment 160 in the preparation of a medicament for treating cancer.

Provided herein as Embodiment 166 is a use of the compound according to any one of Embodiments 1-159 or a pharmaceutically acceptable salt thereof, or the pharmaceutical composition according to Embodiment 160 in the preparation of a medicament for treating cancer, wherein one or more cells express KRAS G12D. G12V, G12A, G12S or G12C mutant protein.

Provided herein as Embodiment 167 is the use according to Embodiment 165 or 166, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 168 is a method of treating cancer in a subject in need thereof, the method comprising administering to the subject a therapeutically effective amount of the compound according to any one of to any one of Embodiments 1-159 or a pharmaceutically acceptable salt thereof.

Provided herein as Embodiment 169 is a method of treating cancer in a subject in need thereof, the method comprising administering to the subject a therapeutically effective amount of the compound according to any one of to any one of Embodiments 1-159 or a pharmaceutically acceptable salt thereof, wherein one or more cells express KRAS G12D, G12V, G12A. G12S or G12C mutant protein.

Provided herein as Embodiment 170 is the method according to Embodiment 168 or 169, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Provided herein as Embodiment 171 is the method according to Embodiment 168 or 169, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

Provided herein as Embodiment 172 is the method according to Embodiment 171, wherein the cancer is non-small cell lung cancer.

Provided herein as Embodiment 173 is the method according to Embodiment 171, wherein the cancer is colorectal cancer.

Provided herein as Embodiment 174 is the method according to Embodiment 171, wherein the cancer is pancreatic cancer.

Provided herein as Embodiment 175 is the method according to anyone of Embodiments 170-174, wherein the subject has a cancer that was determined to have one or more cells expressing the KRAS G12D, G12V, G12A, G12S or G12C mutant protein prior to administration of the compound or a pharmaceutically acceptable salt thereof.

### Combination Therapy

The present disclosure also provides methods for combination therapies in which an agent known to modulate other pathways, or other components of the same pathway, or even overlapping sets of target enzymes are used in combination with a compound of the present disclosure or a pharmaceutically acceptable salt thereof. In one aspect, such therapy includes but is not limited to the combination of one or more compounds of the disclosure with chemotherapeutic agents, therapeutic antibodies, and radiation treatment, to provide a synergistic or additive therapeutic effect. See, e.g., U.S. Pat. No. 10,519,146 B2, issued Dec. 31, 2019; specifically, the sections from column 201 (line 37) to column 212 (line 46) and column 219 (line 64) to column 220 (line 39), which are herewith incorporated by reference.

Provided herein as Embodiment 176 is the method according to anyone of Embodiments 168-175, which further comprises simultaneous, separate, or sequential administration of an effective amount of a second compound, wherein the second compound is an Aurora kinase A inhibitor, AKT inhibitor, arginase inhibitor, CDK4/6 inhibitor, ErbB family inhibitor, ERK inhibitor, FAK inhibitor, FGFR inhibitor, glutaminase inhibitor, IGF-1R inhibitor, KIF18A inhibitor, MCL-1 inhibitor, MEK inhibitor, mTOR inhibitor, PD-1 inhibitor, PD-L1 inhibitor, PI3K inhibitor, Raf kinase inhibitor, SHP2 inhibitor, SOS1 inhibitor, Src kinase inhibitor, or one or more chemotherapeutic agent.

In one embodiment, the second compound is administered as a pharmaceutically acceptable salt. In another embodiment the second compound is administered as a pharmaceutical composition comprising the second compound or a pharmaceutically acceptable salt thereof and a pharmaceutically acceptable excipient.

### Aurora Kinase A Inhibitors

Provided herein is the method according to anyone of Embodiments 168-175, which further comprises simultaneous, separate, or sequential administration of an effective amount of a second compound, wherein the second compound is an Aurora kinase A inhibitor.

Exemplary Aurora kinase A inhibitors for use in the methods provided herein include, but are not limited to, alisertib, cenisertib, danusertib, tozasertib, LY3295668 ((2R,4R)-1-[(3-chloro-2-fluorophenyl)methyl]-4-[[3-fluoro-6-[(5-methyl-1H-pyrazol-3-yl)amino]pyridin-2-yl]methyl]-2-methylpiperidine-4-carboxylic acid), ENMD-2076 (6-(4-methylpiperazin-1-yl)-N-(5-methyl-1H-pyrazol-3-yl)-2-[(E)-2-phenylethenyl]pyrimidin-4-amine), TAK-901 (5-(3-ethylsulfonylphenyl)-3,8-dimethyl-N-(1-methylpiperidin-4-yl)-9H-pyrido[2,3-b]indole-7-carboxamide), TT-00420 (4-[9-(2-chlorophenyl)-6-methyl-2,4,5,8,12-pentazatricyclo[8.4.0.03,7]tetradeca-1(14),3,6,8,10,12-hexaen-13-yl]morpholine), AMG 900 (N-[4-[3-(2-aminopyrimidin-4-yl)pyridin-2-yl]oxyphenyl]-4-(4-methylthiophen-2-yl)phthalazin-1-amine), MLN8054 (4-[[9-chloro-7-(2,6-difluorophenyl)-5H-pyrimido[5,4-d][2]benzazepin-2-yl]amino]benzoic acid), PF-03814735 (N-[2-[(1R,8S)-4-[[4-(cyclobutylamino)-5-(trifluoromethyl)pyrimidin-2-yl]amino]-11-azatricyclo[6.2.1.02,7]undeca-2(7),3,5-trien-11-yl]-2-oxoethyl]acetamide), SNS-314 (1-(3-chlorophenyl)-3-[5-[2-(thieno[3,2-d]pyrimidin-4-ylamino)ethyl]-1,3-thiazol-2-yl]urea), CYC116 (4-methyl-5-[2-(4-morpholin-4-ylanilino)pyrimidin-4-yl]-1,3-thiazol-2-amine), TAS-119, BI 811283, and TTP607.

### AKT Inhibitors

Provided herein is the method according to anyone of Embodiments 168-175, which further comprises simultaneous, separate, or sequential administration of an effective amount of a second compound, wherein the second compound is an AKT inhibitor.

Exemplary AKT inhibitors for use in the methods provided herein include, but are not limited to, afuresertib, capivasertib, ipatasertib, uprosertib, BAY 1125976 (2-[4-(1-aminocyclobutyl)phenyl]-3-phenylimidazo[1,2-b]pyridazine-6-carboxamide), ARQ 092 (3-[3-[4-(l-aminocyclobutyl)phenyl]-5-phenylimidazo[4,5-b]pyridin-2-yl]pyridin-2-amine), MK2206 (8-[4-(I-aminocyclobutyl)phenyl]-9-phenyl-2H-[1,2,4]triazolo[3,4-f][1,6]naphthyridin-3-one), SR13668 (indolo[2,3-b]carbazole-2,10-dicarboxylic acid, 5,7-dihydro-6-methoxy-, 2,10-diethyl ester), ONC201 (11-benzyl-7-[(2-methylphenyl)methyl]-2,5,7,11-tetrazatricyclo[7.4.0.02,6]trideca-1(9),5-dien-8-one), ARQ 751 (N-(3-aminopropyl)-N-[(1R)-1-(3-anilino-7-chloro-4-oxoquinazolin-2-yl)but-3-ynyl]-3-chloro-2-fluorobenzamide), RX-0201, and LY2780301.

### Arginase Inhibitors

Provided herein is the method according to anyone of Embodiments 168-175, which further comprises simultaneous, separate, or sequential administration of an effective amount of a second compound, wherein the second compound is an arginase inhibitor.

Exemplary arginase inhibitors for use in the methods provided herein include, but are not limited to, numidargistat and CB 280.

### CDK4/6 Inhibitors

Provided herein is the method according to anyone of Embodiments 168-175, which further comprises simultaneous, separate, or sequential administration of an effective amount of a second compound, wherein the second compound is a CDK4/6 inhibitor.

The term “CDK 4/6” as used herein refers to cyclin dependent kinases (“CDK”) 4 and 6, which are members of the mammalian serine/threonine protein kinases.

The term “CDK 4/6 inhibitor” as used herein refers to a compound that is capable of negatively modulating or inhibiting all or a portion of the enzymatic activity of CDK 4 and/or 6.

Exemplary CDK 4/6 inhibitors for use in the methods provided herein include, but are not limited to, abemaciclib, palbociclib, ribociclib, trilaciclib, and PF-06873600 ((pyrido[2,3-d]pyrimidin-7(8H)-one, 6-(difluoromethyl)-8-[(1R,2R)-2-hydroxy-2-methylcyclopentyl]-2-[[1-(methylsulfonyl)-4-piperidinyl]amino]).

In one embodiment, the CDK4/6 inhibitor is palbociclib.

### ErbB Family Inhibitors

Provided herein is the method according to anyone of Embodiments 168-175, which further comprises simultaneous, separate, or sequential administration of an effective amount of a second compound, wherein the second compound is an ErbB family inhibitor.

The term “ErbB family” as used herein refers to a member of a mammalian transmembrane protein tyrosine kinase family including: ErbB1 (EGFR HER1), ErbB2 (HER2), ErbB3 (HER3), and ErbB4 (HER4).

The term “ErbB family inhibitor” as used herein refers to an agent, e.g., a compound or antibody, that is capable of negatively modulating or inhibiting all or a portion of the activity of at least one member of the ErbB family. The modulation or inhibition of one or more ErbB tyrosine kinase may occur through modulating or inhibiting kinase enzymatic activity of one or more ErbB family member or by blocking homodimerization or heterodimerization of ErbB family members.

In one embodiment, the ErbB family inhibitor is an EGFR inhibitor, e.g., an anti-EGFR antibody. Exemplary anti-EGFR antibodies for use in the methods provided herein include, but are not limited to, zalutumumab, nimotuzumab, matuzumab, necitumumab, panitumumab, and cetuximab. In one embodiment, the anti-EGFR antibody is cetuximab. In one embodiment, the anti-EGFR antibody is panitumumab.

In another embodiment the ErbB family inhibitor is a HER2 inhibitor, e.g., an anti-HER2 antibody. Exemplary anti-HER-2 antibodies for use in the methods provided herein include, but are not limited to, pertuzumab, trastuzumab, and trastuzumab emtansine.

In yet another embodiment the ErbB family inhibitor is a HER3 inhibitor, e.g., an anti-HER3 antibody, such as HMBD-001 (Hummingbird Bioscience).

In one embodiment, the ErbB family inhibitor is a combination of an anti-EGFR antibody and anti-HER2 antibody.

In one embodiment, the ErbB family inhibitor is an irreversible inhibitor. Exemplary irreversible ErbB family inhibitors for use in the methods provided herein include, but are not limited to, afatinib, dacomitinib, canertinib, poziotinib, AV 412 ((N-[4-[(3-chloro-4-fluorophenyl)amino]-7-[3-methyl-3-(4-methyl-1-piperazinyl)-1-butyn-1-yl]-6-quinazolinyl]-2-propenamide)), PF 6274484 ((N-[4-[(3-chloro-4-fluorophenyl)amino]-7-methoxy-6-quinazolinyl]-2-propenamide), and HKI 357 ((E)-N-[4-[3-chloro-4-[(3-fluorophenyl)methoxy]anilino]-3-cyano-7-ethoxyquinolin-6-yl]-4-(dimethylamino)but-2-enamide).

In one embodiment, the irreversible ErbB family inhibitor is afatinib. In one embodiment, the irreversible ErbB family inhibitor is dacomitinib.

In one embodiment, the ErbB family inhibitor is a reversible inhibitor. Exemplary reversible ErbB family inhibitors for use in the methods provided herein include, but are not limited to erlotinib, gefitinib, sapitinib, varlitinib, tarloxotinib, TAK-285 (N-(2-(4-((3-chloro-4-(3-(trifluoromethyl)phenoxy)phenyl)amino)-5H-pyrrolo[3,2-d]pyrimidin-5-yl)ethyl)-3-hydroxy-3-methylbutanamide), AEE788 ((S)-6-(4-((4-ethylpiperazin-1-yl)methyl)phenyl)-N-(1-phenylethyl)-7H-pyrrolo[2,3-d]pyrimidin-4-amine), BMS 599626 ((3S)-3-morpholinylmethyl-[4-[[1-[(3-fluorophenyl)methyl]-1H-indazol-5-yl]amino]-5-methylpyrrolo[2,1-f][1,2,4]triazin-6-yl]-carbamate), and GW 583340 (N-[3-chloro-4-[(3-fluorophenyl)methoxy]phenyl]-6-[2-[(2-methylsulfonylethylamino)methyl]-1,3-thiazol-4-yl]quinazolin-4-amine).

In one embodiment, the reversible ErbB family inhibitor is sapitinib. In one embodiment, the reversible ErbB family inhibitor is tarloxotinib.

### ERK Inhibitors

Provided herein is the method according to anyone of Embodiments 168-175, which further comprises simultaneous, separate, or sequential administration of an effective amount of a second compound, wherein the second compound is an ERK inhibitor.

Exemplary ERK inhibitors for use in the methods provided herein include, but are not limited to, ulixertinib, ravoxertinib, CC-90003 (N-[2-[[2-[(2-methoxy-5-methylpyridin-4-yl)amino]-5-(trifluoromethyl)pyrimidin-4-yl]amino]-5-methylphenyl]prop-2-enamide), LY3214996 (6,6-dimethyl-2-[2-[(2-methylpyrazol-3-yl)amino]pyrimidin-4-yl]-5-(2-morpholin-4-ylethyl)thieno[2,3-c]pyrrol-4-one), KO-947 (1,5,6,8-tetrahydro-6-(phenylmethyl)-3-(4-pyridinyl)-7H-pyrazolo[4,3-g]quinazolin-7-one), ASTX029, LTT462, and JSI-1187.

### FAK Inhibitors

Provided herein is the method according to anyone of Embodiments 168-175, which further comprises simultaneous, separate, or sequential administration of an effective amount of a second compound, wherein the second compound is a FAK inhibitor.

Exemplary FAK inhibitors for use in the methods provided herein include, but are not limited to, GSK2256098 (2-[[5-chloro-2-[(5-methyl-2-propan-2-ylpyrazolo-3-yl)amino]pyridin-4-yl]aminol-N-methoxybenzamide), PF-00562271 (N-methyl-N-[3-[[[2-[(2-oxo-1,3-dihydroindol-5-yl)amino]-5-(trifluoromethyl)pyrimidin-4-yl]amino]methyl]pyridin-2-yl]methanesulfonamide), VS-4718 (2-[2-(2-methoxy-4-morpholin-4-ylanilino)-5-(trifluoromethyl)pyridin-4-yl]amino]-N-methylbenzamide), and APG-2449.

### FGFR Inhibitors

Provided herein is the method according to anyone of Embodiments 168-175, which further comprises simultaneous, separate, or sequential administration of an effective amount of a second compound, wherein the second compound is an FGFR inhibitor.

Exemplary FGFR inhibitors for use in the methods provided herein include, but are not limited to, futibatinib, pemigatinib, ASP5878 (2-[4-[[5-[(2,6-difluoro-3,5-dimethoxyphenyl)methoxy]pyrimidin-2-yl]amino]pyrazol-1-yl]ethanol), AZD4547 (N-[5-[2-(3,5-dimethoxyphenyl)ethyl]-1H-pyrazol-3-yl]-4-[(3S,5R)-3,5-dimethylpiperazin-1-yl]benzamide), Debio 1347 ([5-amino-1-(2-methyl-3H-benzimidazol-5-yl)pyrazol-4-yl]-(1H-indol-2-yl)methanone), INCB062079, H3B-6527 (N-[2-[[6-[(2,6-dichloro-3,5-dimethoxyphenyl)carbamoyl-methylamino]pyrimidin-4-yl]amino]-5-(4-ethylpiperazin-1-yl)phenyl]prop-2-enamide), ICP-105, CPL304110, HMPL-453, and HGS1036.

### Glutaminase Inhibitors

Provided herein is the method according to anyone of Embodiments 168-175, which further comprises simultaneous, separate, or sequential administration of an effective amount of a second compound, wherein the second compound is a glutaminase inhibitor.

Exemplary glutaminase inhibitors for use in the methods provided herein include, but are not limited to, telaglenastat, IPN60090, and OP 330.

### IGF-1R Inhibitors

Provided herein is the method according to anyone of Embodiments 168-175, which further comprises simultaneous, separate, or sequential administration of an effective amount of a second compound, wherein the second compound is an IGF-1R inhibitor.

Exemplary IGF-1R inhibitors for use in the methods provided herein include, but are not limited to, cixutumumab, dalotuzumab, linsitinib, ganitumab, robatumumab, BMS-754807 ((2S)-1-[4-[(5-cyclopropyl-1H-pyrazol-3-yl)amino]pyrrolo[2,1-f][,2,4]triazin-2-yl]-N-(6-fluoropyridin-3-yl)-2-methylpyrrolidine-2-carboxamide), KW-2450 (N-[5-[[4-(2-hydroxyacetyl)piperazin-1-yl]methyl]-2-[(E)-2-(1H-indazol-3-yl)ethenyl]phenyl]-3-methylthiophene-2-carboxamide), PL225B, AVE1642, and BIIB022.

### KIF18A Inhibitors

Provided herein is the method according to anyone of Embodiments 168-175, which further comprises simultaneous, separate, or sequential administration of an effective amount of a second compound, wherein the second compound is a KIF18A inhibitor.

Exemplary KIF18A inhibitors for use in the methods provided herein include, but are not limited to, the inhibitors disclosed in US 2020/0239441, WO 2020/132649, WO 2020/132651, and WO 2020/132653, each of which is herewith incorporated by reference in its entirety.

### MCL-1 Inhibitors

Provided herein is the method according to anyone of Embodiments 168-175, which further comprises simultaneous, separate, or sequential administration of an effective amount of a second compound, wherein the second compound is an MCL-1 inhibitor.

Exemplary MEK inhibitors for use in the methods provided herein include, but are not limited to, murizatoclax, tapotoclax, AZD 5991 ((3aR)-5-chloro-2,11,12,24,27,29-hexahydro-2,3,24,33-tetramethyl-22H-9,4,8-(metheniminomethyno)-14,20:26,23-dimetheno-10H,20H-pyrazolo[4,3-1][2,15,22,18,19]benzoxadithiadiazacyclohexacosine-32-carboxylic acid). MIK 665 ((αR)-α-[[(5S)-5-[3-Chloro-2-methyl-4-[2-(4-methyl-1-piperazinyl)ethoxy]phenyl]-6-(4-fluorophenyl)thieno[2,3-d]pyrimidin-4-yl]oxy]-2-[[2-(2-methoxyphenyl)-4-pyrimidinyl]methoxy]benzenepropanoic acid), and ABBV-467.

In one embodiment, the MCL-1 inhibitor is murizatoclax. In another embodiment, the MCL-1 inhibitor is tapotoclax.

### MEK Inhibitors

Provided herein is the method according to anyone of Embodiments 168-175, which further comprises simultaneous, separate, or sequential administration of an effective amount of a second compound, wherein the second compound is MEK inhibitor.

Exemplary MEK inhibitors for use in the methods provided herein include, but are not limited to, trametinib, cobimetinib, selumetinib, pimasertib, refametinib, PD-325901 (N-[(2R)-2.3-dihydroxypropoxy]-3,4-difluoro-2-(2-fluoro-4-iodoanilino)benzamide), AZD8330 (2-(2-fluoro-4-iodoanilino)-N-(2-hydroxyethoxy)-1,5-dimethyl-6-oxopyridine-3-carboxamide), GDC-0623 (5-(2-fluoro-4-iodoanilino)-N-(2-hydroxyethoxy)imidazo[1,5-a]pyridine-6-carboxamide), RO4987655 (3,4-difluoro-2-(2-fluoro-4-iodoanilino)-N-(2-hydroxyethoxy)-5-[(3-oxooxazinan-2-yl)methyl]benzamide), TAK-733 (3-[(2R)-2,3-dihydroxypropyl]-6-fluoro-5-(2-fluoro-4-iodoanilino)-8-methylpyrido[2,3-d]pyrimidine-4,7-dione), PD0325901 (N-[(2R)-2,3-dihydroxypropoxy]-3,4-difluoro-2-(2-fluoro-4-iodoanilino)benzamide), C₁₋₁₀40 (2-(2-chloro-4-iodophenylamino)-N-(cyclopropylmethoxy)-3,4-difluorobenzamide), PD318088 (5-bromo-N-(2,3-dihydroxypropoxy)-3,4-difluoro-2-(2-fluoro-4-iodophenylamino)benzamide), PD98059 (2-(2-amino-3-methoxyphenyl)-4H-chromen-4-one), PD334581 (N-[5-[3,4-Difluoro-2-[(2-fluoro-4-iodophenyl)amino]phenyl]-1,3,4-oxadiazol-2-yl]-4-morpholineethanamine), FCN-159, CS3006, HL-085, SHR 7390, and WX-554.

In one embodiment, the MEK inhibitor is trametinib.

mTOR Inhibitors

Provided herein is the method according to anyone of Embodiments 168-175, which further comprises simultaneous, separate, or sequential administration of an effective amount of a second compound, wherein the second compound is an mTOR inhibitor.

Exemplary mTOR inhibitors for use in the methods provided herein include, but are not limited to, everolimus, rapamycin, zotarolimus (ABT-578), ridaforolimus (deforolimus, MK-8669), sapanisertib, buparlisib, pictilisib, vistusertib, dactolisib. Torin-1 (1-(4-(4-propionylpiperazin-1-yl)-3-(trifluoromethyl)cyclohexyl)-9-(quinolin-3-yl)benzo[h][1,6]naphthyridin-2(1H)-one), GDC-0349 ((S)-1-ethyl-3-(4-(4-(3-methylmorpholino)-7-(oxetan-3-yl)-5,6,7,8-tetrahydropyrido[3,4-d]pyrimidin-2-yl)phenyl)urea), and VS-5584 (SB2343, (5-(8-methyl-2-morpholin-4-yl-9-propan-2-ylpurin-6-yl)pyrimidin-2-amine).

In one embodiment, the mTOR inhibitor is everolimus.

### PD-1 Inhibitors

Provided herein is the method according to anyone of Embodiments 168-175, which further comprises simultaneous, separate, or sequential administration of an effective amount of a second compound, wherein the second compound is a PD-1 inhibitor.

Exemplary PD-1 inhibitors for use in the methods provided herein include, but are not limited to, pembrolizumab, nivolumab, cemiplimab, spartalizumab (PDR001), camrelizumab (SHR1210), sintilimab (IB1308), tislelizumab (BGB-A317), toripalimab (JS 001), dostarlimab (TSR-042, WBP-285), INCMGA00012 (MGA012), AMP-224, AMP-514, and the anti-PD-1 antibody as described in U.S. Pat. No. 10,640,504 B2 (the “Anti-PD-1 Antibody A,” column 66, line 56 to column 67, line 24 and column 67, lines 54-57), which is incorporated herein by reference.

In one embodiment, the PD-1 inhibitor is pembrolizumab. In another embodiment the PD-1 inhibitor is the Anti-PD-1 Antibody A.

### PD-L1 Inhibitors

Provided herein is the method according to anyone of Embodiments 168-175, which further comprises simultaneous, separate, or sequential administration of an effective amount of a second compound, wherein the second compound is a PD-L1 inhibitor.

Exemplary PD-L1 inhibitors for use in the methods provided herein include, but are not limited to, atezolizumab, avelumab, durvalumab, ZKAB001, TG-1501, SHR-1316, MSB2311. MDX-1105, KN035, IMC-001, HLX20, FAZ053, CS1001, CK-301, CBT-502, BGB-A333, BCD-135, and A167.

In one embodiment, the PD-L1 inhibitor is atezolizumab.

### PI3K Inhibitors

Provided herein is the method according to anyone of Embodiments 168-175, which further comprises simultaneous, separate, or sequential administration of an effective amount of a second compound, wherein the second compound is a PI3K inhibitor.

Exemplary PI3K inhibitors for use in the methods provided herein include, but are not limited to, idelalisib, copanlisib, duvelisib, alpelisib, taselisib, perifosine, buparlisib, umbralisib, pictilisib, dactolisib, voxtalisib, sonolisib, tenalisib, serabelisib, acalisib, CUDC-907 (N-hydroxy-2-[[2-(6-methoxypyridin-3-yl)-4-morpholin-4-ylthieno[3,2-d]pyrimidin-6-yl]methyl-methylamino]pyrimidine-5-carboxamide). ME-401 (N-[2-methyl-1-[2-(1-methylpiperidin-4-yl)phenyl]propan-2-yl]-4-(2-methylsulfonylbenzimidazol-1-yl)-6-morpholin-4-yl-1,3,5-triazin-2-amine), IPI-549 (2-amino-N-[(1S)-1-[8-[2-(1-methylpyrazol-4-yl)ethynyl]-1-oxo-2-phenylisoquinoline-3-yl]ethyl]pyrazolo[1,5-a]pyrimidine-3-carboxamide), SF1126 ((2S)-2-[[(2S)-3-carboxy-2-[[2-[[(2S)-5-(diaminomethylideneamino)-2-[[4-oxo-4-[[4-(4-oxo-8-phenylchromen-2-yl)morpholin-4-ium-4-yl]methoxy]butanoyl]amino]pentanoyl]amino]acetyl]amino]propanoyl]amino]-3-hydroxypropanoate). XL147 (N-[3-(2,1,3-benzothiadiazol-5-ylamino)quinoxalin-2-yl]-4-methylbenzenesulfonamide), GSK1059615 ((5Z)-5-[(4-pyridin-4-ylquinolin-6-yl)methylidene]-1,3-thiazolidine-2,4-dione), and AMG 319 (N-[(1S)-1-(7-fluoro-2-pyridin-2-ylquinolin-3-yl)ethyl]-7H-purin-6-amine).

### Raf Kinase Inhibitors

Provided herein is the method according to anyone of Embodiments 168-175, which further comprises simultaneous, separate, or sequential administration of an effective amount of a second compound, wherein the second compound is a Raf kinase inhibitor.

The term “RAF kinase” as used herein refers to a member of a mammalian serine/threonine kinases composed of three isoforms (C-Raf, B-Raf and A-Raf) and includes homodimers of each isoform as well as heterodimers between isoforms, e.g., C-Raf/B-Raf heterodimers.

The term “Raf kinase inhibitor” as used herein refers to a compound that is capable of negatively modulating or inhibiting all or a portion of the enzymatic activity of one or more member of the Raf family kinases or is capable of disrupting Raf homodimer or heterodimer formation to inhibit activity.

In one embodiment, the Raf kinase inhibitor includes, but is not limited to, encorafenib, sorafenib, lifirafenib, vemurafenib, dabrafenib, PLX-8394 (N-(3-(5-(2-cyclopropylpyrimidin-5-yl)-3a,7a-dihydro-1H-pyrrolo[2,3-b]pyridine-3-carbonyl)-2,4-difluorophenyl)-3-fluoropyrrolidine-1-sulfonamide), Raf-709 (N-(2-methyl-5,-morpholino-6′-((tetrahydro-2H-pyran-4-yl)oxy)-[3,3′-bipyridin]-5-yl)-3-(trifluoromethyl)benzamide), LXH254 (N-(3-(2-(2-hydroxyethoxy)-6-morpholinopyridin-4-yl)-4-methylphenyl)-2-(trifluoromethyl)isonicotinamide), LY3009120 (1-(3,3-dimethylbutyl)-3-(2-fluoro-4-methyl-5-(7-methyl-2-(methylamino)pyrido[2,3-d]pyrimidin-6-yl)phenyl)urea), Tak-632 (N-(7-cyano-6-(4-fluoro-3-(2-(3-(trifluoromethyl)phenyl)acetamido)phenoxy)benzo[d]thiazol-2-yl)cyclopropanecarboxamide), CEP-32496 (1-(3-((6,7-dimethoxyquinazolin-4-yl)oxy)phenyl)-3-(5-(1,1,1-trifluoro-2-methylpropan-2-yl)isoxazol-3-yl)urea), CCT196969 (1-(3-(tert-butyl)-1-phenyl-1H-pyrazol-5-yl)-3-(2-fluoro-4-((3-oxo-3,4-dihydropyrido[2,3-b]pyrazin-8-yl)oxy)phenyl)urea), and RO5126766 (N-[3-fluoro-4-[[4-methyl-2-oxo-7-(2-pyrimidinyloxy)-2H-1-benzopyran-3-yl]methyl]-2-pyridinyl]-N′-methyl-sulfamide).

In one embodiment, the Raf kinase inhibitor is encorafenib. In one embodiment, the Raf kinase inhibitor is sorafenib. In one embodiment, the Raf kinase inhibitor is lifirafenib.

### SHP2 Inhibitors

Provided herein is the method according to anyone of Embodiments 168-175, which further comprises simultaneous, separate, or sequential administration of an effective amount of a second compound, wherein the second compound is a SHP2 inhibitor.

Exemplary SHP2 inhibitors for use in the methods provided herein include, but are not limited to, SHP-099 (6-(4-amino-4-methylpiperidin-1-yl)-3-(2,3-dichlorophenyl)pyrazin-2-amine dihydrochloride), RMC-4550 ([3-[(3S,4S)-4-amino-3-methyl-2-oxa-8-azaspiro[4.5]decan-8-yl]-6-(2,3-dichlorophenyl)-5-methylpyrazin-2-yl]methanol), TNO155, (3S,4S)-8-[6-amino-5-(2-amino-3-chloropyridin-4-yl)sulfanylpyrazin-2-yl]-3-methyl-2-oxa-8-azaspiro[4.5]decan-4-amine), and RMC-4630 (Revolution Medicine). In one embodiment, the SHP inhibitor for use in the methods provided herein is RMC-4630 (Revolution Medicine).

In another embodiment, exemplary SHP2 inhibitors for use in the methods provided herein include, but are not limited to, 3-[(1R,3R)-1-amino-3-methoxy-8-azaspiro[4.5]dec-8-yl]-6-(2,3-dichlorophenyl)-5-methyl-2-pyrazinemethanol (CAS 2172651-08-8), 3-[(3S,4S)-4-amino-3-methyl-2-oxa-8-azaspiro[4.5]dec-8-yl]-6-[(2,3-dichlorophenyl)thio]-5-methyl-2-pyrazinemethanol (CAS 2172652-13-8), 3-[(3S,4S)-4-amino-3-methyl-2-oxa-8-azaspiro[4.5]dec-8-yl]-6-[[3-chloro-2-(3-hydroxy-1-azetidinyl)-4-pyridinyl]thio]-5-methyl-2-pyrazinemethanol (CAS 2172652-38-7), and 6-[(2-amino-3-chloro-4-pyridinyl)thio]-3-[(3S,4S)-4-amino-3-methyl-2-oxa-8-azaspiro[4.5]dec-8-yl]-5-methyl-2-pyrazinemethanol (CAS 2172652-48-9).

In another embodiment, exemplary SHP2 inhibitors for use in the methods provided herein include, but are not limited to, 1-[5-(2,3-dichlorophenyl)-6-methylimidazo[1,5-a]pyrazin-8-yl]-4-methyl-4-piperidinamine (CAS 2240981-75-1), (1R)-8-[5-(2,3-dichlorophenyl)-6-methylimidazo[1,5-a]pyrazin-8-yl]-8-azaspiro[4.5]decan-1-amine (CAS 2240981-78-4), (3S,4S)-8-[7-(2,3-dichlorophenyl)-6-methylpyrazolo[1,5-a]pyrazin-4-yl]-3-methyl-2-oxa-8-azaspiro[4.5]decan-4-amine (CAS 2240982-45-8), (3S,4S)-8-[7-[(2-amino-3-chloro-4-pyridinyl)thio]pyrazolo[1,5-a]pyrazin-4-yl]-3-methyl-2-oxa-8-azaspiro[4.5]decan-4-amine (CAS 2240982-57-2), 4-[(3S,4S)-4-amino-3-methyl-2-oxa-8-azaspiro[4.5]dec-8-yl]-7-(2,3-dichlorophenyl)-6-methyl-pyrazolo[1,5-a]pyrazine-2-methanol (CAS 2240982-69-6), 7-[(2-amino-3-chloro-4-pyridinyl)thio]-4-[(3S,4S)-4-amino-3-methyl-2-oxa-8-azaspiro[4.5]dec-8-yl]-6-methyl-pyrazolo[1,5-a]pyrazine-2-methanol (CAS 2240982-73-2), and (3S,4S)-8-[7-[(2-amino-3-chloro-4-pyridinyl)thio]-6-methylpyrazolo[1,5-a]pyrazin-4-yl]-3-methyl-2-oxa-8-azaspiro[4.5]decan-4-amine (CAS 2240982-77-6).

In one embodiment, the SHP inhibitor for use in the methods provided herein is (1R)-8-[5-(2,3-dichlorophenyl)-6-methylimidazo[1,5-a]pyrazin-8-yl]-8-azaspiro[4.5]decan-1-amine (CAS 2240981-78-4).

In another embodiment, exemplary SHP2 inhibitors for use in the methods provided herein include, but are not limited to 3-[(1R)-1-amino-8-azaspiro[4.5]dec-8-yl]-6-(2,3-dichlorophenyl)-5-hydroxy-2-pyridinemethanol (CAS 2238840-54-3), 3-[(1R)-1-amino-8-azaspiro[4.5]dec-8-yl]-6-[(2,3-dichlorophenyl)thio]-5-hydroxy-2-pyridinemethanol (CAS 2238840-56-5), 5-[(1R)-1-amino-8-azaspiro[4.5]dec-8-yl]-2-(2,3-dichlorophenyl)-3-pyridinol (CAS 2238840-58-7), 3-[(1R)-1-amino-8-azaspiro[4.5]dec-8-yl]-6-(2,3-dichlorophenyl)-5-methyl-2-pyridinemethanol (CAS 2238840-60-1), (1R)-8-[6-(2,3-dichlorophenyl)-5-methyl-3-pyridinyl]-8-azaspiro[4.5]decan-1-amine (CAS 2238840-62-3), 3-[(1R)-1-amino-8-azaspiro[4.5]dec-8-yl]-6-[(2,3-dichlorophenyl)thio]-5-methyl-2-pyridinemethanol (CAS 2238840-63-4), (1R)-8-[6-[(2,3-dichlorophenyl)thio]-5-methyl-3-pyridinyl]-8-azaspiro[4.5]decan-1-amine (CAS 2238840-64-5), 5-(4-amino-4-methyl-1-piperidinyl)-2-[(2,3-dichlorophenyl)thio]-3-pyridinol (CAS 2238840-65-6), 5-[(1R)-1-amino-8-azaspiro[4.5]dec-8-yl]-2-[(2,3-dichlorophenyl)thio]-3-pyridinol (CAS 2238840-66-7), 6-[(2-amino-3-chloro-4-pyridinyl)thio]-3-[(3S,4S)-4-amino-3-methyl-2-oxa-8-azaspiro[4.5]dec-8-yl]-5-hydroxy-2-pyridinemethanol (CAS 2238840-67-8), 3-(4-amino-4-methyl-1-piperidinyl)-6-(2,3-dichlorophenyl)-5-hydroxy-2-pyridinemethanol (CAS 2238840-68-9), 3-[(3S,4S)-4-amino-3-methyl-2-oxa-8-azaspiro[4.5]dec-8-yl]-6-(2,3-dichlorophenyl)-5-methyl-2-pyridinemethanol (CAS 2238840-69-0), 6-[(2-amino-3-chloro-4-pyridinyl)thio]-3-[(3S,4S)-4-amino-3-methyl-2-oxa-8-azaspiro[4.5]dec-8-yl]-5-methyl-2-pyridinemethanol (CAS 2238840-70-3), 3-(4-amino-4-methyl-1-piperidinyl)-6-(2,3-dichlorophenyl)-5-methyl-2-pyridinemethanol (CAS 2238840-71-4), 6-[(2-amino-3-chloro-4-pyridinyl)thio]-3-(4-amino-4-methyl-1-piperidinyl)-2-pyridinemethanol (CAS 2238840-72-5), 5-[(2-amino-3-chloro-4-pyridinyl)thio]-2-[(3S,4S)-4-amino-3-methyl-2-oxa-8-azaspiro[4.5]dec-8-yl]-6-methyl-3-pyridinemethanol (CAS 2238840-73-6), 2-[(3S,4S)-4-amino-3-methyl-2-oxa-8-azaspiro[4.5]dec-8-yl]-5-(2,3-dichlorophenyl)-6-methyl-3-pyridinemethanol (CAS 2238840-74-7), 3-[(3S,4S)-4-amino-3-methyl-2-oxa-8-azaspiro[4.5]dec-8-yl]-6-(2,3-dichlorophenyl)-5-hydroxy-2-pyridinemethanol (CAS 2238840-75-8), and 2-[(2-amino-3-chloro-4-pyridyl)sulfanyl]-5-[(3S,4S)-4-amino-3-methyl-2-oxa-8-azaspiro[4.5]decan-8-yl]-6-(hydroxymethyl)pyridin-3-ol.

In one embodiment, the SHP inhibitor for use in the methods provided herein is 3-[(1R)-1-amino-8-azaspiro[4.5]dec-8-yl]-6-[(2,3-dichlorophenyl)thio]-5-hydroxy-2-pyridinemethanol (CAS 2238840-56-5).

In one embodiment, the SHP2 inhibitor for use in the methods provided herein is an inhibitor disclosed in U.S. Pat. No. 10,590,090 B2, US 2020/017517 A1, US 2020/017511 A1, or WO 2019/075265 A1, each of which is herewith incorporated by reference in its entirety.

### SOS1 Inhibitors

Provided herein is the method according to anyone of Embodiments 168-175, which further comprises simultaneous, separate, or sequential administration of an effective amount of a second compound, wherein the second compound is an SOS1 inhibitor.

Exemplary SOS1 inhibitors for use in the methods provided herein include, but are not limited to, BI 3406 (N-[(1R)-1-[3-amino-5-(trifluoromethyl)phenyl]ethyl]-7-methoxy-2-methyl-6-[(3S)-oxolan-3-yl]oxoquinazolin-4-amine), and BI 1701963.

### Src Kinase Inhibitors

Provided herein is the method according to anyone of Embodiments 168-175, which further comprises simultaneous, separate, or sequential administration of an effective amount of a second compound, wherein the second compound is a Src kinase inhibitor.

The term “Src kinase” as used herein refers to a member of a mammalian nonreceptor tyrosine kinase family including: Src, Yes, Fyn, and Fgr (SrcA subfamily), Lek, Hck, Blk, and Lyn (SrcB subfamily), and Frk subfamily.

The term “Src kinase inhibitor” as used herein refers to a compound that is capable of negatively modulating or inhibiting all or a portion of the enzymatic activity of one or more member of the Src kinases.

Exemplary Src kinase inhibitors for use in the methods provided herein include, but are not limited to, dasatinib, ponatinib, vandetanib, bosutinib, saracatinib, KX2-391 (N-benzyl-2-(5-(4-(2-morpholinoethoxy)phenyl)pyridin-2-yl)acetamide), SU6656 ((Z)—N,N-dimethyl-2-oxo-3-((4,5,6,7-tetrahydro-H-indol-2-yl)methylene)indoline-5-sulfonamide), PP 1 (1-(tert-butyl)-3-(p-tolyl)-1H-pyrazolo[3,4-d]pyrimidin-4-amine), WH-4-023 (2,6-dimethylphenyl(2,4-dimethoxyphenyl)(2-((4-(4-methylpiperazin-1-yl)phenyl)amino)pyrimidin-4-yl)carbamate), and KX-01 (N-benzyl-2-(5-(4-(2-morpholinoethoxy)phenyl)pyridin-2-yl)acetamide).

In one embodiment, the Src kinase inhibitor is dasatinib. In one embodiment, the Src kinase inhibitor is saracatinib. In one embodiment, the Src kinase inhibitor is ponatinib. In one embodiment, the Src kinase inhibitor is vandetanib. In one embodiment, the Src kinase inhibitor is KX-01.

### Chemotherapeutic Agents

Provided herein is the method according to anyone of Embodiments 168-175, which further comprises simultaneous, separate, or sequential administration of an effective amount of a second compound, wherein the second compound is one or more chemotherapeutic agent.

Exemplary chemotherapeutic agents for use in the methods provided herein include, but are not limited to, leucovorin calcium (calcium folinate), 5-fluorouracil, irinotecan, oxaliplatin, cisplatin, carboplatin, pemetrexed, docetaxel, paclitaxel, gemcitabine, vinorelbine, chlorambucil, cyclophosphamide, and methotrexate.

### Definitions

The following definitions are provided to assist in understanding the scope of this disclosure.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the standard deviation found in their respective testing measurements.

As used herein, if any variable occurs more than one time in a chemical formula, its definition on each occurrence is independent of its definition at every other occurrence. If the chemical structure and chemical name conflict, the chemical structure is determinative of the identity of the compound.

### Stereoisomers

The compounds of the present disclosure may contain, for example, double bonds, one or more asymmetric carbon atoms, and bonds with a hindered rotation, and therefore, may exist as stereoisomers, such as double-bond isomers (i.e., geometric isomers (E/Z)), enantiomers, diastereomers, and atropisomers. Accordingly, the scope of the instant disclosure is to be understood to encompass all possible stereoisomers of the illustrated compounds, including the stereoisomerically pure form (for example, geometrically pure, enantiomerically pure, diastereomerically pure, and atropoisomerically pure) and stereoisomeric mixtures (for example, mixtures of geometric isomers, enantiomers, diastereomers, and atropisomers, or mixture of any of the foregoing) of any chemical structures disclosed herein (in whole or in part), unless the stereochemistry is specifically identified.

If the stereochemistry of a structure or a portion of a structure is not indicated with, for example, bold or dashed lines, the structure or portion of the structure is to be interpreted as encompassing all stereoisomers of it. If the stereochemistry of a structure or a portion of a structure is indicated with, for example, bold or dashed lines, the structure or portion of the structure is to be interpreted as encompassing only the stereoisomer indicated, unless otherwise noted.

For example,

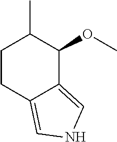

represents

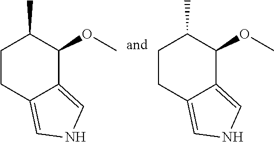

Similarly, for example, the chemical name (4R)-4-methoxy-5-methyl-4,5,6,7-tetrahydro-2H-isoindole represents (4R,5R)-4-methoxy-5-methyl-4,5,6,7-tetrahydro-2H-isoindole and (4R,5S)-4-methoxy-5-methyl-4,5,6,7-tetrahydro-2H-isoindole.

Similarly, for example, the chemical name 7-chloro-6-fluoro-1-(2-isopropyl-4-methylpyridin-3-yl)pyrido[2,3-d]pyrimidine-2,4(1H,3H)-dione represents (M)-7-chloro-6-fluoro-1-(2-isopropyl-4-methylpyridin-3-yl)pyrido[2,3-d]pyrimidine-2,4(1H,3H)-dione and (P)-7-chloro-6-fluoro-1-(2-isopropyl-4-methylpyridin-3-yl)pyrido[2,3-d]pyrimidine-2,4(1H,3H)-dione.

In certain instances, a bond drawn with a wavy line indicates that both stereoisomers are encompassed. This is not to be confused with a wavy line drawn perpendicular to a bond which indicates the point of attachment of a group to the rest of the molecule.

The term “stereoisomer” or “stereoisomerically pure” compound as used herein refers to one stereoisomer (for example, geometric isomer, enantiomer, diastereomer and atropisomer) of a compound that is substantially free of other stereoisomers of that compound. For example, a stereoisomerically pure compound having one chiral center will be substantially free of the mirror image enantiomer of the compound and a stereoisomerically pure compound having two chiral centers will be substantially free of other enantiomers or diastereomers of the compound. A typical stereoisomerically pure compound comprises greater than about 80% by weight of one stereoisomer of the compound and equal or less than about 20% by weight of other stereoisomers of the compound, greater than about 90% by weight of one stereoisomer of the compound and equal or less than about 10% by weight of the other stereoisomers of the compound, greater than about 95% by weight of one stereoisomer of the compound and equal or less than about 5% by weight of the other stereoisomers of the compound, or greater than about 97% by weight of one stereoisomer of the compound and equal or less than about 3% by weight of the other stereoisomers of the compound.

This disclosure also encompasses the pharmaceutical compositions comprising stereoisomerically pure forms and the use of stereoisomerically pure forms of any compounds disclosed herein. Further, this disclosure also encompasses pharmaceutical compositions comprising mixtures of stereoisomers of any compounds disclosed herein and the use of said pharmaceutical compositions or mixtures of stereoisomers. These stereoisomers or mixtures thereof may be synthesized in accordance with methods well known in the art and methods disclosed herein. Mixtures of stereoisomers may be resolved using standard techniques, such as chiral columns or chiral resolving agents. Further, this disclosure encompasses pharmaceutical compositions comprising mixtures of any of the compounds disclosed herein and one or more other active agents disclosed herein. See, for example, Jacques et al., Enantiomers, Racemates and Resolutions (Wiley-Interscience, New York, 1981); Wilen et al., Tetrahedron 33:2725; Eliel, Stereochemistry of Carbon Compounds (McGraw-Hill, NY, 1962); and Wilen, Tables of Resolving Agents and Optical Resolutions, page 268 (Eliel, Ed., Univ. of Notre Dame Press, Notre Dame, IN, 1972).

### Tautomers

As known by those skilled in the art, certain compounds disclosed herein may exist in one or more tautomeric forms. Because one chemical structure may only be used to represent one tautomeric form, it will be understood that for convenience, referral to a compound of a given structural formula includes other tautomers of said structural formula. Accordingly, the scope of the instant disclosure is to be understood to encompass all tautomeric forms of the compounds disclosed herein.

### Isotopically Labelled Compounds

Further, the scope of the present disclosure includes all pharmaceutically acceptable isotopically-labelled compounds of the compounds disclosed herein, such as the compounds of Formula I, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²⁵I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S. Certain isotopically-labelled compounds of Formula I, for example, those incorporating a radioactive isotope, are useful in drug and/or substrate tissue distribution studies. The radioactive isotopes tritium (³H) and carbon-14 (⁴C) are particularly useful for this purpose in view of their case of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. Substitution with positron emitting isotopes, such as ¹¹C, ¹⁸F, ¹⁵O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying General Synthetic Schemes and Examples using an appropriate isotopically-labelled reagent in place of the non-labelled reagent previously employed.

### Solvates

As discussed above, the compounds disclosed herein and the stereoisomers, tautomers, and isotopically labelled forms thereof or a pharmaceutically acceptable salt of any of the foregoing may exist in solvated or unsolvated forms.

The term “solvate” as used herein refers to a molecular complex comprising a compound, or a pharmaceutically acceptable salt thereof as described herein and a stoichiometric or non-stoichiometric amount of one or more pharmaceutically acceptable solvent molecules. If the solvent is water, the solvate is referred to as a “hydrate.”

Accordingly, the scope of the instant disclosure is to be understood to encompass all solvents of the compounds disclosed herein and the stereoisomers, tautomers and isotopically labelled forms thereof or a pharmaceutically acceptable salt of any of the foregoing.

### Miscellaneous Definitions

This section will define additional terms used to describe the scope of the compounds, compositions and uses disclosed herein.

The term “aryl” refers to an aromatic hydrocarbon group having 6-20 carbon atoms in the ring portion. Typically, aryl is monocyclic, bicyclic or tricyclic aryl having 6-20 carbon atoms. Furthermore, the term “aryl” as used herein, refers to an aromatic substituent which can be a single aromatic ring, or multiple aromatic rings that are fused together. Non-limiting examples include phenyl, naphthyl or tetrahydronaphthyl, each of which may optionally be substituted with 1-4 substituents, such as alkyl, trifluoromethyl, cycloalkyl, halogen, hydroxy, alkoxy, acyl, alkyl-C(O)—O—, aryl-O—, heteroaryl-O—, amino, thiol, alkyl-S—, aryl-S— nitro, cyano, carboxy, alkyl-O—C(O)—, carbamoyl, alkyl-S(O)—, sulfonyl, sulfonamido, phenyl, and heterocyclyl.

The terms “C₁₋₄alkyl,” and “C₁₋₆alkyl” as used herein refer to a straight or branched chain hydrocarbon containing from 1 to 4, and 1 to 6 carbon atoms, respectively. Representative examples of C₁₋₄alkyl or C₁₋₆alkyl include, but are not limited to, methyl, ethyl, n-propyl, iso-propyl, n-butyl, see-butyl, iso-butyl, tert-butyl, pentyl and hexyl.

The terms “C₁₋₄alkylene” and “C₄alkylene” refer to a straight or branched divalent alkyl group as defined herein containing 1 to 4, and 1 to 6 carbon atoms, respectively. Representative examples of alkylene include, but are not limited to, methylene, ethylene, n-propylene, iso-propylene, n-butylene, sec-butylene, iso-butylene, tert-butylene, n-pentylene, isopentylene, neopentylene, n-hexylene and the like.

The term “C₂₋₄alkenyl” as used herein refers to a saturated hydrocarbon containing 2 to 4 carbon atoms having at least one carbon-carbon double bond. Alkenyl groups include both straight and branched moieties. Representative examples of C₂₋₄alkenyl include, but are not limited to, 1-propenyl, 2-propenyl, 2-methyl-2-propenyl, and butenyl.

The term “C₂₋₄alkynyl” as used herein refers to a saturated hydrocarbon containing 2 to 4 carbon atoms having at least one carbon-carbon triple bond. The term includes both straight and branched moieties. Representative examples of C₃₋₆alkynl include, but are not limited to, ethynyl, 1-propynyl, 2-propynyl, 2-butynyl and 3-butynyl.

The term “C₁₋₄alkoxy” or “C₁₋₆alkoxy” as used herein refers to —OR, wherein R^{a} represents a C₁₋₄ alkyl group or C₁₋₆ alkyl group, respectively, as defined herein. Representative examples of C₁₋₄alkoxy include, but are not limited to, methoxy, ethoxy, propoxy, iso-propoxy, and butoxy. Representative examples of C₁₋₆alkoxy include, but are not limited to, ethoxy, propoxy, iso-propoxy, and butoxy.

The term “C₃₋₈cycloalkyl” as used herein refers to a saturated carbocyclic molecule wherein the cyclic framework has 3 to 8 carbons. Representative examples of C₃₋₈cycloalkyl include, but are not limited to, cyclopropyl and cyclobutyl.

The term “deutero” as used herein as a prefix to another term for a chemical group refers to a modification of the chemical group, wherein one or more hydrogen atoms are substituted with deuterium (“D” or “²H”). For example, the term “C₁₋₄deuteroalkyl” refers to a C₁₋₄alkyl as defined herein, wherein one or more hydrogen atoms are substituted with D. Representative examples of C₁₋₄deuteroalkyl include, but are not limited to, —CH₂D, —CHD₂, —CD₃, —CH₂CD₃, —CDHCD₃, —CD₂CD₃, —CH(CD₃)₂, —CD(CHD₂)₂, and —CH(CH₂D)(CD₃).

The term “halogen” as used herein refers to —F, —Cl, —Br, or —I.

The term “halo” as used herein as a prefix to another term for a chemical group refers to a modification of the chemical group, wherein one or more hydrogen atoms are substituted with a halogen as defined herein. The halogen is independently selected at each occurrence. For example, the term “C₁₋₄haloalkyl” refers to a C₁₋₄alkyl as defined herein, wherein one or more hydrogen atoms are substituted with a halogen. Representative examples of C₁₋₄haloalkyl include, but are not limited to: —CH₂F, —CHF₂, —CF, —CHFCl, —CH₂CF₃, —CFHCF₃, —CF₂CF₃, —CH(CF₃)₂, —CF(CHF₂)₂, and —CH(CH₂F)(CF₃).

As used herein, the term “heteroaryl” refers to a 5-20 membered monocyclic- or bicyclic- or tricyclic-aromatic ring system, having 1 to 8 heteroatoms selected from N, O and S. In certain preferred aspects, the heteroaryl is a 5-10 membered ring system (e.g., 5-7 membered monocycle, an 8-10 membered bicycle or a 11-14 membered tricycle) or a 5-7 membered ring system. Exemplary monocyclic heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, and 2-, 4-, and 5-pyrimidinyl. Exemplary bicyclic heteroaryl groups include 1-, 3-, 4-, 5-, 6-, 7-, or 8-isoquinolinyl, 2-, 3-, 4-, 5-, 6-, 7-, or 8-quinolinyl, 1-, 3-, 4-, 5-, 6-, 7-, or 8-isoquinolinyl, 1-, 2-, 4-, 5-, 6-, 7-, or 8-benzimidazolyl and 1-, 2-, 3-, 4-, 5-, 6-, 7-, or 8-indolyl.

The term “heteroaryl” also refers to a group in which a heteroaromatic ring is fused to one or more aryl, cycloaliphatic, or heterocyclyl rings.

As used herein, the term “heterocycle,” “heterocycloalkyl” or “heterocyclo” refers to a saturated or unsaturated non-aromatic ring or ring system, e.g., which is a 4-, 5-, 6-, or 7-membered monocyclic, 7-, 8-, 9-, 10-, 11-, or 12-membered bicyclic or 10-, 11-, 12-, 13-, 14- or 15-membered tricyclic ring system and contains at least one heteroatom selected from O, S and N, where the N and S can also optionally be oxidized to various oxidation states. The heterocyclic group can be attached to a heteroatom or a carbon atom. The heterocyclyl can include fused or bridged rings as well as spirocyclic rings. Examples of heterocycles include tetrahydrofuran, dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, azetidine, thiazolidine, morpholine, and the like.

The term “pharmaceutically acceptable” as used herein refers to generally recognized for use in subjects, particularly in humans.

The term “pharmaceutically acceptable salt” as used herein refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, and the like; or (2) salts formed when an acidic proton present in the parent compound either is replaced by a metal ion, for example, an alkali metal ion, an alkaline earth ion, or an aluminum ion; or coordinates with an organic base such as ethanolamine, diethanolamine, triethanolamine, N-methylglucamine, dicyclohexylamine, and the like. Additional examples of such salts can be found in Berge et al., J. Pharm. Sci. 66(1):1-19 (1977). See also Stahl et al., Pharmaceutical Salts: Properties, Selection, and Use, 2^{nd} Revised Edition (2011).

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherents, glidants, disintegrants, flavoring agents, coatings, binders, sweeteners, lubricants, sorbents, preservatives, and the like.

The term “subject” as used herein refers to humans and mammals, including, but not limited to, primates, cows, sheep, goats, horses, dogs, cats, rabbits, rats, and mice. In one embodiment the subject is a human.

The term “therapeutically effective amount” as used herein refers to that amount of a compound disclosed herein that will elicit the biological or medical response of a tissue, a system, or subject that is being sought by a researcher, veterinarian, medical doctor or other clinician.

### General Synthetic Procedures

The compounds provided herein can be synthesized according to the procedures described in this and the following sections. The synthetic methods described herein are merely exemplary, and the compounds disclosed herein may also be synthesized by alternate routes utilizing alternative synthetic strategies, as appreciated by persons of ordinary skill in the art. It should be appreciated that the general synthetic procedures and specific examples provided herein are illustrative only and should not be construed as limiting the scope of the present disclosure in any manner.

Generally, the compounds of Formula I can be synthesized according to the following schemes. Any variables used in the following schemes are the variables as defined for Formula I, unless otherwise noted. All starting materials are either commercially available, for example, from Merck Sigma-Aldrich Inc., Fluorochem Ltd, and Enamine Ltd. or known in the art and may be synthesized by employing known procedures using ordinary skill. Starting material may also be synthesized via the procedures disclosed herein. Suitable reaction conditions, such as, solvent, reaction temperature, and reagents, for the Schemes discussed in this section, may be found in the examples provided herein.

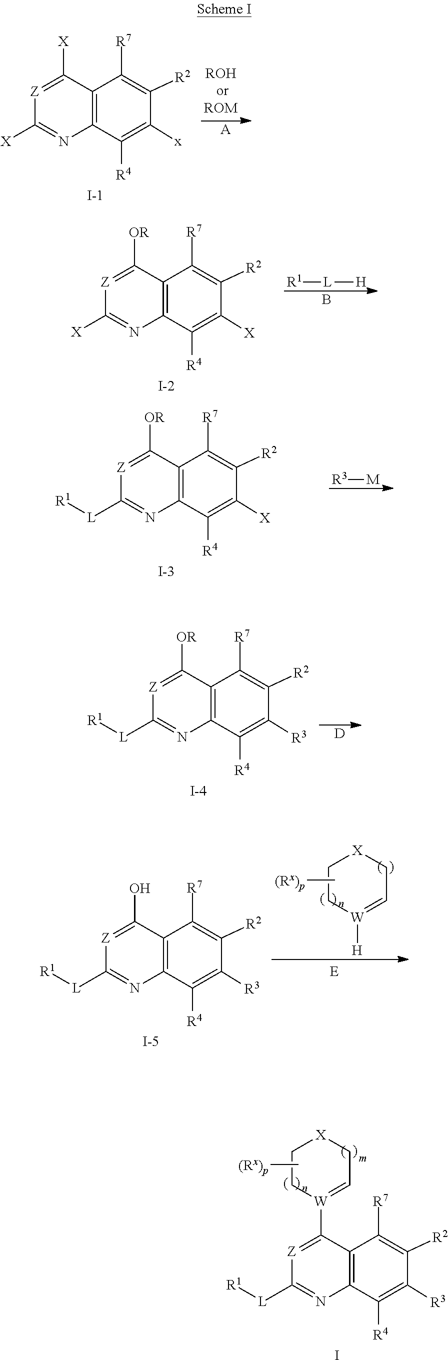

Compounds of Formula (I) can be prepared according to Scheme I. In step A, compound (I-1) is treated with an aliphatic alcohol, such as benzyl alcohol, and a base, such as Hunig's base, or metal alkoxide, such as potassium tert-butoxide, in a solvent such as 1,4-dioxane to give compound (1-2). In step B, compound (1-2) undergoes S_{N}Ar reaction with a nucleophile having the formula R¹-L-H in a solvent such as acetonitrile, in the presence of a base such as Hunig's base, to give compound (1-3). In step C, compound (I-3) is coupled with an organometallic reagent or a boronic acid (ester) to provide compound (I-4). This coupling reaction proceeds in a solvent or mixture of solvents such as 1,4-dioxane and water, and a catalyst such as cataCXium A Pd G3, with or without a base such as potassium phosphate. In step D, compound (1-4) is treated with a suitable set of reagents, such as Pd/C with H₂ to remove the alkyl group R, giving compound (I-5). In Step E, compound (I-5) is treated with an optionally substituted cyclic amine in the presence of coupling reagent such as HATU, and a base such as Hunig's base, in a solvent such as DMA to give compounds of Formula (I). In some cases, the species R³ will contain protecting group(s), which can be removed in step D or after step E in the synthetic sequence.

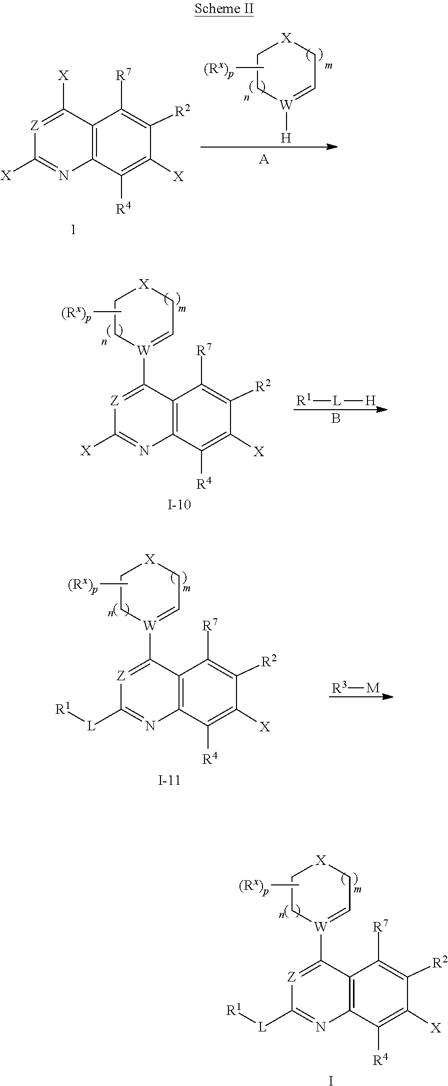

Compounds of Formula (I) can also be prepared according to Scheme 11. In step A, compound (1) undergoes S_{N}Ar reaction with an optionally substituted cyclic amine in a solvent such as dichloromethane and in the presence of a base such as Hunig's base to give compound (I-10). In step B, compound (I-10) undergoes S_{N}Ar reaction with a nucleophile having the formula R¹-L-H in a solvent such as acetonitrile, in the presence of a base such as Hunig's base to give compound (1-11). In step C, compound (1-11) is coupled with an organometallic reagent or a boronic acid (ester) to provide compounds of formula (I). This coupling reaction proceeds in a solvent or mixture of solvents such as 1,4-dioxane and water, and a catalyst such as cataCXium A Pd G3, with or without a base such as potassium phosphate. In some cases, group R² will undergo further transformations. In some cases, the species R³ will contain protecting group(s), which can be removed after step C in the synthetic sequence.

### EXAMPLES

This section provides specific examples of compounds of Formula I and methods of making the same.

### List of Abbreviations

TABLE 1

| | |
|---|---|
| Ac | acetyl |
| AcOH | acetic acid |
| aq or aq. | aqueous |
| Bn | benzyl |
| B₂pin₂ | bis(pinacolato)diboron |
| BOC or Boc | tert-butyloxycarbonyl |
| BrettPhos Pd G4 | (SP-4-3)-[dicyclohexyl[3,6-dimethoxy-2′,4′,6′-tris(]- |
| | methylethyl)[1,1′-biphenyl]-2-yl]phosphine- |
| | κP](methanesulfonato-κO)[2′-(methylamino-κN)[1,1′- |
| | biphenyl]-2-yl-κC]palladium |
| cataCXium A Pd G3 | mesylate[(di(1-adamantyl)-n-butylphosphine)-2-(2′- |
| | amino-1,1′-biphenyl)]palladium(II) |
| COD or cod | 1,5-cyclooctadiene |
| DABCO | 1,4-diazabicyclo[2.2.2]octane |
| DBU | 1,8-diazabicyclo[5.4.0]undec-7-ene |
| DCM | dichloromethane |
| DEA | diethylamine |
| DMA | N,N-dimethylacetamide |
| DMF | N,N-dimethylformamide |
| DMSO | dimethyl sulfoxide |
| Dppf, DPPF or dppf | 1, 1′-bis(diphenylphosphino)ferrocene |
| dtbbpy | 4,4′-di-tert-butyl-2,2′-dipyridyl |
| eq or eq. or equiv. | equivalent |
| ESI or ES | electrospray ionization |
| Et | ethyl |
| EtOAc | ethyl acetate |
| g | gram(s) |
| h | hour(s) |
| HATU | 1-[bis(dimethylamino)methylene]-1H-1,2,3- |
| | triazolo[4,5-b]pyridinium 3-oxide hexafluorophosphate |
| HBpin | 4,4,5,5-tetramethyl-1,3,2-dioxaborolane |
| HMPÅ | hexamethylphosphoramide |
| HOAc | acetic acid |
| HPLC | high pressure liquid chromatography |
| iPr | iso-propyl |
| iPr₂NEt or DIPEA | N-ethyl diisopropylamine (Hunig′s base) |
| KOAc | potassium acetate |
| LC MS, LCMS, | liquid chromatography mass spectroscopy |
| LC-MS or LC/MS | |
| LHMDS or LiHMDS | lithium hexamethyldisilazide |
| m/z | mass divided by charge |
| Me | methyl |
| MeCN | acetonitrile |
| MeOH | methanol |
| mg | milligrams |
| min | minutes |
| mL | milliliters |
| MS | mass spectra |
| NMR | nuclear magnetic resonance |
| Pd(dppf)Cl₂•DCM, | [1,1′-bis(diphenylphosphino)ferrocene] |
| Pd(dppf)Cl₂ | dichloropalladium(II), complex with dichloromethane |
| Pd(dtbpf)Cl₂ | [1,1′-bis(di-tert-butylphosphino)ferrocene] |
| | dichloropalladium(II) |
| Pd(PPh₃)₄ | tetrakis(triphenylphosphine)palladium(0) |
| Ph | phenyl |
| PhMe | toluene |
| pin | pinacolato |
| PMB | 4-methoxybenzyl |
| rbf | round-bottom flask |
| RP-HPLC | reverse phase high pressure liquid chromatography |
| RT or rtor r.t. | room temperature |
| sat. or satd. | saturated |
| SFC | supercritical fluid chromatography |
| SPhos Pd G4 | (SP-4-3)-[dicyclohexyl(2′,6′-dimethoxy[1,1′-biphenyl]- |
| | 2-yl)phosphine-κP](methanesulfonato-κO)[2′- |
| | (methylamino-κN)[1,1′-biphenyl]-2-yl-κC]palladium |
| TBAF | tetra-n-butylammonium fluoride |
| TBDPS | tert-butyldiphenylsilyl |
| TBDPSCl | tert-butyldiphenylsilyl chloride |
| tBu | tert-butyl |
| tBuXPhos Pd G3 | [(2-di-tert-butylphosphino-2′,4′,6′-triisopropyl-1,1′- |
| | biphenyl)-2-(2′-amino-1,1′-biphenyl)]palladium(II) |
| | methanesulfonate |
| TC | thiophene-2-carboxylate |
| TEA or Et₃N | triethylamine |
| Tf | trifluoromethanesulfonate |
| TFA | trifluoroacetic acid |
| THF | tetrahydrofuran |
| TIPS | triisopropylsilyl |
| UV | ultraviolet |
| XantPhos Pd G3 | [(4,5-bis(diphenylphosphino)-9,9-dimethylxanthene)- |
| | 2-(2′-amino-1,1′-biphenyl)]palladium(II) |
| | methanesulfonate |
| XPhos | 2-dicyclohexylphosphino-2′,4′,6′-triisopropyl-1,1′- |
| | biphenyl |
| XPhos Pd G3 | (2-dicyclohexylphosphino-2′,4′,6′-triisopropyl-1,1′- |
| | biphenyl)[2-(2′-amino-1,1′-biphenyl)]palladium(II) |
| | methanesulfonate |

### General Analytical and Purification Methods

Provided in this section are descriptions of the general analytical and purification methods used to prepare the specific examples provided herein.
Chromatography: Unless otherwise indicated, crude product-containing residues were purified by passing the crude material or concentrate through either a Biotage or ISCO brand silica gel column pre-packed with flash silica (SiO₂) and eluting the product from the column with a solvent gradient as indicated.
Preparative HPLC Method: Where indicated, the compounds described herein were purified via reverse phase HPLC using Waters FractionLynx or Gilson semi-preparative HPLC-MS system utilizing one of the following two HPLC columns: (a) Phenomenex Gemini column (5 micron, C18, 150×30 mm) or (b) Waters X-select CSH column (5 micron, C18, 100×30 mm). A typical run through the instrument included: eluting at 45 mL/min with a linear gradient of 10% (v/v) to 100% MeCN (0.1% v/v formic acid) in water (0.1% formic acid) over 10 minutes; conditions can be varied to achieve optimal separations.
Proton NMR Spectra: Unless otherwise indicated, all ¹H NMR spectra were collected on a Bruker NMR instrument at 300,400 or 500 MHz. All observed protons are reported as parts-per-million (ppm) downfield from tetramethylsilane (TMS) using the internal solvent peak as reference. Some ¹H signals may be missing due to exchange with D from MeOD, or due to signal suppression.
Mass Spectra (MS): Unless otherwise indicated, all mass spectral data for starting materials, intermediates and/or exemplary compounds are reported as mass/charge (m/z), having an [M+H]+ molecular ion. The molecular ion reported was obtained by electrospray detection method (commonly referred to as an ESI MS) utilizing a Waters Acquity UPLC/MS system. Compounds having an isotopic atom, such as bromine and the like, are generally reported according to the detected isotopic pattern, as appreciated by those skilled in the art.

### Preparation of Intermediates

### 6-Methyl-1,4-oxazepan-6-ol hydrochloride Isomer 1 (Intermediate A1) and Isomer 2 (Intermediate A2)

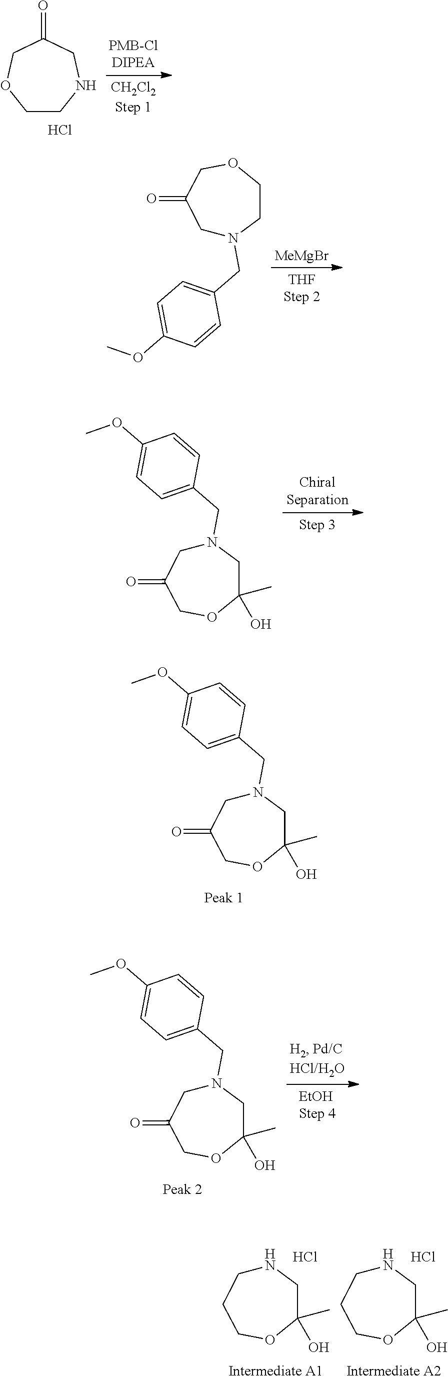

Step 1: 4-(4-Methoxybenzyl)-1,4-oxazepan-6-one. To a 20-mL vial was added 1,4-oxazepan-6-one hydrochloride (0.30 g, 1.98 mmol, AA BLOCKS LLC), 4-methoxybenzyl chloride (0.37 g, 0.32 mL, 2.38 mmol, TCI America), DIPEA (0.77 g, 1.0 mL, 5.94 mmol. Sigma-Aldrich Corporation) and DCM (10 mL). The reaction was stirred at rt overnight. The crude material was purified by column chromatography on a silica gel column, eluting with a gradient of 0-80% 3:1 EtOAc/EtOH (with 1% TEA) in heptane to provide 4-(4-methoxybenzyl)-1,4-oxazepan-6-one (0.43 g, 1.83 mmol, 92% yield) as colorless oil. m/z (ESI): 236.2 (M+H)⁺.

Step 2: 4-(4-Methoxybenzyl)-6-methyl-1,4-oxazepan-6-ol. To a 100-mL round-bottomed flask was added 4-(4-methoxybenzyl)-1,4-oxazepan-6-one (0.87 g, 3.70 mmol) in THF (15 mL). The mixture was cooled to 0° C. before methylmagnesium bromide solution (3 M in Et₂O, 3.7 mL, 11.09 mmol, Sigma-Aldrich Corporation) was added. The reaction was stirred for 1 h. The reaction mixture was diluted with saturated NH₄Cl (15 mL) and extracted with EtOAc (2×15 mL). The organic extract was washed with saturated NaCl (15 mL) and dried over MgSO₄. The solution was filtered and concentrated in vacuo to give the crude material. The crude material was purified by column chromatography on silica gel, eluting with a gradient of 0-50% 3:1 EtOAc/EtOH in heptane to provide 4-(4-methoxybenzyl)-6-methyl-1,4-oxazepan-6-ol (0.65 g, 2.59 mmol, 70% yield) as yellow oil. m/z (ESI): 252.1 (M+H)⁺.

Step 3: Chiral separation, 4-(4-Methoxybenzyl)-6-methyl-1,4-oxazepan-6-ol (0.65 g, 2.59 mmol) was purified via SFC using a Chiralpak AD, 30×150 mm, 5 μm, column with a mobile phase of 20% methanol with 0.2% triethylamine using a flowrate of 200 mL/min to generate 246 mg of peak 1 with an ee of >99% and 292 mg of peak 2 with an ee of >99%.

Step 4: 6-Methyl-1,4-oxazepan-6-ol hydrochloride, 4-(4-methoxybenzyl)-6-methyl-1,4-oxazepan-6-ol (0.24 g, 0.96 mmol, Peak 1) was dissolved in ethanol (5.8 mL). Palladium on activated carbon (0.25 g, 0.23 mmol, Sigma-Aldrich Corporation) and aqueous HCl solution (2 N, 0.7 mL, 1.33 mmol, Sigma-Aldrich Corporation) were added and the mixture stirred at it under an atmosphere of H₂ for 5 h. The catalyst was removed, and the solution was concentrated to provide 6-methyl-1,4-oxazepan-6-ol hydrochloride (quant. yield, isomer 1, Intermediate A1). Isomer 2, Intermediate A2 was obtained by the same method.

### 6-Fluoro-4-(4,4,5,5-tetramethyl-1,3,2-dioxaborolan-2-yl)-5-((triisopropylsilyl)ethynyl)naphthalen-2-ol (Intermediate B1)

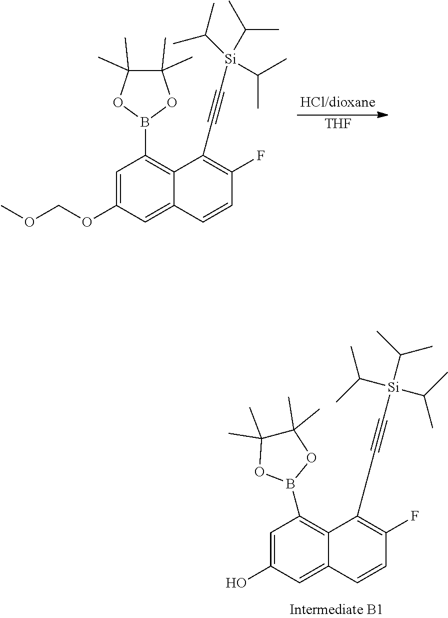

((2-Fluoro-6-(methoxymethoxy)-8-(4,4,5,5-tetramethyl-1,3,2-dioxaborolan-2-yl)naphthalen-1-yl)ethynyl)triisopropylsilane (1.00 g, 1.95 mmol, LabNetwork) was dissolved in tetrahydrofuran (4 mL). HCl (4 M in dioxane, 0.71 g, 0.71 mL, 19.51 mmol, Sigma-Aldrich Corporation) was added. The reaction mixture was stirred at rt for 5 h and then the volatiles were removed in vacuo. The residue was purified by column chromatography on silica gel, eluting with a gradient of 0-50% EtOAc/heptane to yield 6-fluoro-4-(4,4,5,5-tetramethyl-1,3,2-dioxaborolan-2-yl)-5-((triisopropylsilyl)ethynyl)naphthalen-2-ol (0.89 g, 1.90 mmol, 97% yield). m/z (ESI): 469.0 (M+H)⁺.

TABLE 2
Additional Intermediates. Prepared in an Analogous Manner to Intermediate B1.

| | | | | MS |
|---|---|---|---|---|
| | | | | m/z |
| | | | | (ESI): |
| Intermediate # | Structure | Name | Reagent | (M + H)⁺ |
| B2 | 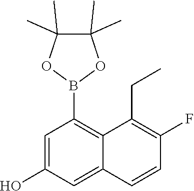 [SMILES: CCc1c(F)ccc2cc(O)cc(B3OC(C)(C)C(C)(C)O3)c12] | 5-Ethyl-6-fluoro-4- (4,4,5,5-tetramethyl- 1,3,2-dioxaborolan- 2-yl)naphthalen-2-ol | 2-(8-ethyl-7-fluoro-3- (methoxymethoxy) naphthalen-1-y1)-4,4, 5,5-tetramethyl-1,3,2- dioxaborolane (CAS#: 2621932-48-5, PharmaBlock) | 317.2 |
| B3 | 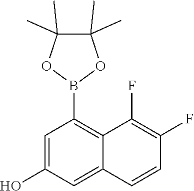 [SMILES: CC1(C)OB(c2cc(O)cc3ccc(F)c(F)c23)OC1(C)C] | 5,6-Difluoro-4- (4,4,5,5-tetramethyl- 1,3,2-dioxaborolan- 2-yl)naphthalen-2-ol | 2-(7,8-difluoro-3- (methoxymethoxy) naphthalen-1-y1)-4,4, 5,5-tetramethyl-1,3,2- dioxaborolane (CAS#: 2621935-35-9, LabNetwork) | 307.0 |
| B4 | 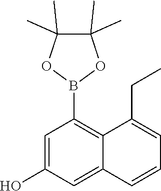 [SMILES: CCc1cccc2cc(O)cc(B3OC(C)(C)C(C)(C)O3)c12] | 5-Ethyl-4-(4,4,5,5- tetramethyl-1,3,2- dioxaborolan-2- yl)naphthalen-2-ol | 2-(8-Ethyl-3- (methoxymethoxy) naphthalen-1-y1)-4,4, 5,5-tetramethyl-1,3,2- dioxaborolane (CAS#: 2621932-60-1, LabNetwork) | 299.2 |

### 7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-ol (Intermediate C)

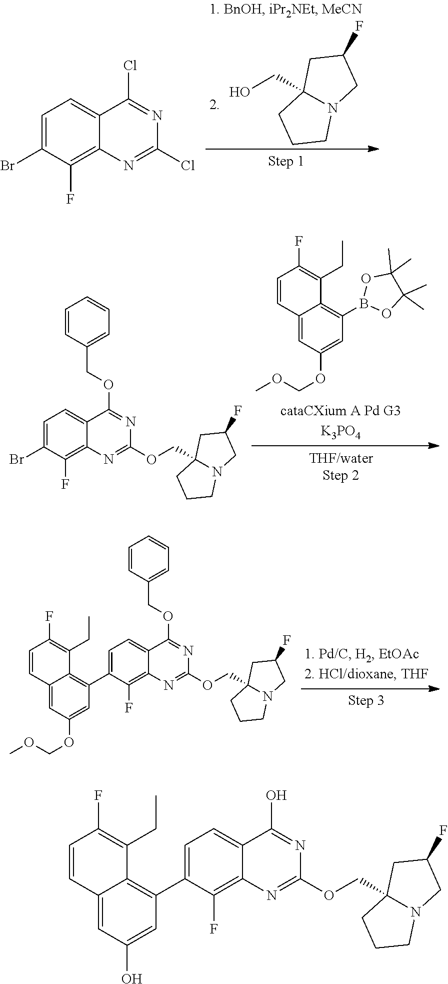

Step 1: 4-(Benzyloxy)-7-bromo-8-fluoro-2-(((2R,7aS)-2-fluorotetrahydro-1H-pyrrolizin-7a(5H)-yl)methoxy)quinazoline, 7-Bromo-2,4-dichloro-8-fluoroquinazoline (92 mg, 0.31 mmol, PharmaBlock) was dissolved in acetonitrile (0.6 mL) and benzyl alcohol (0.34 g, 0.3 mL, 3.10 mmol) and DIPEA (0.12 g, 0.2 mL, 0.93 mmol) were added. The mixture was stirred at rt for 2 h. The crude mixture was directly purified by column chromatography on silica gel, eluting with a gradient of 0-100% 3:1 EtOAc:EtOH in heptanes. The product was redissolved in acetonitrile (0.6 mL), ((2R,7aS)-2-fluorohexahydro-1H-pyro-7a-yl)methanol (49 mg, 0.31 mmol, LabNetwork) and DIPEA (0.12 g, 0.2 mL, 0.93 mmol) was added. The mixture was stirred at 80° C. overnight. The crude mixture was purified via reverse phase HPLC to yield 4-(benzyloxy)-7-bromo-8-fluoro-2-(((2R,7aS)-2-fluorotetrahydro-1H-pyrrolizin-7a(5H)-yl)methoxy)quinazoline (0.13 g, 0.27 mmol, 85% yield) as colorless oil. m/z (ESI): 491.0 (M+H)⁺.

Step 2: 4-(Benzyloxy)-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazoline. To a solution of 4-(benzyloxy)-7-bromo-8-fluoro-2-(((2R,7aS)-2-fluorotetrahydro-1H-pyrrolizin-7a(5H)-yl)methoxy)quinazoline (0.14 g, 0.29 mmol) and 2-(8-ethyl-7-fluoro-3-(methoxymethoxy)naphthalen-1-yl)-4,4,5,5-tetramethyl-1,3,2-dioxaborolane (0.21 g, 0.59 mmol, PharmaBlock) in degassed tetrahydrofuran (2.7 mL) and water (0.3 mL) were added potassium phosphate (0.19 g, 0.88 mmol) and cataCXium A Pd G3 (43 mg, 0.06 mmol). The reaction mixture was stirred at 70° C. overnight. The crude mixture was purified via reverse phase HPLC to yield 4-(benzyloxy)-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazoline (0.20 g, 0.30 mmol, quant.) as yellow oil, which was used in the next step without further manipulation. m/z (EST): 644.0 (M+H)⁺.

Step 3: 7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-ol. 4-(Benzyloxy)-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazoline (0.20 g, 0.30 mmol) was dissolved in EtOAc (3.0 mL) in a pressure tube with palladium on activated carbon (63 mg, 0.06 mmol). The reaction was stirred at rt under 15 psi of H₂ overnight. The mixture was filtered over celite. The filtrate was concentrated under reduced pressure. The residue was redissolved in THF (3.0 mL) and HCl solution (4 M in 1,4-dioxane, 2.0 mL). The reaction was stirred at rt for 3 h. Volatiles were removed in vacuo and co-evaporated with DCM (3×5 mL) to yield 7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-ol (0.14 g, 0.27 mmol, 90% yield) as yellow solid that was used without further purification. m/z (EST): 510.1 (M+H)⁺.

### 7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-ol (Intermediate D)

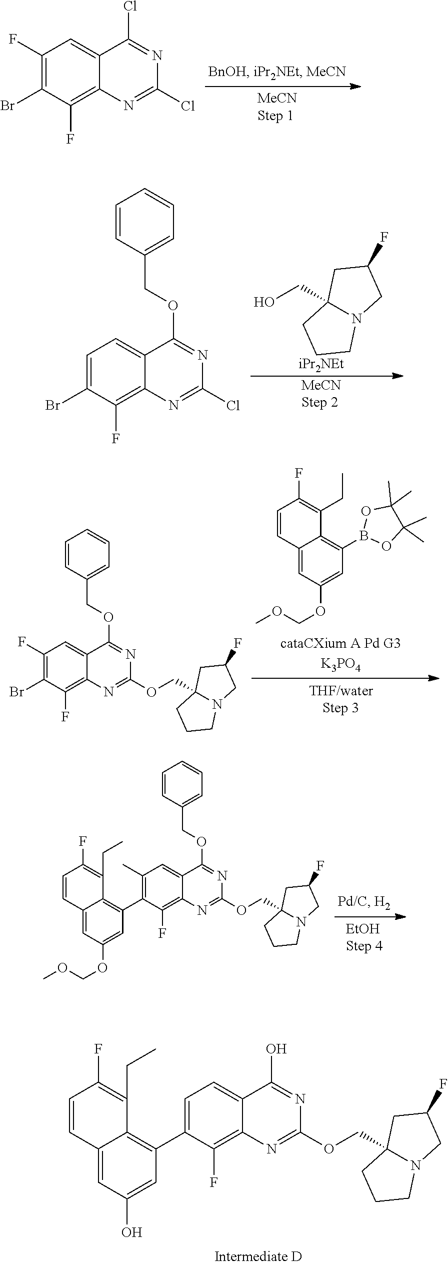

Step 1: 4-(Benzyloxy)-7-bromo-2-chloro-6,8-difluoroquinazoline. 7-Bromo-2,4-dichloro-6,8-difluoroquinazoline (1.00 g, 3.19 mmol, PharmaBlock) was dissolved in acetonitrile (12 mL) and benzyl alcohol (3.44 g, 3.3 mL, 31.9 mmol) and DIPEA (1.24 g, 1.7 mL 9.56 mmol) were added. The mixture was stirred at 35° C. for 2 h. The crude mixture was purified by column chromatography, eluting with a gradient of 0-30% EtOAc in heptanes to yield 4-(benzyloxy)-7-bromo-2-chloro-6,8-difluoroquinazoline (0.30 g, 0.78 mmol, 24% yield). m/z (ESI): 384.8 (M+H)⁺.

Step 2: 4-(Benzyloxy)-7-bromo-6,8-difluoro-2-(((2R,7aS)-2-fluorotetrahydro-1H-pyrrolizin-7a(5H)-yl)methoxy)quinazoline. 4-(Benzyloxy)-7-bromo-2-chloro-6,8-difluoroquinazolin (0.30 g, 0.77 mmol) was dissolved in acetonitrile (1.5 mL) and ((2R,7aS)-2-fluorotetrahydro-1H-pyrrolizin-7a(5H)-yl)methanol (0.12 g, 0.77 mmol) and DIPEA (0.30 g, 0.4 mL, 2.30 mmol) were added. The mixture was stirred at 80° C. for 24 h. The mixture purified via reverse phase HPLC to yield 4-(benzyloxy)-7-bromo-6,8-difluoro-2-(((2R,7aS)-2-fluorotetrahydro-1H-pyrrolizin-7a(5H)-yl)methoxy)quinazoline (25 mg, 0.05 mmol, 6% yield). m/z (ESI): 508.1 (M+H)⁺.

Step 3: 4-(4-(Benzyloxy)-6,8-difluoro-2-(((2R,7aS)-2-fluorotetrahydro-1H-pyrrolizin-7a(5H)-yl)methoxy)quinazolin-7-yl-5-ethyl-6-fluoronaphthalen-2-ol. 4-(Benzyloxy)-7-bromo-6.8-difluoro-2-(((2R,7aS)-2-fluorotetrahydro-1H-pyrrolizin-7a(5H)-yl)methoxy)quinazoline (50 mg, 0.10 mmol), cataCXium A Pd G3 (14 mg, 0.02 mmol), 5-ethyl-6-fluoro-4-(4,4,5,5-tetramethyl-1,3,2-dioxaborolan-2-yl)naphthalen-2-ol (62 mg, 0.20 mmol) and potassium phosphate monohydrate (68 mg, 0.30 mmol) were dissolved in tetrahydrofuran (0.9 mL) and water (90 μL) and degassed for 10 min. The mixture was stirred at 70° C. overnight. The crude mixture was then purified via reverse phase HPLC to yield 4-(4-(benzyloxy)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol (30 mg, 0.05 mmol, 49% yield). m/z (ESI) 618.0 (M+H)⁺.

Step 4: 7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-ol. 4-(4-(Benzyloxy)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol (30 mg, 0.05 mmol) was dissolved in EtOH (1.5 mL). Palladium on activated carbon (21 mg, 0.02 mmol) was added and the mixture was stirred under 50 psi of H₂ for 16 h. The reaction was filtered, and volatiles were removed in vacuo to yield 7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-ol (25 mg, 0.05 mmol, quant.) as colorless glass that was used without further purification. m/z (ESI): 528.0 (M+H)⁺.

### (R)-1-Oxa-3,7-diazaspiro[4.5]decan-2-one (Intermediate H)

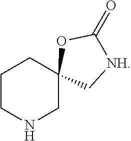 [SMILES: O=C1NC[C@]2(CCCNC2)O1]

Step 1: Benzyl (S)-2-oxo-1-oxa-3,7-diazaspiro[4.5]decane-7-carboxylate. A mixture of 1-oxa-3,7-diazaspiro[4.5]decan-2-one hydrochloride (1.48 g, 7.66 mmol, J&W Pharmlab), benzyl carbonochloridate (1.3 mL, 9.2 mmol), and sodium bicarbonate solution (1 N, 23 mL, 23 mmol) in 2-methyltetrahydrofuran (22 mL) was stirred at rt for 16 h. The reaction mixture was extracted with EtOAc and the organic layer was washed with brine, separated, dried over anhydrous Na₂SO₄, and concentrated in vacuo to give benzyl 2-oxo-1-oxa-3,7-diazaspiro[4.5]decane-7-carboxylate (2.16 g, 7.44 mmol, 97% yield). 1.07 g was purified via SFC using a ChiralPak IC, 2×15 cm, 5 μm column with a mobile phase of 40% MeOH with 0.2% DEA using a flowrate of 100 mL/min. to generate 484 mg of peak 1 as benzyl (R)-2-oxo-1-oxa-3,7-diazaspiro[4.5]decane-7-carboxylate with an cc of 99% and 536 ng of peak 2 as benzyl (S)-2-oxo-1-oxa-3,7-diazaspiro[4.5]decane-7-carboxylate with an ee of 99%

Step 2: (R)-1-Oxa-3,7-diazaspiro[4.5]decan-2-one. A mixture of benzyl (S)-2-oxo-1-oxa-3,7-diazaspiro[4.5]decane-7-carboxylate (0.58 g, 1.99 mmol), formic acid, ammonia salt (0.63 g, 9.94 mmol), and palladium, 10%, wet (0.64 g, 0.60 mmol) in ethyl acetate (5 mL) was stirred at rt for 16 h. The reaction mixture was filtered through Celite, washed with EtOAc and EtOH. The filtrate was concentrated in vacuo to give (S)-1-oxa-3,7-diazaspiro[4.5]decan-2-one (0.26 g, 1.66 mmol, 83% yield) as white solid. m/z (ESI): 157.1 (M+H)⁺.

### EXAMPLES

### (R)-1-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol (Example 1)

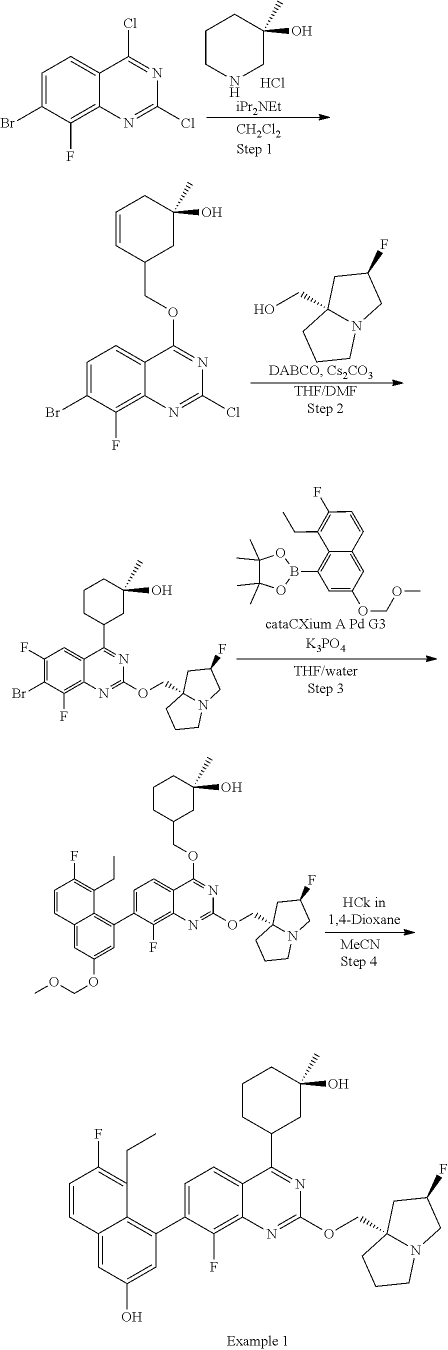

Step 1: (R)-1-(7-Bromo-2-chloro-8-fluoroquinazolin-4-yl)-3-methylpiperidin-3-ol. To a stirred solution of 7-bromo-2,4-dichloro-8-fluoroquinazoline (2.00 g, 6.76 mmol, Enamine) in acetonitrile (34 mL) was added (3R)-3-methylpiperidin-3-ol hydrochloride (1.08 g, 7.10 mmol. PharmaBlock) and DIPEA (2.62 g, 3.54 mL, 20.28 mmol, Sigma-Aldrich Corporation). The reaction was stirred at rt overnight. The reaction was then diluted with water (1.5 mL) and brine (1.5 mL) and the aqueous layer was extracted with CH₂Cl₂. The combined organic layers were dried with anhydrous sodium sulfate, filtered and concentrated under reduced pressure to provide (R)-1-(7-bromo-2-chloro-8-fluoroquinazolin-4-yl)-3-methylpiperidin-3-ol as orange solid. The product was used without further purification. m/z (ESI): 374.0 (M+H)⁺.

Step 2: (R)-1-(7-Bromo-8-fluoro-2-(((2R,7aS)-2-fluorotetrahydro-1H-pyrrolizin-7a(5H)-yl)methoxy)quinazolin-4-yl)-3-methylpiperidin-3-ol. To a 250-mL vial was added 1,4-diazabicyclo[2.2.2]octane (0.16 g, 1.44 mmol, Sigma-Aldrich Corporation), cesium carbonate (7.04 g, 21.62 mmol, Sigma-Aldrich Corporation), (R)-1-(7-bromo-2-chloro-8-fluoroquinazolin-4-yl)-3-methylpiperidin-3-ol from Step 1 and ((2R,7aS)-2-fluorotetrahydro-1H-pyrrolizin-7a(5H)-yl)methanol (2.30 g, 14.41 mmol, LabNetwork). The solids were then suspended in N,N-dimethylformamide (24 mL) and tetrahydrofuran (48 mL). The reaction mixture was stirred at 40° C. overnight. The reaction was then diluted with water and transferred to a separatory funnel. The layers were separated, and the aqueous layer was extracted with EtOAc. The organic layers were combined, dried with sodium sulfate, filtered, and concentrated under reduced pressure. The crude reaction mixture was then purified by column chromatography on silica gel, eluting with a gradient of 0-50% 3:1 EtOAc/EtOH in heptane with 2% triethylamine to provide (R)-1-(7-bromo-8-fluoro-2-(((2R,7aS)-2-fluorotetrahydro-1H-pyrrolizin-7a(5H)-yl)methoxy)quinazolin-4-yl)-3-methylpiperidin-3-ol (2.48 g, 4.99 mmol, 69% combined yield over 2 steps). m/z (ESI): 498.0 (M+H)⁺.

Step 3: (R)-1-(7-(8-Ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol. An 8-mL vial was charged with cataCXium A Pd G4 (30 mg, 0.04 mmol, Sigma Aldrich), potassium phosphate tribasic (0.17 g, 0.80 mmol, Acros Organics), 1,3,2-dioxaborolane, 2-[8-ethyl-7-fluoro-3-(methoxymethoxy)-1-naphthalenyl]4,4,5,5-tetramethyl-1,3,2-dioxaborolane (0.15 g, 0.40 mmol, PharmaBlock), (R)-1-(7-bromo-8-fluoro-2-(((2R,7aS)-2-fluorotetrahydro-1H-pyrrolizin-7a(5H)-yl)methoxy)quinazolin-4-yl)-3-methylpiperidin-3-ol (0.10 g, 0.20 mmol), water (0.25 mL) and tetrahydrofuran (0.75 mL). The reaction was stirred at 70° C. for 1 h. The crude mixture was purified by column chromatography on silica gel, eluting with a gradient of 0-50% 3:1 EtOAc/EtOH in heptane with 2% triethylamine additive to yield (R)-1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol (77 mg, 0.12 mmol, 59% yield). m/z (ESI): 651.2 (M+H)⁺.

Step 4: (R)-1-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol. (R)-1-(7-(8-Ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol (77 mg, 0.12 mmol) was stirred in hydrogen chloride solution (4.0 M in dioxane, 0.59 mL, 2.37 mmol, Sigma-Aldrich Corporation) and methanol (0.6 mL) at rt for 1 h. Volatiles were removed under reduced pressure. The crude product was purified by reverse phase HPLC to yield (R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol as 2,2,2-trifluoroacetate and as off-white solid (79 mg, 0.11 mmol, 93% yield). m/z (EST): 607.0 (M+H)⁺. ¹H NMR (400 MHz, METHANOL-d₄) δ ppm 8.13-8.23 (m, 1H), 7.65-7.73 (m, 1H), 7.48-7.57 (m, 1H), 7.23-7.32 (m, 2H), 6.96-7.01 (m, 1H), 5.49-5.68 (m, 1H), 4.73-4.79 (m, 2H), 4.64-4.73 (m, 1H), 4.38-4.51 (m, 1H), 3.80-4.12 (m, 3H), 3.61-3.73 (m, 1H), 3.42-3.60 (m, 2H), 2.55-2.84 (m, 2H), 2.32-2.53 (m, 5H), 2.13-2.29 (m, 2H), 1.77-1.95 (m, 3H), 1.26-1.40 (m, 3H), 0.77-0.89 (m, 3H).

TABLE 3
Additional Examples 2 to 43, 76 to 85. Prepared in an Analogous Manner to Example 1.

| Ex. | | | Salt | | Method |
|---|---|---|---|---|---|
| # | Structure | Name | Form | Reagent | Change |
| 2 | 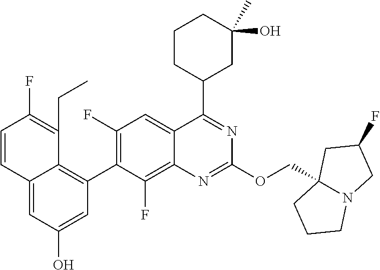 [SMILES: CCc1c(F)ccc2cc(O)cc(-c3c(F)cc4c(C5CCC[C@@](C)(O)C5)nc(OC[C@@]56CCCN5C[C@H](F)C6)nc4c3F)c12] | (3R)-1-(7-(8- Ethyl-7-fluoro-3- hydroxynaphthalen- 1-yl)-6,8- difluoro-2- (((2R,7aS)-2- fluorotetrahydro- 1H-pyrrolizin- 7a(5H)- yl)methoxy) quinazolin-4-yl)-3- methylpiperidin- 3-ol Isomer 1 | Free base | Step 1: 7- Bromo-2,4- dichloro-6,8- difluoroquinazoline (CAS#: 2248318-27-4, Enamine) | Chiral separation after final step. Details included below. |
| 3 | 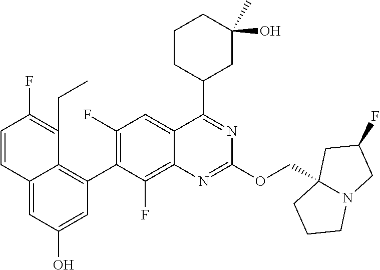 [SMILES: CCc1c(F)ccc2cc(O)cc(-c3c(F)cc4c(C5CCC[C@@](C)(O)C5)nc(OC[C@@]56CCCN5C[C@H](F)C6)nc4c3F)c12] | (3R)-1-(7-(8- Ethyl-7-fluoro-3- hydroxynaphthal en-1-yl)-6,8- difluoro-2- (((2R,7a.S)-2- fluorotetrahydro- 1H-pyrrolizin- 7a(5H)- yl)methoxy) quinazolin-4-yl)-3- methylpiperidin- 3-ol Isomer 2 | Free base | Step 1: 7- Bromo-2,4- dichloro-6,8- difluoroquinazoline (CAS#: 2248318-27-4 Enamine) | Chiral separation after final step. Details included below. |
| 4 | 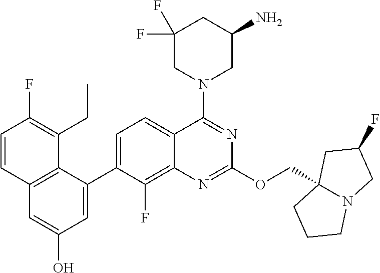 [SMILES: CCc1c(F)ccc2cc(O)cc(-c3ccc4c(N5C[C@H](N)CC(F)(F)C5)nc(OC[C@@]56CCCN5C[C@H](F)C6)nc4c3F)c12] | 4-(4-((R)-5- Amino-3,3- difluoropiperidin- 1-yl)-8-fluoro-2- (((2R,7aS)-2- fluorotetrahydro- 1H-pyrrolizin- 7a(5H)- yl)methoxy) quinazolin-7-yl)-5- etbyl-6- fluoronaphthalen- 2-ol | Bis (2,2,2- trifluoro- aceate) | Step 1: tert- Butyl (R)- (5,5- difluoropiperidin- 3- yl)carbamate (CAS#: 2089320-98-7, Advanced ChemBlocks Inc.) | Additional step after Step 2. Details included below. |
| 5 | 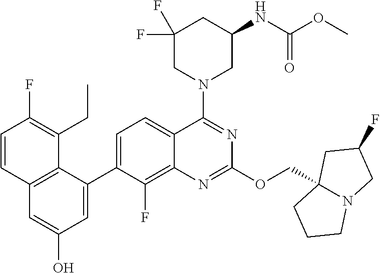 [SMILES: CCc1c(F)ccc2cc(O)cc(-c3ccc4c(N5C[C@H](NC(=O)OC)CC(F)(F)C5)nc(OC[C@@]56CCCN5C[C@H](F)C6)nc4c3F)c12] | Methyl ((R)-1-(7- (8-ethyl-7-fluoro- 3- hydroxynaphthalen- 1-yl)-8-fluoro- 2-(((2R,7aS)-2- fluorotetrahydro- 1H-pyrrolizin- 7a(5H)- yl)methoxy) quinazolin-4-yl)-5,5- difluoropiperidin- 3-yl)carbamate | Bis (2,2,2- trifluoro- aceate) te) | Step 1: tert- Butyl (R)- (5,5- difluoropiperidin- 3- yl)carbamate (CAS#: 2089320-98-7, Advanced ChemBlocks Inc.) | Additional steps after Step 2. Details included below. |
| 6 | 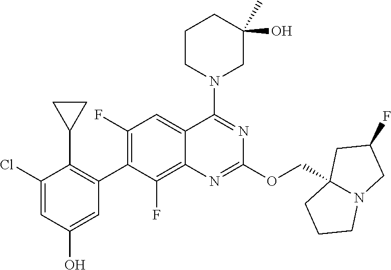 [SMILES: C[C@@]1(O)CCCN(c2nc(OC[C@@]34CCCN3C[C@H](F)C4)nc3c(F)c(-c4cc(O)cc(Cl)c4C4CC4)c(F)cc23)C1] | (3R)-1-(7-(3- Chloro-2- cyclopropyl-5- hydroxyphenyl)- 6,8-difluoro-2- (((2R,7aS)-2- fluorotetrahydro- 1H-pyrrolizin- 7a(5H)- yl)methoxy) quinazolin-4-yl)-3- methylpiperidin- 3-ol | 2,2,2- trifluoro- aceate | Step 1: 7- Bromo-2,4- dichloro-6,8- difluoroquinazoline (CAS#: 2248318-27-4, Enamine) Step 3: 2-(3- Chloro-2- cyclopropyl-5- (methoxymethoxy) phenyl)- 4,4,5,5- tetramethyl- 1,3,2- dioxaborolane (CAS# 2621936-26-1, LabNetwork) | |
| 7 | 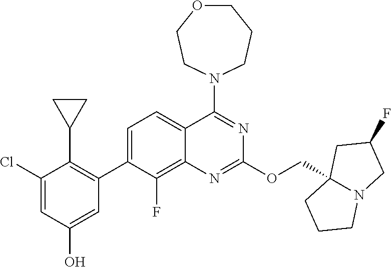 [SMILES: Oc1cc(Cl)c(C2CC2)c(-c2ccc3c(N4CCCOCC4)nc(OC[C@@]45CCCN4C[C@H](F)C5)nc3c2F)c1] | 3-Chloro-4- cyclopropyl-5-(8- fluoro-2- (((2R,7a.S)-2- fluorotetrahydro- 1H-pyrrolizin- 7a(5H)- yl)methoxy)-4- (1,4-oxazepan-4- yl)quinazolin-7- yl)phenol | Bis(2, 2,2- trifluoro- aceate) | Step 1: 1,4- Oxazepane (CAS#: 5638- 60-8, Oakwood Products, Inc.) Step 3: 2-(3- Chloro-2- cyclopropyl-5- (methoxymethoxy) phenyl)- 4,4,5,5- tetramethyl- 1,3,2- dioxaborolane (CAS#: 2621936-26-1, LabNetwork) | |
| 8 | 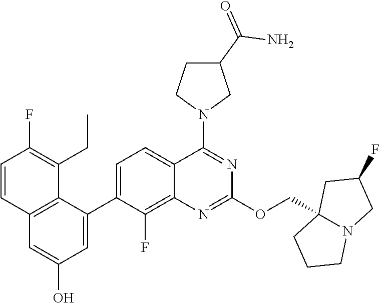 [SMILES: CCc1c(F)ccc2cc(O)cc(-c3ccc4c(N5CCC(C(N)=O)C5)nc(OC[C@@]56CCCN5C[C@H](F)C6)nc4c3F)c12] | 1-(7-(8-Ethyl-7- fluoro-3- hydroxynaphthalen- 1-yl)-8-fluoro- 2-(((2R,7aS)-2- fluorotetrahydro- 1H-pyrrolizin- 7a(5H)- yl)methoxy)quina quinazolin-4- yl)pyrrolidine-3- carboxamide | 2,2,2- trifluoro- acetate | Step 1: Pyrrolidine-3- carboxamide hydrochloride (CAS#: 644972-57-6, AChem Block) | |
| 9 | 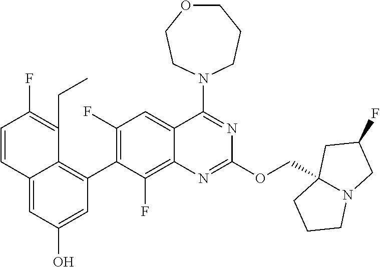 [SMILES: CCc1c(F)ccc2cc(O)cc(-c3c(F)cc4c(N5CCCOCC5)nc(OC[C@@]56CCCN5C[C@H](F)C6)nc4c3F)c12] | 4-(6,8-Difluoro- 2-(((2R,7aS)-2- fluorotetrahydro- 1H-pyrrolizin- 7a(5H)- yl)methoxy)-4- (1,4-oxazepan-4- yl)quinazolin-7- yl)-5-ethyl-6- fluoronaphthalen- 2-ol | 2,2,2- trifluoro- acetate | Step 1: 1,4 Oxazepane (CAS#: 5638- 60-8, Oakwood) and 7-bromo-2,4- dichloro-6,8- difluoroquinazoline (CAS#: 2248318-27-4, Enamine) | |
| 10 | 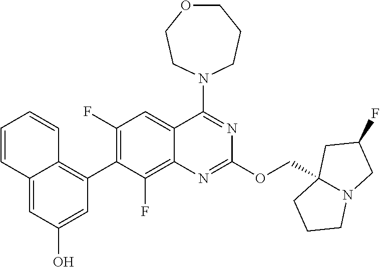 [SMILES: Oc1cc(-c2c(F)cc3c(N4CCCOCC4)nc(OC[C@@]45CCCN4C[C@H](F)C5)nc3c2F)c2ccccc2c1] | 4-(6,8-Difluoro- 2-(((2R,7aS)-2- fluorotetrahydro- 1H-pyrrolizin- 7a(5H)- yl)methoxy)-4- (1,4-oxazepan-4- yl)quinazolin-7- yl)naphthalen-2- ol | 2,2,2- trifluoro- acetate | Step 1: 1,4 Oxazepane (CAS#: 5638- 60-8, Oakwood) and 7-bromo-2,4- dichloro-6,8- difluoroquinaz oline (CAS#: 2248318-27-4, Enamine) Step 3: 4- (4,4,5,5. tetramethyl- 1,3,2- dioxaborolan- 2- yl)naphthalen- 2-ol (CAS#: 2043962-01-0, Aurum) | [1,1′- Bis(diphenyl- phosphino) ferrocene] dichloro- palladium(II) as catalyst was used in Step 3 in 1,4- dioxane/ water. No Step 4. |
| 11 | 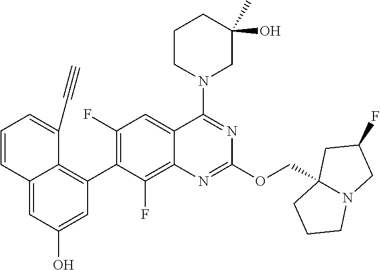 [SMILES: C#Cc1cccc2cc(O)cc(-c3c(F)cc4c(N5CCC[C@@](C)(O)C5)nc(OC[C@@]56CCCN5C[C@H](F)C6)nc4c3F)c12] | (3R)-1-(7-(8- Ethynyl-3- hydroxynaphthalen- 1-yl)-6,8- difluoro-2- fluorotetrahydro- 1H-pyrrolizin- 7a(5H)- yl)methoxy) quinazolin-4-yl)-3- methylpiperidin- 3-ol Isomer 1 | Free base | Step 1: 7- Bromo-2,4- dichloro-6,8- difluoroquinazoline (CAS#: 2248318-27-4, Enamine) Step 3: 2-13. (methoxymethoxy)- 8-[2- [tris(1- methylethyl) silyl]ethynyl]-1- naphthalenyl]- 4,4,5,5- tetramethyl- 1,3,2- Dioxaborolane (CAS#: 2621932-42-9, LabNetwork) | Chiral separation after Step 4 Details included below. Additional step before Step 4. Details included below. |
| 12 | 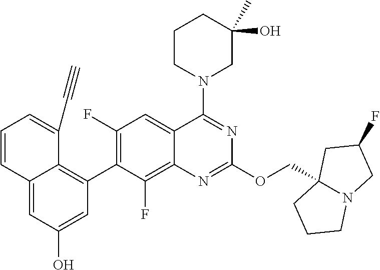 [SMILES: C#Cc1cccc2cc(O)cc(-c3c(F)cc4c(N5CCC[C@@](C)(O)C5)nc(OC[C@@]56CCCN5C[C@H](F)C6)nc4c3F)c12] | (3R)-1-(7-(8- Ethynyl-3- hydroxynaphthalen- 1-yl)-6,8- difluoro-2- (((2R,7aS)-2- fluorotetrahydro- 1H-pyrrolizin- 7a(5H)- yl)methoxy) quinazolin-4-yl)-3- methylpiperidin- 3-ol Isomer 2 | Free base | Step 1: 7- Bromo-2,4- dichloro-6,8- difluoroquinazoline (CAS#: 2248318-27-4, Enamine) Step 3: 2-[3- (methoxymethoxy)- 8-[2- [tris(1- methylethyl) silyl]ethynyl]-1- naphthalenyl]- 4,4,5,5- tetramethyl- 1,3,2- dioxaborolane (CAS#: 2621932-42-9, LabNetwork) | Chiral separation after Step 4. Details included below. Additional step before Step 4 similar to Example 11 |
| 13 | 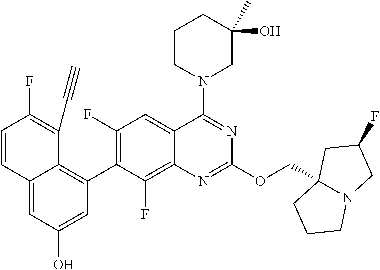 [SMILES: C#Cc1c(F)ccc2cc(O)cc(-c3c(F)cc4c(N5CCC[C@@](C)(O)C5)nc(OC[C@@]56CCCN5C[C@H](F)C6)nc4c3F)c12] | (3R)-1-(7-(8- Ethynyl-7-fluoro- 3- hydroxynaphthalen- 1-yl)-6,8- difluoro-2- (((2R,7aS)-2- fluorotetrahydro- 1H-pyrrolizin- 7a(5H)- yl )methoxy) quinazolin-4-yl)-3- methylpiperidin- 3-ol Isomer 1 | Free base | Step 1: 7- Bromo-2,4- dichloro-6,8- difluoroquinazoline (CAS#: 2248318-27-4, Enamine) Step 3: 2-[7- fluoro-3- (methoxymethoxy)- 8-[2- [tris(1- methylethyl) silyl]ethynyl]-1- naphthalenyl]- 4,4,5,5- tetramethyl- 1,3,2- dioxaborolane (CAS#: 2621932-37-2, LabNetwork) | Chiral separation after Step 3. Details included below. Additional step before Step 4 similar to Example 11. Final purification performed on silica gel column, eluting with 0 - 100% 3:1 EtOAc/EtOH in heptane with 2% TEA |
| 14 | 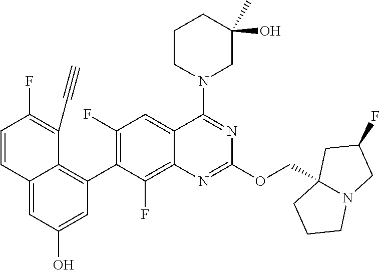 [SMILES: C#Cc1c(F)ccc2cc(O)cc(-c3c(F)cc4c(N5CCC[C@@](C)(O)C5)nc(OC[C@@]56CCCN5C[C@H](F)C6)nc4c3F)c12] | (3R)-1-(7-(8- Ethynyl-7-fluoro- 3- hydroxynaphthalen- 1-yl)-6,8- difluoro-2- (((2R,7aS)-2- fluorotetrahydro- 1H-pyrrolizin- 7a(5H)- yl)methoxy) quinazolin-4-yl)-3- methylpiperidin- 3-ol Isomer 2 | Free base | Step 1: 7- Bromo-2,4- dichloro-6,8- difluoroquinazoline (CAS#: 2248318-27-4, Enamine) Step 3: 2-[7- fluoro-3- (metboxymeth oxy)-8-12- [tris(1- methylethyl) silyl]ethynyl]-1- naphthalenyl]- 4,4,5,5- tetramethyl- 1,3,2- dioxaborolane CAS#: 2621932-37-2, LabNetwork) | Chiral separation after Step 3. Details included below. Additional step before Step 4 similar to Example 11. Final purification performed on silica gel column, eluting with 0 - 100% 3:1 EtOAc/EtOH in heptane with 2% TEA. |
| 15 | 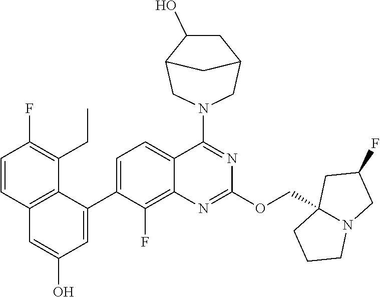 [SMILES: CCc1c(F)ccc2cc(O)cc(-c3ccc4c(N5CC6CC(O)C(C6)C5)nc(OC[C@@]56CCCN5C[C@H](F)C6)nc4c3F)c12] | 3-(7-(8-Ethyl-7- fluoro-3- hydroxynaphthalen- 1-yl)-8-fluoro- 2-(((2R,7aS)-2- fluorotetrahydro- 1H-pyrrolizin- 7a(5H)- yl)methoxy) quinazolin-4-yl)-3- azabicyclo[3.2.1] octan-6-ol Isomer 1 | 2,2,2- trifluoro- acetate | Step 1: 3- Azabicyclo[3. 2.1]octan-6-ol hydrochloride (CAS#: 2305252-97-3, Enamine) | Chiral separation after Step 1. Details included below. |
| 16 | 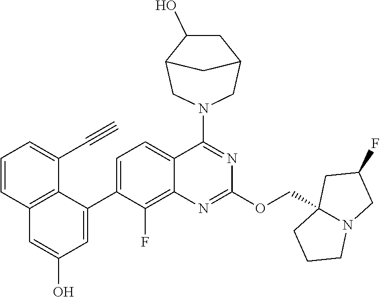 [SMILES: C#Cc1cccc2cc(O)cc(-c3ccc4c(N5CC6CC(O)C(C6)C5)nc(OC[C@@]56CCCN5C[C@H](F)C6)nc4c3F)c12] | 3-(7-(8-Ethynyl- 3- hydroxynaphthalen- 1-yl)-8-fluoro- 2-(((2R,7aS)-2- fluorotetrahydro- 1H-pyrrolizin- 7a(5H)- yl)methoxy) quinazolin-4-yl)-3- azabicyclo[3.2.1] octan-6-ol Isomer 1 | Free base | Step 1: 3- Azabicyclo[3. 2.1]octan-6-ol hydrochloride (CAS#: 2305252-97-3, Enamine) Step 3: 2-[3- (methoxymethoxy)- 8-[2- [tris(1- methylethyl) silyl]ethynyl]-1- naphthalenyl]- 4,4,5,5- tetramethyl- 1,3,2- dioxaborolane (CAS#: 2621932-42-9, LabNetwork | Chiral separation after Step 1. Details included below. Additional step before Step 4 similar to Example 11. |
| 17 | 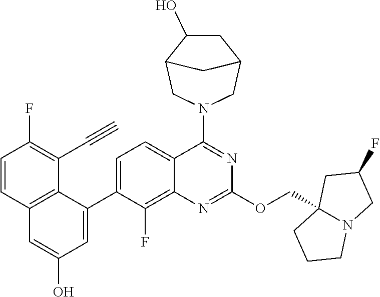 [SMILES: C#Cc1c(F)ccc2cc(O)cc(-c3ccc4c(N5CC6CC(O)C(C6)C5)nc(OC[C@@]56CCCN5C[C@H](F)C6)nc4c3F)c12] | 3-(7-(8-Ethynyl- 7-fluoro-3- hydroxynaphthalen- 1-yl)-8-fluoro- 2-(((2R,7aS)-2- fluorotetrahydro- 1H-pyrrolizin- 7a(5H)- yl)methoxy) quinazolin-4-yl)-3- azabicyclo[3.2.1] octan-6-ol Isomer 1 | 2,2,2- trifluoro- acetate | Step 1: 3- Azabicyclo[3. 2.1]octan-6-ol hydrochloride (CAS#: 2305252-97-3, Enamine) Step 3: 2-17- fluoro-3- (metboxymethoxy)- 8-12- [tris(1- methylethyl) silyl]ethynyl]-1- naphthalenyl]- 4,4,5,5- tetramethyl- 1,3,2- dioxaborolane (CAS#: 2621932-37-2, LabNetwork) | Chiral separation after Step 1. Details included below. Additional step before Step 4 similar to Example 11. |
| 18 | 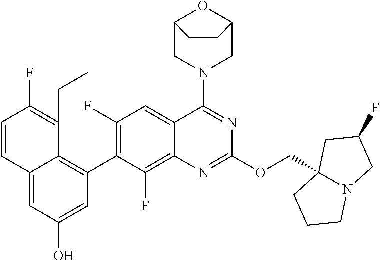 [SMILES: CCc1c(F)ccc2cc(O)cc(-c3c(F)cc4c(N5CC6CCC(C5)O6)nc(OC[C@@]56CCCN5C[C@H](F)C6)nc4c3F)c12] | rel-4-(4-((1R,5S)- 8-Oxa-3- azabicyclo[3.2.1] octan-3-yl)-6,8- difluoro-2- (((2R,7aS)-2- fluorotetrahydro- 1H-pyrrolizin- 7a(5H)- yl)methoxy ) quinazolin-7-yl)-5- ethyl-6- fluoronaphthalen- 2-ol | bis(2, 2,2- trifluoro- acetate) | Step 1: 7- Bromo-2,4- dichloro-6,8- difluoroquinazoline (CAS#: 2248318-27-4, Enamine) and 8-oxa-3- aza- bicyclo[3.2.1] octane (CAS#: 280-13-7, J & W Pharmlab, LLC) | |
| 19 | 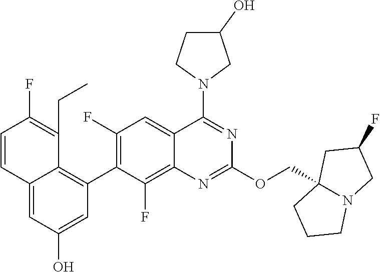 [SMILES: CCc1c(F)ccc2cc(O)cc(-c3c(F)cc4c(N5CCC(O)C5)nc(OC[C@@]56CCCN5C[C@H](F)C6)nc4c3F)c12] | 1-(7-(8-Ethyl-7- fluoro-3- hydroxynaphthalen- 1-yl)-6,8- difluoro-2- (((2R,7a.S)-2- fluorotetrahydro- 1H-pyrrolizin- 7a(5H)- yl)methoxy) quinazolin-4- yl)pyrrolidin-3-ol | Bis(2, 2,2- trifluoro- acetate) | Step 1: 7- Bromo-2,4- dichloro-6,8- difluoroquinazoline (CAS#: 2248318-27-4 Enamine) and 3- pyrrolidinol (CAS#: 40499-83-0, Synthonix Inc.) | |
| 20 | 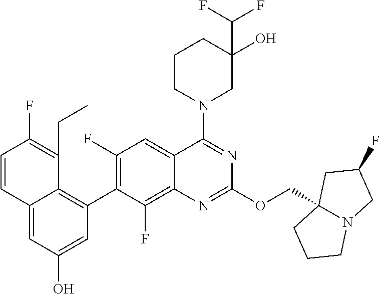 [SMILES: CCc1c(F)ccc2cc(O)cc(-c3c(F)cc4c(N5CCCC(O)(C(F)F)C5)nc(OC[C@@]56CCCN5C[C@H](F)C6)nc4c3F)c12] | 3- (Difluoromethyl)- 1-(7-(8-ethyl-7- fluoro-3- hydroxynaphthalen- 1-yl)-6,8- difluoro-2- (((2R,7aS)-2- fluorotetrahydro- 1H-pyrrolizin- 7a(5H)- yl)methoxy) quinazolin-4- yl)piperidin-3-ol Isomer 1 | 2,2,2- Trifluoro- acetate | Step 1: 7- Bromo-2,4- dichloro-6,8- difluoro- quinazoline (CAS#: 2248318-27-4, PharmaBlock) and 3- (difluorometh yl)piperidin-3- ol hydrochloride (CAS#: 1909325-03-6, Enamine) | Chiral separation after Step 1 and after Step 3. Details included below. |
| 21 | 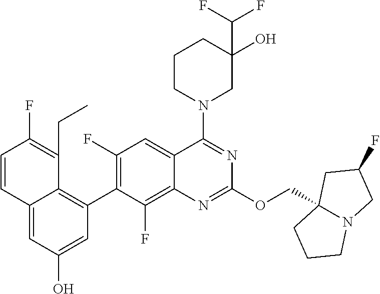 [SMILES: CCc1c(F)ccc2cc(O)cc(-c3c(F)cc4c(N5CCCC(O)(C(F)F)C5)nc(OC[C@@]56CCCN5C[C@H](F)C6)nc4c3F)c12] | 3- (Difluoromethyl)- 1-(7-(8-ethyl-7- fluoro-3- hydroxynaphthalen- 1-yl)-6,8- difluoro-2- (((2R,7aS)-2- fluorotetrahydro- 1H-pyrrolizin- 7a(5H)- yl)methoxy) quinazolin-4- yl)piperidin-3-ol Isomer 2 | 2,2,2- Trifluoro- acetate | Step 1: 7- Bromo-2,4- dichloro-6,8- difluoro- quinazoline (CAS#: 2248318-27-4, PharmaBlock) and 3- (difluoromethyl) piperidin-3- ol hydrochloride (CAS#: 1909325-03-6, Enamine) | Chiral separation after Step 1 and after Step 3. Details included below. |
| 22 | 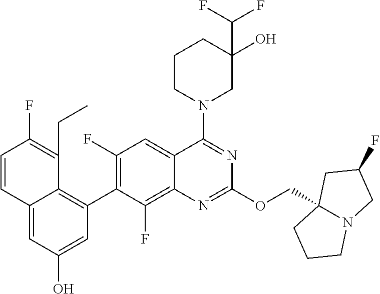 [SMILES: CCc1c(F)ccc2cc(O)cc(-c3c(F)cc4c(N5CCCC(O)(C(F)F)C5)nc(OC[C@@]56CCCN5C[C@H](F)C6)nc4c3F)c12] | 3- (Difluoromethyl)- 1-(7-(8-ethyl-7- fluoro-3- hydroxynaphthalen- 1-yl)-6,8- difluoro-2- (((2R,7a.S)-2- fluorotetrahydro- 1H-pyrrolizin- 7a(5H)- yl)methoxy ) quinazolin-4- yl)piperidin-3-ol Mixture of Isomer 3 and 4 | 2,2,2- Trifluoro- acetate | Step 1: 7- Bromo-2,4- dichloro-6,8- difluoro- quinazoline (CAS#: 2248318-27-4, PharmaBlock) and 3- (difluoromethyl) piperidin-3- ol hydrochloride (CAS#: 1909325-03-6, Enamine) | Chiral separation after Step 1. Details included below. |
| 23 | 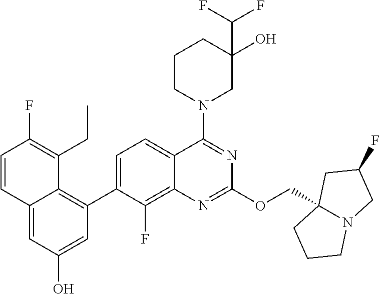 [SMILES: CCc1c(F)ccc2cc(O)cc(-c3ccc4c(N5CCCC(O)(C(F)F)C5)nc(OC[C@@]56CCCN5C[C@H](F)C6)nc4c3F)c12] | 3- (Difluoromethyl)- 1-(7-(8-ethyl-7- fluoro-3- hydroxynaphthalen- 1-yl)-8-fluoro- 2-(((2R,7aS)-2- fluorotetrahydro- 1H-pyrrolizin- 7a(5H)- yl)methoxy) quinazolin-4- yl)piperidin-3-ol Isomer 1 | 2,2,2- trifluoro- acetate | Step 1: 3- (Difluoromethyl) piperidin-3- ol hydrochloride (CAS#: 1909325-03-6, Enamine) | Chiral separation after Step 1. Details included below. |
| 24 | 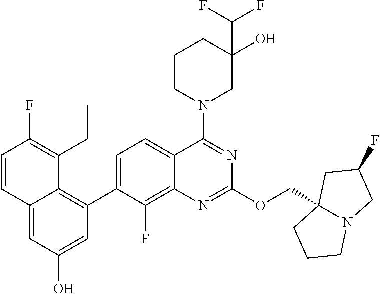 [SMILES: CCc1c(F)ccc2cc(O)cc(-c3ccc4c(N5CCCC(O)(C(F)F)C5)nc(OC[C@@]56CCCN5C[C@H](F)C6)nc4c3F)c12] | 3- (Difluoromethyl)- 1-(7-(8-ethyl-7- fluoro-3- hydroxynaphthalen- 1-yl)-8-fluoro- 2-(((2R,7aS)-2- fluorotetrahydro- 1H-pyrrolizin- 7a(5H)- yl)methoxy) quinazolin-4- yl)piperidin-3-ol Isomer 2 | 2,2,2- trifluoro- acetate | Step 1: 3- (Difluoromethyl) piperidin-3- ol hydrochloride (CAS#: 1909325-03-6, Enamine) | Chiral separation after Step 1. Details included below. |
| 25 | 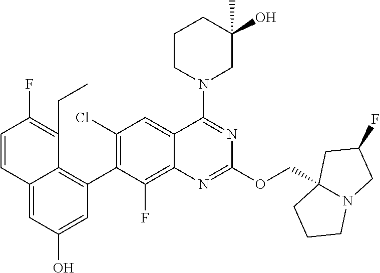 [SMILES: CCc1c(F)ccc2cc(O)cc(-c3c(Cl)cc4c(N5CCC[C@@](C)(O)C5)nc(OC[C@@]56CCCN5C[C@H](F)C6)nc4c3F)c12] | (3R)-1-(6- Chloro-7-(8- ethyl-7-fluoro-3- hydroxynaphthalen- 1-yl)-8-fluoro- 2-(((2R,7aS)-2- fluorotetrahydro- 1H-pyrrolizin- 7a(5H)- yl)methoxy) quinazolin-4-yl)-3- methylpiperidin- 3-ol | 2,2,2- trifluoro- acetate | Step 1: 7- Bromo-2,4,6- trichloro-8- fluoroquinazoline (CAS#: 1698028-11-3, WuXi) | 1,1′-Bis(di- tert- butyl- phosphino) ferrocene- palladium dichloride was used in Step 3. |
| 26 | 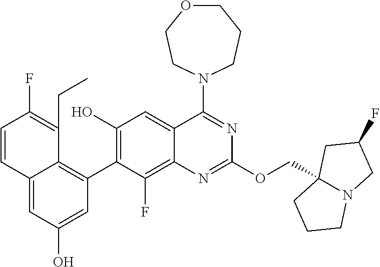 [SMILES: CCc1c(F)ccc2cc(O)cc(-c3c(O)cc4c(N5CCCOCC5)nc(OC[C@@]56CCCN5C[C@H](F)C6)nc4c3F)c12] | 7-(8-Ethyl-7- fluoro-3- hydroxynaphthalen- 1-yl)-8-fluoro- 2-(((2R,7aS)-2- fluorotetrahydro- 1H-pyrrolizin- 7a(5H)- yl)methoxy)-4- (1,4-oxazepan-4- yl)quinazolin-6- ol | 2,2,2- trifluoro- acetate | Step 1: 7- Bromo-2,4,6- trichloro-8- fluoroquinazoline (CAS#: 1698028-11-3, WuXi) and 1,4 oxazepane (CAS#: 5638- 60-8, Oakwood) | 1,1′-Bis(di- tert- butyl- phosphino) ferrocene- palladium dichloride was used in Step 3. Additional step after Step 3. Details included below |
| 27 | 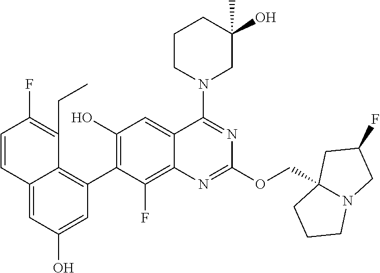 [SMILES: CCc1c(F)ccc2cc(O)cc(-c3c(O)cc4c(N5CCC[C@@](C)(O)C5)nc(OC[C@@]56CCCN5C[C@H](F)C6)nc4c3F)c12] | 7-(8-Ethyl-7- fluoro-3- hydroxynaphthalen- 1-yl)-8-fluoro- 2-(((2R,7aS)-2- fluorotetrahydro- 1H-pyrrolizin- 7a(5H)- yl)methoxy)-4- ((R)-3-hydroxy- 3- methylpiperidin- 1-yl)quinazolin- 6-ol Isomer 1 | 2,2,2- trifluoro- acetate | Step 1: 7- Bromo-2,4,6- trichloro-8- fluoroquinazoline (CAS#: 1698028-11-3, WuXi) | 1,1′-Bis(di- tert- butyl- phosphino) ferrocene- palladium dichloride was used in Step 3. Additional step after Step 3 similar to Example 26 followed by chiral separation. |
| 28 | 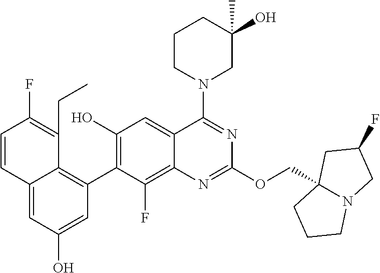 [SMILES: CCc1c(F)ccc2cc(O)cc(-c3c(O)cc4c(N5CCC[C@@](C)(O)C5)nc(OC[C@@]56CCCN5C[C@H](F)C6)nc4c3F)c12] | 7-(8-Ethyl-7- fluoro-3- hydroxynaphthalen- 1-yl)-8-fluoro- 2-(((2R,7aS)-2- fluorotetrahydro- 1H-pyrrolizin- 7a(5H)- yl)methoxy)-4- ((R)-3-hydroxy- 3- methylpiperidin- 1-yl)quinazolin- 6-ol Isomer 2 | 2,2,2- trifluoro- acetate | Step 1: 7- Bromo-2,4,6- trichloro-8- fluoroquinazoline (CAS#: 1698028-11-3, WuXi) | 1,1′-Bis(di- tert- butyl- phosphino) ferrocene- palladium dichloride was used in Step 3. Additional step after Step 3 similar to Example 26 followed by chiral separation |
| 29 | 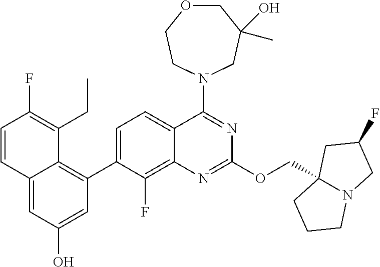 [SMILES: CCc1c(F)ccc2cc(O)cc(-c3ccc4c(N5CCOCC(C)(O)C5)nc(OC[C@@]56CCCN5C[C@H](F)C6)nc4c3F)c12] | 4-(7-(8-Ethyl-7- fluoro-3- hydroxynaphthalen- 1-yl)-8-fluoro- 2-(((2R,7aS)-2- fluorotetrahydro- 1H-pyrrolizin- 7a(5H)- yl)methoxy) quinazolin-4-yl)-6- methyl-1,4- oxazepan-6-ol Isomer 1 | 2,2,2- trifluoro- acetate | Step 1: 6- Methyl-1,4- oxazepan-6-ol hydrochloride Isomer 1 (Intermediate A1) | |
| 30 | 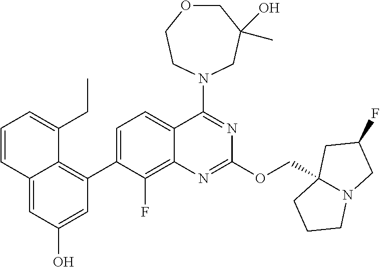 [SMILES: CCc1cccc2cc(O)cc(-c3ccc4c(N5CCOCC(C)(O)C5)nc(OC[C@@]56CCCN5C[C@H](F)C6)nc4c3F)c12] | 4-(7-(8-Ethyl-3- hydroxynaphthalen- 1-yl)-8-fluoro- 2-(((2R,7aS)-2- fluorotetrahydro- 1H-pyrrolizin- 7a(5H)- yl)methoxy) quinazolin-4-yl)-6- methyl-1,4- oxazepan-6-ol Isomer 1 | 2,2,2- trifluoro- acetate | Step 1: 6- Methyl-1,4- oxazepan-6-ol hydrochloride Isomer 1 (Intermediate A1) Step 3: 2-(8- Ethyl-3- (methoxymethoxy) naphthalen-1-yl)- 4,4,5,5- tetramethyl- 1,3,2- dioxaborolane (CAS#: 2621932-60-1, LabNetwork) | |
| 31 | 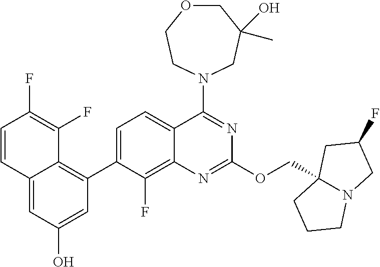 [SMILES: CC1(O)COCCN(c2nc(OC[C@@]34CCCN3C[C@H](F)C4)nc3c(F)c(-c4cc(O)cc5ccc(F)c(F)c45)ccc23)C1] | 4-(7-(7,8- Difluoro-3- hydroxynaphthalen- 1-yl)-8-fluoro- 2-(((2R,7aS)-2- fluorotetrahydro- 1H-pyrrolizin- 7a(5H)- yl)methoxy) quinazolin-4-yl)-6- methyl-1,4- oxazepan-6-ol Isomer 1 | 2,2,2- trifluoro- acetate | Step 1: 6- Methyl-1,4- oxazepan-6-ol hydrochloride Isomer 1 (Intermediate A1) Step 3: 2-(7,8- difluoro-3- (methoxymethoxy) naphthalen- 1-yl)- 4,4,5,5- tetramethyl- 1,3,2- dioxaborolane (CAS#: 2621935-35-9, LabNetwork) | |
| 32 | 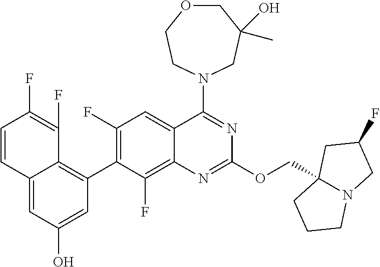 [SMILES: CC1(O)COCCN(c2nc(OC[C@@]34CCCN3C[C@H](F)C4)nc3c(F)c(-c4cc(O)cc5ccc(F)c(F)c45)c(F)cc23)C1] | 4-(7-(7,8- Difluoro-3- hydroxynaphthalen- 1-yl)-6,8- difluoro-2- (((2R,7a.S)-2- fluorotetrahydro- 1H-pyrrolizin- 7a(5H)- yl)methoxy) quinazolin-4-yl)-6- methyl-1,4- oxazepan-6-ol Isomer 1 | 2,2,2- trifluoro- acetate | Step 1: 6- Methyl-1,4- oxazepan-6-ol hydrochloride Isomer 1 (Intermediate A1) and 7- bromo-2,4- dichloro-6,8- difluoro- quinazoline (CAS#: 2248318-27-4. PharmaBlock) Step 3: 2-(7,8- difluoro-3- (methoxymethoxy) naphthalen- 1-yl)- 4,4,5,5- tetramethyl- 1,3,2- dioxaborolane (CAS#: 2621935-35-9, LabNetwork) | |
| 33 | 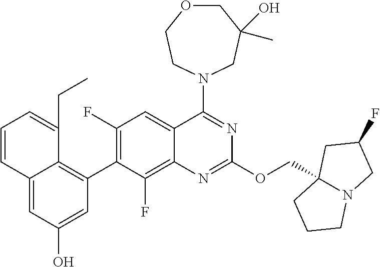 [SMILES: CCc1cccc2cc(O)cc(-c3c(F)cc4c(N5CCOCC(C)(O)C5)nc(OC[C@@]56CCCN5C[C@H](F)C6)nc4c3F)c12] | 4-(7-(8-Ethyl-3- hydroxynaphthalen- 1-yl)-6,8- difluoro-2- (((2R,7aS)-2- fluorotetrahydro- 1H-pyrrolizin- 7a(5H)- yl)methoxy) quinazolin-4-yl)-6- methyl-1,4- oxazepan-6-ol Isomer 1 | 2,2,2- trifluoro- acetate | Step 1: 6- Methyl-1,4- oxazepan-6-ol hydrochloride Isomer 1 (Intermediate A1) and 7-bromo-2,4- dichloro-6,8- difluoro- quinazoline (CAS#: 2248318-27-4. PharmaBlock) Step 3: 2-(8- ethyl-3- (methoxymethoxy) naphthalen- 1-yl)- 4,4,5,5- tetramethyl- 1,3,2- dioxaborolane (CAS#: 2621932-60-1, LabNetwork) | |
| 34 | 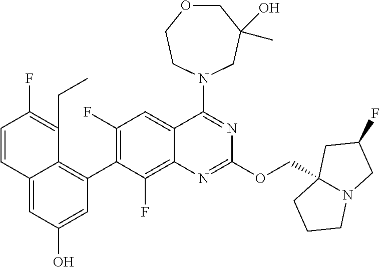 [SMILES: CCc1c(F)ccc2cc(O)cc(-c3c(F)cc4c(N5CCOCC(C)(O)C5)nc(OC[C@@]56CCCN5C[C@H](F)C6)nc4c3F)c12] | 4-(7-(8-Ethyl-7- fluoro-3- hydroxynaphthalen- 1-yl)-6,8- difluoro-2- (((2R,7aS)-2- fluorotetrahydro- 1H-pyrrolizin- 7a(5H)- yl)methoxy) quinazolin-4-yl)-6- methyl-1,4- oxazepan-6-ol Isomer 1 | 2,2,2- trifluoro- acetate | Step 1: 6- Methyl-1,4- oxazepan-6-ol hydrochloride Isomer 1 (Intermediate A1) and 7- bromo-2,4- dichloro-6,8- difluoro- quinazoline (CAS#: 2248318-27-4. PharmaBlock) | |
| 35 | 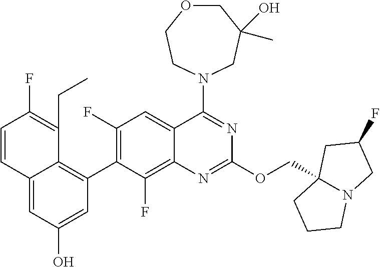 [SMILES: CCc1c(F)ccc2cc(O)cc(-c3c(F)cc4c(N5CCOCC(C)(O)C5)nc(OC[C@@]56CCCN5C[C@H](F)C6)nc4c3F)c12] | 4-(7-(8-Ethyl-7- fluoro-3- hydroxynaphthalen- 1-yl)-6,8- difluoro-2- (((2R,7aS)-2- fluorotetrahydro- 1H-pyrrolizin- 7a(5H)- yl)methoxy) quinazolin-4-yl)-6- methyl-1,4- oxazepan-6-ol Isomer 2 | 2,2,2- trifluoro- acetate | Step 1: 6- Methyl-1,4- oxazepan-6-ol hydrochloride Isomer 1 (Intermediate A1) and 7- bromo-2,4- dichloro-6,8- difluoro- quinazoline (CAS#: 2248318-27-4. PharmaBlock) | |
| 36 | 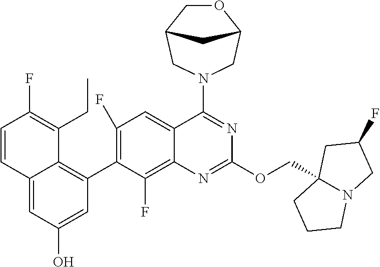 [SMILES: CCc1c(F)ccc2cc(O)cc(-c3c(F)cc4c(N5C[C@@H]6CO[C@@H](C6)C5)nc(OC[C@@]56CCCN5C[C@H](F)C6)nc4c3F)c12] | rac-4-(4- ((1R,5S)-6-Oxa- 3- azabicyclo[3.2.1] octan-3-yl)-6,8- difluoro-2- (((2R,7aS)-2- fluorotetrahydro- 1H-pyrrolizin- 7a(5H)- yl)methoxy) quinazolin-7-yl)-5- ethyl-6- fluoronaphthalen- 2-ol | 2,2,2- trifluoro- acetate | Step 1: 6- Oxa-3- azabicyclo[3.2.1] octane hydrochloride (CAS#: 1461708-41-7, Enamine) and 7-bromo-2,4- dichloro-6,8- difluoro- quinazoline (CAS#: 2248318-27-4, PharmaBlock) | |
| 37 | 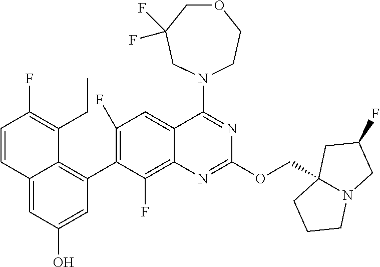 [SMILES: CCc1c(F)ccc2cc(O)cc(-c3c(F)cc4c(N5CCOCC(F)(F)C5)nc(OC[C@@]56CCCN5C[C@H](F)C6)nc4c3F)c12] | 4-(4-(6,6- Difluoro-1,4- oxazepan-4-yl)- 6,8-difluoro-2- (((2R,7aS)-2- fluorotetrahydro- 1H-pyrrolizin- 7a(5H)- yl)methoxy) quinazolin-7-yl)-5- ethyl-6- fluoronaphthalen- 2-ol | 2,2,2- trifluoro- acetate | Step 1: 6,6- Difluoro-1,4- oxazepane hydrochloride (CAS#: 1341039-23-3, Enamine) and 7-bromo-2,4- dichloro-6,8- difluoro- quinazoline (CAS#: 2248318-27-4. PharmaBlock) | |
| 38 | 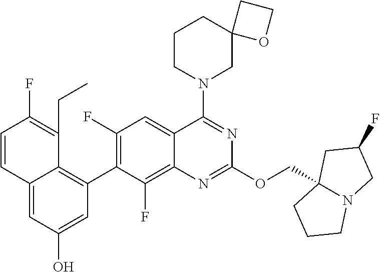 [SMILES: CCc1c(F)ccc2cc(O)cc(-c3c(F)cc4c(N5CCCC6(CCO6)C5)nc(OC[C@@]56CCCN5C[C@H](F)C6)nc4c3F)c12] | 4-(6,8-Difluoro- 2-(((2R,7aS)-2- fluorotetrahydro- 1H-pyrrolizin- 7a(5H)- yl)methoxy)-4- (1-oxa-6- azaspiro[3.5] nonan-6- yl)quinazolin-7- yl)-5-ethyl-6- fluoronaphthalen- 2-ol Isomer 1 | Bis- 2,2,2- trifluoro- aceate | Step 1: 1- Oxa-6- azaspiro[3.5] nonane hydrochloride (CAS#: 1956354-49-6, Enamine) and 7-bromo-2,4- dichloro-6,8- difluoro- quinazoline (CAS#: 2248318-27-4. PharmaBlock) Step 3: 5- Ethyl-6- fluoro-4- (4,4,5,5- tetramethyl- 1,3,2- dioxaborolan- 2- yl)naphthalen- 2-ol (Intermediate B2) | Chiral separation after Step 2. Details included below. No Step 4. |
| 39 | 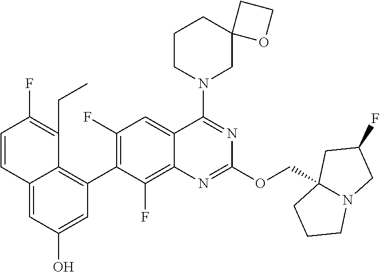 [SMILES: CCc1c(F)ccc2cc(O)cc(-c3c(F)cc4c(N5CCCC6(CCO6)C5)nc(OC[C@@]56CCCN5C[C@H](F)C6)nc4c3F)c12] | 4-(6,8-Difluoro- 2-(((2R,7aS)-2- fluorotetrahydro- 1H-pyrrolizin- 7a(5H)- yl)methoxy)-4- (1-oxa-6- azaspiro[3.5] nonan-6- yl)quinazolin-7- yl)-5-ethyl-6- fluoronaphthalen- 2-ol Isomer 2 | Bis(2, 2,2- trifluoro- aceate) | Step 1: 1- Oxa-6- azaspiro[3.5] nonane hydrochloride (CAS#: 1956354-49-6, Enamine) and 7-bromo-2.4- dichloro-6,8- difluoro- quinazoline (CAS#: 2248318-27-4, PharmaBlock) Step 3: 5- ethyl-6-fluoro- 4-(4,4,5,5- tetramethyl- 1,3,2- dioxaborolan- 2- yl)naphthalen- 2-ol (Intermediate B2) | Chiral separation after Step 2. Details included below. No Step 4. |
| 40 | 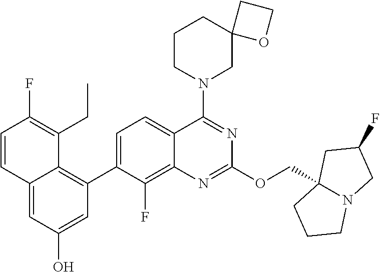 [SMILES: CCc1c(F)ccc2cc(O)cc(-c3ccc4c(N5CCCC6(CCO6)C5)nc(OC[C@@]56CCCN5C[C@H](F)C6)nc4c3F)c12] | 5-Ethyl-6-fluoro- 4-(8-fluoro-2- (((2R,7aS)-2- fluorotetrahydro- 1H-pyrrolizin- 7a(5H)- yl)methoxy)-4- (1-oxa-6- azaspiro[3.5] nonan-6- yl)quinazolin-7- yl)naphthalen-2- ol | Bis(2, 2,2- trifluoro- aceate) | Step 1: 1- Oxa- 6azaspiro[3.5] nonanehemioxalate (CAS#: 1523606-44-1, PharmaBlock) Step 3: 5- ethyl-6-fluoro- 4-(4,4,5,5- tetramethyl- 1,3,2- dioxaborolan- 2- yl)naphthalen- 2-ol (Intermediate B2) | No Step 4. |
| 41 | 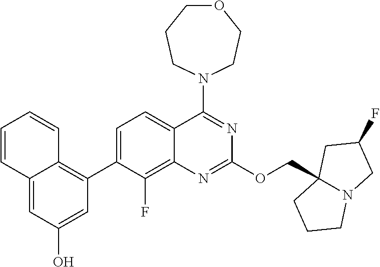 [SMILES: Oc1cc(-c2ccc3c(N4CCCOCC4)nc(OC[C@]45CCCN4C[C@H](F)C5)nc3c2F)c2ccccc2c1] | 4-(8-Fluoro-2- (((2R,7aR)-2- fluorotetrahydro- 1H-pyrrolizin- 7a(5H)- yl)methoxy)-4- (1,4-oxazepan-4- yl)quinazolin-7- yl)naphthalen-2- ol | 2,2,2- trifluoro- acetate | Step 1: [1,4]Oxazepane (CAS#: 5638- 60-8, Oakwood Products) Step 2: ((2R,7aR)-2- fluorotetrabydro- 1H- pyrrolizin- 7a(5H)- yl)methanol (CAS#: 2454490-66-3, WuXi) Step 3: 4- (4,4,5,5- tetramethyl- 1,3,2- dioxaborolan- 2-yl)-2- naphthol (CAS#: 2043962-01-0, Aurum Pharmatech LLC) | Pd(dtbpf)Cl₂ was used in Step 3 as catalyst. No Step 4. |
| 42 | 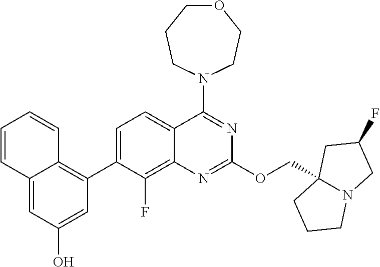 [SMILES: Oc1cc(-c2ccc3c(N4CCCOCC4)nc(OC[C@@]45CCCN4C[C@H](F)C5)nc3c2F)c2ccccc2c1] | 4-(8-Fluoro-2- (((2R,7aS)-2- fluorotetrahydro- 1H-pyrrolizin- 7a(5H)- yl)methoxy)-4- (1,4-oxazepan-4- yl)quinazolin-7- yl)naphthalen-2- ol | 2,2,2- trifluoro- acetate | Step 1: [1,4]Oxazepane (CAS#: 5638- 60-8, Oakwood Products) Step 3: 4- (4,4,5,5- tetramethyl- 1,3,2- dioxaborolan- 2-yl)-2- naphthol (CAS#: 2043962-01-0, Aurum Pharmatech LLC) | Pd(dtbpf)Cl₂ was used in Step 3 as catalyst. No Step 4. |
| 43 | 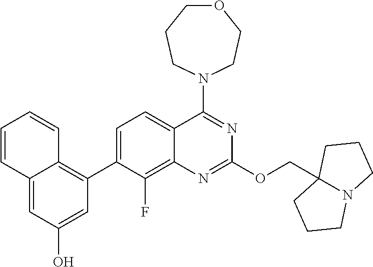 [SMILES: Oc1cc(-c2ccc3c(N4CCCOCC4)nc(OCC45CCCN4CCC5)nc3c2F)c2ccccc2c1] | 4-(8-Fluoro-4- (1,4-oxazepan-4- yl)-2- ((tetrahydro-1H- pyrrolizin- 7a(5H)- yl)methoxy) quinazolin-7- yl)naphthalen-2- ol | Bis(2, 2,2- trifluoro- aceate) | Step 1: [1,4]Oxazepane (CAS#: 5638- 60-8, Oakwood Products, Inc.) Step 2: 1,2,3,5,6,7- hexahydro- pyrrolizin-8- ylmethanol (CAS#: 78449-72-6, Advanced Chem Blocks ) Step 3: 4- (4,4,5,5- tetramethyl- 1,3,2- dioxaborolan- 2-yl)-2- naphthol (CAS#: 2043962-01-0, Aurum Pharmatech LLC) | Pd(dtbpf)Cl₂ was used in Step 3 as catalyst. No Step 4. |
| 76 | 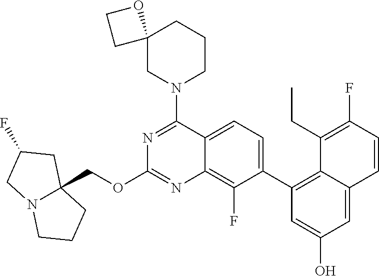 [SMILES: CCc1c(F)ccc2cc(O)cc(-c3ccc4c(N5CCC[C@]6(CCO6)C5)nc(OC[C@@]56CCCN5C[C@H](F)C6)nc4c3F)c12] | 5-Ethyl-6-fluoro- 4-(8-fluoro-2- (((2R,7aS)-2- fluorotetrahydro- 1H-pyrrolizin- 7a(5H)- yl)methoxy)-4- ((S)-1-oxa-6- azaspiro[ 3.5] nonan-6- yl)quinazolin-7- yl)naphthalen-2- ol | | Step 1: 1- Oxa-6- azaspiro[3.5] nonane (CAS#: 204388-70-5, PharmaBlock) Step 3: 5- ethyl-6-fluoro- 4-(4,4,5,5- tetramethyl- 1,3,2- dioxaborolan- 2- yl)naphthalen- 2-ol (Intermediate B2) | Chiral separation after Step 2. Details included below. No Step 4. |
| 77 | 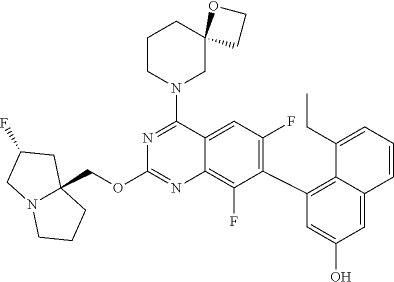 [SMILES: CCc1cccc2cc(O)cc(-c3c(F)cc4c(N5CCC[C@]6(CCO6)C5)nc(OC[C@@]56CCCN5C[C@H](F)C6)nc4c3F)c12] | 4-(6,8-Difluoro- 2-(((2R,7aS)-2- fluorotetrahydro- 1H-pyrrolizin- 7a(5H)- yl)methoxy)-4- ((S)-1-oxa-6- azaspiro[3.5] nonan-6- yl)quinazolin-7- yl)-5- ethylnaphthalen- 2-ol | | Step 1: (4S)- 1-Oxa-6- azaspiro[3.5] nonane (CAS#: 1527519-18-1, PharmaBlock) Step 3: 5- ethyl-6-fluoro- 4-(4,4,5,5- tetramethyl- 1,3,2- dioxaborolan- yl)naphthalen- 2-ol (Intermediate B2) | No Step 4. |
| 78 | 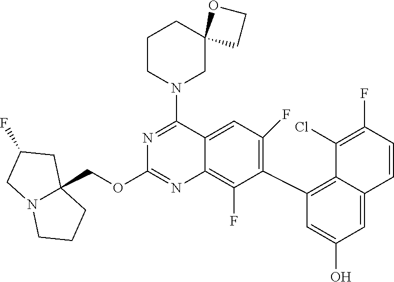 [SMILES: Oc1cc(-c2c(F)cc3c(N4CCC[C@]5(CCO5)C4)nc(OC[C@@]45CCCN4C[C@H](F)C5)nc3c2F)c2c(Cl)c(F)ccc2c1] | 5-Chloro-4-(6,8- difluoro-2- (((2R,7aS)-2- fluorotetrahydro- 1H-pyrrolizin- 7a(5H)- yl)methoxy)-4- ((S)-1-oxa-6- azaspiro[3.5] nonan-6- yl)quinazolin-7- yl)-6- fluoronaphthalen- 2-ol | | Step 1: (4S)- 1-Oxa-6- azaspiro[3.5] nonane (CAS#: 1527519-18-1, PharmaBlock) Step 3: (8- Chloro-7- fluoro-3- hydroxy- naphthalen-1- yl)boronic acid (CAS#: 2757096-73-2, PharmaBlock) | No Step 4. |
| 79 | 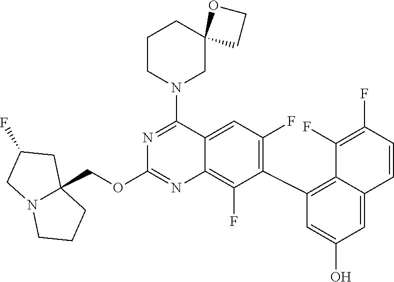 [SMILES: Oc1cc(-c2c(F)cc3c(N4CCC[C@]5(CCO5)C4)nc(OC[C@@]45CCCN4C[C@H](F)C5)nc3c2F)c2c(F)c(F)ccc2c1] | 4-(6,8-Difluoro- 2-(((2R,7aS)-2- fluorotetrahydro- 1H-pyrrolizin- 7a(5H)- yl)methoxy)-4- ((S)-1-oxa-6- azaspiro[3.5] nonan-6- yl)quinazolin-7- yl)-5,6- difluoronaphthalen- 2-ol | | Step 1: (4S)- 1-Oxa-6- azaspiro[3.5] nonane (CAS#: 1527519-18-1, PharmaBlock) Step 3: 5- ethyl-6-fluoro- 4-(4,4,5,5- tetramethyl- 1,3,2- dioxaborolan- 2- yl)naphthalen- 2-ol (Intermediate B3) | No Step 4. |
| 80 | 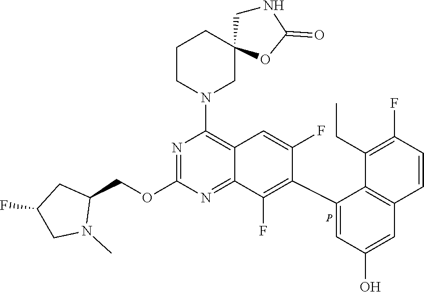 | (55)-7-(7-(8- Ethyl-7-fluoro-3- hydroxynaphthalen- 1-yl)-6,8- difluoro-2- (((2S,4R)-4- fluoro-1- methylpyrrolidin- 2-yl) methoxy)quinazolin- 4-yl)-1- oxa-3,7- diazaspiro[4.5] decan-2-one | | Step 1: 1- Oxa-3.7- diazaspiro[4.5] decan-2-one (CAS#: 1308384-36-2, ChemSpace) Step 2: ((2S,4R)-4- Fluoro-1- methylpyrrolidin- 2- yl)methanol (CAS#: 2206737-78-0, PharmaBlock) Step 3: 5- ethyl-6-fluoro- 4-(4,4,5,5- tetramethyl- 1,3,2- dioxaborolan- 2- yl)naphthalen- 2-ol (Intermediate B2) | Chiral separation after Step 3. Details included below. No Step 4. |
| 81 | 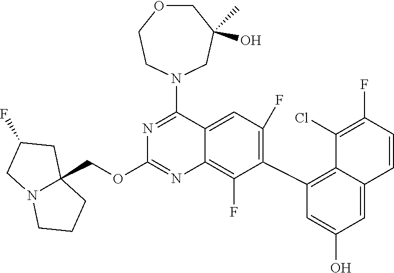 [SMILES: C[C@@]1(O)COCCN(c2nc(OC[C@@]34CCCN3C[C@H](F)C4)nc3c(F)c(-c4cc(O)cc5ccc(F)c(Cl)c45)c(F)cc23)C1] | 4-(7-(8-Chloro-7- fluoro-3- hydroxynaphthalen- 1-yl)-6,8- difluoro-2- (((2R,7aS)-2- fluorotetrahydro- 1H-pyrrolizin- 7a(5H)- yl)methoxy) quinazolin-4-yl)-6- methyl-1,4- oxazepan-6-ol Isomer 1 | Bis(2, 2,2- trifluoro- aceate) | Step 1: 6- Methyl-1,4- oxazepan-6-ol hydrochloride Isomer 1 (Intermediate A1) Step 3: (8- Chloro-7- fluoro-3- hydroxy- naphthalen-1- yl)boronic acid (CAS#: 2757096-73-2, PharmaBlock) | |
| 82 | 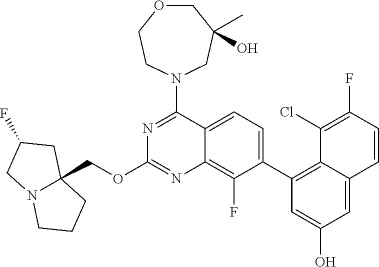 [SMILES: C[C@@]1(O)COCCN(c2nc(OC[C@@]34CCCN3C[C@H](F)C4)nc3c(F)c(-c4cc(O)cc5ccc(F)c(Cl)c45)ccc23)C1] | 4-(7-(8-Chloro-7- fluoro-3- hydroxynaphthalen- 1-yl)-8-fluoro- 2-(((2R, 7a.S)-2- fluorotetrahydro- 1H-pyrrolizin- 7a(5H)- yl)methoxy) quinazolin-4-yl)-6- methyl-1,4- oxazepan-6-ol Isomer 1 | Bis(2, 2,2- trifluoro- aceate) | Step 1: 6- Methyl-1,4- oxazepan-6-ol hydrochloride Isomer 1 (Intermediate A1) Step 3: (8- Chloro-7- fluoro-3- hydroxy- naphthalen-1- yl)boronic acid (CAS#: 2757096-73-2. PharmaBlock) | |
| 83 | 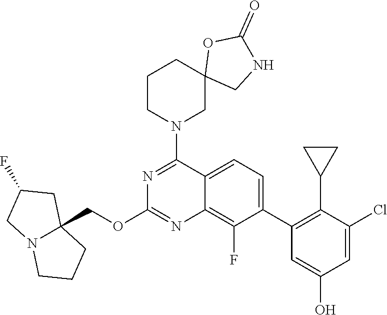 [SMILES: O=C1NCC2(CCCN(c3nc(OC[C@@]45CCCN4C[C@H](F)C5)nc4c(F)c(-c5cc(O)cc(Cl)c5C5CC5)ccc34)C2)O1] | 7-(7-(3-Chloro-2- cyclopropyl-5- hydroxyphenyl)- 8-fluoro-2- (((2R,7a5)-2- fluorotetrahydro- 1H-pyrrolizin- 7a(5H)- yl)methoxy) quinazolin-4-yl)-1- oxa-3,7- diazaspiro[4.5] decan-2-one | Bis(2, 2,2- trifluoro- aceate) | Step 1: 1- Oxa-3.7- diazaspiro[4.5] decan-2-one (CAS#: 1308384-36-2, ChemSpace) Step 3: 3. Chloro-4- cyclopropyl-5- (4,4,5,5- tetramethyl- 1,3,2- dioxaborolan- 2-yl)phenol (CAS#: 2773311-45-6, Labnetwork) | |
| 84 | 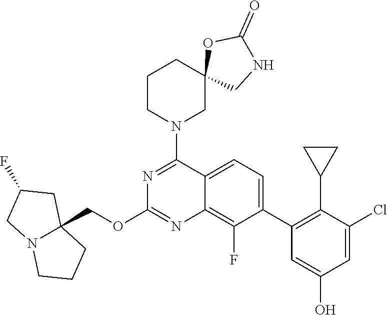 [SMILES: O=C1NC[C@]2(CCCN(c3nc(OC[C@@]45CCCN4C[C@H](F)C5)nc4c(F)c(-c5cc(O)cc(Cl)c5C5CC5)ccc34)C2)O1] | (S)-7-(7-(3- Chloro-2- cyclopropyl-5- hydroxyphenyl)- 8-fluoro-2- (((2R,7aS)-2- fluorotetrahydro- 1H-pyrrolizin- 7a(5H)- yl)methoxy) quinazolin-4-yl)-1- oxa-3,7- diazaspiro[4.5] decan-2-one | Bis(2, 2,2- trifluoro- aceate) | Step 1: (S)-1- Oxa-3,7- diazaspiro[4.5] decan-2-one (Intermediate H) Step 3: 3- Chloro-4- cyclopropyl-5- (4,4,5,5- tetramethyl- 1,3,2- dioxaborolan- 2-yl)phenol (CASH: 2773311-45-6, Labnetwork) | |
| 85 | 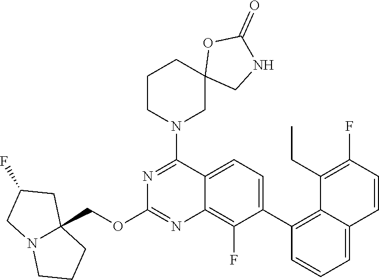 [SMILES: CCc1c(F)ccc2cccc(-c3ccc4c(N5CCCC6(CNC(=O)O6)C5)nc(OC[C@@]56CCCN5C[C@H](F)C6)nc4c3F)c12] | 7-(7-(8-Ethyl-7- fluoronaphthalen- 1-yl)-8-fluoro-2- (((2R,7aS)-2- fluorotetrahydro- 1H-pyrrolizin- 7a(5H)- yl)methoxy) quinazolin-4-yl)-1- oxa-3,7- diazaspiro[4.5] decan-2-one | 2,2,2- trifluoro- aceate | Step 1: 1- Oxa-3,7- diazaspiro[4.5] decan-2-one (CAS#: 1308384-36-2, ChemSpace) Step 3: 2-(8- ethyl-7-fluoro- 1-naphthyl)- 4,4,5,5- tetramethyl- 1,3,2- dioxaborolane (CAS#: 2848567-14-4, PharmaBlock) | |

### Additional Step for Example 4

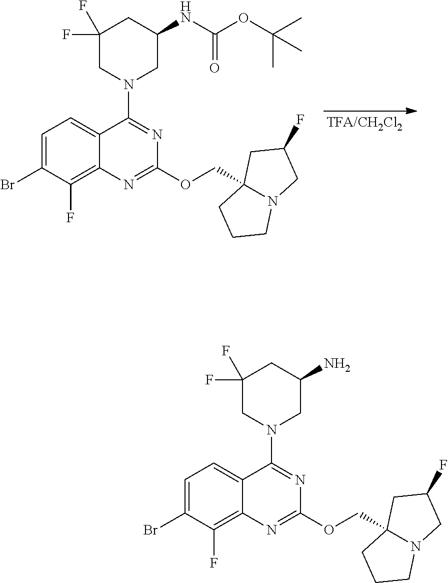 [SMILES: CC(C)(C)OC(=O)N[C@H]1CN(c2nc(OC[C@@]34CCCN3C[C@H](F)C4)nc3c(F)c(Br)ccc23)CC(F)(F)C1 ; N[C@H]1CN(c2nc(OC[C@@]34CCCN3C[C@H](F)C4)nc3c(F)c(Br)ccc23)CC(F)(F)C1]

To a stirred solution of tert-butyl ((R)-1-(7-bromo-8-fluoro-2-(((2R,7aS)-2-fluorotetrahydro-1H-pyrrolizin-7a(5H)-yl)methoxy)quinazolin-4-yl)-5,5-difluoropiperidin-3-yl)carbamate (0.24 g, 0.39 mmol) in DCM (2 mL) was added trifluoroacetic acid (1.53 g, 1 mL, 13.42 mmol. Sigma-Aldrich Corporation). The resulting mixture was stirred at rt for 1 h. The reaction mixture was concentrated. The residue was diluted with DCM and washed with saturated sodium bicarbonate aqueous solution. The organic layer was dried over Na₂SO₄ and evaporated in vacuo and the product was used assuming 100% yield. m/z (ESI): 519.0 (M+H)⁺.

### Additional Step for Example 5

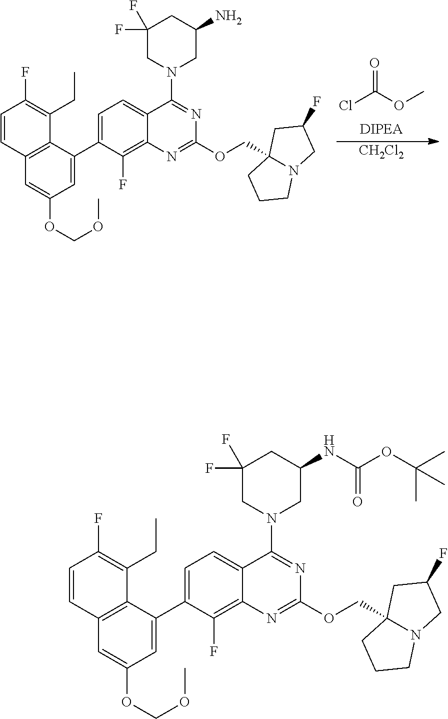

To a solution of (R)-1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-5,5-difluoropiperidin-3-amine (55 mg, 0.082 mmol) in DCM (0.5 mL) at 0° C. was added Hunig's base (32 mg, 0.043 mL, 0.25 mmol, Sigma-Aldrich Corporation), followed by methyl chloroformate (7.7 mg, 7.7 μL, 0.082 mmol, Sigma-Aldrich Corporation). The reaction mixture was stirred at rt for 15 min. The reaction mixture was concentrated and purified by HPLC to afford methyl ((R)-1-(7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-5,5-difluoropiperidin-3-yl)carbamate (28 mg, 0.038 mmol, 47% yield) as yellow solid. m/z (ESI): 730.2 (M+H)⁺.

Additional step for Example 11

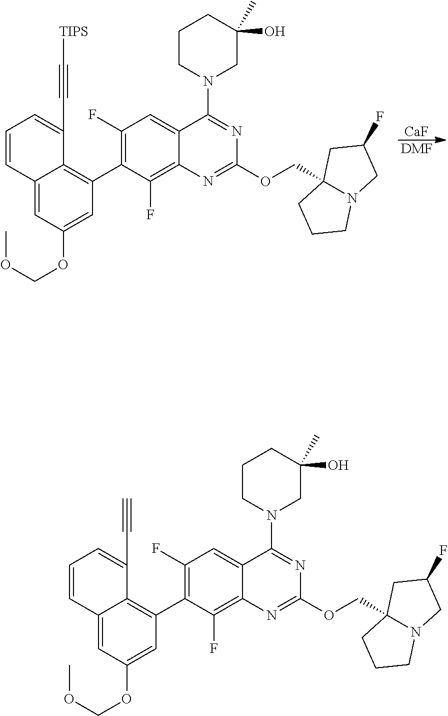

To a 10 mL round-bottom flask was added (3R)-1-(6,8-difluoro-2-(((2R,7aS)-2-fluorotetrahydro 1H-pyrrolizin-7a(5H)-yl)methoxy)-7-(3-(methoxymethoxy)-8-((triisopropylsilyl)ethynyl)naphthalen-1-yl)quinazolin-4-yl)-3-methylpiperidin-3-ol (0.20 g, 0.25 mmol) and cesium fluoride (76 mg, 0.50 mmol, Aldrich) in N,N-dimethylformamide (2.5 mL). The reaction was stirred at rt for 2 h and 20 min and then stirred at 45° C. for 1 h. The reaction was diluted with brine (10 mL), extracted with EtOAc (3×10 mL), dried over MgSO₄, filtered and concentrated in vacuo to give (3R)-1-(7-(8-ethynyl-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol as dark oil, which was used directly in the next step without further purification. m/z (ESI): 647.2 (M+H)⁺.

### Additional Step for Example 26

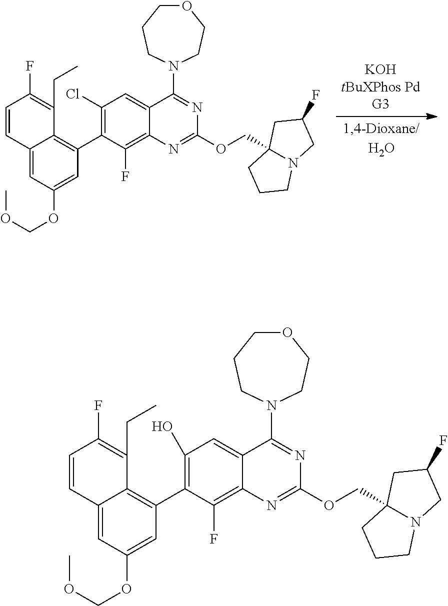 [SMILES: CCc1c(F)ccc2cc(OCOC)cc(-c3c(Cl)cc4c(N5CCCOCC5)nc(OC[C@@]56CCCN5C[C@H](F)C6)nc4c3F)c12 ; CCc1c(F)ccc2cc(OCOC)cc(-c3c(O)cc4c(N5CCCOCC5)nc(OC[C@@]56CCCN5C[C@H](F)C6)nc4c3F)c12]

A vial was charged with 4-(6-chloro-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-4-yl)-1,4-oxazepane (60 mg, 0.09 mmol), [(2-di-tert-butylphosphino-2′,4′,6′-triisopropyl-1,1′-biphenyl)-2-(2′-amino-1,1′-biphenyl)]palladium(IT) methanesulfonate (14 mg, 0.02 mmol, Sigma-Aldrich Corporation), and potassium hydroxide (15 mg, 0.27 mmol, VWR International, LLC). The solids were then suspended in degassed 1,4-dioxane (0.2 mL) and water (0.2 mL). The reaction was sealed and heated to 100° C. After 1.5 h, the reaction was concentrated under reduced pressure to afford a crude black oil. The crude residue was then purified by column chromatography on silica gel, eluting with a gradient of 0-75% 3:1 EtOAc/EtOH (with 2% triethylamine) to afford 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-4-(1,4-oxazepan-4-yl)quinazolin-6-ol as light yellow solid, which was carried forward to the next step without further purification. m/z (ESI): 653.2 (M+H)⁺.

TABLE 4
Conditions for Chiral SFC Separations.

| Compound | SFC Conditions | Peak to Ex.# |
|---|---|---|
| 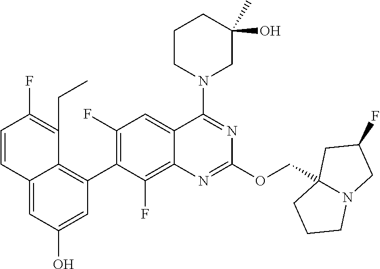 | Column: ChiralPak AD, 2 × 25 cm, 5 μm Mobile phase: 35% iPrOH with 0.2% DEA Flowrate: 80 mL/min. Yield: 100 mg sample was submitted to generate 37 mg of peak 1 with an ee of 99% and 33 mg of peak 2 with an ee of 99%. | Peak 1: Example 2 Peak 2: Example 3 |
| 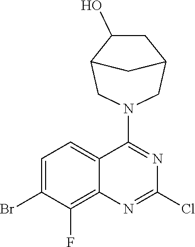 [SMILES: OC1CC2CC1CN(c1nc(Cl)nc3c(F)c(Br)ccc13)C2] | Column: ChiralPak AD 2 × 15 cm, 5 μm Mobile phase: 30% MeOH Flowrate: 80 mL/min. Yield: 2.40 g sample was submitted to generate 0.64 g of peak 1 with an ee of 99% and 0.59 g of peak 2 with an ee of 95%. | Peak 1: Example 15 Example 16 Peak 2: Example 17 |
| 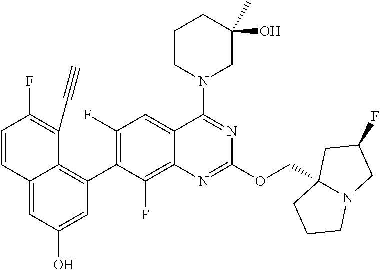 [SMILES: C#Cc1c(F)ccc2cc(O)cc(-c3c(F)cc4c(N5CCC[C@@](C)(O)C5)nc(OC[C@@]56CCCN5C[C@H](F)C6)nc4c3F)c12] | Column: ChiralPak IG 2 × 25 cm, 5 μm Mobile phase: 45% iPrOH with 0.2% DEA. Flowrate: 70 mL/min. Yield: 98 mg sample was submitted to generate 52 mg of peak. 1 with an ee of 99% and 35 mg of peak 2 with an ee of 98%. | Peak 1: Example 11 Peak 2: Example 12 |
| 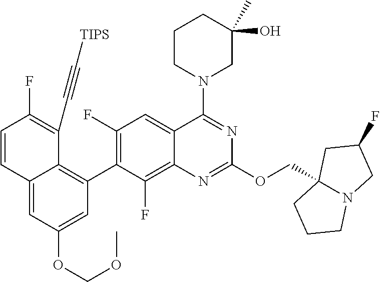 | Column: Whelk-01 (S, S), 21 × 250 mm, 5 μm Mobile phase: 25% MeOH with 0.2% TEA. Flowrate: 80 mL/min. Yield: 75 mg sample was submitted to generate 12 mg of peak 1 with an ee of >99% and 15 mg of peak 2 with an ee of >99%. | Peak 1: Example 13 Peak 2: Example 14 |
| 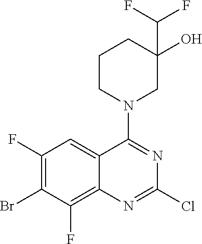 [SMILES: OC1(C(F)F)CCCN(c2nc(Cl)nc3c(F)c(Br)c(F)cc23)C1] | Column: ChiralPak IG, 2 × 25 cm, 5 μm Mobile phase: 25% MeOH Flowrate: 80 mL/min. Yield: 1.15 g sample was submitted to generate 543 mg of peak 1 with an ee of 99% and 586 mg of peak 2 with an ee of 99%. | Peak 1: Example 20 Example 21 |
| 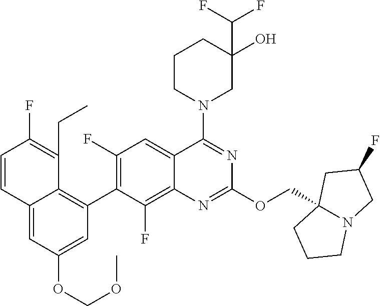 [SMILES: CCc1c(F)ccc2cc(OCOC)cc(-c3c(F)cc4c(N5CCCC(O)(C(F)F)C5)nc(OC[C@@]56CCCN5C[C@H](F)C6)nc4c3F)c12] | Column: ChiralPak IC, 2 × 25 cm, 5 μm Mobile phase: 25% iPrOH with 0.2% DEA Flowrate: 80 mL/min Yield: 113 mg sample was submitted to generate 32 mg of peak 1 with an ee of 99% and achiral purity of 91% and 33 mg of peak 2 with an ee of 91% and achiral purity of 99%. | Peak 1: Example 20 Peak 2: Example 21 |
| 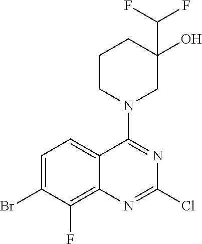 [SMILES: OC1(C(F)F)CCCN(c2nc(Cl)nc3c(F)c(Br)ccc23)C1] | Column: Chiralpak IG, 21 × 250 mm, 5 μm Mobile phase: 25% methanol Flowrate: 100 mL/min Yield: 887 mg sample was submitted to generate 346 mg of peak 1 with an ee of >99% and 380 mg of peak 2 with an ee of >96%. | Peak 1: Example 23 Peak 2: Example 24 |
| 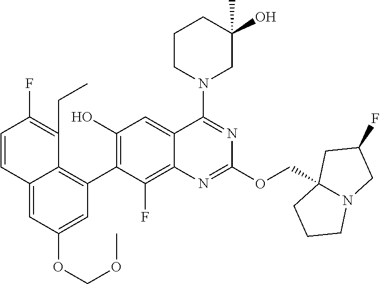 [SMILES: CCc1c(F)ccc2cc(OCOC)cc(-c3c(O)cc4c(N5CCC[C@@](C)(O)C5)nc(OC[C@@]56CCCN5C[C@H](F)C6)nc4c3F)c12] | Column: ChiralPak AD, 2 × 25 cm, 5 μm Mobile phase: 25% iPrOH with 0.2% DEA Flowrate: 80 mL/min Yield: 300 mg sample was submitted to generate 104 mg of peak 1 with an ee of 99% and 102 mg of peak 2 with an ee of 99%. | Peak 1: Example 27 Peak 2: Example 28 |
| 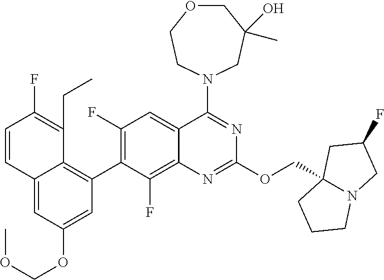 [SMILES: CCc1c(F)ccc2cc(OCOC)cc(-c3c(F)cc4c(N5CCOCC(C)(O)C5)nc(OC[C@@]56CCCN5C[C@H](F)C6)nc4c3F)c12] | Column: ChiralPak IG, 2 × 25 cm, 5 μm Mobile phase: 35% iPrOH with 0.2% DEA Flowrate: 70 mL/min Yield: 140 mg of sample was submitted to generate 74 mg of peak 1 with an ee of 99% and 45 mg of peak 2 with an ee of 97%. | Peak 1: Example 34 Peak 2: Example 35 |
| 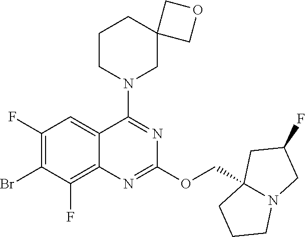 [SMILES: Fc1cc2c(N3CCCC4(COC4)C3)nc(OC[C@@]34CCCN3C[C@H](F)C4)nc2c(F)c1Br] | Column: ChiralPak AS, 2 × 15 cm, 5 μm Mobile phase: 20% MeOH Flowrate: 80 mL/min. Yield: 640 mg of sample was submitted to generate generate 294 mg of peak 1 with an ee of 99% and 306 mg of peak 2 with an ee of 99%. | Peak 1: Example 38 Peak 2: Example 39 |
| 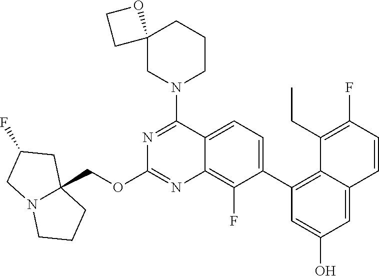 [SMILES: CCc1c(F)ccc2cc(O)cc(-c3ccc4c(N5CCC[C@]6(CCO6)C5)nc(OC[C@@]56CCCN5C[C@H](F)C6)nc4c3F)c12] | Column: ChiralPak AS, 21 × 250 cm, 5 μm Mobile phase: 25% MeOH with 0.2% Et₂NH Flowrate: 150 mL/min. Yield: 3.96 g of sample was submitted to generate generate 1.82 g of peak 1 with an ee of 99% and 1.83 g of peak 2 with an ee of 96% | Peak 2: Example 76 |
| 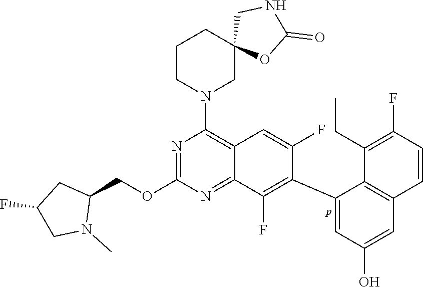 [SMILES: CCc1c(F)ccc2cc(O)cc(-c3c(F)cc4c(N5CCC[C@]6(CNC(=O)O6)C5)nc(OC[C@@H]5C[C@@H](F)CN5C)nc4c3F)c12] | Column: ChiralPak AS, 2 × 25 cm, 5 μm Mobile phase: 35% MeOH with 0.2% DEA Flowrate: 80 mL/min Yield: 180 mg of sample was submitted to generate 31 mg of peak 1 with an ee of 99%, 29.7 mg of peak 2 with an ee of 99%, and 70 mg of Peak 3 and Peak 4 combined. The mixture of Peak 3 and Peak 4 was purified via SFC using a Chiralpak ID, 2 × 25 mm, 5 μm column with a mobile phase of 40% IPrOH with 0.2% DEA using a flowrate of 80 mL/min to generate 28.5 mg of peak 3 with an ee of 99% and 25.8 mg of peak 4 with an ee of 99%. | Peak 2: Example 80 |

TABLE 5
Analytical Data for Examples 2 to 43, 76 to 85.

| | MS | |
|---|---|---|
| Ex. | m/z (ESI): | |
| # | (M + H)⁺ | ¹H NMR |
| 2 | 625.3 | ¹H NMR (400 MHz, METHANOL-d₄) δ ppm 7.85 (dd, J = 9.9, 1.6 |
| | | Hz, 1 H), 7.69 (dd, J = 9.0, 5.9 Hz, 1 H), 7.31 (d, J = 2.7 Hz, 1 H), |
| | | 7.26 (t, J = 9.3 Hz, 1 H), 7.00 (d, J = 2.5 Hz, 1 H), 5.27-5.53 (m, 1 H), |
| | | 4.37-4.56 (m, 2 H), 4.28 (br d, J = 13.4 Hz, 1 H), 4.11 (br d, |
| | | J = 13.2 Hz, 1 H), 3.38-3.61 (m, 5 H), 3.12-3.29 (m, 1 H), |
| | | 1.72-2.66 (m, 12 H), 1.31 (s, 3 H), 0.84 (t, J = 7.4 Hz, 3 H). |
| 3 | 625.3 | ¹H NMR (400 MHz, METHANOL-d₄) δ ppm 7.78 (d, J = 9.7 Hz, 1 H), |
| | | 7.69 (dd, J = 9.0, 5.7 Hz, 1 H), 7.31 (d, J = 2.7 Hz, 1 H), 7.26 (t, |
| | | J = 9.3 Hz, 1 H), 7.00 (d, J = 2.7 Hz, 1 H), 5.21-5.43 (m, 1 H), 4.19- |
| | | 4.37 (m, 3 H), 4.07 (br d, J = 13.3 Hz, 1 H), 3.39-3.58 (m, 2 H), |
| | | 3.14-3.28 (m, 2 H), 3.02 (td, J = 9.1, 5.6 Hz, 1 H), 1.67-2.68 (m, |
| | | 13 H), 1.28 (s, 3 H), 0.82 (t, J = 7.4 Hz, 3 H). |
| 4 | 628.0 | ¹H NMR (400 MHz, METHANOL-d₄) δ ppm 7.98 (d, J = 8.6 Hz, 1 H), |
| | | 7.69 (dd, J = 9.0, 5.9 Hz, 1 H), 7.54 (ddd, J = 8.7, 6.7, 2.3 Hz, 1 H), |
| | | 7.24-7.31 (m, 2 H), 6.97-7.00 (m, 1 H), 5.50-5.68 (m, 1 H), |
| | | 4.67-4.75 (m, 2 H), 4.39-4.54 (m, 2 H), 3.88-4.15 (m, 5 H), |
| | | 3.74-3.86 (m, 1 H), 3.46-3.54 (m, 1 H), 2.57-2.87 (m, 3 H), |
| | | 2.33-2.53 (m, 6 H), 2.13-2.26 (m, 1 H), 0.75-0.82 (m, 3 H). |
| 5 | 685.8 | ¹H NMR (400 MHz, METHANOL-d₄) δ ppm 8.00 (d, J-8.6 Hz, 1 H), |
| | | 7.69 (dd, J = 9.1, 6.0 Hz, 1 H), 7.49 (ddd, J = 8.6, 6.8, 3.3 Hz, 1 H), |
| | | 7.23-7.30 (m, 2 H), 7.00 (dd, J = 10.6, 2.6 Hz, 1 H), 5.49-5.72 |
| | | (m, 1 H), 4.99 (br t, J = 11.0 Hz, 1 H), 4.63-4.75 (m, 3 H), |
| | | 4.27-4.38 (m, 1 H), 3.84-4.14 (m, 4 H), 3.71 (s, 3 H), 3.42-3.54 |
| | | (m, 1 H), 3.12-3.31 (m, 1 H), 2.61-2.88 (m, 2 H), 2.33-2.59 |
| | | (m, 6 H), 2.09-2.30 (m. 2 H), 0.74-0.83 (m, 3 H). |
| 6 | 603.1 | ¹H NMR (400 MHz, METHANOL-d₄) δ ppm 7.84-7.97 (m, 1 H), |
| | | 6.97 (d, J = 2.5 Hz, 1 H), 6.65-6.67 (m, 1 H), 5.48-5.67 (m, 1 H), |
| | | 4.64-4.73 (m, 2 H), 4.39-4.52 (m, 1 H), 4.18-4.26 (m, 1 H), |
| | | 3.96-4.08 (m, 1 H), 3.83-3.96 (m, 2 H), 3.33-3.59 (m, 3 H), |
| | | 2.52-2.79 (m, 2 H), 2.29-2.48 (m, 3 H), 2.07-2.23 (m, 2 H), |
| | | 1.71-1.91 (m, 4 H), 1.25-1.32 (m, 3 H), 0.63 (br d, J = 6.6 Hz, 2 H), |
| | | 0.18 (br s, 2 H). |
| 7 | 571.0 | ¹H NMR (400 MHz, METHANOL-d₄) δ ppm 8.00-8.07 (m, 1 H), |
| | | 7.30-7.40 (m, 1 H), 6.91-7.00 (m, 1 H), 6.65-6.72 (m, 1 H), |
| | | 5.46-5.67 (m, 1 H), 4.64-4.73 (m, 3 H), 4.20-4.32 (m, 4 H), |
| | | 4.02-4.08 (m, 2 H), 3.82-3.98 (m, 4 H), 3.41-3.55 (m, 1 H), |
| | | 2.52-2.79 (m, 2 H), 2.29-2.50 (m, 3 H), 2.08-2.26 (m, 3 H), |
| | | 1.74-1.89 (m, 1 H), 0.43-0.84 (m, 2 H), -0.05-0.29 (m, 2 H) |
| 8 | 606.0 | ¹H NMR (400 MHz, METHANOL-d₄) δ ppm 8.23-8.32 (m, 1 H), |
| | | 7.64-7.74 (m, 1 H), 7.46-7.57 (m, 1 H), 7.29 (d, J = 2.7 Hz, 2 H), |
| | | 6.94-7.00 (m, 1 H), 5.45-5.68 (m, 1 H), 4.70-4.77 (m, 2 H), |
| | | 4.12-4.40 (m, 4 H), 3.79-4.10 (m, 3 H), 3.41-3.54 (m, 1 H), |
| | | 3.34-3.42 (m, 1 H), 2.53-2.80 (m, 2 H), 2.28-2.52 (m, 7 H), |
| | | 2.12-2.27 (m, 1 H), 0.81 (br d, J = 2.1 Hz, 3 H). |
| 9 | 611.2 | ¹H NMR (400 MHz, METHANOL-d₄) δ ppm 7.85-7.92 (m, 1 H), |
| | | 7.67-7.76 (m, 1 H), 7.33 (d, J = 2.5 Hz, 1 H), 7.24-7.31 (m, 1 H), |
| | | 7.01 (s, 1 H), 5.44-5.70 (m, 1 H), 4.68 (dd, J = 11.2, 3.9 Hz, |
| | | 2 H), 4.22-4.34 (m, 4 H), 3.81-4.10 (m, 7 H), 3.43-3.53 (m, 1 H), |
| | | 2.59 (br s, 3 H), 2.31-2.51 (m, 4 H), 2.21 (br d, J = 5.4 Hz, 3 H), |
| | | 0.80-0.88 (m, 3 H). |
| 10 | 564.9 | ¹H NMR (400 MHz, METHANOL-d₄) δ ppm 7.87-7.96 (m, 1 H), |
| | | 7.76-7.84 (m, 1 H), 7.43-7.50 (m, 1 H), 7.34-7.40 (m, 1 H), |
| | | 7.28-7.33 (m, 1 H), 7.22-7.28 (m, 1 H), 7.12-7.19 (m, 1 H), |
| | | 5.51 (s, 1 H), 4.63-4.76 (m, 2 H), 4.27 (s, 4 H), 3.87 (s, 7 H), |
| | | 3.41-3.55 (m, 1 H), 2.54-2.77 (m, 2 H), 2.36 (br d, J = 5.9 Hz, 3H), |
| | | 2.10-2.28 (m, 3 H). |
| 11 | 603.1 | ¹H NMR (400 MHz, METHANOL-d₄) δ ppm 7.82 (d, J = 8.3 Hz, 1 H), |
| | | 7.60-7.68 (m, 1 H), 7.51 (d, J = 6.4 Hz, 1 H), 7.36-7.43 (m, 1 H), |
| | | 7.32 (d, J = 2.5 Hz, 1 H), 7,08 (d, J = 2.5 Hz, 1 H), 5.16-5.45 |
| | | (m, 1 H), 4.16-4.38 (m, 3 H), 4.03 (br d, J = 13.5 Hz, 1 H), 3.51 |
| | | (d, ,J = 13.5 Hz, 1 H), 3.39-3.46 (m, 1 H), 3.30 (br s, 1 H), 3.18-3.28 |
| | | (m, 2 H), 3.02 (td, J = 9.4, 5.9 Hz, 1 H), 2.94 (s, 1 H), 2.08-2.45 |
| | | (m, 4 H), 1.69-2.06 (m, 6 H), 1.29 (s, 3 H). |
| 12 | 603.3 | ¹H NMR (400 MHz, METHANOL-d₄) δ ppm 7.81 (d, J = 7.3 Hz, 1 H), |
| | | 7.63 (dd, J = 9.7, 1.5 Hz, 1 H), 7.50 (dd, J = 7.2, 1.1 Hz, 1 H), |
| | | 7.36-7.42 (m, 1 H), 7.32 (d, J = 2.7 Hz, 1 H), 7.09 (d, J = 2.5 Hz, |
| | | 1 H), 5.17-5.47 (m, 1 H), 4.18-4.37 (m, 3 H), 4.08 (br d, J = 13.3 |
| | | Hz, 1 H), 3.46 (d, J = 13.3 Hz, 1 H), 3.38 (br s, 1 H), 3.13-3.30 |
| | | (m, 3 H), 2.96-3.09 (m, 2 H), 2.11-2.44 (m, 4 H), 1.66-2.09 |
| | | (m, 6 H), 1.27 (s, 3 H). |
| 13 | 621.1 | ¹H NMR (400 MHz, METHANOL-d₄) δ ppm 7.86 (dd, J = 9.1, 5.7 Hz, |
| | | 1 H), 7.66 (dd, J = 9.8, 1.7 Hz, 1 H), 7.27-7.38 (m, 2 H), 7.13 |
| | | (d, J = 2.3 Hz, 1 H), 5.21-5.44 (m, 1 H), 4.27-4.37 (m, 1 H), 4.21 |
| | | (d, J = 10.5 Hz, 2 H), 4.03 (d, J = 13.2 Hz, 1 H), 3.51 (d, J = 13.4 Hz, |
| | | 1 H), 3.37-3.47 (m, 1 H), 3.14-3.28 (m, 4 H), 3.02 (td, J = 9.4, |
| | | 5.9 Hz, 1 H), 2.10-2.44 (m, 4 H), 1.70-2.06 (m, 7 H), 1.29 (s, 3 H) |
| 14 | 621.0 | ¹H NMR (400 MHz, METHANOL-d₄) δ ppm 7.85 (dd, J = 9.2, 5.9 |
| | | Hz, 1 H), 7.64 (dd, J = 9.8, 1.7 Hz, 1 H), 7.27-7.39 (m, 2 H), 7.15 |
| | | (d, J = 2.5 Hz, 1 H), 5.18-5.44 (m, 1 H), 4.19-4.39 (m, 3 H), 4,09 |
| | | (br d, J = 13.2 Hz, 1 H), 3.48 (d, J = 13.2 Hz, 1 H), 3.36-3.43 (m, 2 H), |
| | | 3.13-3.29 (m, 3 H), 3.02 (td, J = 9.4, 6.0 Hz, 1 H), 2.11-2.45 |
| | | (m, 4 H), 1.74-2.04 (m, 6 H), 1.27 (s, 3 H). |
| 15 | 619.2 | ¹H NMR (400 MHz, METHANOL-d₄) δ ppm 8.16 (dd, 1 H, J = 8.5, |
| | | 14.7 Hz), 7.68 (dd, 1 H, J = 5.9, 9.2 Hz), 7.4-7.5 (m, 1 H), 7.2-7.3 |
| | | (m, 2 H), 6.97 (dd, 1 H, J = 2.6, 4.5 Hz), 5.5-5.7 (m, 1 H), 4.8-5.0 |
| | | (m, 2 H), 4.6-4.7 (m, 2 H), 4.37 (br dd, 1 H, J = 5.5, 10.3 Hz), 4.0-4.1 |
| | | (m, 1 H), 3.7-4.0 (m, 3 H), 3.4-3.6 (m, 2 H), 1.8-2.7 (m, 14 H), 1.4-1.5 |
| | | (m, 1 H), 0.7-0.9 (m, 3H). |
| 16 | 597.3 | ¹H NMR (400 MHz, METHANOL-d₄) δ ppm 7.80-7.88 (m, 1 H), |
| | | 7.78-7.80 (m, 1 H), 7.4-7.5 (m, 1 H), 7.3-7.4 (m, 1 H), 7.27 (d, 1 H, |
| | | J = 2.5 Hz), 7.18 (dt, 1 H, J = 6.8, 8.6 Hz), 7.0-7.1 (m, 1 H), 5.2-5.4 |
| | | (m, 1 H), 4.9-5.0 (m, 1 H), 4.6-4.8 (m, 1 H), 4.2-4.4 (m, 2 H), 4.2-4.2 |
| | | (m, 1 H), 3.74 (dd, 1 H, J = 1.6, 12.6 Hz), 3.5-3.6 (m, 1 H), 3.2-3.3 |
| | | (m, 2 H), 3.02 (dt, 1 H, J = 5.7, 9.4 Hz), 2.1-2.4 (m, 6 H), 1.8-2.1 |
| | | (m, 5 H), 1.4-1.6 (m, 1 H), 0.8-1.0 (m. 2 H). |
| 17 | 615.3 | ¹H NMR ( 400 MHz, METHANOL-d₄) δ ppm 8.0-8.1 (m, 1 H), |
| | | 7.8-7.9 (m, 1 H), 7.3-7.4 (m, 3 H), 7.1-7.2 (m, 1 H), 5.4-5.7 (m, 1 H), |
| | | 5.1-5.2 (m, 1 H), 4.6-4.8 (m, 3 H), 4.3-4.4 (m, 1 H), 3.8-4.0 (m, 4 H), |
| | | 3.4-3.5 (m, 2 H), 2.1-2.6 (m, 10 H), 1.9-2.0 (m, 1 H), 1.8-1.9 (m, |
| | | 1 H), 1.4-1.5 (m, 1 H). |
| 18 | 623.2 | ¹H NMR (400 MHz, METHANOL-d₄) δ ppm 7.68-7.80 (m, 2 H), |
| | | 7.24-7.37 (m, 2 H), 7.00 (d, J = 2.5 Hz, 1 H), 5.45-5.68 (m, 1 H), |
| | | 4.61-4.75 (m, 2 H), 4.40-4.59 (m, 4 H), 3.86-4.11 (m, 3 H), |
| | | 3.67-3.85 (m, 3 H), 3.42-3.54 (m, 1 H), 2.55-2.67 (m, 3 H), |
| | | 2.40-2.48 (m, 2 H), 2.06-2.06 (m, 1 H), 1.87-2.04 (m, 5 H), |
| | | 0.82 (td, J = 7.4, 1.7 Hz, 3 H), |
| 19 | 597.3 | ¹H NMR (400 MHz, METHANOL-d₄) δ ppm 7.93-8.14 (m, 1 H), |
| | | 7.70 (dd, J = 9.2, 5.9 Hz, 1 H), 7.20-7.40 (m, 2 H), 6.92-7.03 (m, 1 H), |
| | | 5.44-5.68 (m, 1 H), 4.58-4.71 (m, 3 H), 3,77-4.34 (m, 7 H), |
| | | 3.41-3.56 (m, 2 H), 2.18-2.65 (m, 9 H), 0.84 (dt, J = 10.5, 7.6 Hz, 3 H). |
| 20 | 661.0 | ¹H NMR (400 MHz, METHANOL-d₄) δ ppm 7.88-7.96 (m, 1 H), |
| | | 7.64-7.74 (m, 1 H), 7.31-7.35 (m, 1 H), 7.23-7.31 (m, 1 H), |
| | | 6.98-7.03 (m, 1 H), 5.65-5.95 (m, 1 H), 5.49-5.65 (m, 1 H), |
| | | 4.64-4.75 (m, 2 H), 4.50-4.59 (m, 1 H), 4.36-4.45 (m, 1 H), |
| | | 3.81-4.08 (m, 3 H), 3.55-3.63 (m, 1 H), 3.44-3.54 (m, 1 H), |
| | | 3.35-3.43 (m, 1 H), 2.52-2.79 (m, 3 H), 2.42-2.51 (m, 2 H), |
| | | 2.30-2.41 (m, 2 H), 2.10-2.29 (m, 2 H), 1.84-1.99 (m, 3 H), |
| | | 0.78-0.89 (m, 3 H) |
| 21 | 661.0 | ¹H NMR (400 MHz, METHANOL-d₄) δ ppm 7.86-7.93 (m, 1 H), |
| | | 7.67-7.74 (m, 1 H), 7.32-7.35 (m, 1 H), 7.24-7.31 (m, 1 H), |
| | | 6.99-7.03 (m, 1 H), 5.48-5.94 (m, 2 H), 4.62-4.75 (m, 2 H), |
| | | 4.48-4.57 (m, 1 H), 4.34-4.42 (m, 1 H), 3.82-4.10 (m, 3 H), |
| | | 3.60-3.70 (m, 1 H), 3.44-3.54 (m, 1 H), 3.35-3.44 (m, 1 H), |
| | | 2.50-2.80 (m, 3 H), 2.29-2.49 (m, 4 H), 2.10-2.29 (m, 2 H), |
| | | 1.81-1.98 (m, 3 H), 0.78-0.87 (m, 3 H). |
| 22 | 661.3 | ¹H NMR (400 MHz, METHANOL-d₄) 6 ppm 7.87-7.96 (m, 1 H), |
| | | 7.66-7.75 (m, 1 H), 7.31-7.36 (m, 1 H), 7.22-7.31 (m, 1 H), |
| | | 6.98-7.04 (m, 1 H), 5.47-5.96 (m, 2 H), 4.64-4.76 (m, 2 H), |
| | | 4.48-4.59 (m. 1 H), 4.35-4.45 (m, 1 H), 3.81-4.10 (m, 3 H), |
| | | 3.55-3.69 (m, 1 H), 3.44-3.54 (m, 1 H), 3.35-3.44 (m, 1 H), |
| | | 2.51-2.78 (m, 3 H), 2.31-2.51 (m, 4 H), 2.11-2.29 (m, 2 H), |
| | | 1.82-2.00 (m, 3 H), 0.78-0.88 (m, 3 H). |
| 23 | 642.9 | ¹H NMR (400 MHz, METHANOL-d₄) δ ppm 8.08-8.16 (m, 1 H), |
| | | 7.65-7.71 (m, 1 H), 7.45-7.53 (m, 1 H), 7.23-7.31 (m, 2 H), |
| | | 6.95-7.00 (m, 1 H), 5.65-5.96 (m, 1 H), 5.49-5.65 (m, 1 H), |
| | | 4.75 (s, 3 H), 4.49-4.60 (m, 1 H), 3.81-4.10 (m, 3 H), 3.63- |
| | | 3.76 (m, 1 H), 3.42-3.54 (m, 2 H), 2.55-2.82 (m, 2 H), 2.31- |
| | | 2.52 (m, 5 H), 2.13-2.30 (m, 2 H), 1.86-2.05 (m, 3 H), 0.81 (d, |
| | | J = 7.5 Hz, 3 H). |
| 24 | 642.9 | ¹H NMR (400 MHz, METHANOL-d₄) δ ppm 8.08-8.16 (m, 1 H), |
| | | 7.65-7.72 (m, 1 H), 7.44-7.52 (m, 1 H), 7.22-7.32 (m, 2 H), |
| | | 6.95-7.01 (m, 1 H), 5.65-5.96 (m, 1 H), 5.47-5.65 (m, 1 H), |
| | | 4.69-4.77 (m, 2 H), 4.62-4.69 (m, 1 H), 4.49-4.59 (m, 1 H), |
| | | 3.80-4.09 (m, 3 H), 3.64-3.75 (m, 1 H), 3.42-3.53 (m, 2 H), |
| | | 2.54-2.79 (m, 2 H), 2.41-2.51 (m, 3 H), 2.30-2.41 (m, 2 H), |
| | | 2.12-2.30 (m, 2 H), 1.87-2.03 (m, 3 H), 0.81 (d, J = 7.1 Hz, 3 H). |
| 25 | 640.9 | ¹H NMR (400 MHz, METHANOL-d₄) δ ppm 8.22-8.29 (m, 1 H), |
| | | 7.66-7.74 (m, 1 H), 7.30-7.34 (m, 1 H), 7.23-7.30 (m, 1 H), |
| | | 6.90-6.94 (m, 1 H), 5.47-5.67 (m, 1 H), 4.63-4.75 (m, 2 H), |
| | | 4.42-4.52 (m, 1 H), 4.18-4.28 (m, 1 H), 3.80-4.10 (m, 3 H), |
| | | 3.53-3.66 (m, 1 H), 3.41-3.52 (m, 2 H), 2.53-2.81 (m, 3 H), |
| | | 2.25-2.48 (m, 4 H), 2.11-2.25 (m, 2 H), 1.74-1.93 (m, 3 H), |
| | | 1.32 (d, J = 10.9 Hz, 3 H), 0.77-0.89 (m, 3 H). |
| 26 | 609.2 | ¹H NMR (400 MHz, METHANOL-d₄) δ ppm 7.63-7.70 (m, 1 H) |
| | | 7.46-7.49 (m, 1 H) 7.27-7.29 (m, 1 H) 7.20-7.26 (m, 1 H) 6.93 |
| | | (s, 1 H) 5.42-5.66 (m, 1 H) 4.57-4.71 (m, 3 H) 4.18-4.34 (m, 4 H) |
| | | 4.04-4.11 (m, 2 H) 3.79-4.02 (m, 5 H) 3.41-3.52 (m, 1 H) |
| | | 2.49-2.76 (m, 4 H) 2.31-2.49 (m, 3 H) 2.07-2.29 (m, 3 H) |
| | | 0.81-0.88 (m, 3 H). |
| 27 | 623.0 | ¹H NMR (400 MHz, METHANOL-d₄) δ ppm 7.62-7.71 (m, 1 H), |
| | | 7.45-7.49 (m, 1 H), 7.27-7.29 (m, 1 H), 7.20-7.26 (m, 1 H), |
| | | 6.91-6.95 (m, 1 H), 5.45-5.65 (m, 1 H), 4.63-4.75 (m, 2 H), |
| | | 4.34-4.44 (m, 1 H), 4.20-4.31 (m, 1 H), 3.79-4.06 (m, 3 H), |
| | | 3.52-3.61 (m, 1 H), 3.41-3.52 (m, 2 H), 2.50-2.78 (m, 4 H), |
| | | 2.30-2.49 (m, 3 H), 2.08-2.26 (m, 2 H), 1.77-1.94 (m, 3 H), |
| | | 1.29-1.36 (m, 3 H), 0.80-0.91 (m, 3 H). |
| 28 | 623.0 | ¹H NMR (400 MHz, METHANOL-d₄) δ ppm 7.63-7.71 (m, 1 H), |
| | | 7.43-7.48 (m, 1 H), 7.27-7.29 (m, 1 H), 7.20-7.27 (m, 1 H), |
| | | 6.91-6.95 (m, 1 H), 5.44-5.67 (m, 1 H), 4.62-4.73 (m, 2 H), |
| | | 4.33-4.44 (m, 1 H), 4.16-4.26 (m, 1 H), 3.91-4.07 (m, 1 H), |
| | | 3.79-3.90 (m, 2 H), 3.56-3.64 (m, 1 H), 3.42-3.54 (m, 2 H), |
| | | 2.31-2.77 (m, 7 H), 2.08-2.28 (m, 2 H), 1.75-1.93 (m, 3 H), |
| | | 1.31 (s, 3 H), 0.83 (t, J = 7.4 Hz, 3 H). |
| 29 | 623.0 | ¹H NMR (400 MHz, METHANOL-d₄) δ ppm 8.33-8.52 (m, 1 H), |
| | | 7.60-7.84 (m, 1 H), 7.40-7.53 (m, 1 H), 7.29 (d, J = 2.7 Hz, 2 H), |
| | | 6.98 (s, 1 H), 5.46-5.71 (m, 1 H), 4.69-4.80 (m, 7 H), 4.50- |
| | | 4.68 (m, 2 H), 4.14-4.33 (m, 1 H), 3.82-4.13 (m, 7 H), 3.61- |
| | | 3.79 (m, 2 H), 3.39-3.58 (m, 1 H), 3.19-3.39 (m, 1 H), 2.54- |
| | | 2.79 (m, 2 H), 2.15-2.54 (m, 7 H), 1.30 (d, J = 5.6 Hz, 3 H), 0.82 |
| | | (s, 3 H). |
| 30 | 605.2 | ¹H NMR (400 MHz, METHANOL-d₄) δ ppm 8.24-8.49 (m, 1 H), |
| | | 7.54-7.86 (m, 1 H), 7.33-7.50 (m, 2 H), 7.13-7.30 (m, 2 H), |
| | | 6.90-7.01 (m, 1 H), 5.44-5.69 (m, 1 H), 4.65-4.75 (m, 2 H), |
| | | 4.47-4.62 (m, 2 H), 3.81-4.25 (m, 7 H), 3.63-3.76 (m, 2 H), |
| | | 3.38-3.58 (m, 1 H), 2.30-2.82 (m, 7 H), 2.04-2.30 (m, 1 H), |
| | | 1.30 (d, J = 5.9 Hz, 3 H), 0.80-0.95 (m, 3 H). |
| 31 | 613.9 | ¹H NMR (400 MHz, METHANOL-d₄) δ ppm 8.20-8.51 (m, 1 H), |
| | | 7.59-7.71 (m, 1 H), 7.36-7.52 (m, 2 H), 7.26-7.34 (m, 1 H), |
| | | 6.90-7.23 (m, 1 H), 5.38-5.75 (m, 1 H), 4.65-4.76 (m, 2 H), |
| | | 4.37-4.63 (m, 2 H), 3.82-4.25 (m, 7 H), 3.63-3.77 (m, 2 H), |
| | | 3.39-3.56 (m, 1 H), 2.57-2.86 (m, 2 H), 2.28-2.54 (m, 3 H), |
| | | 2.06-2.25 (m, 1 H), 1.28 -1.32 (m, 3 H). |
| 32 | 631.2 | ¹H NMR (400 MHz, METHANOL-d₄) δ ppm 8.14-8.34 (m, 1 H), |
| | | 7.57-7.70 (m, 1 H), 7.32-7.48 (m, 2 H), 7.09-7.24 (m, 1 H), |
| | | 5.35-5.72 (m, 1 H), 4.61-4.77 (m, 2 H), 4.39-4.60 (m, 2 H), |
| | | 3.98-3.97 (m, 7 H), 3.60-3.78 (m, 2 H), 3.41-3.58 (m, 1 H), |
| | | 2.26-2.80 (m, 5 H), 2.12-2.25 (m, 1 H), 1.30 (s, 3 H). |
| 33 | 622.9 | ¹H NMR (400 MHz, METHANOL-d₄) δ ppm 8.16-8.36 (m, 1 H), |
| | | 7.50-7.81 (m, 1 H), 7.27-7.44 (m, 2 H), 7.09-7.21 (m, 1 H), |
| | | 6.90-7.02 (m, 1 H), 5.37-5.80 (m, 1 H), 4.38-4.75 (m, 4 H), |
| | | 3.83-4.23 (m, 7 H), 3.61-3.79 (m, 2 H), 3.40-3.58 (m, 1 H), |
| | | 2.29-2.82 (m, 7 H), 2.09-2.29 (m, 1 H), 1.28-1.32 (m, 3 H), |
| | | 0.81-1.02 (m, 3 H). |
| 34 | 640.9 | ¹H NMR (400 MHz, METHANOL-d₄) δ ppm 8.22-8.46 (m, 1 H), |
| | | 7.60-7.89 (m, 1 H), 7.19-7.52 (m, 2 H), 6.99-7.10 (m, 1 H), |
| | | 5.38-5.75 (m, 1 H), 4.63-4.76 (m, 2 H), 4.45-4.62 (m, 2 H), |
| | | 3.81-4.21 (m, 7 H), 3.63-3.77 (m, 2 H), 3.40-3.55 (m, 1 H), |
| | | 2.30-2.79 (m, 7 H), 2.12-2.26 (m, 1 H), 1.30 (s, 3 H), 0.70- |
| | | 0.92 (s, 3 H). |
| 35 | 640.9 | ¹H NMR (400 MHz, METHANOL-d₄) δ ppm 8.15-8.40 (m, 1 H), |
| | | 7.57-7.79 (m, 1 H), 7.22-7.41 (m, 2 H), 6.90-7.07 (m, 1 H), |
| | | 5.45-5.75 (m, 1 H), 4.62-4.74 (m, 2 H), 4.39-4.59 (m, 2 H), |
| | | 3.81-4.21 (m, 7 H), 3.65-3.72 (m, 2 H), 3.40-3.55 (m, 1 H), |
| | | 2.30-2.81 (m, 7 H), 2.08-2.24 (m, 1 H), 1.28 (s, 3 H), 0.77-0.98 |
| | | (m, 3 H). |
| 36 | 623.0 | ¹H NMR (400 MHz, DMSO-d₆) δ ppm 10.59-10.86 (m, 1 H) 9,79- |
| | | 10.12 (m, 1 H) 7.78-7.87 (m, 2 H) 7.37 (s, 1 H) 7.35-7.37 (m, 1 H) |
| | | 7.01 (dd, J = 12.02, 2.61 Hz, 1 H) 5.43-5.70 (m, 1 H) 4.65-4.76 |
| | | (m, 1 H) 4.55-4.59 (m, 2 H) 4.41 (br d, J = 3.55 Hz, 1 H) 4.37-4.44 |
| | | (m, 1 H) 4.26-4.35 (m, 1 H) 3.61-3.65 (m, 3 H) 3.25-3.40 |
| | | (m, 2 H) 2.70 (br s, 1 H) 2.58-2.74 (m, 1 H) 2.28-2.42 (m, 3 H) |
| | | 2.12-2.26 (m, 3 H) 2.02 (s, 4 H) 0.76 (q, J = 7.46 Hz, 4 H). |
| 37 | 647.0 | ¹H NMR (400 MHz, DMSO-d₆) δ ppm 10.70 (br s, 1 H) 10.00 (br |
| | | s, 1 H) 7.85 (d, J = 10.03 Hz, 1 H) 7.80 (dd, J = 9.20, 6.06 Hz, 1 H) |
| | | 7.35-7.41 (m, 2 H) 7.02 (d, J = 2.72 Hz, 1 H) 5.43-5.73 (m, 1 H) |
| | | 4.60 (br t, J = 5.43 Hz, 5 H) 4.21 (br s, 3 H) 4.11 (br s, 3 H) 3.84-3.89 |
| | | (m, 2 H) 3.25-3.43 (m, 1 H) 2.59-2.69 (m, 1 H) 2.53-2.56 (m, 1 H) |
| | | 2.46 (br d, J = 6.48 Hz, 1 H) 2.30-2.42 (m, 2 H) 2.00-2.27 (m, 3 H) |
| | | 0.76 (t, J = 7.32 Hz, 3 H). |
| 38 | 637.0 | ¹H NMR (400 MHz, METHANOL-d₄) δ ppm 7.93 (br s, 1 H), |
| | | 7.69 (dd, J = 8.9, 6.0 Hz, 1 H), 7.30-7.33 (m, 1 H), 7.23-7.29 (m, |
| | | 1 H), 7.00 (s, 1 H), 5.47-5.64 (m, 1 H), 4.52-4.73 (m, 5 H), 4.27- |
| | | 4.39 (m, 1 H), 3.80-4.14 (m, 3 H), 3.69 (s, 1 H), 3.34-3.52 (m, |
| | | 2 H), 2.39-2.78 (m, 7 H), 2.25-2.38 (m, 3 H), 1.98-2.22 (m, 2 H), |
| | | 1.87-1.97 (m, 1 H), 1.75-1.86 (m, 1 H), 0.78-0.85 (m, 3 H) |
| 39 | 637.0 | ¹H NMR (400 MHz, METHANOL-d₄) δ ppm 7.94 (br t, J = 7.9 Hz, |
| | | 1 H), 7.69 (dd, J = 8.8, 5.9 Hz, 1 H), 7.30-7.34 (m, 1 H), 7.23-7.30 |
| | | (m, 1 H), 7.01 (s, 1 H), 5.47-5.66 (m, 1 H), 4.49-4.73 (m, 5 H), |
| | | 4.32 (br s, 1 H), 3.79-4.10 (m, 3 H), 3.69 (br t, J = 13.2 Hz, 1 H), |
| | | 3.46 (br dd, J = 10.7, 4.7 Hz, 1 H), 3.34-3.41 (m, 1 H), 2.62-2.77 |
| | | (m, 1 H), 2.28-2.60 (m, 9 H), 2.11-2.21 (m, 1 H), 2.04 (br s, 1 H), |
| | | 1.88-1.98 (m, 1 H), 1.81 (br d, J = 9.3 Hz, 1 H), 0.82 (br t, |
| | | J = 7.2 Hz, 3 H) |
| 40 | 385.9 | ¹H NMR (400 MHz, METHANOL-d₄) δ ppm 7.97-8.20 (m, 1 H), |
| | | 7.63-7.71 (m, 1 H), 7.39-7.50 (m, 1 H), 7.21-7.29 (m, 2 H), |
| | | 6.94-7.00 (m, 1 H), 5.46-5.64 (m, 1 H), 4.65-4.73 (m, 3 H), |
| | | 4.45-4.60 (m, 2 H), 4.00-4.14 (m, 1 H), 3.79-3.95 (m, 3 H), |
| | | 3.67-3.77 (m, 1 H), 3.41-3.56 (m, 2 H), 2.56-2.79 (m, 2 H), |
| | | 2.40-2.55 (m, 5 H), 2.24-2.39 (m, 4 H), 1.76-2.23 (m, 4 H), |
| | | 0.80 (t. J = 7.3 Hz, 3 H). |
| 41 | 547.6 | ¹H NMR (400 MHz, METHANOL-d₄) δ ppm 8.15 (d, J = 8.6 Hz, |
| | | 1 H), 7.75-7.82 (m, 1 H), 7.41-7.54 (m, 3 H), 7.21-7.32 (m, 2 H), |
| | | 7.11-7.17 (m, 1 H), 5.42-5.66 (m, 1 H), 4.91-5.00 (m, 1 H), |
| | | 4.74-4.83 (m, 1 H), 4.27-4.46 (m, 4 H), 4.02-4.21 (m, 3 H), |
| | | 3.82-3.97 (m, 2 H), 3.66-3.78 (m, 1 H), 3.42-3.62 (m, 2 H), |
| | | 2.68-2.81 (m, 1 H), 2.39-2.59 (m, 2 H), 2.13-2.37 (m, 5 H). |
| 42 | 547.9 | ¹H NMR (400 MHz, METHANOL-d₄) δ ppm 8.14 (dd, J = 8.8, 0.8 |
| | | Hz, 1 H), 7.78 (d, J = 7.9 Hz, 1 H), 7.40-7.54 (m, 3 H), 7.20-7.33 |
| | | (m, 2 H), 7.09-7.17 (m, 1 H), 5.47-5.68 (m, 1 H), 4.76 (br s, 2 H), |
| | | 4.26-4.42 (m, 4 H), 3.81-4.15 (m, 7 H), 3.42-3.55 (m, 1 H), |
| | | 2.56-2.81 (m, 2 H), 2.30-2.53 (m, 3 H), 2.10-2.30 (m, 3 H). |
| 43 | 529.3 | ¹H NMR (400 MHz, DMSO-d₆) δ ppm 8.22 (s, 1 H), 7.95-7.97 |
| | | (m, 1 H), 7.79-7.80 (m, 1 H), 7.44-7.45 (m, 2 H), 7.25-7.26 |
| | | (m, 3 H), 7.08-7.09 (m, 1 H), 4.07-4.10 (m, 5 H), 3.00-3.02 (m, |
| | | 2 H), 2.61-2.64 (m, 1 H), 2.09-2.11 (m, 2 H), 1.92-1.95 (m, 2 H), |
| | | 1.79-1.84 (m, 4 H), 1.60-1.63 (m, 2 H). |
| 76 | 619.2 | ¹H NMR (400 MHz, METHANOL-d₄) δ ppm 8.03 (dd, J = 7.9, 6.1 Hz, |
| | | 1 H), 7.73 (dd, J = 9.1, 6.0 Hz, 1 H), 7.28-7.40 (m, 3 H), 7.05-7.09 |
| | | (m, 1 H), 5.28-5.49 (m, 1 H), 4.57-4.68 (m, 2 H), 4.38-4.52 (m, 2 H), |
| | | 4.30-4.36 (m, 1 H), 4.11-4.25 (m, 1 H), 3.81-3.93 (m, 1 H), |
| | | 3.49-3.64 (m, 1 H), 3.39 (br d, J = 2.5 Hz, 1 H), 3.21-3.28 |
| | | (m, 1 H), 3.09 (td, J = 9.3, 5.9 Hz, 1 H), 2.41-2.64 (m, 5 H), |
| | | 2.20-2.41 (m, 4 H), 1.95-2.17 (m, 5 H), 1.84-1.93 (m, 1 H), |
| | | 0.87 (t, J = 7.3 Hz, 3 H), ¹⁹F NMR (376 MHz, METHANOL-d₄) |
| | | δ ppm −121.20 (m, 1 F), −128.98 (m, 1 F), 173.48 (m, 1 F). |
| 77 | 619.0 | ¹H NMR (400 MHz, METHANOL-d₄) δ ppm 7.88 (ddd, J = 13.8, |
| | | 10.0, 1.5 Hz, 1 H), 7.71 (d, J = 7.9 Hz, 1 H), 7.43 (t, J = 7.7 Hz, 1 H), |
| | | 7.38 (d, J = 2.3 Hz, 1 H), 7.23 (d, J = 7.1 Hz, 1 H), 7.07 (dd, J = 2.5, |
| | | 1.5 Hz, 1 H), 5.29-5.48 (m, 1 H), 4.64 (td, J = 7.7, 3.6 Hz, 2 H), |
| | | 4.29-4.49 (m, 3 H), 4.09-4.28 (m, 1 H), 3.62-3.78 (m, 1 H), 3.36- |
| | | 3.49 (m, 2 H), 3.20-3.29 (m, 1 H), 3.09 (td, J = 9.3, 5.9 Hz, 1 H), |
| | | 2.51-2.59 (m, 4 H), 2.36-2.49 (m, 1 H), 2.18-2.36 (m, 4 H), 2.0 |
| | | 2.14 (m, 3 H), 1.92-2.01 (m, 2 H), 1.87 (br dd, J = 8.9, 4.3 Hz, 1 H), |
| | | 1.00 (td, J = 7.4, 4.9 Hz, 3 H). ¹⁹F NMR (376 MHz, METHANOL-d₄) |
| | | δ ppm −118.24 (t, J = 3.5 Hz, 1 F), −124.79 (dd, J = 38.6, 4.8 Hz, 1 F), |
| | | −173.57 (m, 1 F). m/z (ESI): 619 (M + H)⁺. |
| 78 | 643.0 | ¹H NMR (400 MHz, METHANOL-d₄) δ ppm 7.82-7.90 (m, 2 H), |
| | | 7.39-7.51 (m, 2 H), 7.25 (d, J = 2.5 Hz, 1 H), 5.29-5.47 (m, 1 H), |
| | | 4.60-4.70 (m, 2 H), 4.44 (br d, J = 13.4 Hz, 1 H), 4.29-4.41 (m, 2 H), |
| | | 4.11-4.23 (m, 1 H), 3.71 (dd, J = 13.4, 11.1 Hz, 1 H), 3.36-3.50 |
| | | (m, 2 H), 3.21-3.29 (m, 1 H), 3.09 (td, J = 9.2, 5,7 Hz, 1 H), 2.51- |
| | | 2.61 (m, 2 H), 2.36-2.49 (m, 1 H), 2.27-2.35 (m, 2 H), 2.19-2.26 |
| | | (m, 1 H), 1.91-2.16 (m, 6 H), 1.83-1.91 (m, 1 H). ¹⁹F NMR (376 |
| | | MHz, METHANOL-d₄) δ ppm −116.31 (d, J = 7.8 Hz, 1 F), −118.93 |
| | | (m, 1 F), −125.76 (m, 1 F), −173.58 (m, 1 F). m/z (ESI): 643 (M + H)⁺ |
| 79 | 643.0 | ¹H NMR (400 MHz, METHANOL-d₄) δ ppm 7.79 (ddd, J = 10.1, |
| | | 5.7, 1.8 Hz, 1 H), 7.59 (br dd, J = 8.9, 4.3 Hz, 1 H), 7.33-7.42 (m, 1 H), |
| | | 7.28-7.33 (m, 1 H), 7.16 (d, J = 2.3 Hz, 1 H), 5.20-5.39 (m, 1 H), |
| | | 4.50-4.63 (m, 2 H), 4.27-4.39 (m, 2 H), 4.19-4.27 (m, 1 H), |
| | | 4.01-4.16 (m, 1 H), 3.59-3.67 (m, 1 H), 3.34-3.42 (m, 1 H), 3.12-3.27 |
| | | (m, 3 H), 3.00 (td, J = 9.2, 6.0 Hz, 1 H), 2.42-2.52 (m, 2 H), 2.28-2.40 |
| | | (m, 1 H), 2.17-2.26 (m, 2 H), 2.10-2.17 (m, 1 H), 1.85-2.05 (m, 5 H), |
| | | 1.75-1.83 (m, 1 H). ¹⁹F NMR (376 MHz, METHANOL-d₄) δ ppm |
| | | −120.22 (m, 1 F), −126.67 (dd, J = 9.1, 3,9 Hz, 1 F), −146.99 (m, 1 F), |
| | | −147.39 (m, 1 F), -174.01 (m, 1 F) |
| 80 | 640.2 | ¹H NMR (600 MHz, DMSO-d₆) δ ppm 9.99 (s, 1 H), 7.79 (dd, |
| | | J = 9.1, 6.0 Hz, 1 H), 7.67 (d, J = 9.5 Hz, 1 H), 7.61 (s, 1 H), 7.34- |
| | | 7.38 (m, 2 H), 7.03 (d, J = 2.6 Hz, 1 H), 5.23 (br s, 1 H), 5.14 (br s, |
| | | 1 H), 4.43 (dd, J = 11.2, 4.4 Hz, 1 H), 4.30 (dd, J = 11.1, 5.6 Hz, 1 H), |
| | | 4.08-4.19 (m, 2 H), 3.68 (d, J = 13.6 Hz, 1 H), 3.41-3.49 (m, 1 H), |
| | | 3.30-3.35 (m, 1 H), 2.90-2.95 (m, 1 H), 2.39-2.40 (m, 2 H), 2.31- |
| | | 2.39 (m, 1 H), 2.08-2.19 (m, 1 H), 1.88-2.05 (m, 4 H), 1.76-1.84 |
| | | (m, 1 H), 1.10-1.18 (m, 1 H), 0.75 (t, J = 7.4 Hz, 3 H). |
| 81 | 647.2 | ¹H NMR (400 MHz, METHANOL-d₄) δ ppm 7.92-8.44 (m, 1 H), |
| | | 7.76-7.88 (m, 1 H), 7.34-7.55 (m, 2 H), 7.06-7.26 (m, 1 H), 5.24- |
| | | 5.81 (m, 1 H), 4.64-4.75 (m, 2 H), 4.43-4.62 (m, 2 H), 3.96-4.26 |
| | | (m, 3 H), 3.83-3.95 (m, 4 H), 3.62-3.77 (m, 2 H), 3.42-3.55 |
| | | (m, 1 H), 2.52-2.81 (m, 2 H), 2.31-2.50 (m, 3 H), 2.09-2.24 (m, 1 H), |
| | | 1.30 (s, 3 H), |
| 82 | 629.2 | ¹H NMR (400 MHz, METHANOL-d₄) δ ppm 8.24-8.42 (m, 1 H), |
| | | 7.74-7.87 (m, 1 H), 7.36 (d, J = 2.5 Hz, 3 H), 6.89-7.20 (m, 1 H), |
| | | 5.42-5.72 (m, 1 H), 4.73 (s, 2 H), 4.44-4.60 (m, 2 H), 3.81-4.27 |
| | | (m, 7 H), 3.65-3.77 (m, 2 H), 3.39-3.54 (m, 1 H), 2.56-2.80 (m, 2 H), |
| | | 2.29-2.52 (m, 3 H), 2.09-2.25 (m, 1 H), 1.30 (d, J = 5.4 Hz, 3 H). |
| 83 | 626.0 | ¹H NMR (400 MHz, METHANOL-d₄) δ ppm 7.95 (d, J = 8.5 Hz, 1 H), |
| | | 7.35-7.41 (m, 1 H), 6.96 (d, J-2.7 Hz, 1 H), 6.70 (d, J = 2.5 Hz, 1 H), |
| | | 5.44-5.76 (m, 1 H), 4.65-4.87 (m, 3 H), 4.55 (br d, J = 13.5 Hz, 1 H), |
| | | 3.86-4.12 (m, 3 H), 3.65-3.75 (m, 2 H), 3.42-3.54 (m, 3 H), 2.56-2.80 |
| | | (m, 2 H), 2.31-2.50 (m, 3 H), 2.01-2.27 (m, 4 H), 1.78-1.96 (m, 2 H), |
| | | 0.66-0.79 (m, 1 H), 0.51-0.66 (m, 1 H), 0.14-0.24 (m, 1 H), |
| | | 0.04 (br d, J = 1.7 Hz, 1 H). |
| 84 | 626.0 | ¹H NMR (400 MHz, METHANOL-d₄) δ ppm 7.91-7.96 (m, 1 H), |
| | | 7.37 (t, J = 6.9 Hz, 1 H), 6.95-6.98 (m, 1 H), 6.70 (s, 1 H), 5.47- |
| | | 5.72 (m, 1 H), 4.68-4.71 (m, 2 H), 4.51 (br dd, J = 13.7, 3.7 Hz, 1 H), |
| | | 3.86-4.15 (m, 3 H), 3.63-3.81 (m, 3 H), 3.35-3.59 (m, 3 H), |
| | | 2.55-2.85 (m, 2 H), 2.31-2.51 (m, 3 H), 2.04-2.28 (m, 4 H), |
| | | 1.88-1.96 (m, 1 H), 1.82 (br t, J = 6.4 Hz, 1 H), 0.47-0.80 |
| | | (m, 2 H), −0.02-0.24 (m, 2 H). |
| 85 | 632.2 | ¹H NMR (400 MHz, METHANOL-d₄) δ ppm 7.92-8.05 (m, 3 H), |
| | | 7.44-7.57 (m, 2 H), 7.35-7.41 (m, 2 H), 5.47-5.72 (m, 1 H), |
| | | 4.64-4.84 (m, 3 H), 4.47-4.63 (m, 1 H), 3.80-4.19 (m, 3 H), |
| | | 3.63-3.78 (m, 2 H), 3.42-3.53 (m, 3 H), 2.58-2.83 (m, 2 H), |
| | | 2.32-2.56 (m, 5 H), 2.02-2.26 (m, 4 H), 1.88-1.97 (m, 1 H), |
| | | 0.79-0.86 (m, 3 H). |

### 8-Fluoro-2-(((2R,7aS)-2-fluorotetrahydro-1H-pyrrolizin-7a(5H)-yl)methoxy)-7-(3-hydroxynaphthalen-1-yl)-4-(1,4-oxazepan-4-yl)quinazoline-6-carbonitrile

### Example 44

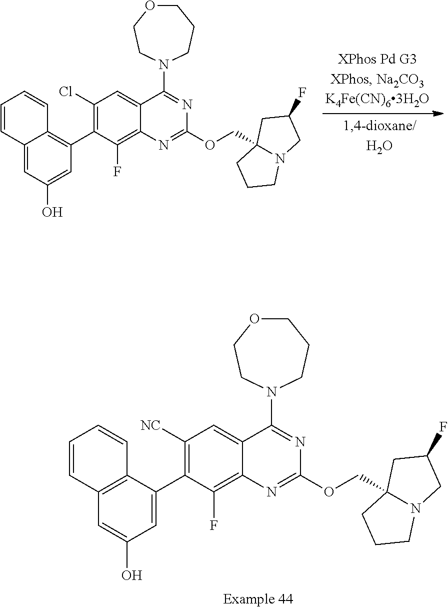 [SMILES: Oc1cc(-c2c(Cl)cc3c(N4CCCOCC4)nc(OC[C@@]45CCCN4C[C@H](F)C5)nc3c2F)c2ccccc2c1 ; N#Cc1cc2c(N3CCCOCC3)nc(OC[C@@]34CCCN3C[C@H](F)C4)nc2c(F)c1-c1cc(O)cc2ccccc12]

To a 10-mL round-bottom flask was added 4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)naphthalen-2-ol (0.11 g, 0.19 mmol), potassium ferrocyanide trihydrate (41 mg, 0.10 mmol, Toronto Research Chemicals), Na₂CO₃ (2.6 mg, 0.02 mmol, Sigma-Aldrich Corporation), and 2-dicyclohexylphosphino-2′,4′,6′,-triisopropylbiphenyl (18 mg, 0.04 mmol, Sigma-Aldrich Corporation) in dioxane (1.0 mL) and water (1.0 mL). ((2-dicyclohexylphosphino-2′,4′,6′-triisopropyl-1,1′-biphenyl)[2-(2′-amino-1,1′-biphenyl)]palladium(II) methanesulfonate (16 mg, 0.02 mmol, Sigma-Aldrich Corporation) was then added. The vial was purged with argon and the reaction was stirred at 90° C. After 1 h, the reaction was diluted with NaHCO₃ (5 mL), extracted with EtOAc (3-5 mL), dried over MgSO₄, filtered and concentrated under reduced pressure. The crude material was purified by HPLC. The fractions containing the product were combined, diluted with sat'd NaHCO₃ (10 mL), extracted with EtOAc (3-10 mL), dried over MgSO₄, filtered and concentrated in vacuo to give 8-fluoro-2-(((2R,7aS)-2-fluorotetrahydro-1H-pyrrolizin-7a(5H)-yl)methoxy)-7-(3-hydroxynaphthalen-1-yl)-4-(1,4-oxazepan-4-yl)quinazoline-6-carbonitrile (50 mg, 0.09 mmol, 45% yield) as tan solid. m/z (ESI): 572.2 (M+H)⁺. ¹H NMR (400 MHz, DMSO-d₆) δ ppm 10.03 (s, 1H), 8.47 (s, 1H), 7.84 (d, J=8.2 Hz, 1H), 7.48 (ddd, J=8.2, 6.4, 1.7 Hz, 1H), 7.34 (d, J=2.3 Hz, 1H), 7.24-7.32 (m, 2H), 7.17 (dd, J=2.3, 0.6 Hz, 1H), 5.16-5.41 (m, 1H), 4.12-4.20 (m, 5H), 4.07 (dd, J=10.5, 2.9 Hz, 1H), 3.91-3.97 (m, 2H), 3.75 (br t, J=4.1 Hz, 2H), 3.01-3.17 (m, 3H), 2.79-2.90 (m, 1H), 2.04-2.19 (m, 4H), 1.93-2.04 (m, 1H), 1.74-1.91 (m, 3H).

### 7-(8-Ethyl-3-hydroxynaphthalen-1-yl)-8-fluoro-2-(((2R,7aS)-2-fluorotetrahydro-1H-pyrrolizin-7a(5H)-yl)methoxy)-4-(1,4-oxazepan-4-yl)quinazoline-6-carbonitrile (Example 45)

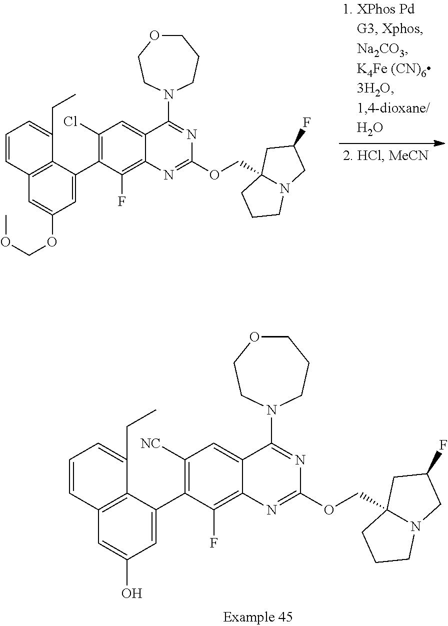 [SMILES: CCc1cccc2cc(OCOC)cc(-c3c(Cl)cc4c(N5CCCOCC5)nc(OC[C@@]56CCCN5C[C@H](F)C6)nc4c3F)c12 ; CCc1cccc2cc(O)cc(-c3c(C#N)cc4c(N5CCCOCC5)nc(OC[C@@]56CCCN5C[C@H](F)C6)nc4c3F)c12]

Synthesized in an analogous manner to Example 44. m/z (ESI): 600.3 (M+H)⁺. ¹H NMR (400 MHz, DMSO-d₆) δ ppm 9.95 (s, 1H), 8.44 (s, 1H), 7.71 (d, J=7.3 Hz, 1H), 7.37-7.45 (m, 1H), 7.34 (d, J=2.5 Hz, 1H), 7.17 (d, J=6.7 Hz, 1H), 6.96 (d, J=2.1 Hz, 1H), 5.09-5.46 (m, 1H), 4.11-4.22 (m, 5H), 4.04-4.10 (m, 1H), 3.90-3.98 (m, 2H), 3.71-3.79 (m, 2H), 2.96-3.17 (m, 3H), 2.78-2.88 (m, 1H), 2.24-2.41 (m, 2H), 2.03-2.20 (m, 4H), 1.94-2.03 (m, 1H), 1.72-1.90 (m, 3H), 0.89 (t, J=7.3 Hz, 3H).

### 4-(6-Amino-8-fluoro-2-(((2R,7aS)-2-fluorotetrahydro-1H-pyrrolizin-7a(5H)-yl)methoxy)-4-(1,4-oxazepan-4-yl)quinazolin-7-yl)-5-ethyl-6-fluoronaphthalen-2-ol (Example 46)

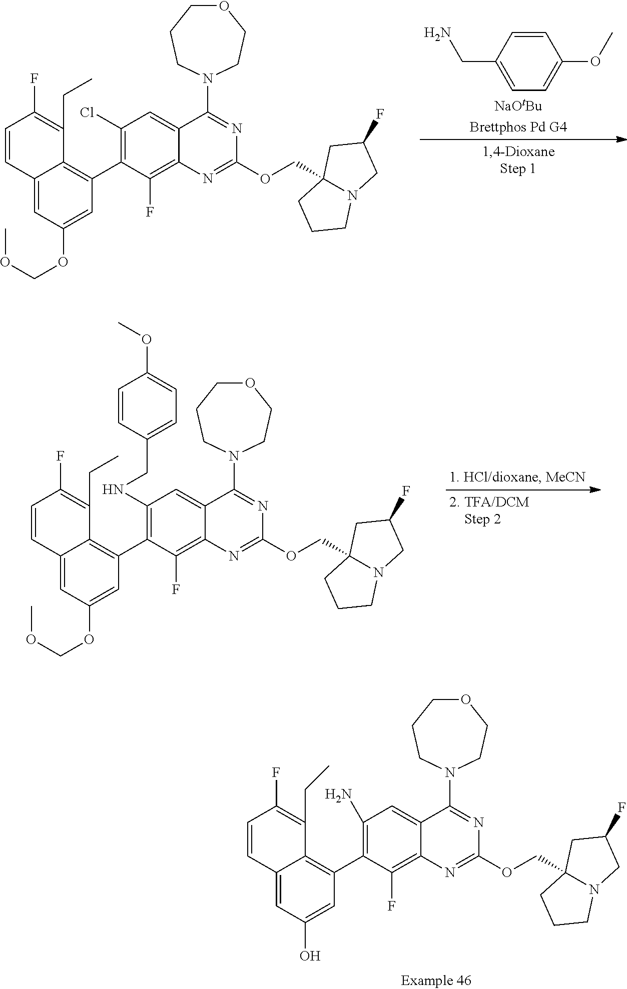

Step 1: 7-(8-Ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-N-(4-methoxybenzyl)-4-(1,4-oxazepan-4-yl)quinazolin-6-amine. A vial was charged with 4-methoxybenzylamine (20 mg, 20 μL, 0.15 mmol, Sigma-Aldrich Corporation), sodium tert-butoxide (21 mg, 0.22 mmol, Sigma-Aldrich Corporation), 4-(6-chloro-7-(8-ethyl-7-fluoro-3-(methoxymethoxy)naphthalen-yl)-8-fluoro-2-(((2R,7aS)-2-fluorotetrahydro-1H-pyrrolizin-7a(5H)-yl)methoxy)quinazolin-4-yl)-1,4-oxazepane (50 mg, 0.07 mmol), BrettPhos Pd G4 (14 mg, 0.02 mmol, Sigma-Aldrich Corporation) and 1,4-dioxane (0.75 mL). The reaction was then heated to 90° C. for 3 h. The reaction was then cooled to rt and concentrated under reduced pressure to afford a crude black oil. The crude residue was then purified by column chromatography on silica gel eluting with a gradient of 0-75% 3:1 EtOAc/EtOH (with 2% triethylamine) in heptane to provide 7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-N-(4-methoxybenzyl)-4-(1,4-oxazepan-4-yl)quinazolin-6-amine m/z (ESI): 772.2 (M+H)⁺. The product was then carried forward to the next step without further purification.

Step 2: 4-(6-Amino-8-fluoro-2-(((2R,7aS)-2-fluorotetrahydro-1H-pyrrolizin-7a(5H)-yl)methoxy)-4-(1,4-oxazepan-4-yl)quinazolin-7-yl)-5-ethyl-6-fluoronaphthalen-2-ol. 7-(8-Ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-N-(4-methoxybenzyl)-4-(1,4-oxazepan-4-yl)quinazolin-6-amine was stirred in MeCN (2.3 mL) and HCl (4 M in 1,4-dioxane, 0.5 mL, 1.86 mmol, Sigma-Aldrich Corporation). The reaction was stirred at it. After 30 min, the reaction was concentrated under reduced pressure and the resultant solid was suspended in DCM (1.3 mL) and 1,1,1-trifluoroacetic acid (0.2 mL, 2.24 mmol, Apollo Scientific Ltd.). The reaction was stirred at rt for 5 h. The reaction was then concentrated under reduced pressure, and purified sequentially via reverse phase HPLC and then by purification performed with 0.1% NH₄OH in H₂O and MeCN as mobile phase. XBridge column (19×100 mm, 5 μm) to provide 4-(6-amino-8-fluoro-2-(((2R,7aS)-2-fluorotetrahydro-1H-pyrrolizin-7a(5H)-yl)methoxy)-4-(1,4-oxazepan-4-yl)quinazolin-7-yl)-5-ethyl-6-fluoronaphthalen-2-ol (6.1 mg, 0.01 mmol, 13% yield) as yellow solid. m/z (ESI): 607.8 (M+H)⁺. ¹H NMR (500 MHz, DMSO-d₆) δ ppm 7.71-7.78 (m, 1H), 7.27-7.35 (m, 2H), 7.10-7.14 (m, 1H), 6.86-6.89 (m, 1H), 5.14-5.36 (m, 1H), 4.76-4.86 (m, 2H), 3.84-4.14 (m, 9H), 3.69-3.83 (m, 2H), 2.98-3.15 (m, 3H), 2.77-2.88 (m, 1H), 2.50 (d, J=1.8 Hz, 2H), 1.95-2.17 (m, 5H), 1.70-1.85 (m, 3H), 0.75 (t, J=7.4 Hz, 3H).

### (R)-1-(7-(8-Ethynyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol (Example 47)

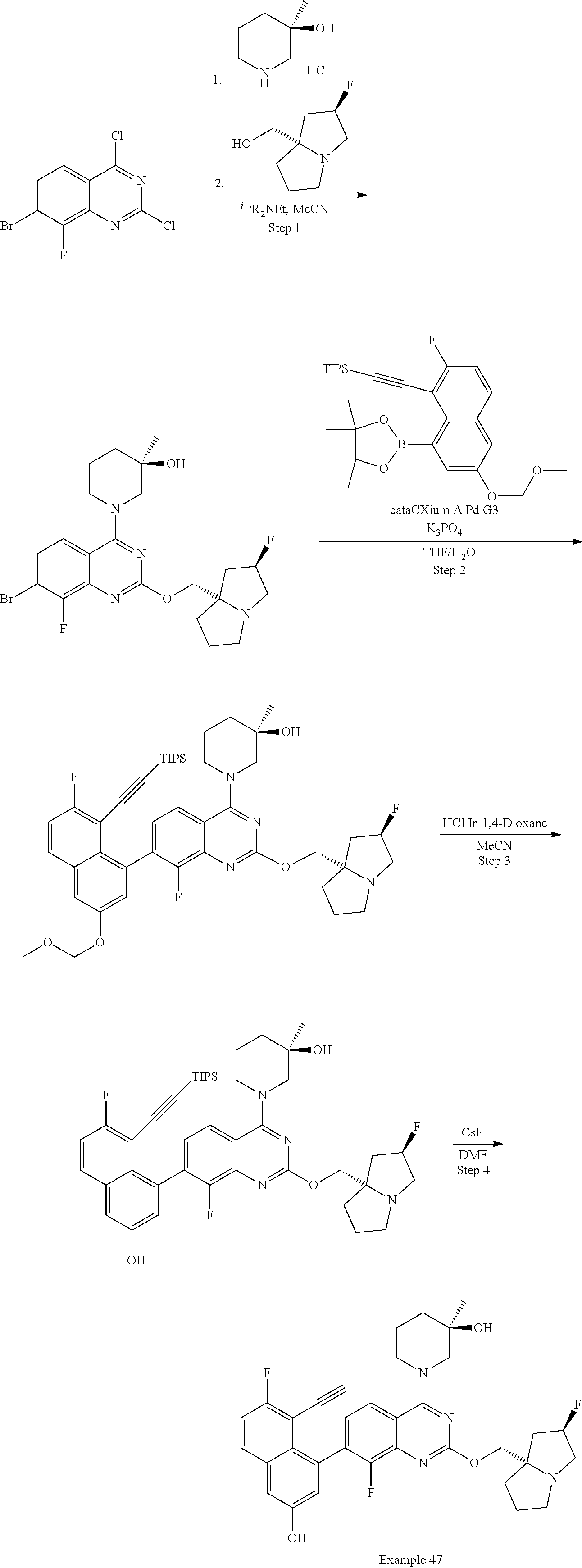

Step 1: (R)-1-(7-Bromo-8-fluoro-2-(((2R,7aS)-2-fluorotetrahydro-1H-pyrrolizin-7a(H)-yl)methoxy)quinazolin-4-yl)-3-methylpiperidin-3-ol. To a vial was added 2,4,7-trichloro-8-fluoroquinazoline (1.00 g, 3.98 mmol, Enamine) and DIPEA (2.06 g, 2.78 mL, 15.91 mmol, Sigma-Aldrich) in acetonitrile (20 mL). The solution was cooled to 0° C. and (R)-3-methylpiperidin-3-ol hydrochloride (0.60 g, 3.98 mmol, PharmaBlock) was added, and the reaction was left stirring. After 40 min ((2R,7aS)-2-fluorotetrahydro-5H-pyrrolizin-7a(5H)-yl)methanol (2 in MeCN, 2.1 mL, 4.18 mmol, LabNetwork), cesium carbonate (3.89 g, 11.93 mmol, Aldrich) and DABCO (89 mg, 0.80 mmol, Aldrich) were added. The reaction was left stirring at 40° C. for 20 h. The reaction was diluted with brine (15 mL), extracted with EtOAc (3×15 mL), dried over MgSO₄, filtered and concentrated under reduced pressure. The crude material was absorbed onto a plug of silica gel and purified by column chromatography on silica gel column, eluting with a gradient of 0-100% 3:1 EtOAc/EtOH in heptane to provide (R)-1-(7-bromo-8-fluoro-2-(((2R,7aS)-2-fluorotetrahydro-1H-pyrrolizin-7a(5H)-yl)methoxy)quinazolin-4-yl)-3-methylpiperidin-3-ol (0.70 g, 1.40 mmol, 35% yield) as yellow solid. m/z (ESI): 497.2 (M+H)⁺. ¹H NMR (400 MHz, CHLOROFORM-d) δ ppm 7.59 (br d, J=9.0 Hz, 1H), 7.36 (dd, J=9.0, 6.1 Hz, 1H), 5.23-5.41 (in, 1H), 4.34 (d, J=10.5 Hz, 1H), 4.22 (s, 1H), 4.18 (br d, J=4.6 Hz, 1H), 3.29-3.39 (m, 2H), 3.19-3.29 (m, 2H), 3.13 (br d, J=13.6 Hz, 1H), 2.95-3.07 (m, 1H), 2.26-2.41 (m, 1H), 2.12-2.26 (m, 2H), 1.92-2.03 (m, 4H), 1.85-1.92 (m, 1H), 1.52-1.80 (in, 7H).

Step 2: (R)-1-(8-Fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)-3-methylpiperidin-3-ol. To a vial was added (R)-1-(7-bromo-8-fluoro-2-(((2R,7aS)-2-fluorotetrahydro-1H-pyrrolizin-7a(5H)-yl)methoxy)quinazolin-4-yl)-3-methylpiperidin-3-01 (0.24 g, 0.47 mmol) and ((2-fluoro-6-(methoxymethoxy)-8-(4,4,5,5-tetramethyl-1,3,2-dioxaborolan-2-yl)naphthalen-1-yl)ethynyl)triisopropylsilane (0.36 g, 0.71 mmol. WuXi) in 1,4-dioxane (4.3 mL) and water (0.4 mL). Potassium phosphate (0.33 g, 1.42 mmol, Sigma-Aldrich Corporation) and cataCXium A Pd G3 (69 mg, 0.09 mmol, Aldrich) were then added. The mixture was then stirred at 60° C. After 20 h the reaction was concentrated and loaded onto a plug of silica gel and purified by column chromatography on silica gel, eluting with a gradient of 0-100% 3:1 EtOAc/EtOH in heptane to provide (R)-1-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)-3-methylpiperidin-3-ol (0.32 g, 0.40 mmol, 84% yield) as brown solid. m/z (ESI): 803.4 (M+H)⁺.

Step 3: (R)-1-(8-Fluoro-7-(7-fluoro-3-hydroxy-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)-3-methylpiperidin-3-ol. To a vial was added (R)-1-(8-fluoro-7-(7-fluoro-3-(methoxymethoxy)-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)-3-methylpiperidin-3-ol (0.32 g, 0.40 mmol) and HCl solution (4.0 M in dioxane, 1.0 mL, 3.96 mmol, Sigma-Aldrich Corporation) in acetonitrile (4.0 mL). The mixture was stirred at 40° C. After 55 min, MeOH (3-5 mL) was added and the solution was concentrated in vacuo to give (R)-1-(8-fluoro-7-(7-fluoro-3-hydroxy-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)-3-methylpiperidin-3-ol (0.30 g, 0.40 mmol, 100% yield) as pale-brown solid that was used in the next step without further purification. m/z (ESI): 759.3 (M+H)⁺.

Step 4: (R)-1-(7-(8-Ethynyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol. To a vial was added (R)-1-(8-fluoro-7-(7-fluoro-3-hydroxy-8-((triisopropylsilyl)ethynyl)naphthalen-1-yl)-2-(((2R,7aS)-2-fluorotetrahydro-1H-pyrrolizin-7a(5H)-yl)methoxy)quinazolin-4-yl)-3-methylpiperidin-3-ol (60 mg, 0.08 mmol) and cesium fluoride (36 mg, 0.24 mmol, Aldrich) in acetonitrile (0.8 mL) and N,N-dimethylformamide (0.2 mL). The mixture was stirred at 40° C. After 75 min, additional DMF (0.6 mL) and 3 equiv. of CsF were added and the temperature was increased to 60° C. After another 30 min the reaction was diluted with sat'd NaHCO₃ (5 mL), extracted with EtOAc (3×5 mL), dried over MgSO₄, filtered and concentrated under reduced pressure. The crude material was absorbed onto a plug of silica gel and purified by column chromatography on silica gel, eluting with a gradient of 0-100% 3:1 EtOAc/EtOH with 2% TEA in heptane to provide (R)-1-(7-(8-ethynyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol (30 mg, 0.05 mmol, 63% yield) as light-yellow solid. m/z (ESI): 603.3 (M+H)⁺. ¹H NMR (400 MHz, METHANOL-d₄) δ ppm 7.78-7.93 (m, 2H), 7.27-7.36 (m, 2H), 7.19-7.27 (m, 1H), 7.11 (dd, J=9.2, 2.4 Hz, 1H), 5.17-5.45 (m, 1H), 4.18-4.38 (m, 3H), 4.03-4.17 (m, 1H), 3.51-3.63 (m, 1H), 3.37-3.51 (m, 1H), 3.14-3.29 (m, 3H), 3.02 (td, J=9.3, 5.7 Hz, 1H), 2.11-2.45 (m, 4H), 1.69-2.07 (m, 7H), 1.27 (d, J=8.1 Hz, 3H).

TABLE 6
Examples 48 to 50. Synthesized in an analogous manner to Example 47.

| Ex. | | | Salt | | Method |
|---|---|---|---|---|---|
| # | Structure | Name | Form | Reagent | Change |
| 48 | 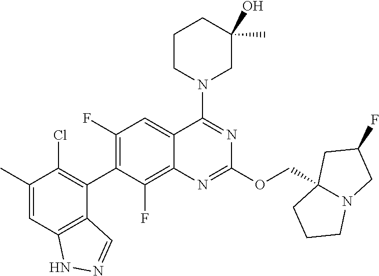 [SMILES: Cc1cc2[nH]ncc2c(-c2c(F)cc3c(N4CCC[C@@](C)(O)C4)nc(OC[C@@]45CCCN4C[C@H](F)C5)nc3c2F)c1Cl] | (3R)-1-(7-(5- Chloro-6-methyl- 1H-indazol-4-yl)- 6,8-difluoro-2- (((2R,7a.S)-2- fluorotetrahydro- 1H-pyrrolizin- 7a(5H)- yl)methoxy)quinazo- lin-4-y1)-3- methylpiperidin-3- ol | 2,2,2- trifluo- roacea- te | Step 1: 7-Bromo- 2,4-dichloro-6,8- difluoroquinazoline (CAS#: 2248318-27-4, Enamine) Step 2: 5-chloro- 6-methyl-1- (tetrahydro-2H- pyran-2-yl)-4- (4,4,5,5- tetramethyl- 1,3,2- dioxaborolan-2- yl)-1H-indazole (CAS#: 2374152- 77-7, | SPhos Pd G3 was used in Step 2. TFA/ DCM was used in Step 3. No Step 4. |
| | | | | PharmaBlock) | |
| 49 | 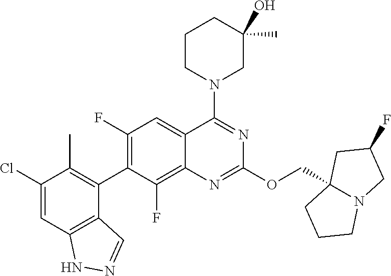 [SMILES: Cc1c(Cl)cc2[nH]ncc2c1-c1c(F)cc2c(N3CCC[C@@](C)(O)C3)nc(OC[C@@]34CCCN3C[C@H](F)C4)nc2c1F] | (3R)-1-(7-(6- Chloro-5-methyl- 1H-indazol-4-yl)- 6,8-difluoro-2- (((2R,7aS)-2- fluorotetrahydro- 1H-pyrrolizin- 7a(5H)- yl)methoxy)quinazo- lin-4-yl)-3- methylpiperidin-3- ol | 2,2.2- trifluo- roaceate | Step 1: 7-Bromo- 2,4-dichloro-6,8- difluoroquinazoline (CAS#: 2248318-27-4, Enamine) Step 2: 6-chloro- 5-methyl-1- (tetrahydro-2H- pyran-2-yl)-4- (4,4,5,5- tetramethyl- 1,3,2- dioxaborolan-2- yl)-1H-indazole (CAS#: 2374152- 83-5, | TFA/ DCM was used in Step 3. No Step 4. |
| | | | | PharmaBlock) | |
| 50 | 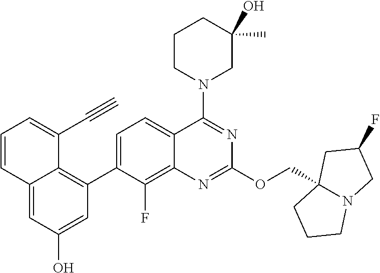 [SMILES: C#Cc1cccc2cc(O)cc(-c3ccc4c(N5CCC[C@@](C)(O)C5)nc(OC[C@@]56CCCN5C[C@H](F)C6)nc4c3F)c12] | (R)-1-(7-(8- Ethynyl-3- hydroxynaphthalen- 1-yl)-8-fluoro-2- (((2R,7a.S)-2- fluorotetrahydro- 1H-pyrrolizin- 7a(5H)- yl)methoxy)quinazo- lin-4-yl)-3- methylpiperidin-3- ol | Free base | Step 2: 2-[3- (Methoxymethoxy)- 8-[2-[tris(1- methylethyl)silyl] ethynyl]-1- naphthalenyl]- 4,4,5,5- tetramethyl- 1,3,2- Dioxaborolane (CAS#: 2621932- 42-9, LabNetwork) | |

TABLE 7
Analytical data for Examples 48 to 50.

| | MS | |
|---|---|---|
| Ex. | m/z (ESI): | |
| # | (M + H)⁺ | ¹H NMR |
| 48 | 601.0 | ¹H NMR (400 MHz, METHANOL-d₄) δ ppm 7.98 (br d, J = 10.03 Hz, 1 |
| | | H) 7.69 (s, 1 H) 7.65 (d, J = 2.93 Hz, 1 H) 5.47-5.71 (m, 1 H) 4.69-4.77 |
| | | (m, 2 H) 4.40-4.55 (m, 1 H) 4.24 (br d, J = 13.17 Hz, 1 H) 3.80-4.12 |
| | | (m, 3 H) 3.37-3.63 (m, 3 H) 2.62 (s, 5 H) 2.37 (br s, 3 H) 2.10-2.28 |
| | | (m, 2 H) 1.72-1.92 (m, 4 H) 1.31 (s, 3 H). |
| 49 | 601.0 | ¹H NMR (400 MHz, METHANOL-d₄) δ ppm 7.94-8.05 (m, 1 H) 7.83 |
| | | (s, 1 H) 7.63 (d, J = 3.76 Hz, 1 H) 5.45-5.72 (m, 1 H) 4.70 (d, J = 9.62 |
| | | Hz, 2 H) 4.40-4.55 (m, 1 H) 4.18-4.29 (m, 1 H) 3.80-4.10 (m, 3 H) |
| | | 3.57 (d, J = 13.38 Hz, 3 H) 2.51-2.86 (m, 2 H) 2.33 (d, J = 4.60 Hz, 8 H) |
| | | 1.72-1.98 (m, 4 H) 1.31 (s, 3 H). |
| 50 | 585.3 | ¹H NMR (400 MHz, DMSO-d₆) δ ppm 10.07 (s, 1 H), 7.75-7.90 (m, 2 |
| | | H), 7.38-7.49 (m, 2 H), 7.31 (d, J = 2.5 Hz, 1 H), 7.16-7.26 (m, 1 H), |
| | | 7.04 (dd, J = 11.3, 2.5 Hz, 1 H), 5.18-5.41 (m, 1 H), 4.56-4.66 (m, 1 H), |
| | | 4.08-4.15 (m, 1 H), 3.93-4.07 (m, 2 H), 3.73-3.92 (m, 1 H), 3.40- |
| | | 3.55 (m, 2 H), 2.99-3.20 (m, 3 H), 2.84 (br d, J = 6.5 Hz, 1 H), 1.97- |
| | | 2.23 (m, 5 H), 1.74-1.92 (m, 3 H), 1.57-1.74 (m, 3 H), 1.14-1.20 (m, |
| | | 3 H). |

### 6-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-1,6-diazaspiro[3.5]nonan-2-one (Example 51)

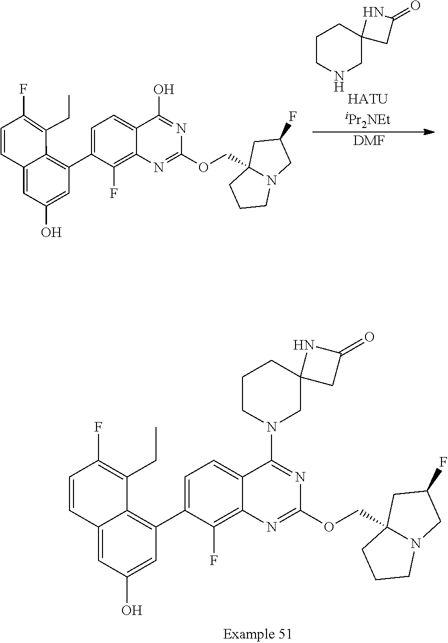

7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-ol (30 mg, 0.06 mmol, Intermediate C) was dissolved in N,N-dimethylformamide (0.4 mL), HATU (45 mg, 0.12 mmol. CombiBlocks Inc.) and DIPEA (30 mg, 41 μL, 0.24 mmol, Sigma-Aldrich Corporation) were added. The mixture was stirred at rt for 10 min. 1,6-Diazaspiro[3.5]nonan-2-one (8.3 mg, 0.06 mmol, Activate Scientific GmbH) was then added and the mixture stirred at rt for 3-d. The crude mixture was purified via reverse phase HPLC to yield 6-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-1,6-diazaspiro[3.5]nonan-2-one as its 2,2,2-trifluoroacetate salt (5.0 mg, 6.7 μmol, 11% yield). m/z (ESI): 632.2 (M+H)⁺. ¹H NMR (400 MHz, METHANOL-d₄) δ ppm 7.95 (d, J=9.1 Hz, 1H), 7.69 (dd, J=9.0, 6.1 Hz, 1H), 7.45 (t, J=7.8 Hz, 1H), 7.28 (d, J=2.5 Hz, 2H), 6.97 (d, J=1.9 Hz, 1H), 5.64 (br s, 1H), 4.70-4.78 (m, 1H), 4.61-4.69 (m, 1H), 4.15-4.31 (m, 2H), 3.99-4.10 (m, 1H), 3.86-3.98 (m, 3H), 3.76-3.86 (m, 1H), 3.50 (br s, 1H), 2.87 (d, J=14.9 Hz, 1H), 2.75-2.83 (m, 1H), 2.68 (s, 1H), 2.59-2.64 (m, 1H), 2.40-2.50 (m, 3H), 2.31-2.39 (m, 2H), 2.09-2.24 (m, 2H), 1.97-2.09 (m, 3H), 0.79 (t, J=7.4 Hz, 3H).

TABLE 8
Examples 52 to 54, 86 to 89. Prepared in an Analogous Manner to Example 51.

| Ex. | | | Salt | | Method |
|---|---|---|---|---|---|
| # | Structure | Name | Form | Reagent | Change |
| 52 | 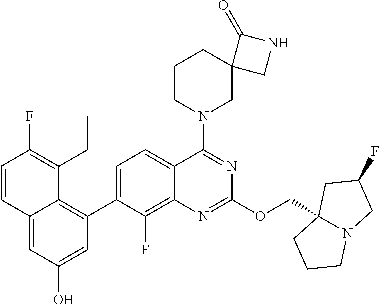 [SMILES: CCc1c(F)ccc2cc(O)cc(-c3ccc4c(N5CCCC6(CNC6=O)C5)nc(OC[C@@]56CCCN5C[C@H](F)C6)nc4c3F)c12] | 6-(7-(8-Ethyl-7- fluoro-3- hydroxynaphthalen- 1-y1)-8-fluoro-2- (((2R,7a.S)-2- fluorotetrahydro- 1H-pyrrolizin- 7a(5H)- yl)methoxy)quinazo- lin-4-yl)-2,6- diazaspiro[3.5]nonan- 1-one | 2,2,2- trifluo- roacetate | 2,8- Diazaspiro[3.5] nonan- 3-one (CAS#: 1422062- 20-1, PharmaBlock) | |
| 53 | 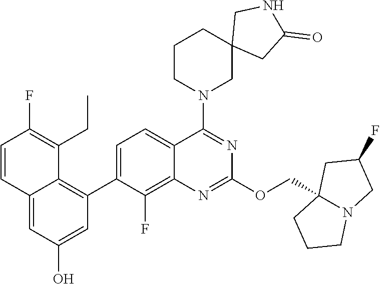 [SMILES: CCc1c(F)ccc2cc(O)cc(-c3ccc4c(N5CCCC6(CNC(=O)C6)C5)nc(OC[C@@]56CCCN5C[C@H](F)C6)nc4c3F)c12] | 7-(7-(8-Ethyl-7- fluoro-3- hydroxynaphthalen- 1-yl)-8-fluoro-2- (((2R,7aS)-2- fluorotetrahydro- 1H-pyrrolizin- 7a(5H)- yl)methoxy)quinazo- lin-4-yl)-2,7- diazaspiro[4.5]decan- 3-one | 2,2,2- trifluo- roacetate | 2,7- Diazaspiro[4.5] decan-3- one (CAS#: 1158750- 89-0, CombiBlocks) | |
| 54 | 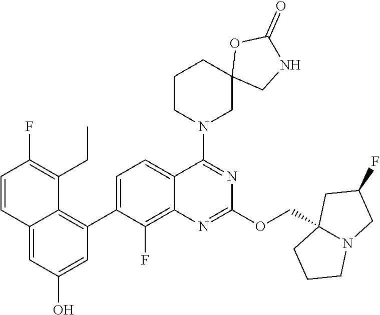 [SMILES: CCc1c(F)ccc2cc(O)cc(-c3ccc4c(N5CCCC6(CNC(=O)O6)C5)nc(OC[C@@]56CCCN5C[C@H](F)C6)nc4c3F)c12] | 7-(7-(8-Ethyl-7- fluoro-3- hydroxynaphthalen- 1-yl)-8-fluoro-2- (((2R,7aS)-2- fluorotetrahydro- 1H-pyrrolizin- 7a(5H)- yl)methoxy)quinazo- lin-4-yl)-1-oxa- 3,7- diazaspiro[4.5]decan- 2-one | bis(2, 2,2- trifluo- roacetate) | 1-Oxa-3,7- diazaspiro[4.5] decan-2- one (CAS#: 1308384- 36-2, ChemSpace) | |
| 86 | 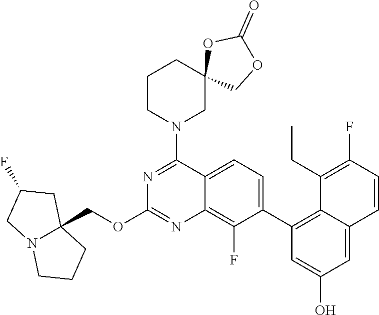 [SMILES: CCc1c(F)ccc2cc(O)cc(-c3ccc4c(N5CCC[C@]6(COC(=O)O6)C5)nc(OC[C@@]56CCCN5C[C@H](F)C6)nc4c3F)c12] | (R)-7-(7-(8-Ethyl- 7-fluoro-3- hydroxynaphthalen- 1-yl)-8-fluoro-2- (((2R,7aS)-2- fluorotetrahydro- 1H-pyrrolizin- 7a(5H)- yl)methoxy)quinazo- lin-4-yl)-1,3- dioxa-7- azaspiro[4.5]decan- 2-one | 2,2,2- trifluo- roacetate | 1,3-Dioxa- 7- azaspiro[4.5] decan-2- one (CAS#: 2386032- 05-7, Enamine) and precursor to Intermediate C | Chiral separation performed prior to MOM group deprotection. Details included below. |
| 87 | 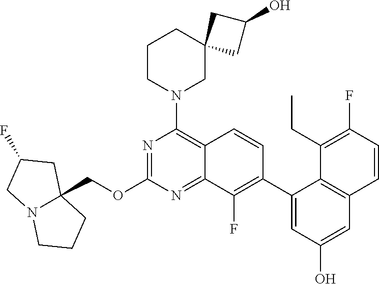 [SMILES: CCc1c(F)ccc2cc(O)cc(-c3ccc4c(N5CCC[C@]6(C5)C[C@@H](O)C6)nc(OC[C@@]56CCCN5C[C@H](F)C6)nc4c3F)c12] | (2S,4s)-6-(7-(8- Ethyl-7-fluoro-3- hydroxynaphthalen- 1-yl)-6,8- difluoro-2- (((2R,7a.S)-2- fluorotetrahydro- 1H-pyrrolizin- 7a(5H)- yl)methoxy)quinazo- lin-4-yl)-6- azaspiro[3.5]nonan- 2-ol | 2,2,2- trifluo- roacetate | 6- Azaspiro[3.5] nonan2 olhydrochlo- ride (CAS#: 2306270- 07-3, Ambeed, Inc.) | |
| 88 | 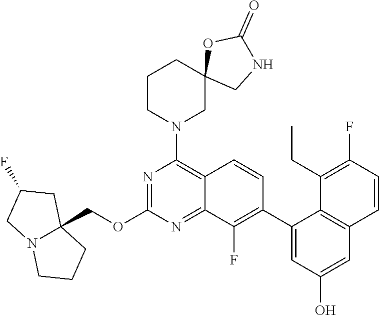 [SMILES: CCc1c(F)ccc2cc(O)cc(-c3ccc4c(N5CCC[C@]6(CNC(=O)O6)C5)nc(OC[C@@]56CCCN5C[C@H](F)C6)nc4c3F)c12] | (S)-7-(7-(8-Ethyl- 7-fluoro-3- hydroxynaphthalen- 1-yl)-8-fluoro-2- (((2R,7aS)-2- fluorotetrahydro- 1H-pyrrolizin- 7a(5H)- yl)methoxy)quinazo- lin-4-yl)-1-oxa- 3,7- diazaspiro[4.5]decan- 2-one | | 1-Oxa-3,7- diazaspiro[4.5] decan-2- one (CAS#: 1308384- 36-2, ChemSpace) | Chiral separation performed. Details included below. |
| 89 | 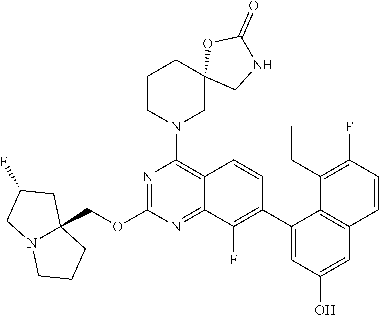 [SMILES: CCc1c(F)ccc2cc(O)cc(-c3ccc4c(N5CCC[C@@]6(CNC(=O)O6)C5)nc(OC[C@@]56CCCN5C[C@H](F)C6)nc4c3F)c12] | (R)-7-(7-(8-Ethyl- 7-fluoro-3- hydroxynaphthalen- 1-yl)-8-fluoro-2- (((2R,7aS)-2- fluorotetrahydro- 1H-pyrrolizin- 7a(5H)- yl)methoxy)quinazo- lin-4-yl)-1-oxa- 3,7- diazaspiro[4.5]decan- 2-one | | 1-Oxa-3,7- diazaspiro[4.5] decan-2- one (CAS#: 1308384- 36-2, ChemSpace) | Chiral separation performed. Details included below. |

TABLE 9
Conditions for Chiral SFC Separations.

| Compound | SFC Conditions | Peak to Ex.# |
|---|---|---|
| 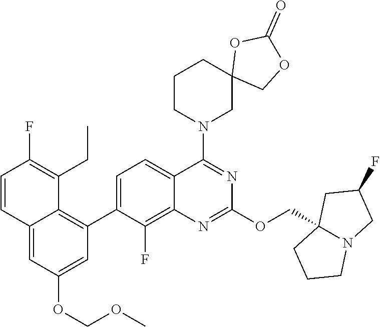 [SMILES: CCc1c(F)ccc2cc(OCOC)cc(-c3ccc4c(N5CCCC6(COC(=O)O6)C5)nc(OC[C@@]56CCCN5C[C@H](F)C6)nc4c3F)c12] | Column: Chiralcel OD, 2 × 25 cm, 5 μm Mobile phase: 40% iPrOH with 0.2% DEA Flowrate: 80 mL/min. Yield: 115 mg sample was submitted to generate 44 mg of peak 1 which racemized to produce both peak 2 and 3 in the ratio 60%:20%:20%, 25.5 mg of peak 2 which racemized to produce a 1:1 mixture of Peak 1 and 2, and 24.3 mg of Peak 3 which racemized to produce a 1:1 mixture of Peak 1 and 3. | Peak 3: Example 86 |
| 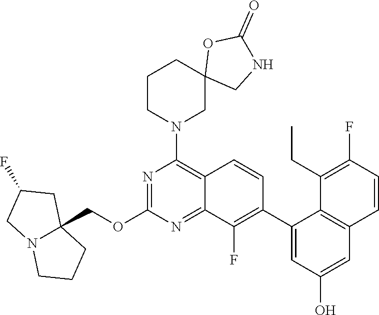 [SMILES: CCc1c(F)ccc2cc(O)cc(-c3ccc4c(N5CCCC6(CNC(=O)O6)C5)nc(OC[C@@]56CCCN5C[C@H](F)C6)nc4c3F)c12] | Column: ChiralPak IC, 2 × 25 cm, 5 μm Mobile phase: 50% iPrOH with 0.2% DEA Flowrate: 80 mL/min. Yield: 50 mg sample was submitted to generate 23 mg of peak 1 with 99% ee and 24 mg of peak 2 with 99% ee. | Peak 1: Example 88 Peak 2: Example 89 |

TABLE 10
Analytical Data for Examples 52 to 54, 86 to 89.

| | MS | |
|---|---|---|
| Ex. | m/z (ESI): | |
| # | (M + H)⁺ | ¹H NMR |
| 52 | 632.1 | ¹H NMR (400 MHz, METHANOL-d₄) δ ppm 7.94 (d, J = 8.6 Hz, 1 H), |
| | | 7.68 (dd, J = 9.0, 5.9 Hz, 1 H), 7.45 (t, J = 7.8 Hz, 1 H), 7.29 (d, J = 2.7 |
| | | Hz, 2 H), 6.97-6.99 (m, 1 H), 5.46-5.68 (m, 1 H), 4.61-4.74 (m, 2 |
| | | H), 4.18-4.38 (m, 2 H), 4.03 (br s, 2 H), 3.88 (br d, J = 15.0 Hz, 3 H), |
| | | 3.42-3.53 (m, 1 H), 3.18-3.29 (m, 2 H), 2.66-2.80 (m, 1 H), 2.57- |
| | | 2.65 (m, 1 H), 2.41-2.51 (m, 3 H), 2.30-2.41 (m, 2 H), 2.14-2.26 |
| | | (m, 3 H), 2.02-2.14 (m, 1 H), 1.89-2.01 (m, 1 H), 0.80 (td, J = 7.3, |
| | | 3.1 Hz, 3 H) |
| 53 | 646.0 | ¹H NMR (400 MHz, METHANOL-d₄) δ ppm 7.98 (d, J = 8.7 Hz, 1 H), |
| | | 7.69 (dd, J = 9.1, 5.8 Hz, 1 H), 7.40-7.50 (m, 1 H), 7.22-7.31 (m, 2 |
| | | H), 6.93-7.01 (m, 1 H), 5.49-5.68 (m, 1 H), 4.56-4.81 (m, 2 H), |
| | | 4.41-4.50 (m, 1 H), 4.24-4.36 (m, 1 H), 3.82-4.14 (m, 4 H), 3.66- |
| | | 3.78 (m, 1 H), 3.43-3.55 (m, 1 H), 2.57-2.81 (m, 2 H), 2.41-2.53 |
| | | (m, 4 H), 2.31-2.40 (m, 3 H), 2.16-2.28 (m, 1 H), 1.83-1.98 (m, 4 |
| | | H), 0.76-0.88 (m, 3 H). |
| 54 | 648.0 | ¹H NMR (400 MHz, METHANOL-d₄) δ ppm 7.96 (dd, J = 8.6, 2.1 Hz, |
| | | 1 H), 7.63-7.72 (m, 1 H), 7.40-7.50 (m, 1 H), 7.21-7.31 (m, 2 H), |
| | | 6.98 (s, 1 H), 5.49-5.68 (m, 1 H), 4.62-4.76 (m, 3 H), 4.46-4.61 (m, |
| | | 1 H), 3.80-4.12 (m, 3 H), 3.60-3.78 (m, 2 H), 3.40-3.55 (m, 3 H), |
| | | 2.58-2.82 (m, 2 H), 2.30-2.54 (m, 5 H), 2.13-2.28 (m, 3 H), 2.00- |
| | | 2.09 (m, 1 H), 1.87-1.95 (m, 1 H), 0.80 (td, J = 7.3, 4.7 Hz, 3 H). |
| 86 | 649.2 | ¹H NMR (400 MHz, METHANOL-d₄) δ ppm 7.89-7.98 (m, 1 H), |
| | | 7.64-7.74 (m, 1 H), 7.40-7.49 (m, 1 H), 7.29 (s, 2 H), 6.89-7.01 (m, |
| | | 1 H), 5.45-5.71 (m, 1 H), 4.60-4.80 (m, 3 H), 4.41-4.56 (m, 2 H), |
| | | 4.28-4.41 (m, 1 H), 3.94-4.13 (m, 1 H), 3.64-3.94 (m, 4 H), 3.43- |
| | | 3.53 (m, 1 H), 2.53-2.81 (m, 2 H), 2.30-2.53 (m, 5 H), 2.07-2.30 |
| | | (m, 4 H), 1.88-1.99 (m, 1 H), 0.80 (d, J = 8.09 Hz, 3 H). |
| 87 | 633.2 | ¹H NMR (400 MHz, METHANOL-d₄) δ ppm 8.01 (d, J = 8.6 Hz, 1 H), |
| | | 7.69 (dd, J = 9.0, 5.9 Hz, 1 H), 7.54 (t, J = 7.8 Hz, 1 H), 7.31 (d, J = 2.5 |
| | | Hz, 1 H), 7.27 (t, J = 9.4 Hz, 1 H), 6.99 (d, J = 1.9 Hz, 1 H), 5.50-5.68 |
| | | (m, 1 H), 4.30 (br d, J = 2.1 Hz, 1 H), 4.17-4.26 (m, 2 H), 3.94-4.16 |
| | | (m, 5 H), 3.81-3.92 (m, 1 H), 3.48 (td, J = 10.5, 6.2 Hz, 1 H), 2.61- |
| | | 2.83 (m, 2 H), 2.44-2.53 (m, 3 H), 2.32-2.42 (m, 3 H), 2.21-2.31 |
| | | (m, 2 H), 1.87 (br s, 4 H), 1.69-1.79 (m, 2 H), 0.82 (t, J = 7.4 Hz, 3 H). |
| | | ¹⁹F NMR (376 MHz, METHANOL-d₄) δ ppm −77.14 (s, 3 F) −120.80 |
| | | (m, 1 F), −130.67 (m, 1 F), −174.15 (m, 1 F). |
| 88 | 648.0 | ¹H NMR (500 MHz, METHANOL-d₄) δ ppm 7.81-7.89 (m, 1 H), |
| | | 7.64-7.70 (m, 1 H), 7.29-7.35 (m, 1 H), 7.19-7.29 (m, 2 H), 6.97 |
| | | (d, J = 2.5 Hz, 1 H), 5.25-5.42 (m, 1 H), 4.18-4.45 (m, 4 H), 3.77- |
| | | 3.90 (m, 1 H), 3.58-3.72 (m, 1 H), 3.41-3.55 (m, 2 H), 3.16-3.30 |
| | | (m, 3 H), 3.00-3.08 (m, 2 H), 2.31-2.53 (m, 3 H), 2.19 (br dd, J = 7.8, |
| | | 4.1 Hz, 4 H), 1.99-2.10 (m, 3 H), 1.83-1.95 (m, 2 H), 0.75-0.84 (m, |
| | | 3 H). ¹⁹F NMR (471 MHz, METHANOL-d₄) δ ppm −121.28 (br d, |
| | | J = 12.9 Hz, 1 F), −128.80 (br dd, J = 73.7, 12.2 Hz, 1 F), −174.32 (m, 1 F). |
| 89 | 648.0 | ¹H NMR (400 MHz, METHANOL-d₄) δ ppm 7.85 (br d, J = 8.3 Hz, 1 |
| | | H), 7.64-7.69 (m, 1 H), 7.32 (s, 1 H), 7.21-7.28 (m, 2 H), 6.97 (d, |
| | | J = 2.5 Hz, 1 H), 5.25-5.43 (m, 1 H), 4.20-4.45 (m, 4 H), 3.77-3.86 |
| | | (m, 1 H), 3.40-3.54 (m, 2 H), 3.20-3.31 (m, 3 H), 2.99-3.10 (m, 2 |
| | | H), 2.37-2.53 (m, 3 H), 2.15-2.34 (m, 5 H), 1.88-2.10 (m, 5 H), 0.79 |
| | | (t, J = 7.3 Hz, 3 H). ¹⁹F NMR (376 MHz, METHANOL-d₄) δ ppm |
| | | −121.63 (m, 1 F), −129.25 (m, 1 F), −162.27 (m, 1 F), −173.60 (s, 1 F). |

### rac-(1S,5R)-8-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-8-azabicyclo[3.2.1]octan-2-ol Example 55

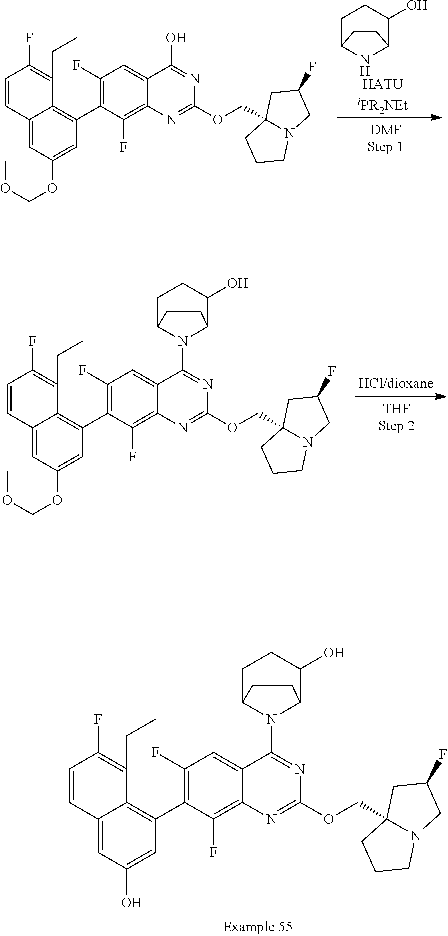

Step 1: rac-(1R,2S,5S)-8-(7-(8-Ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-8-azabicyclo[3.2.1]octan-2-ol. To a solution of 7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-ol (0.10 g, 0.18 mmol, MOM-protected Intermediate D) and DIPEA (57 mg, 76 μL, 0.44 mmol, Aldrich) in N,N-dimethylformamide (1.5 mL) was added HATU (67 mg, 0.18 mmol, CombiBlocks). The reaction was stirred at rt for 15 min, and then exo-azabicyclo[3.2.1]octan-2-ol hydrochloride (24 mg, 0.15 mmol, PharmaBlock) was added. The reaction was stirred at rt for 16 h. The reaction mixture was partitioned between water and ethyl acetate; the organic layer was separated and concentrated under reduced pressure. The crude product was purified by column chromatography on silica gel, eluting with a gradient of 0-100% 3:1 EtOAc/EtOH with 2% TEA in heptane, to provide rac-(1R,2S,5S)-8-(7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-8-azabicyclo[3.2.1]octan-2-ol as a colorless oil, which was used directly in the next step.

Step 2: rac-(1S,5R)-8-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-8-azabicyclo[3.2.1]octan-2-ol. To a solution of rac-(R,2S,5S)-8-(7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-8-azabicyclo[3.2.1]octan-2-ol in acetonitrile (1.5 mL) was added HCl solution (4.0 M in dioxane, 0.4 mL, 1.47 mmol, Sigma-Aldrich Corporation). The reaction was stirred at rt. After 1 h, the reaction mixture was neutralized with saturated aqueous sodium bicarbonate and extracted with ethyl acetate: the organic layer was separated and concentrated under reduced pressure.

The crude product was purified by column chromatography, eluting with a gradient of 0-100% 3:1 EtOAc/EtOH with 2% TEA in heptane, to provide rac-(1S,5R)-8-(7-(8-ethyl-7-fluoro-3-hydroxynaphthalen-1-yl)-6,8-difluoro-2-(((2R,7aS)-2-fluorotetrahydro-11H-pyrrolizin-7a(5H)-yl)methoxy)quinazolin-4-yl)-8-azabicyclo[3.2.1]octan-2-ol (69 mg, 0.11 mmol, 74% combined yield over 2 steps) as white powder. m/z (ESI): 637.2 (M+H)⁺. ¹H NMR (400 MHz, METHANOL-d₄) δ ppm 8.19 (t, J=9.3 Hz, 1H), 8.10 (t, J=9.3 Hz, 1H), 7.68 (dd, J=9.1, 6.0 Hz, 1H), 7.22-7.31 (m, 2H), 7.00 (d, J=2.5 Hz, 1H), 5.19-5.43 (m, 1H), 5.07 (br s, 2H), 4.29 (dd, J=10.5, 3.1 Hz, 1H), 4.19-4.24 (m, 1H), 3.91-3.95 (m, 1H), 3.29-3.31 (m, 1H), 3.16-3.27 (m, 3H), 3.01 (br d, J=5.2 Hz, 1H), 2.58 (br s, 1H), 2.37-2.50 (m, 3H), 2.26 (br s, 1H), 2.20 (s, 1H), 1.96-2.16 (m, 6H), 1.84-1.93 (m, 3H), 1.68 (br dd, J=15.4, 5.1 Hz, 2H), 0.82 (td, J=7.4, 1.9 Hz, 3H).

### 7-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one (Examples 56, 57, 58, 59)

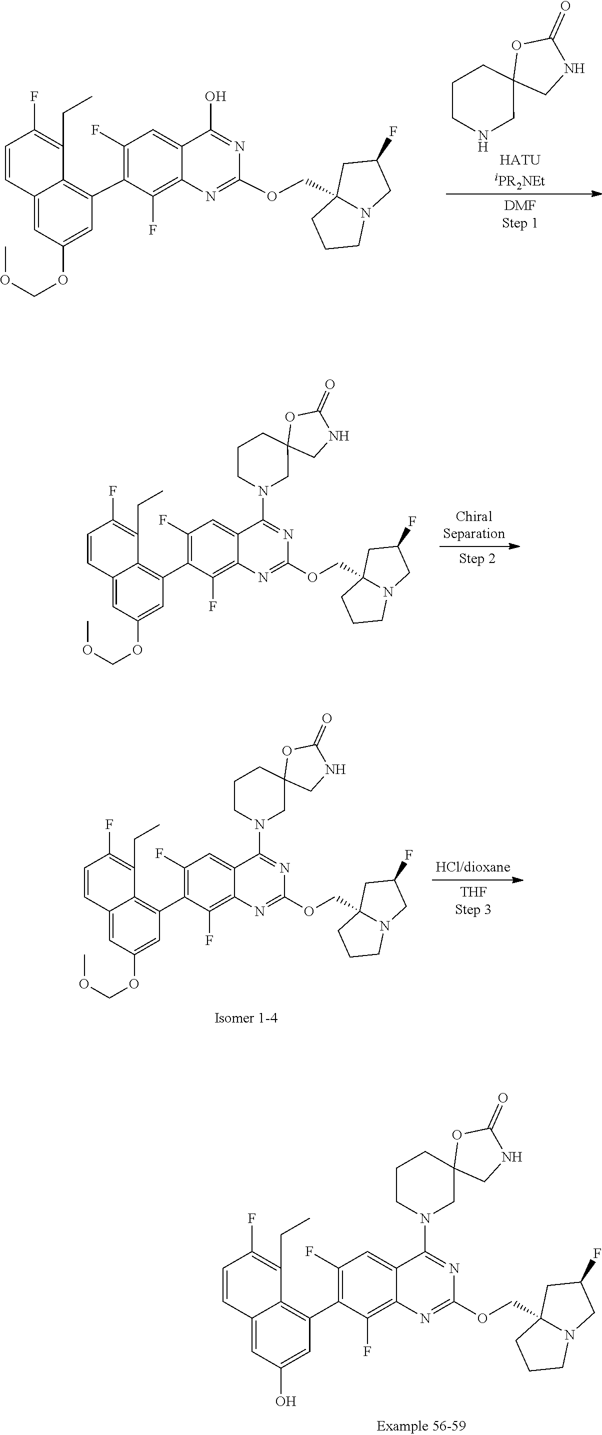 [SMILES: CCc1c(F)ccc2cc(OCOC)cc(-c3c(F)cc4c(O)nc(OC[C@@]56CCCN5C[C@H](F)C6)nc4c3F)c12 ; O=C1NCC2(CCCNC2)O1 ; CCc1c(F)ccc2cc(OCOC)cc(-c3c(F)cc4c(N5CCCC6(CNC(=O)O6)C5)nc(OC[C@@]56CCCN5C[C@H](F)C6)nc4c3F)c12 ; CCc1c(F)ccc2cc(OCOC)cc(-c3c(F)cc4c(N5CCCC6(CNC(=O)O6)C5)nc(OC[C@@]56CCCN5C[C@H](F)C6)nc4c3F)c12 ; CCc1c(F)ccc2cc(O)cc(-c3c(F)cc4c(N5CCCC6(CNC(=O)O6)C5)nc(OC[C@@]56CCCN5C[C@H](F)C6)nc4c3F)c12]

Step 1: 7-(7-(8-Ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one. This compound was synthesized in an analogous manner to Example 55, using 1-oxa-3,7-diazaspiro[4.5]decan-2-one (CAS #: 1308384-36-2, ChemSpace). m/z (ESI): 710.3 (M+H)⁺.

Step 2: Chiral separation, 7-(7-(8-Ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one 2,2,2-trifluoroacetate (0.27 g, 0.33 mmol) was purified via SFC using a Chiralpak AD, 21×250 mm, 5 μm column with a mobile phase of 40% 2-propanol with 0.2% diethylamine using a flowrate of 80 mL/min to generate 37 mg of peak 1 with an ee of >99%, 66 mg of peak 2-3 and 34 mg of peak 4 with an ee of >90%. Peak 2-3 was purified via SFC using a SS Whelk-O1, 21×250 mm, 5 μm column with a mobile phase of 40% methanol with 0.2% diethylamine using a flowrate of 80 mL/min to generate 25 mg of peak 2 with an ee >96% and 29 mg of peak 3 with an ee of >96%.

Step 3: 7-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one. Isomers 1-4 were synthesized in an analogous manner to Example 55, using peak 1-4 from Step 2. Isomer 1, Example 56: isolated as bis(2,2,2-trifluoroacetate), m/z (ESI): 666.0 (M+H)⁺. ¹H NMR (500 MHz, METHANOL-d₄) δ ppm 7.67-7.76 (m, 2H), 7.33 (d, J=2.5 Hz, 1H), 7.28 (t, J=9.3 Hz, 1H), 7.01 (d, J=2.2 Hz, 1H), 5.50-5.66 (m, 1H), 4.74 (d, J=12.5 Hz, 1H), 4.59-4.67 (m, 2H), 4.44 (br d, J=13.4 Hz, 1H), 3.83-4.10 (m, 3H), 3.71 (d, J=13.8 Hz, 1H), 3.60-3.67 (m, 1H), 3.42-3.56 (m, 3H), 2.55-2.77 (m, 3H), 2.45 (br d, J=7.8 Hz, 2H), 2.30-2.40 (m, 2H), 2.19 (br s, 3H), 2.00-2.08 (m, 1H), 1.87-1.96 (m, 1H), 0.83 (t, J=7.4 Hz, 3H). Isomer 2, Example 57: isolated as bis(2,2,2-trifluoroacetate), m/z (ESI): 666.0 (M+H)⁺. ¹H NMR (500 MHz, METHANOL-d₄) δ ppm 7.66-7.77 (m, 2H), 7.33 (d, J=2.3 Hz, 1H), 7.27 (t, J=9.4 Hz, 1H), 7.01 (d, J=2.2 Hz, 1H), 5.51-5.66 (m, 1H), 4.72 (s, 1H), 4.59-4.69 (m, 2H), 4.48 (br d, J=13.6 Hz, 1H), 3.96-4.09 (m, 1H), 3.89 (br d, J=15.1 Hz, 2H), 3.71 (d, J=13.9 Hz, 1H), 3.54-3.64 (m, 1H), 3.39-3.52 (m, 3H), 2.58-2.77 (m, 2H), 2.32-2.57 (m, 5H), 2.15-2.28 (m, 3H), 1.99-2.08 (m, 1H), 1.85-1.94 (m, 1H), 0.82 (t, J=7.4 Hz, 3H). Isomer 3, Example 58: isolated as is bis(2,2,2-trifluoroacetate) salt, m/z (ESI): 666.0 (M+H)⁺. ¹H NMR (500 MHz, METHANOL-d₄) δ ppm 7.66-7.80 (m, 2H), 7.32 (d, J=2.6 Hz, 1H), 7.27 (t, J=9.4 Hz, 1H), 7.01 (d, J=2.5 Hz, 1H), 5.52-5.66 (m, 1H), 4.67 (s, 3H), 4.49 (br d, J=13.1 Hz, 1H), 3.97-4.11 (m, 1H), 3.82-3.96 (m, 2H), 3.70 (d, J=13.9 Hz, 1H), 3.55-3.65 (m, 1H), 3.40-3.52 (m, 3H), 2.66-2.83 (m, 1H), 2.48-2.64 (m, 2H), 2.31-2.47 (m, 4H), 2.14-2.26 (m, 3H), 2.00-2.09 (m, 1H), 1.85-1.93 (m, 1H), 0.82 (t, J=7.4 Hz, 3H). Isomer 4, Example 59: isolated as bis(2,2,2-trifluoroacetate), m/z (ESI): 666.0 (M+H)⁺. ¹H NMR (500 MHz, METHANOL-d₄) δ ppm 7.67-7.76 (m, 2H), 7.32 (d, J=2.6 Hz, 1H), 7.27 (t, J=9.3 Hz, 1H), 7.00 (d, J=2.5 Hz, 1H), 5.51-5.67 (m, 1H), 4.66 (s, 3H), 4.42-4.50 (m, 1H), 3.97-4.12 (m, 1H), 3.84-3.95 (m, 2H), 3.60-3.75 (m, 2H), 3.41-3.54 (m, 3H), 2.66-2.82 (m, 1H), 2.53-2.64 (m, 2H), 2.32-2.48 (m, 4H), 2.12-2.26 (m, 3H), 2.01-2.09 (m, 1H), 1.86-1.94 (m, 1H), 0.82 (t. J=7.4 Hz, 3H).

### 4-(6,8-Difluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)-5-ethyl-6-fluoronaphthalen-2-ol (Example 90)

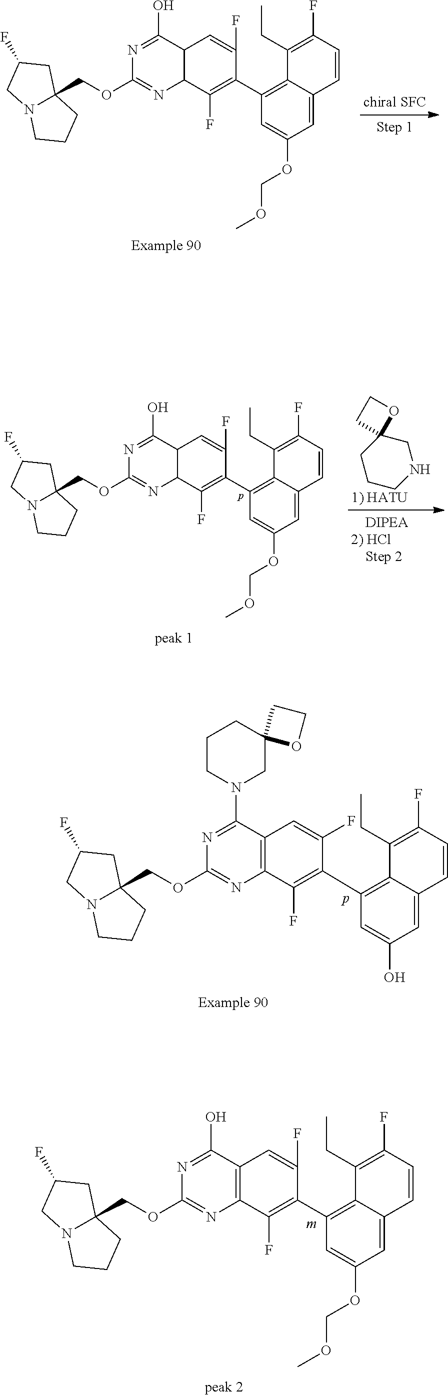

Step 1: Chiral SFC separation of MOM-protected Intermediate D, 2.6 g of material was purified using Chiralcel OD, 2×25 cm, 5 μm column with a mobile phase of 25% MeOH with 03.2% DEA using a flowrate of 120 mL/min to generate 1.18 g of peak 1 with an ee of 99% and 1.34 g of peak 2 with an ee of 96% Peak assignment determined by SFC with Chiralcel OD column with 25% MeOH with 0.2% DEA. m/z (ESI): 528.2 (M+H)⁺.

Step 2: 4-(6,8-Difluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)-5-ethyl-6-fluoronaphthalen-2-ol. To a mixture of peak 1 from the above step (77 mg, 0.146 mmol) and N,N-diisopropylethylamine (0.13 mL, 0.73 mmol) in N,N-dimethylacetamide (0.7 mL) at rt was added HATU (0.11 g, 0.29 mmol). The solution was stirred for 10 min then treated with (4S)-1-oxa-6-azaspiro[3.5]nonane (19 mg, 0.15 mmol, PharmaBlock) and stirred for 0.5 h. The reaction mixture was quenched with water and extracted with EtOAc. The combined organic layers were dried (Na₂SO₄), concentrated and the residue was redissolved in THF (1.5 mL) and treated with 4 M HCl/dioxane (1.5 mL). The mixture was stirred at rt for 4 b. The volatiles were removed in vacuo and the crude residue was purified on a silica gel column using 0-100% 2 M NH₃ in MeOH/DCM to provide p-4-(6,8-difluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)-5-ethyl-6-fluoronaphthalen-2-ol (4.0 mg, 0.062 mmol, 43% yield) as light brown solid. ¹H NMR (400 MHz, METHANOL-d₄) δ ppm 7.87-7.93 (m, 1H), 7.68 (dd, J=9.0, 5.9 Hz, 1H), 7.31 (d, J=2.5 Hz, 1H), 7.25 (t, J=9.3 Hz, 1H), 7.01 (d, J=2.7 Hz, 1H), 5.41-5.61 (m, 1H), 4.59-4.65 (m, 1H), 4.49-4.59 (m, 4H), 4.27 (br d, J=13.5 Hz, 1H), 3.82-3.94 (m, 1H), 3.68-3.82 (m, 2H), 3.62-3.68 (m, 1H), 3.34-3.43 (m, 2H), 2.58-2.70 (m, 1H), 2.45-2.58 (m, 4H), 2.35-2.45 (m, 2H), 2.22-2.33 (m, 3H), 1.98-2.16 (m, 2H), 1.85-1.95 (m, 1H), 1.80 (dt, J=13.4, 3.6 Hz, 1H), 0.81 (t, J=7.4 Hz, 3H). m/z (ESI): 637.1 (M+H)⁺.

### (2S,4s)-6-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-azaspiro[3.5]nonan-2-ol (Example 91)

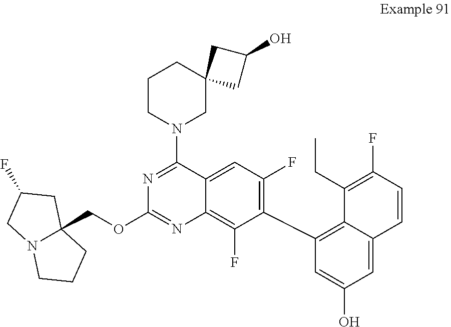 [SMILES: CCc1c(F)ccc2cc(O)cc(-c3c(F)cc4c(N5CCC[C@]6(C5)C[C@@H](O)C6)nc(OC[C@@]56CCCN5C[C@H](F)C6)nc4c3F)c12]

To a solution of 7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-ol (MOM-protected Intermediate D, 0.20 g, 0.35 mmol) in DMF (3.5 mL) was added HATU (0.20 g, 0.53 mmol) and DIPEA (0.18 mL, 1.1 mmol). The reaction mixture was stirred at rt for 10 min. 6-Azaspiro[3.5]-nonan-2-ol hydrochloride (75 mg, 0.42 mmol, Ambeed, Inc.) was added and the mixture was stirred at rt for 14 h. Water was added, the aqueous phase was extracted with EtOAc. The combined organic layers were dried over Na₂SO₄, filtered and volatiles were removed in vacuo. The residue was redissolved in THF (1.5 mL) and treated with 4 M HCl/dioxane (1.5 mL) dropwise. The mixture was stirred at rt for 4 h, volatiles were removed in vacuo and the crude residue was purified via preparative HPLC (10 to 90% MeCN/H₂O+0.1% TFA as a modifier) to yield (2S,4S)-6-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-azaspiro[3.5]nonan-2-ol bis(2,2,2-trifluoroacetate) (peak 1, 40 mg, 0.046 mmol, 13% yield). ¹H NMR (400 MHz, METHANOL-d₄) δ ppm 7.79-7.86 (m, 1H), 7.71 (dd, J=8.9, 5.8 Hz, 1H), 7.34 (d, J=2.5 Hz, 1H), 7.28 (t, J=9.3 Hz, 1H), 7.03 (d, J=2.3 Hz, 1H), 5.50-5.68 (m, 1H), 4.72-4.83 (m, 2H), 4.32 (quin, J=6.4 Hz, 1H), 3.82-4.19 (m, 8H), 3.47 (td, J=10.5, 6.0 Hz, 1H), 2.66 (br s, 3H), 2.43-2.52 (m, 2H), 2.33-2.42 (m, 2H), 2.18-2.29 (m, 3H), 1.84 (br s, 6H), 0.85 (t, J=7.3 Hz, 3H). ¹⁹F NMR (376 MHz, METHANOL-d₄) δ ppm −77.50 (s, 3F), −116.53 (s, 1F), −120.86 (s, 1F), −125.80 (s, 1F), −174.17 (s, 1F). m/z (ESI): 651.3 (M+H)⁺.

### 7-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-1-oxa-7-azaspiro[4.5]decan-2-one (Example 92)

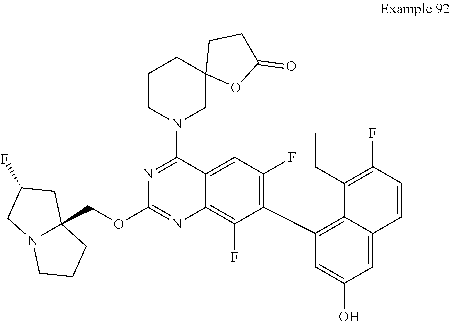 [SMILES: CCc1c(F)ccc2cc(O)cc(-c3c(F)cc4c(N5CCCC6(CCC(=O)O6)C5)nc(OC[C@@]56CCCN5C[C@H](F)C6)nc4c3F)c12]

This compound was prepared in a fashion similar to that described for Example 91 using 1-oxa-7-azaspiro[4.5]decan-2-one (CAS #: 1315303-69-5, PharmaBlock). ¹H NMR (500 MHz, METHANOL-d₄) δ ppm 7.70 (br d, J=9.2 Hz, 2H), 7.32 (d, J=2.6 Hz, 1H), 7.27 (t, J=9.4 Hz, 1H), 7.01 (s, 1H), 5.50-5.64 (m, 1H), 4.64-4.70 (m, 1H), 4.52-4.63 (m, 2H), 4.42-4.52 (m, 1H), 3.91-4.05 (m, 1H), 3.79-3.90 (m, 2H), 3.66-3.75 (m, 1H), 3.55-3.63 (m, 1H), 3.41-3.49 (m, 1H), 2.66-2.79 (m, 3H), 2.53-2.65 (m, 2H), 2.29-2.48 (m, 4H), 2.11-2.28 (m, 5H), 1.96-2.07 (m, 2H), 1.85-1.94 (m, 1H), 0.79-0.86 (m, 3H). m/z (ESI): 655.2 (M+H)⁺.

### (5R)-7-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-1,3-dioxa-7-azaspiro[4.5]decan-2-one (Example 93)

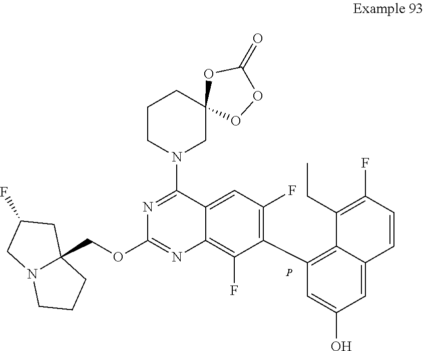

This compound was prepared in a fashion similar to that described for Example 91 using 1,3-dioxa-7-azaspiro[4.5]decan-2-one (CAS #: 2386032-05-7, Enamine). Chiral separation was performed prior to the deprotection step. The sample was purified via SFC using a ChiralPak IC, 2×25 cm, 5 μm column with a mobile phase of 50% iPrOH with 0.2% DEA using a flowrate of 80 mL/imin to generate 46 mg of peak 1 and 2 and 44 mg of peak 3 and 4. Peak assignment determined by SFC with ChiralPak IC column with 50% iPrOH with 0.2% DEA. Step 2: The sample was purified via SFC using a (S,S) Whelk-O, 2×25 cm, 5 μm column with a mobile phase of 45% iPrOH with 0.2% DEA using a flowrate of 80 mL/min to generate 20 mg of peak 1 with an cc of 99% and 20 mg of peak 2 with an cc of 99% Peak 2 gave the desired product ¹H NMR (400 MHz, METHANOL-d₄) δ ppm 7.68-7.75 (m, 2H), 7.23-7.35 (m, 2H), 7.01 (d, J=2.30 Hz, 1H), 5.51-5.67 (m, 1H), 4.67 (s, 3H), 4.25-4.52 (m, 3H), 3.75-4.12 (m, 4H), 3.62-3.73 (m, 1H), 3.44-3.52 (m, 1H), 2.48-2.86 (m, 3H), 2.31-2.47 (m, 4H), 2.03-2.27 (m, 4H), 1.86-1.99 (m, 1H), 0.81 (s, 3H). m/z (ESI): 667.2 (M+H)⁺.

### (5S)-7-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-2-thia-7-azaspiro[4.5]decane 2,2-dioxide (Example 94)

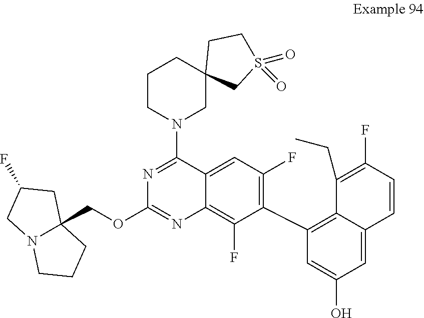 [SMILES: CCc1c(F)ccc2cc(O)cc(-c3c(F)cc4c(N5CCC[C@]6(CCS(=O)(=O)C6)C5)nc(OC[C@@]56CCCN5C[C@H](F)C6)nc4c3F)c12]

This compound was prepared in a fashion similar to that described for Example 91 using 21λ6-thia-7-azaspiro[4.5]decane-2,2-dione hydrochloride. Chiral separation was performed prior to the deprotection step. The sample was purified via SFC using a Chiralcel OJ, 2×25 cm, 5 μm column with a mobile phase of 20% EtOH with 0.2% DEA using a flowrate of 80 mL/min. to generate 164 mg of peak 1 and 2 with an ee of 91%, 31 mg of peak 3 with an cc of 91% and 30 mg of peak 4 with an cc of 95%. Peak 4 gave the desired product ¹H NMR (400 MHz, METHANOL-d₄) δ ppm 7.73-7.80 (m, 1H), 7.67-7.73 (m, 1H), 7.31-7.35 (m, 1H), 7.23-7.31 (m, 1H), 6.96-7.04 (m, 1H), 5.49-5.69 (m, 1H), 4.55-4.78 (m, 3H), 4.26-4.39 (m, 1H), 3.97-4.13 (m, 1H), 3.76-3.96 (m, 3H), 3.59-3.68 (m, 1H), 3.40-3.54 (m, 2H), 2.99-3.07 (m, 1H), 2.53-2.83 (m, 3H), 2.07-2.52 (m, 8H), 1.75-2.06 (m, 5H), 0.79-0.86 (m, 3H). m/z (ESI): 699.2 (M+H)⁺.

### (R)-1-(7-(5-Amino-3-chloro-2-(trifluoromethyl)phenyl)-8-fluoro-2-(((2R,7aS)-2-fluorotetrahydro-1H-pyrrolizin-7a(5H)-yl)methoxy)quinazolin-4-yl)-3-methylpiperidin-3-ol (Example 60)

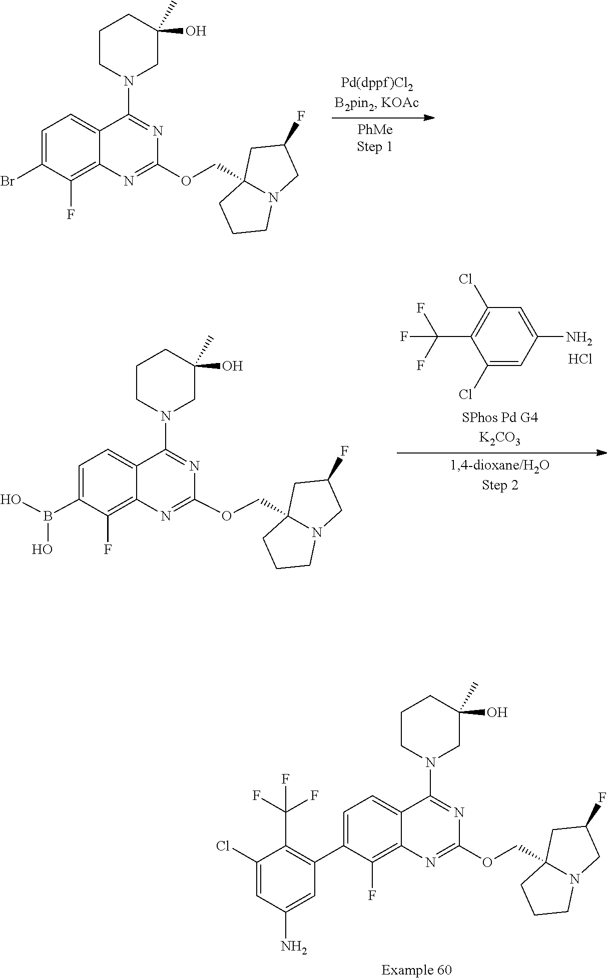 [SMILES: C[C@@]1(O)CCCN(c2nc(OC[C@@]34CCCN3C[C@H](F)C4)nc3c(F)c(Br)ccc23)C1 ; C[C@@]1(O)CCCN(c2nc(OC[C@@]34CCCN3C[C@H](F)C4)nc3c(F)c(B(O)O)ccc23)C1 ; Nc1cc(Cl)c(C(F)(F)F)c(Cl)c1 ; Cl ; C[C@@]1(O)CCCN(c2nc(OC[C@@]34CCCN3C[C@H](F)C4)nc3c(F)c(-c4cc(N)cc(Cl)c4C(F)(F)F)ccc23)C1]

Step 1. (8-Fluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)boronic acid. A 40-mL vial was charged with (R)-1-(7-bromo-8-fluoro-2-(((2R,7aS)-2-fluorotetrahydro-1H-pyrrolizin-7a(5H)-yl)methoxy)quinazolin-yl)-3-methylpiperidin-3-ol (0.50 g, 1.01 mmol, from Example 1) A 40-mL vial was charged with (R)-1-(7-bromo-8-fluoro-2-(((2R,7aS)-2-fluorotetrahydro-1H-pyrrolizin-7a(5H)-yl)methoxy)quinazolin-4-yl)-3-methylpiperidin-3-ol (0.50 g, 1.01 mmol, from Example 1), bis(pinacolato)diboron (0.41 g, 1.61 mmol, Sigma-Aldrich Corporation), [1,1′-bis(diphenylphosphino)ferrocene]dichloropalladium(II) (0.15 g, 0.20 mmol, Sigma-Aldrich Corporation), and potassium acetate (0.35 g, 3.52 mmol, Sigma-Aldrich Corporation) followed by toluene (10 mL). The resulting mixture was stirred at 90° C. for 1 h. The crude mixture was filtered through a layer of celite, and filter cake was washed with EtOAc. The filtrate was concentrated in vacuo to give the crude (8-fluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)boronic acid, which was used directly in the next step without further purification. m/z (EST): 463.0 (M+H)⁺.

Step 2. (R)-1-(7-(5-Amino-3-chloro-2-(trifluoromethyl)phenyl)-8-fluoro-2-(((2R,7aS)-2-fluorotetrahydro-1H-pyrrolizin-7a(5H)-yl)methoxy)quinazolin-4-yl)-3-methylpiperidin-3-ol. A vial was charged with SPhos Pd G3 (30 mg, 35 μmol, Strem Chemicals, Inc.), potassium carbonate (60 mg, 0.43 mmol), (8-fluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)boronic acid (80 mg, 0.17 mmol), 3,5-dichloro-4-(trifluoromethyl)benzenamine hydrochloride (69 mg, 0.26 mmol, CASS #: 1432795-16-8, AstaTech, Inc), water (0.7 mL) and 1,4-dioxane (3.6 mL). The reaction was stirred at 95° C. for 1 h. The crude mixture was filtered through a plug of celite. The filtrate was concentrated under reduced pressure and was purified by reverse phase HPLC to yield (R)-1-(7-(5-amino-3-chloro-2-(trifluoromethyl)phenyl)-8-fluoro-2-(((2R,7aS)-2-fluorotetrahydro-1H-pyrrolizin-7a(5H)-yl)methoxy)quinazolin-4-yl)-3-methylpiperidin-3-ol as bis(2,2,2-trifluoroacetate) (30 mg, 36 μmol, 21% yield) as off-white solid. m/z (ESI): 612.0 (M+H)⁺. ¹H NMR (400 MHz, METHANOL-d₄) δ ppm 8.04-8.13 (m, 1H), 7.29-7.39 (m, 1H), 6.92 (d, J=1.7 Hz, 1H), 6.40-6.47 (m, 1H), 5.49-5.69 (m, 1H), 4.72-4.76 (m, 2H), 4.54-4.65 (m, 1H), 4.30-4.40 (m, 1H), 3.84-4.09 (m, 3H), 3.59-3.67 (m, 1H), 3.41-3.54 (m, 2H), 2.56-2.80 (m, 2H), 2.30-2.49 (m, 3H), 2.10-2.26 (m, 2H), 1.76-1.92 (m, 3H), 1.27-1.35 (m, 3H).

### 4-Bromo-5-ethyl-5,6,7,8-tetrahydronaphthalen-2-yl pivalate (Intermediate I)

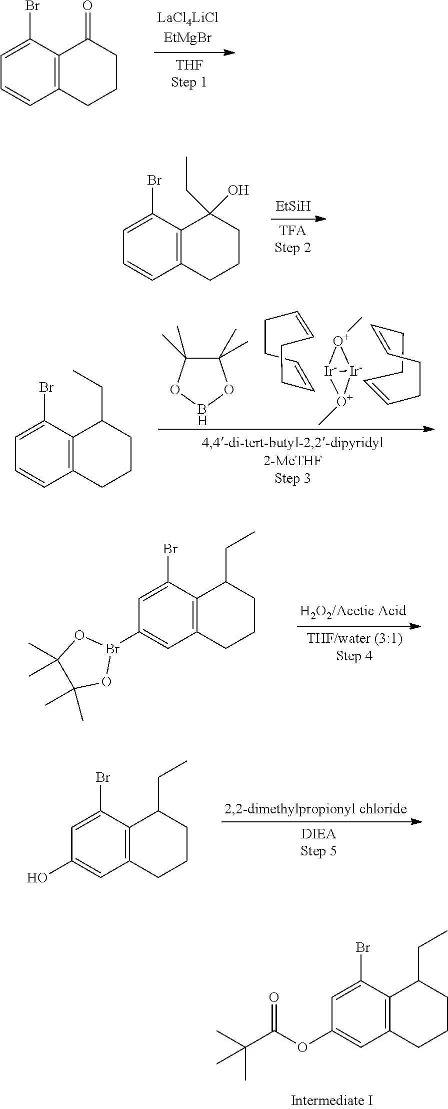

Step 1. 8-Bromo-1-ethyl-1,2,3,4-tetrahydronaphthalen-1-ol. To a 50-mL round-bottomed flask was added 8-bromo-3,4-dihydronaphthalen-1(2H)-one (0.72 g, 3.2 mmol, Ambeed, Inc.) and lanthanum(III) chloride bis(lithium chloride) complex solution (0.6 M in THF, 5.3 mL, 3.2 mmol, Sigma-Aldrich Corporation) in THF (12.8 mL). At 0° C., ethylmagnesium bromide (3.0 M in diethyl ether, 1.3 mL, 3.84 mmol) was added dropwise. The reaction mixture was stirred at 0° C. for 1 h, then was diluted with sat'd NH₄Cl and extracted with EtOAc. The organic extract was washed with satd NaCl solution, dried over MgSO₄, filtered and concentrated in vacuo. The crude material was purified by chromatography through a silica gel column (12 g), eluting with a gradient of 0-50% EtOAc in heptane, to provide 8-bromo-1-ethyl-1,2,3,4-tetrahydronaphthalen-1-ol (0.71 g, 2.78 mmol, 87% yield) as yellow oil. LC/MS: m/z (ESI): 237.2 (M+H−H₂O)⁺.

Step 2. 8-Bromo-1-ethyl-1,2,3,4-tetrahydronaphthalene. To a 25-mL round-bottomed flask was added 8-bromo-1-ethyl-1,2,3,4-tetrahydronaphthalen-1-ol (0.48 g, 1.89 mmol) in DCM (7.5 mL). At −30° C., triethylsilane (1.1 g, 9.4 mmol) was added followed by TFA (0.44 mL, 5.64 mmol). The reaction mixture was stirred at −30° C. to rt for 3 h, then was diluted with water and extracted with EtOAc. The organic extract was washed with saturated NaCl solution, dried over MgSO₄, filtered and concentrated in vacuo. The crude material was purified by chromatography through a silica gel column (12 g), eluting with a gradient of 0-20% EtOAc in heptane, to provide 8-bromo-1-ethyl-1,2,3,4-tetrahydronaphthalene (0.39 g, 1.63 mmol, 87% yield) as colorless oil. ¹H NMR (400 MHz, DMSO-d₆) δ ppm 7.35-7.48 (m, 1H), 7.29-7.58 (m, 2H), 2.64-2.89 (m, 3H), 1.84-2.04 (m, 1H), 1.52-1.85 (m, 4H), 1.30-1.45 (m, 1H), 0.95-1.05 (m, 3H) LC/MS: m/z (ESI): 237.2 (M+H)⁺.

Step 3. 2-(4-Bromo-5-ethyl-5,6,7,8-tetrahydronaphthalen-2-yl)-4,4,5,5-tetramethyl-1,3,2-dioxaborolane. A 20-mL vial was charged with 8-bromo-1-ethyl-1,2,3,4-tetrahydronaphthalene (0.21 g, 0.88 mmol), 4,4,5,5-tetramethyl-1,3,2-dioxaborolane (0.32 mL, 2.2 mmol), 4,4′-di-tert-butyl-2,2′-dipyridyl (28 mg, 0.11 mmol) and di-mu-methoxobis(1,5-cyclootadiene)diiridium(I) (58 mg, 0.088 mmol, Sigma-Aldrich Corporation). The reaction mixture was purged with nitrogen for 5 minutes and then stirred at 65° C. for 3 h. After cooling to rt, the reaction mixture was concentrated and the crude material was purified by chromatography through a silica gel column (12 g), eluting with a gradient of 0-70% EtOAc in hexane, to provide 2-(4-bromo-5-ethyl-5,6,7,8-tetrahydronaphthalen-2-yl)-4,4,5,5-tetramethyl-1,3,2-dioxaborolane (0.20 g, 0.55 mmol, 62% yield) as colorless oil. LC/MS: m/z (ESI): 365.2 (M+H)⁺.

Step 4. 4-Bromo-5-ethyl-5,6,7,8-tetrahydronaphthalen-2-ol. To a 50-mL round-bottomed flask was charged with 2-(4-bromo-5-ethyl-5,6,7,8-tetrahydronaphthalen-2-yl)-4,4,5,5-tetramethyl-1,3,2-dioxaborolane (0.20 g, 0.55 mmol) in tetrahydrofuran (1.6 mL) and water (0.55 mL). At 0° C. hydrogen peroxide (0.5 mL, 4.93 mmol) was slowly added, followed by addition of acetic acid (1.6 mL, 27.4 mmol). The reaction mixture was stirred for 3 h, then diluted with satd Na₂S₂O₃ and extracted with EtOAc. The organic layer was concentrated, and the crude material was purified by chromatography through a silica gel column (12 g), eluting with a gradient of 0-50% EtOAc in hexane, to provide 4-bromo-5-ethyl-5,6,7,8-tetrahydronaphthalen-2-ol (0.12 g, 0.47 mmol, 86% yield). LC/MS: m/z (ESI): 255.0 (M+H)⁺.

Step 5. 4-Bromo-5-ethyl-5,6,7,8-tetrahydronaphthalen-2-yl pivalate. To a 50-mL round-bottomed flask was added 4-bromo-5-ethyl-5,6,7,8-tetrahydronaphthalen-2-ol (0.12 g, 0.47 mmol) and triethylamine (0.16 mL, 0.94 mmol) in tetrahydrofuran (2.4 mL). At 0° C., 2,2-dimethylpropionyl chloride (87 μL, 0.71 mmol) was added slowly. The reaction mixture was stirred at 0° C. for 1 h. The crude material was purified by chromatography through a silica gel column (12 g), eluting with a gradient of 0-25% EtOAc in hexane, to provide 4-bromo-5-ethyl-5,6,7,8-tetrahydronaphthalen-2-yl pivalate (0.12 g, 0.35 mmol, 75% yield). LC/MS: m/z (ESI): 339.0 (M+H).

### 4-Bromo-5-methyl-5,6,7,8-tetrahydronaphthalen-2-yl pivalate (Intermediate J)

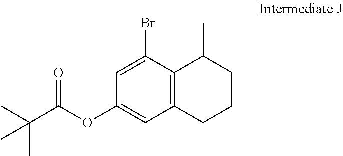 [SMILES: CC1CCCc2cc(OC(=O)C(C)(C)C)cc(Br)c21]

The intermediate was made in a similar fashion as Intermediate I using MeMgBr instead of EtMgBr in the first step. LC/MS: m/z (ESI): 325.0 (M+H)⁺.

### (3R)-1-(7-(8-Ethyl-3-hydroxy-5,6,7,8-tetrahydronaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol (Example 95)

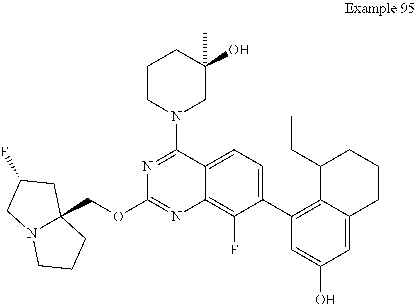 [SMILES: CCC1CCCc2cc(O)cc(-c3ccc4c(N5CCC[C@@](C)(O)C5)nc(OC[C@@]56CCCN5C[C@H](F)C6)nc4c3F)c21]

This compound was prepared in a fashion similar to that described for Example 60 using Intermediate I. ¹H NMR (400 MHz, METHANOL-d₄) δ ppm 7.97-8.23 (m, 1H), 7.19-7.52 (m, 1H), 6.58-6.73 (m, 1H), 6.34-6.55 (m, 1H), 5.51-5.81 (m, 1H), 4.65-4.79 (m, 2H), 4.48-4.60 (m, 1H), 4.20-4.45 (m, 1H), 3.80-4.14 (m, 3H), 3.42-3.69 (m, 3H), 2.32-2.89 (m, 8H), 2.07-2.27 (m, 2H), 1.67-1.93 (m, 7H), 1.30 (br s, 5H), 0.35-0.57 (m, 3H). m/z (ESI): 593.4 (M+H)⁺.

### (3R)-1-(8-Fluoro-2-(((2R,7aS)-2-fluorotetrahydro-1H-pyrrolizin-7a(5H)-yl)methoxy)-7-(3-hydroxy-8-methyl-5,6,7,8-tetrahydronaphthalen-1-yl)quinazolin-4-yl)-3-methylpiperidin-3-ol (Example 96)

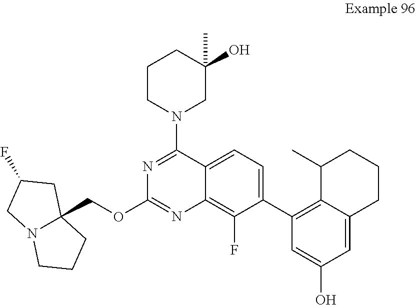 [SMILES: CC1CCCc2cc(O)cc(-c3ccc4c(N5CCC[C@@](C)(O)C5)nc(OC[C@@]56CCCN5C[C@H](F)C6)nc4c3F)c21]

This compound was prepared in a fashion similar to that described for Example 60 using Intermediate J. ¹H NMR (400 MHz, METHANOL-d₄) δ ppm 7.95-8.33 (m, 1H), 7.16-7.44 (m, 1H), 6.63 (s, 1H), 6.33-6.51 (m, 1H), 5.36-5.77 (m, 1H), 4.65-4.76 (m, 2H), 4.41-4.58 (m, 1H), 4.21-4.34 (m, 1H), 3.78-4.10 (m, 3H), 3.57-3.71 (m, 1H), 3.42-3.54 (m, 2H), 2.82 (br s, 5H), 2.30-2.50 (m, 3H), 2.07-2.28 (m, 2H), 1.56-1.98 (m, 7H), 1.30 (s, 3H), 0.83 (br s, 3H). m/z (ESI): 579.2 (M+H)⁺.

### (3R)-1-(7-(3-Amino-8-ethyl-7-fluoronaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Example 61 and Example 62)

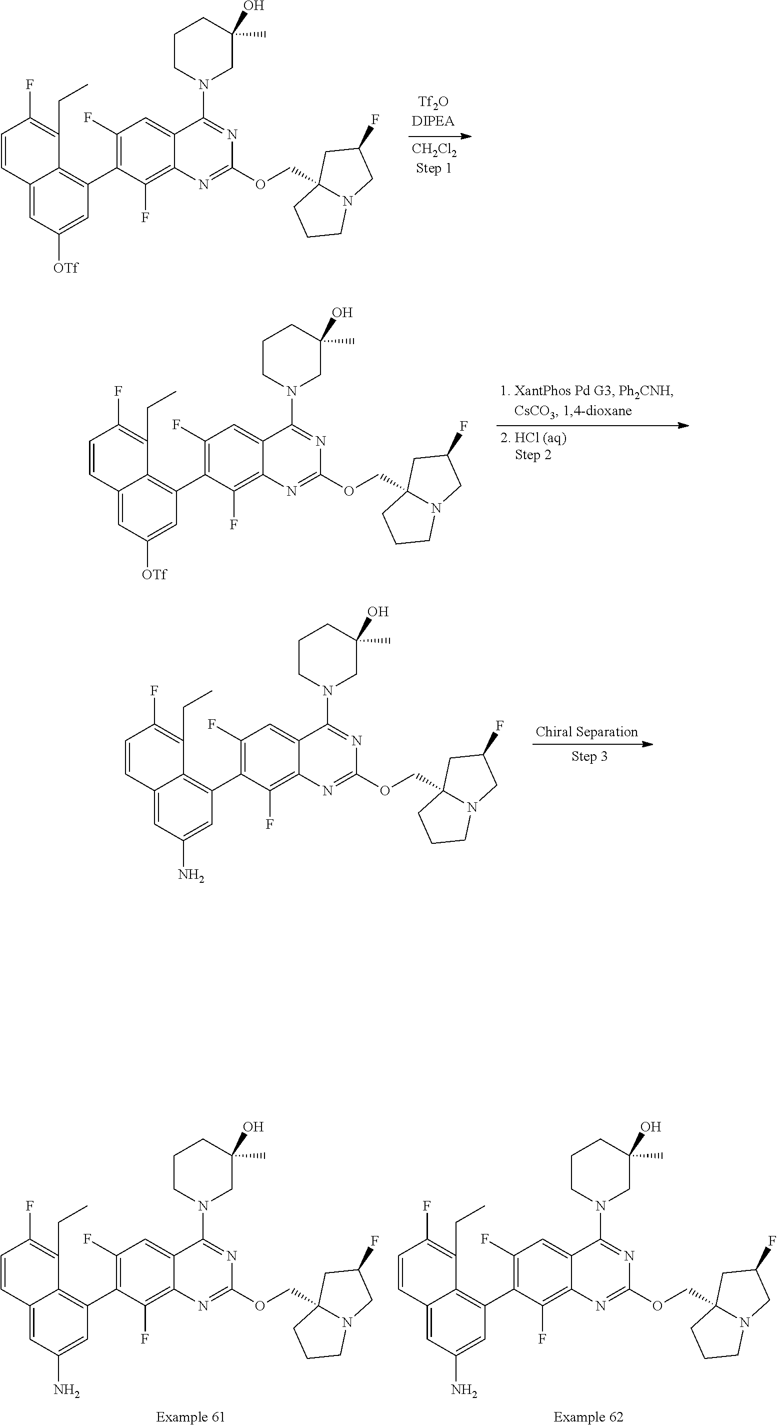

Step 1: 4-(6,8-Difluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)-5-ethyl-6-fluoronaphthalen-2-yl trifluoromethanesulfonate. A vial was charged with (3R)-1-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (0.83 g, 1.32 mmol, Example 2) and DIPEA (0.18 g, 0.24 mL, 1.39 mmol, Aldrich) in DCM (13 mL). The solution was then cooled to 0° C. and trifluoromethanesulfonic anhydride (1.32 mL, 1.32 mmol, Aldrich) was added dropwise. After 20 min the reaction was quenched with sat'd NH₄Cl (15 mL), extracted with DCM (3×15 mL), dried over MgSO₄, filtered and concentrated. The crude material was absorbed onto a plug of silica gel and purified by column chromatography on silica gel, eluting with a gradient of 0-100% 3:1 EtOAc/EtOH in heptanes to provide 4-(6,8-difluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)-5-ethyl-6-fluoronaphthalen-2-yl trifluoromethanesulfonate (0.45 g, 0.60 mmol, 45% yield) as light-yellow solid. m/z (ESI): 757.2 (M+H)⁺.

Step 2: (3R)-1-(7-(3-Amino-8-ethyl-7-fluoronaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol. To a vial was added 4-(6,8-difluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)-5-ethyl-6-fluoronaphthalen-2-yl trifluoromethanesulfonate (0.45 g, 0.60 mmol) and benzophenone imine (0.16 g, 0.15 mL, 0.89 mmol, Sigma-Aldrich Corporation) in 1,4-dioxane (6.0 mL). To the mixture was then added cesium carbonate (0.39 g, 1.19 mmol, Aldrich) followed by methanesulfonato[9,9-dimethyl-4,5-bis(diphenylphosphino)xanthene][2′-amino-1,1′-biphenyl]palladium(II) DCM adduct (61 mg, 0.06 mmol, Strem Chemicals, Inc.). The reaction was then stirred at 60° C. After 1 h, the temperature was increased to 75° C. After another 1 h, the reaction was cooled to it and hydrochloric acid (I N in water, 3.0 mL, 2.97 mmol, Fisher) was added. The mixture was left stirring at rt for 20 min. The reaction was poured into sat'd NaHCO₃ (20 mL), extracted with EtOAc (3×15 mL), dried over MgSO₄, filtered and concentrated. The crude material was absorbed onto a plug of silica gel and purified by column chromatography on silica gel, eluting with a gradient of 0-100% 3:1 EtOAc/EtOH with 2% TEA in heptane to provide (3R)-1-(7-(3-amino-8-ethyl-7-fluoronaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (0.32 g, 0.52 mmol, 87% yield) as yellow solid. m/z (ESI): 624.3 (M+H)⁺.

Step 3: Chiral separation. 70 mg sample was purified via SFC using a Chiralcel OJ, 21×250 mm, 5 μm column with a mobile phase of 20% MeOH using a flowrate of 100 mL/min to generate 19 mg of peak 1 with an ee of >99% and 20 mg of peak 2 with an ee of >99%. Peak 1 (Example 61): m/z, (ESI): 624.3 (M+H)⁺. ¹H NMR (400 MHz, METHANOL-d₄) δ ppm 7.76 (dd, J=9.8, 1.7 Hz, 1H), 7.59 (dd, J=9.0, 5.9 Hz, 1H), 7.15-7.24 (m, 2H), 6.95 (d, J=2.3 Hz, 1H), 5.18-5.46 (m, 1H), 4.17-4.36 (m, 3H), 4.05 (br d, J=13.2 Hz, 1H), 3.39-3.58 (m, 2H), 3.15-3.31 (m, 3H), 3.02 (td, J=9.4, 5.7 Hz, 1H), 2.47-2.64 (m, 1H), 2.12-2.47 (m, 5H), 1.72-2.05 (m, 6H), 1.28 (s, 3H), 0.81 (t, J=7.3 Hz, 3H). Peak 2 (Example 62); m/z (ESI): 624.3 (M+H)⁺. ¹H NMR (400 MHz, METHANOL-d₄) δ ppm 7.79 (dd, J=9.8, 1.7 Hz, 1H), 7.59 (dd, J=9.2, 5.9 Hz, 1H), 7.15-7.22 (m, 2H), 6.95 (d, J=2.5 Hz, 1H), 5.16-5.48 (m, 1H), 4.27-4.35 (m, 1H), 4.17-4.27 (m, 2H), 4.05 (d, J=13.0 Hz, 1H), 3.39-3.51 (m, 2H), 3.16-3.31 (m, 3H), 3.02 (td, J=9.3, 5.9 Hz, 1H), 2.50-2.63 (m, 1H), 2.09-2.49 (m, 5H), 1.70-2.07 (m, 6H), 1.31 (s, 3H), 0.82 (t, J=7.4 Hz, 3H).

TABLE 11
Additional Examples 63 to 65, Prepared in an Analogous Manner to Examples 61 and 62.

| | | | Salt | Method |
|---|---|---|---|---|
| Ex.# | Structure | Name | Form | Change |
| 63 | 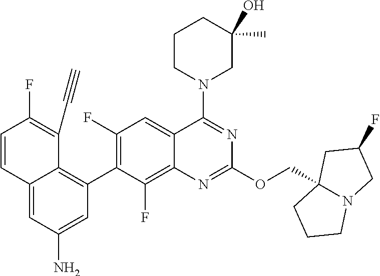 [SMILES: C#Cc1c(F)ccc2cc(N)cc(-c3c(F)cc4c(N5CCC[C@@](C)(O)C5)nc(OC[C@@]56CCCN5C[C@H](F)C6)nc4c3F)c12] | (3R)-1-(7-(3-Amino-8- ethynyl-7- fluoronaphthalen-1-yl)- 6,8-difluoro-2- (((2R,7aS)-2- fluorotetrahydro-1H- pyrrolizin-7a(5H)- yl)methoxy)quinazolin- 4-yl)-3- methylpiperidin-3-ol Isomer 1 | Free base | |
| 64 | 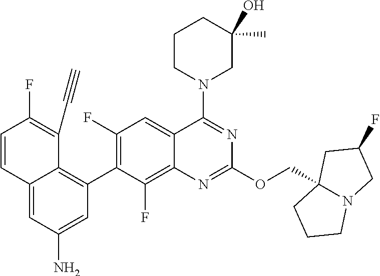 [SMILES: C#Cc1c(F)ccc2cc(N)cc(-c3c(F)cc4c(N5CCC[C@@](C)(O)C5)nc(OC[C@@]56CCCN5C[C@H](F)C6)nc4c3F)c12] | (3R)-1-(7-(3-Amino-8- ethynyl-7- fluoronaphthalen-1-yl)- 6,8-difluoro-2- (((2R,7a.S)-2- fluorotetrahydro-1H- pyrrolizin-7a(5H)- yl)methoxy)quinazolin- 4-yl)-3- methylpiperidin-3-ol Isomer 2 | Free base | |
| 65 | 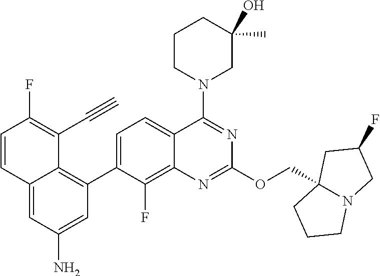 [SMILES: C#Cc1c(F)ccc2cc(N)cc(-c3ccc4c(N5CCC[C@@](C)(O)C5)nc(OC[C@@]56CCCN5C[C@H](F)C6)nc4c3F)c12] | (R)-1-(7-(3-Amino-8- ethynyl-7- fluoronaphthalen-1-yl)- 8-fluoro-2-(((2R,7aS)- 2-fluorotetrahydro-1H- pyrrolizin-7a(5H)- yl)methoxy)quinazolin- 4-yl)-3- methylpiperidin-3-ol | Free base | No Step 3. |

TABLE 12
Conditions for chiral SFC separations.

| | | Peak to |
|---|---|---|
| Compound | SFC Conditions | Ex. # |
| 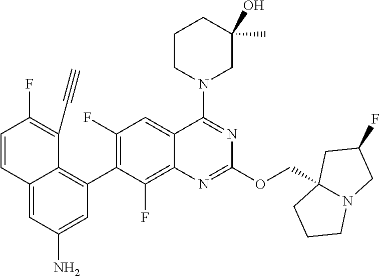 [SMILES: C#Cc1c(F)ccc2cc(N)cc(-c3c(F)cc4c(N5CCC[C@@](C)(O)C5)nc(OC[C@@]56CCCN5C[C@H](F)C6)nc4c3F)c12] | Column: Chiralpak IC, 21 × 250 mm, 5 μm Mobile phase: 45% 2-propanol Flowrate: 90 mL/min Yield: 100 mg sample was submitted to generate 29 mg of peak 1 with an ee of >96% and 30 mg of peak 2 with an ee of >99%. | Peak 1: Example 63 Peak 2: Example 64 |

TABLE 13
Analytical Data for Examples 63 to 65.

| | MS | |
|---|---|---|
| Ex. | m/z (ESI): | |
| # | (M + H)⁺ | ¹H NMR |
| 63 | 620.4 | ¹H NMR (400 MHz, METHANOL-d₄) δ ppm 7.71-7.79 (m, 1 H), |
| | | 7.64 (dd, J = 9.8, 1.7 Hz, 1 H), 7.24 (t, J = 9.0 Hz, 1 H), 7.19 (d, J = 2.3 |
| | | Hz, 1 H), 7.07 (d, J = 2.3 Hz, 1 H), 5.18-5.46 (m, 1 H), 4.28-4.37 (m, |
| | | 1 H), 4.16-4.26 (m, 2 H), 4.02 (d, J = 13.2 Hz, 1 H), 3.51 (d, J = 13.2 |
| | | Hz, 1 H), 3.42 (ddd, J = 13.2, 10.4, 2.7 Hz, 1 H), 3.17-3.29 (m, 3 H), |
| | | 2.97-3.07 (m, 1 H), 2.09-2.43 (m, 4 H), 1.69-2.09 (m, 7 H), 1.29 (s, |
| | | 3 H). |
| 64 | 620.2 | ¹H NMR (400 MHz, METHANOL-d₄) δ ppm 7.74 (dd, J = 9.2, 5.9 Hz, |
| | | 1 H), 7.62 (dd, J = 9.6, 1.7 Hz, 1 H), 7.24 (t, J = 9.0 Hz, 1 H), 7.19 (d, |
| | | J = 2.5 Hz, 1 H), 7.09 (d, J = 2.3 Hz, 1 H), 5.20-5.45 (m, 1 H), 4.17- |
| | | 4.35 (m, 3 H), 4.08 (br d, J = 13.2 Hz, 1 H), 3.47 (d, J = 13.2 Hz, 1 H), |
| | | 3.35-3.43 (m, 3 H), 3.17-3.28 (m, 2 H), 3.02 (td, J = 9.4, 6.0 Hz, 1 |
| | | H), 2.10-2.44 (m, 4 H), 1.71-2.06 (m, 6 H), 1.27 (s, 3 H). |
| 65 | 602.2 | ¹H NMR (400 MHz, METHANOL-d₄) δ ppm 7.79-7.91 (m, 1 H), |
| | | 7.72 (ddd, J = 8.9, 6.1, 2.4 Hz, 1 H), 7.18-7.27 (m, 2 H), 7.15 (d, J = 2.5 |
| | | Hz, 1 H), 7.05 (dd, J = 8.8, 2.1 Hz, 1 H), 5.19-5.46 (m, 1 H), 3.99- |
| | | 4.38 (m, 4 H), 3.36-3.64 (m, 2 H), 3.13-3.29 (m, 3 H), 3.02 (td, |
| | | J = 9.4, 5.7 Hz, 1 H), 2.67 (s, 1 H), 2.08-2.47 (m, 4 H), 1.72-2.04 (m, |
| | | 6 H), 1.25-1.30 (m, 3 H). |

### (3R)-1-(7-(8-Ethynyl-7-fluoronaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Examples 66 and 67)

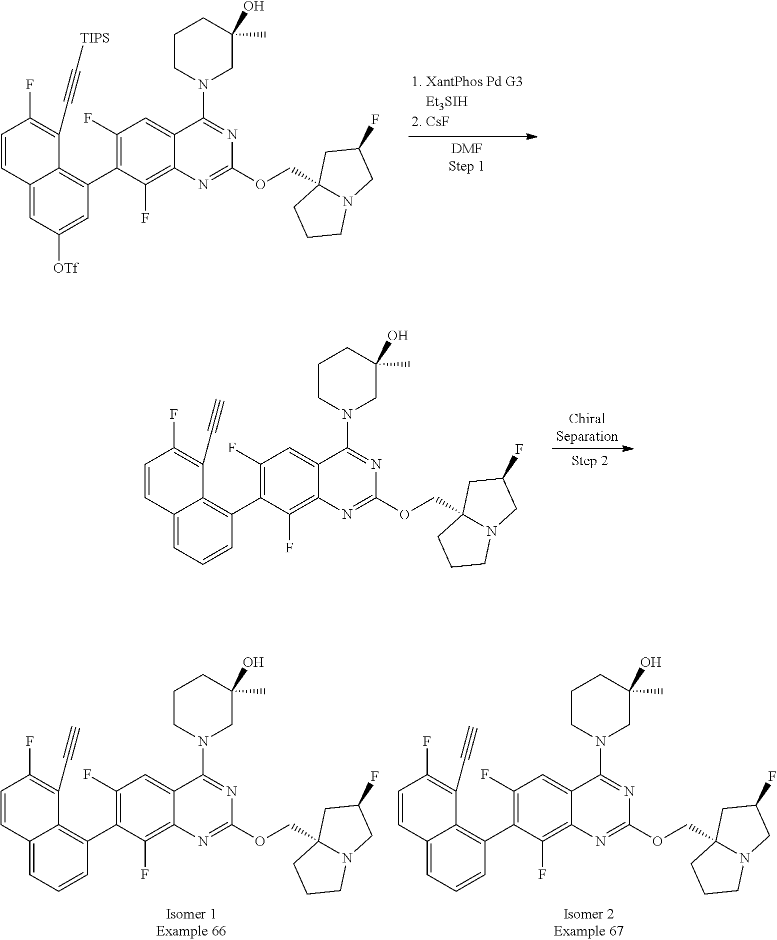

Step 1: (3R)-1-(7-(8-Ethynyl-7-fluoronaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol. To a 10 mL round-bottomed flask was added 4-(6,8-difluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)-6-fluoro-5-((triisopropylsilyl)ethynyl)naphthalen-2-yl trifluoromethanesulfonate (60 mg, 0.07 mmol, synthesized in an analogous manner to Example 61) and triethylsilane (15 mg, 21 μL, 0.13 mmol, Sigma-Aldrich Corporation) in N,N-dimethylformamide (0.7 mL). To the solution was then added methanesulfonato[9,9-dimethyl-4,5-bis(diphenylphosphino)xanthene][2′-amino-1,1′-biphenyl]palladium(II) DCM adduct (6.8 mg, 6.60 μmol, Strem Chemicals. Inc.) and the reaction was stirred at 60° C. After 20 min the reaction was cooled to rt, cesium fluoride (70 mg, 0.46 mmol, Aldrich) was added and the reaction was stirred at 50° C. After 30 min the reaction was directly loaded onto a plug of silica gel and purified by column chromatography on silica gel, eluting with a gradient of 0-100% 3:1 EtOAc/EtOH in heptane to provide (3R)-1-(7-(8-ethynyl-7-fluoronaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (39 mg, 0.06 mmol, 96% yield) as rust colored solid. m/z (ESI): 605.2 (M+H)⁺.

Step 2: Chiral separation. (3R)-1-(7-(8-Ethynyl-7-fluoronaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (39 mg, 0.06 mmol) was purified via SFC using a ChiralPak IC, 2×25 cm, 5 μm column with a mobile phase of 45% MeOH with 0.2% DEA using a flowrate of 80 mL/min. to generate 11 mg of peak 1 with an cc of 99% and 11 mg of peak 2 with an ee of 98%. Peak 1 (isomer 1, Example 66): m/z (ESI): 605.2 (M+H)⁺. ¹H NMR (400 MHz, METHANOL-d₄) δ ppm 8.06-8.17 (m, 2H), 7.62-7.73 (m, 2H), 7.56 (d, J=7.0 Hz, 1H), 7.45 (t. J=9.0 Hz, 1H), 5.18-5.51 (m, 1H), 4.18-4.41 (m, 3H), 4.01-4.11 (m, 1H), 3.52 (d, J=13.5 Hz, 1H), 3.35-3.47 (m, 2H), 3.24 (br dd. J=12.5, 9.6 Hz, 3H), 3.04 (td, J=9.4, 5.6 Hz, 1H), 2.10-2.49 (m, 4H), 1.70-2.08 (m, 6H), 1.29 (s, 3H). Peak 2 (isomer 2, Example 67): m/z (ESI): 605.2 (M+H)⁺. ¹H NMR (400 MHz, METHANOL-d₄) δ ppm 8.08-8.15 (m, 2H), 7.62-7.70 (m, 2H), 7.55-7.61 (m, 1H), 7.45 (t, J=8.9 Hz, 1H), 5.19-5.45 (m, 1H), 4.19-4.36 (m, 3H), 4.10 (br d. J=13.3 Hz, 1H), 3.44-3.52 (m, 2H), 3.30 (br d, J=3.1 Hz, 1H), 3.14-3.28 (m, 3H), 3.02 (td, J=9.2, 5.6 Hz, 1H), 2.11-2.45 (m, 4H), 1.70-2.09 (m, 6H), 1.27 (s, 3H).

### 7-(3-Amino-8-ethyl-7-fluoronaphthalen-1-yl)-6,8-difluoro-2-(((2R,7aS)-2-fluorotetrahydro-1H-pyrrolizin-7a(5H)-yl)methoxy)quinazolin-4-ol (Intermediate E)

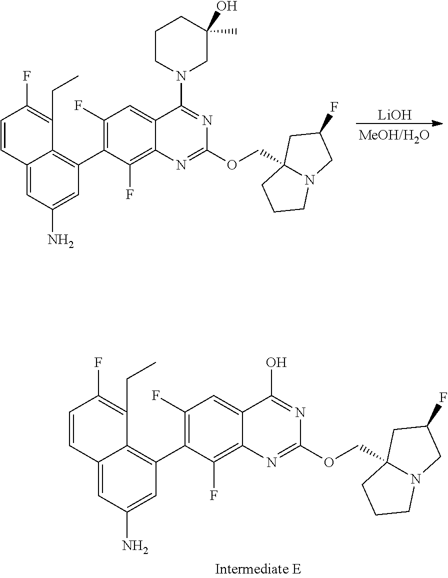

To a vial was added (3R)-1-(7-(3-amino-8-ethyl-7-fluoronaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (0.25 g, 0.40 mmol, Example 61) and lithium hydroxide (88 mg, 3.69 mmol, Aldrich) in water (2.5 mL) and MeOH (4.9 mL). The reaction was then stirred at 80° C. After 3 h additional lithium hydroxide (88 mg, 3.69 mmol, Aldrich) was added and the reaction was stirred at 80° C. for another 1.5 h. Additional lithium hydroxide (88 mg, 3.69 mmol, Aldrich) was added and the reaction was stirred at 80° C. for another 1.5 h. The reaction was diluted with H₂O (10 mL). Volatiles were removed in vacuo. The aqueous layer was extracted with EtOAc (3-10 mL), dried over MgSO₄, filtered and concentrated to give the crude 7-(3-amino-8-ethyl-7-fluoronaphthalen-1-yl)-6,8-difluoro-2-(((2R,7aS)-2-fluorotetrahydro-1H-pyrrolizin-7a(5H)-yl)methoxy)quinazolin-4-ol (0.21 g, 0.40 mmol, 99% yield) as brown solid which was used in the next step without further purification. m/z (ESI): 527.2 (M+H).

### 7-(3-Amino-8-ethyl-7-fluoronaphthalen-1-yl)-8-fluoro-2-(((2R,7aS)-2-fluorotetrahydro-1H-pyrrolizin-7a(5H)-yl)methoxy)quinazolin-4-ol (Intermediate F)

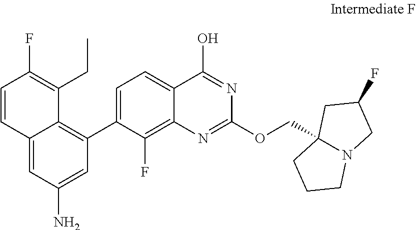 [SMILES: CCc1c(F)ccc2cc(N)cc(-c3ccc4c(O)nc(OC[C@@]56CCCN5C[C@H](F)C6)nc4c3F)c12]

Synthesized in an analogous manner to Intermediate E, using 7-bromo-2,4-dichloro-8-fluoroquinazoline (Enamine). m/z (ESI): 509.1 (M+H)⁺.

### 7-(3-Amino-8-ethynyl-7-fluoronaphthalen-1-yl)-6,8-difluoro-2-(((2R,7aS)-2-fluorotetrahydro-1H-pyrrolizin-7a(5H)-yl)methoxy)quinazolin-4-ol Intermediate G)

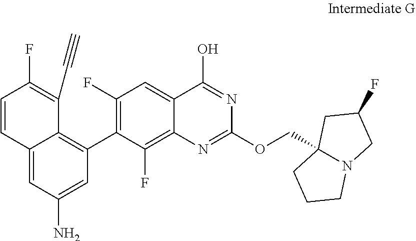 [SMILES: C#Cc1c(F)ccc2cc(N)cc(-c3c(F)cc4c(O)nc(OC[C@@]56CCCN5C[C@H](F)C6)nc4c3F)c12]

Synthesized in an analogous manner to Intermediate E. m/z (ESI): 523.2 (M+H)⁺.

### 1-(7-(3-Amino-8-ethyl-7-fluoronaphthalen-1-yl)-6,8-difluoro-2-(((2R,7aS)-2-fluorotetrahydro-1H-pyrrolizin-7a(5H)-yl)methoxy)quinazolin-4-yl)-3-(difluoromethyl)piperidin-3-ol (Example 68)

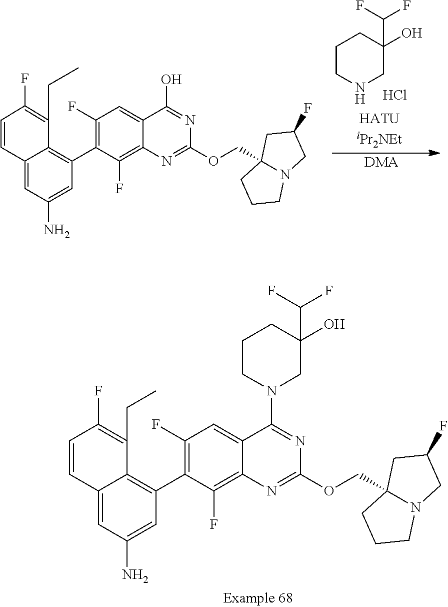

To a vial was added 7-(3-amino-8-ethyl-7-fluoronaphthalen-1-yl)-6,8-difluoro-2-(((2R,7aS)-2-fluorotetrahydro-1H-pyrrolizin-7a(5H)-yl)methoxy)quinazolin-4-ol (50 mg, 0.10 mmol, Intermediate E), DIPEA (49 mg, 66 μL, 0.38 mmol, Sigma-Aldrich Corporation) and N,N-dimethylacetamide (1.0 mL). HATU (43 mg, 0.11 mmol, Combi-Blocks Inc.) was then added and the mixture stirred for 30 min at rt before 3-(difluoromethyl)piperidin-3-ol hydrochloride (23 mg, 0.12 mmol, Enamine) was added. The reaction was left stirring at rt overnight. The crude material was then directly loaded onto a plug of silica gel and purified by column chromatography on silica gel, eluting with a gradient of 0-100% 3:1 EtOAc/EtOH in heptane to provide 1-(7-(3-amino-8-ethyl-7-fluoronaphthalen-1-yl)-6,8-difluoro-2-(((2R,7aS)-2-fluorotetrahydro-1H-pyrrolizin-7a(5H)-yl)methoxy)quinazolin-4-yl)-3-(difluoromethyl)piperidin-3-ol (35 mg, 0.05 mmol, 56% yield) as light-yellow solid. m/z (ESI): 660.1 (M+H)⁺. ¹H NMR (400 MHz, METHANOL-d₄) δ ppm 7.71-7.82 (m, 1H), 7.59 (dd, J=9.0, 5.9 Hz, 1H), 7.14-7.23 (m, 2H), 6.90-6.98 (m, 1H), 5.59-5.96 (m, 1H), 5.17-5.44 (m, 1H), 4.35-4.44 (m, 1H), 4.19-4.35 (m, 3H), 3.48-3.63 (m, 1H), 3.34-3.40 (m, 1H), 3.16-3.32 (m, 3H), 2.97-3.05 (m, 1H), 2.48-2.64 (m, 1H), 2.35-2.48 (m, 1H), 2.12-2.33 (m, 4H), 1.95-2.06 (m, 2H), 1.80-1.95 (m, 4H), 0.81 (q, J=7.1 Hz, 3H).

TABLE 14
Additional Examples 69 to 75, and 97. Prepared in an Analogous Manner to Example 68.

| Ex. | | | Salt | | Method |
|---|---|---|---|---|---|
| # | Structure | Name | Form | Reagent | Change |
| 69 | 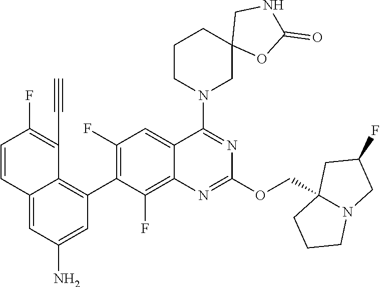 [SMILES: C#Cc1c(F)ccc2cc(N)cc(-c3c(F)cc4c(N5CCCC6(CNC(=O)O6)C5)nc(OC[C@@]56CCCN5C[C@H](F)C6)nc4c3F)c12] | 7-(7-(3-Amino-8- ethynyl-7- fluoronaphthalen- 1-yl)-6,8- difluoro-2- (((2R,7aS)-2- fluorotetrahydro- 1H-pyrrolizin- 7a(5H)- yl)methoxy)quina- zolin-4-yl)-1- oxa-3,7- diazaspiro[4.5]de- can-2-one | Free base | 1-Oxa-3,7- diazaspiro[4.5] decan-2-one (CAS#: 1308384-36-2, ChemSpace) and 7-(3- amino-8- ethynyl-7- fluoronaphthalen- 1-yl)-6,8- difluoro-2- (((2R,7aS)-2- fluorotetrahydro- 1H- pyrrolizin- 7a(5H)- yl)methoxy)quina- | |
| | | | | zolin-4-ol | |
| | | | | (Intermediate | |
| | | | | G) | |
| 70 | 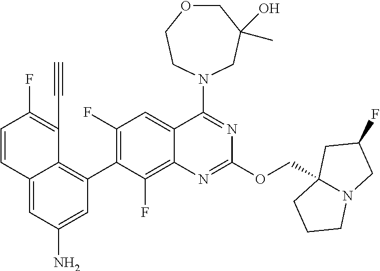 [SMILES: C#Cc1c(F)ccc2cc(N)cc(-c3c(F)cc4c(N5CCOCC(C)(O)C5)nc(OC[C@@]56CCCN5C[C@H](F)C6)nc4c3F)c12] | 4-(7-(3-Amino-8- ethynyl-7- fluoronaphthalen- 1-yl)-6,8- difluoro-2- (((2R,7aS)-2- fluorotetrahydro- 1H-pyrrolizin- 7a(5H)- yl)methoxy)quina- zolin-4-yl)-6- methyl-1,4- oxazepan-6-ol | Free base | 6-Methyl-1,4- oxazepan-6-ol hydrochloride (CAS#: 1823315-50-9, PharmaBlock) and 7-(3- amino-8- ethynyl-7- fluoronaphthalen- 1-yl)-6,8- difluoro-2- (((2R,7aS)-2- fluorotetrahydro- 1H- pyrrolizin- 7a(5H)- | |
| | | | | yl)methoxy)quina- | |
| | | | | zolin-4-ol | |
| | | | | (Intermediate | |
| | | | | G) | |
| 71 | 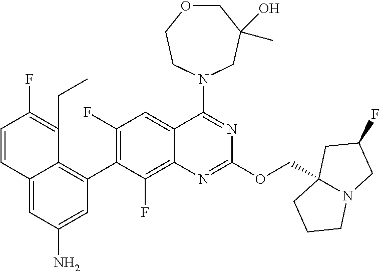 [SMILES: CCc1c(F)ccc2cc(N)cc(-c3c(F)cc4c(N5CCOCC(C)(O)C5)nc(OC[C@@]56CCCN5C[C@H](F)C6)nc4c3F)c12] | 4-(7-(3-Amino-8- ethyl-7- fluoronaphthalen- 1-yl)-6,8- difluoro-2- (((2R,7aS)-2- fluorotetrahydro- 1H-pyrrolizin- 7a(5H)- yl)methoxy)quina- zolin-4-yl)-6- methyl-1,4- oxazepan-6-ol Isomer 1 | Free base | 6-Methyl-1,4- oxazepan-6-ol hydrochloride Isomer 1 (Intermediate A1) | Chiral separation after purification Details included below. |
| 72 | 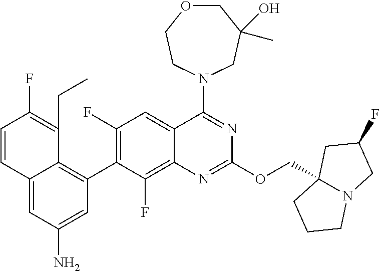 [SMILES: CCc1c(F)ccc2cc(N)cc(-c3c(F)cc4c(N5CCOCC(C)(O)C5)nc(OC[C@@]56CCCN5C[C@H](F)C6)nc4c3F)c12] | 4-(7-(3-Amino-8- ethyl-7- fluoronaphthalen- 1-yl)-6,8- difluoro-2- (((2R,7aS)-2- fluorotetrahydro- 1H-pyrrolizin- 7a(5H)- yl)methoxy)quina- zolin-4-yl)-6- methyl-1,4- oxazepan-6-ol Isomer 2 | Free base | 6-Methyl-1,4- oxazepan-6-ol hydrochloride Isomer 1 (Intermediate A1) | Chiral separation after purification. Details included below. |
| 73 | 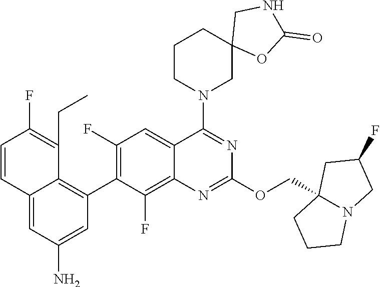 [SMILES: CCc1c(F)ccc2cc(N)cc(-c3c(F)cc4c(N5CCCC6(CNC(=O)O6)C5)nc(OC[C@@]56CCCN5C[C@H](F)C6)nc4c3F)c12] | 7-(7-(3-Amino-8- ehtyl-7- fluoronaphthalen- 1-yl)-6,8- difluoro-2- (((2R,7aS)-2- fluorotetrahydro- 1H-pyrrolizin- 7a(5H)- yl)methoxy)quina- zolin-4-yl)-1- oxa-3,7- diazaspiro[4.5]de- can-2-one | Free base | 1-Oxa-3,7- diazaspiro[4.5] decan-2-one (CAS#: 1308384-36-2, ChemSpace) | |
| 74 | 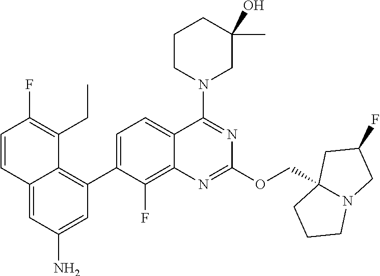 [SMILES: CCc1c(F)ccc2cc(N)cc(-c3ccc4c(N5CCC[C@@](C)(O)C5)nc(OC[C@@]56CCCN5C[C@H](F)C6)nc4c3F)c12] | (R)-1-(7-(3- Amino-8-ethyl-7- fluoronaphthalen- 1-yl)-8-fluoro-2- (((2R,7aS)-2- fluorotetrahydro- 1H-pyrrolizin- 7a(5H)- yl)methoxy)quina- zolin-4-yl)-3- methylpiperidin- 3-ol | Free base | (3R)-3- Methylpiperidin- 3-ol hydrochloride (CAS#: 2305080-34-4, Synnovator, Inc.) and 7-(3- amino-8- ethyl-7- fluoronaphthalen- 1-yl)-8- fluoro-2- (((2R,7aS)-2- fluorotetrahydro- 1H- pyrrolizin- | |
| | | | | 7a(5H)- | |
| | | | | yl)methoxy)quina- | |
| | | | | zolin-4-ol | |
| | | | | (Intermediate | |
| | | | | F) | |
| 75 | 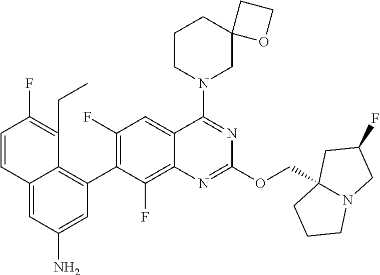 [SMILES: CCc1c(F)ccc2cc(N)cc(-c3c(F)cc4c(N5CCCC6(CCO6)C5)nc(OC[C@@]56CCCN5C[C@H](F)C6)nc4c3F)c12] | 4-(6,8-Difluoro- 2-(((2R,7aS)-2- fluorotetrahydro- 1H-pyrrolizin- 7a(5H)- yl)methoxy)-4- (1-oxa-6- azaspiro[3.5]nonan- 6-yl)quinazolin-7- yl)-5-ethyl-6- fluoronaphthalen- 2-amine | Free base | 1-Oxa-6- azaspiro[3.5]no- nane hydrochloride (CAS#: 1956354-59-6, Enamine) | |
| 97 | 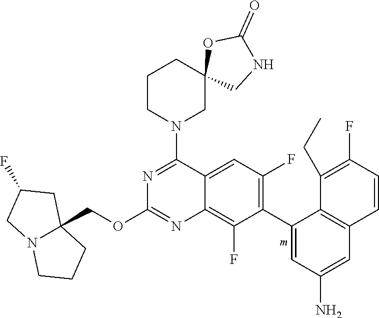 [SMILES: CCc1c(F)ccc2cc(N)cc(-c3c(F)cc4c(N5CCC[C@]6(CNC(=O)O6)C5)nc(OC[C@@]56CCCN5C[C@H](F)C6)nc4c3F)c12] | (5S)-7-(7-(3- Amino-8-ethyl-7- fluoronaphthalen- 1-yl)-6,8- difluoro-2- (((2R,7aS)-2- fluorotetrahydro- 1H-pyrrolizin- 7a(5H)- yl)methoxy)quina- zolin-4-yl)-1- oxa-3,7- diazaspiro[4.5]de- can-2-one | Free base | 1-Oxa-3,7- diazaspiro[4.5] decan-2-one (CAS#: 1308384-36-2, ChemSpace) | Chiral separation after purification. Details included below. |

TABLE 15
SFC conditions for chiral separation.

| | | Peak to |
|---|---|---|
| Compound | SFC Conditions | Ex.# |
| 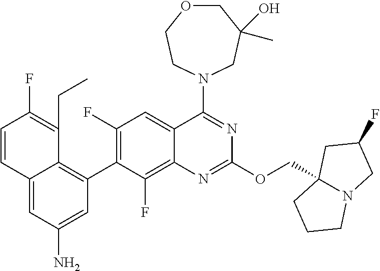 [SMILES: CCc1c(F)ccc2cc(N)cc(-c3c(F)cc4c(N5CCOCC(C)(O)C5)nc(OC[C@@]56CCCN5C[C@H](F)C6)nc4c3F)c12] | Column: Chiralcel OJ, 2 x 25 cm, 5 μm Mobile phase: 25% MeOH with 0.2% DEA. Flowrate: 80 mL/min. Yield: 29 mg sample was submitted to generate 12 mg of peak 1 with an ee of 94% and 11 mg of peak 2 with an ee of 99%. | Peak 1: Example 71 Peak 2: Example 72 |
| 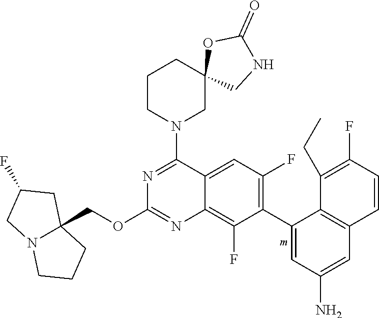 [SMILES: CCc1c(F)ccc2cc(N)cc(-c3c(F)cc4c(N5CCC[C@]6(CNC(=O)O6)C5)nc(OC[C@@]56CCCN5C[C@H](F)C6)nc4c3F)c12] | Column: Chiralpak AS, 2.1 x 25 cm, 5 μm Mobile phase: 30% MeOH with 0.2% DEA. Flowrate: 80 mL/min. Yield: 150 mg sample was submitted to generate 35 mg of peak 1&2 and 45 mg of peak 3&4. Column: Chiralcel OJ, 2.1 x 25 cm, 5 μm. Mobile phase: 35% iPrOH with 0.2% DEA. Flowrate: 100 mL/min. Yield: 45 mg sample was submitted to generate 4.1 mg of peak 1 with ee of 99% and 5.5 mg of peak 2 with ee of 99%. | Peak 2: Example 97 |

TABLE 16
Analytical Data for Examples 69 to 75, and 97.

| | MS | |
|---|---|---|
| Ex. | m/z (ESI): | |
| # | (M + H)⁺ | ¹H NMR |
| 69 | 661.2 | ¹H NMR (400 MHz, METHANOL-d₄) δ ppm 7.75 (dd, J = 9.1, 5.7 Hz, |
| | | 1 H), 7.49 (d, J = 9.6 Hz, 1 H), 7.24 (t, J = 8.9 Hz, 1 H), 7.20 (d, J = 2.3 |
| | | Hz, 1 H), 7.04-7.10 (m, 1 H), 5.13-5.45 (m, 1 H), 4.13-4.45 (m, 4 |
| | | H), 3.79 (br d, J = 13.6 Hz, 1 H), 3.36-3.65 (m, 4 H), 3.13-3.32 (m, 3 |
| | | H), 3.02 (td, J = 9.2, 5.9 Hz, 1 H), 2.11-2.42 (m, 5 H), 1.84-2.06 (m, |
| | | 5 H). |
| 70 | 636.3 | ¹H NMR (400 MHz, METHANOL-d₄) δ ppm 7.83-7.95 (m, 1 H), |
| | | 7.72-7.79 (m, 1 H), 7.17-7.30 (m, 2 H), 7.07 (dd, J = 5.4, 2.3 Hz, 1 |
| | | H), 5.20-5.42 (m, 1 H), 4.39-4.54 (m, 1 H), 4.18-4.39 (m, 3 H), |
| | | 3.97-4.11 (m, 2 H), 3.78-3.95 (m, 2 H), 3.56-3.75 (m, 2 H), 3.13- |
| | | 3.31 (m, 4 H), 3.02 (td, J = 9.2, 5.9 Hz, 1 H), 2.08-2.43 (m, 3 H), 1.83- |
| | | 2.03 (m, 3 H), 1.25-1.31 (m, 3 H). |
| 71 | 640.2 | ¹H NMR (500 MHz, DMSO-d₆) δ ppm 8.22 (d, J = 10.4 Hz, 1 H), 7.58 |
| | | (dd, J = 9.1, 6.1 Hz, 1 H), 7.24 (t, J = 9.4 Hz, 1 H), 7.03 (d, J = 2.4 Hz, 1 |
| | | H), 6.85 (d, J = 2.3 Hz, 1 H), 5.44 (s, 2 H), 5.22-5.34 (m, 1 H), 4.27 |
| | | (dt, J = 14.5, 4.9 Hz, 1 H), 4.08-4.15 (m, 2 H), 4.01 (d, J = 10.4 Hz, 1 |
| | | H), 3.97 (br t, J = 5.4 Hz, 2 H), 3.91 (d, J = 14.8 Hz, 1 H), 3.71-3.79 |
| | | (m, 1 H), 3.04-3.14 (m, 2 H), 3.00-3.04 (m, 1 H), 2.78-2.87 (m, 1 |
| | | H), 2.40-2.49 (m, 1 H), 2.28-2.39 (m, 1 H), 2.04-2.20 (m, 2 H), |
| | | 1.94-2.04 (m, 1 H), 1.74-1.86 (m, 3 H), 1.12 (s, 3 H), 0.76 (t, J = 7.4 |
| | | Hz. 3 H). |
| 72 | 640.2 | ¹H NMR (500 MHz, DMSO-d₆) δ ppm 8.27 (d, J = 10.6 Hz, 1 H), 7.58 |
| | | (dd, J = 9.1, 6.1 Hz, 1 H), 7.24 (t, J = 9.4 Hz, 1 H), 7.03 (d, J = 2.4 Hz, 1 |
| | | H), 6.87 (d, J = 2.3 Hz, 1 H), 5.44 (s, 2 H), 5.32 (br s, 1 H), 4.31 (dt, |
| | | J = 14.5, 4.7 Hz, 1 H), 4.17 (d, J = 14.8 Hz, 1 H), 4.07-4.14 (m, 1 H), |
| | | 4.02 (d, J = 10.4 Hz, 1 H), 3.96 (t, J = 5.1 Hz, 2 H), 3.81 (d, J = 14,8 Hz, 1 |
| | | H), 3.70 (dt, J = 14.6, 5,5 Hz, 1 H), 3.50-3.60 (m, 2 H), 3,04-3.15 (m, |
| | | 2 H), 2.96-3.04 (m, 1 H), 2.77-2.87 (m, 1 H), 2.37-2.47 (m, 1 H), |
| | | 2.29 (ddd, J = 14.0, 7.2, 2.7 Hz, 1 H), 1.95-2.20 (m, 3 H), 1.72-1.89 |
| | | (m, 3 H), 1.15 (s, 3 H), 0.73 (t, J = 7.4 Hz, 3 H). |
| 73 | 665.2 | ¹HNMR (400 MHz, METHANOL-d₄) δ ppm 7.54-7.64 (m, 2 H), |
| | | 7.14-7.24 (m, 2 H), 6.96 (t, J = 2.6 Hz, 1 H), 5.19-5.43 (m, 1 H), 4.30- |
| | | 4.37 (m, 1 H), 4.22-4.30 (m, 2 H), 4.18-4.22 (m, 1 H), 3.79 (dd, |
| | | J = 13.2, 10.5 Hz, 1 H), 3.55-3.67 (m, 1 H), 3.47-3.54 (m, 1 H), 3.38- |
| | | 3.45 (m, 1 H), 3.16-3.28 (m, 3 H), 2.96-3.07 (m, 1 H), 2.48-2.61 |
| | | (m, 1 H), 2.33-2.46 (m, 2 H), 2.13-2.32 (m, 5 H), 1.97-2.03 (m, 3 |
| | | H), 1.89 (br d, J = 3.6 Hz, 1 H), 0.81 (brt, J = 7.3 Hz, 3 H). |
| 74 | 606.3 | ¹H NMR (400 MHz, METHANOL-d₄) δ ppm 7.94 (d, J = 8.6 Hz, 1 H), |
| | | 7.57 (dd, J = 9.0, 5.9 Hz, 1 H), 7.28 (t, J = 7.6 Hz, 1 H), 7.13-7.20 (m, 2 |
| | | H), 6.92 (d, J = 2.5 Hz, 1 H), 5.16-5.46 (m, 1 H), 4.30-4.35 (m, 1 H), |
| | | 4.24 (br d, J = 9.2 Hz, 2 H), 4.05 (s, 1 H), 3.52-3.62 (m, 2 H), 3.16- |
| | | 3.31 (m, 3 H), 3.01 (s, 1 H), 2.27 (s, 3 H), 2.11-2.21 (m, 3 H), 1.96- |
| | | 2.05 (m, 3 H), 1.75-1.86 (m, 4 H), 1.29 (d, J = 13.2 Hz, 3 H), 0.79 (q, |
| | | J = 7.3 Hz, 3 H). |
| 75 | 636.3 | ¹H NMR (400 MHz, METHANOL-d₄) δ ppm 7.82 (br d, J = 9.6 Hz, 1 |
| | | H), 7.59 (dd, J = 9.1, 6.0 Hz, 1 H), 7.16-7.22 (m, 2 H), 6.97 (d, J = 2.1 |
| | | Hz, 1 H), 5.20-5.45 (m, 1 H), 4.55-4.62 (m, 2 H), 4.21-4.45 (m, 3 |
| | | H), 4.13 (br s, 1 H), 3.64-3.73 (m, 1 H), 3.35-3.46 (m, 1 H), 3.14- |
| | | 3.31 (m, 4 H), 2.98-3.06 (m, 1 H), 2.46-2.60 (m, 3 H), 2.35-2.46 |
| | | (m, 2 H), 2.09-2.31 (m, 4 H), 1.88-2.09 (m, 5 H), 1.82 (br d, J = 15.0 |
| | | Hz, 1 H), 0.78-0.86 (m, 3 H). |
| 97 | 665.2 | ¹H NMR (400 MHz, METHANOL-d₄) δ ppm 7.54-7.69 (m, 2 H), |
| | | 7.12-7.26 (m, 2 H), 6.95 (d, J = 2.5 Hz, 1 H), 5.21-5.45 (m, 1 H), |
| | | 4.19-4.41 (m, 4 H), 3.78 (d, J = 13.7 Hz, 1 H), 3.47-3.63 (m, 2 H), |
| | | 3.39-3.45 (m, 1 H), 3.20-3.28 (m, 2 H), 2.96-3.09 (m, 2 H), 2.47- |
| | | 2.61 (m, 1 H), 2.35-2.47 (m, 1 H), 2.15-2.30 (m, 4 H), 1.98-2.07 |
| | | (m, 3 H), 1.87-1.96 (m, 2 H), 0.91 (br dd, J = 11.6, 4.1 Hz, 1 H), 0.82 |
| | | (br t, J = 7.4 Hz, 3 H). ¹⁹F NMR (376 MHz, METHANOL-d₄) δ ppm |
| | | −117.70 (br d, J = 4.3 Hz, 1 F), −122.61 (s, 1 F), −124.29 (br d, J = 4.3 Hz, |
| | | 1 F), −173.61 (s, 1 F). |

### 4-Bromo-6-chloro-5-cyclopropyl-1-(tetrahydro-2H-pyran-2-yl)-1H-indazole (Intermediate K)

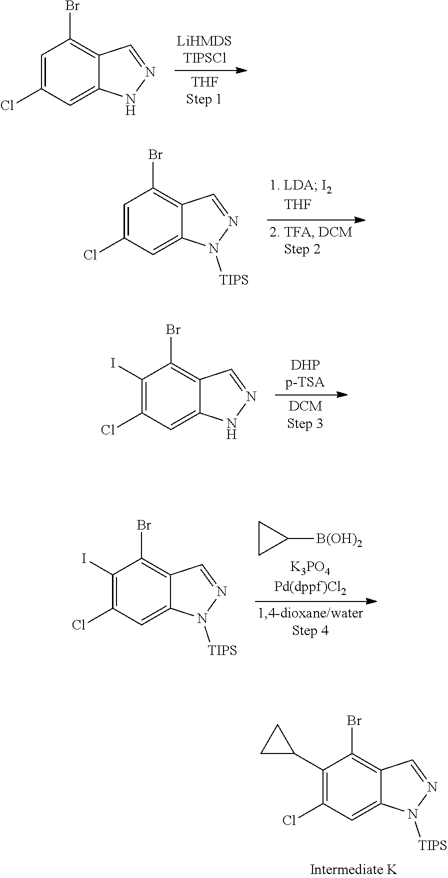

Step 1. 4-Bromo-6-chloro-1-(triisopropylsilyl)-1H-indazole. A vial was charged with 4-bromo-6-chloro-1H-indazole (2.00 g, 8.64 mmol, CombiBlocks Inc.) and tetrahydrofuran (3.0 mL). The reaction mixture was then cooled to −78° C. LiHMDS (1.0 M in THF, 10.4 mL, 10.4 mmol) was added dropwise, and the mixture was stirred for 20 min at −78° C. Triisopropylchlorosilane (2.00 g, 2.2 mL, 10.4 mmol) was added dropwise, and the mixture was stirred for 20 min at −78° C. before warming to rt. Upon completion (as indicated by TLC), the reaction was carefully quenched by the addition of water. The aqueous layer was extracted with EtOAc, and the combined organic phases were washed with brine, dried over anhydrous sodium sulfate, filtered, and concentrated under reduced pressure. The crude material was purified by column chromatography on silica gel, eluting with a gradient of 0-2% (3:1 EtOAc/EtOH) in heptane, to provide 4-bromo-6-chloro-1-(triisopropylsilyl)-1H-indazole (2.75 g, 7.09 mmol, 82% yield) as orange solid. m/z (ESI): 231.0/233.0 (M-TIPS+H)⁺. ¹H NMR (400 MHz, DMSO-d₆) δ ppm 8.34-8.39 (m, 1H), 7.67-7.70 (m, 1H), 7.51-7.55 (m, 1H), 1.70-1.88 (m, 3H), 1.08 (d, J=7.46 Hz, 18H).

Step 2. 4-Bromo-6-chloro-5-iodo-1-(triisopropylsilyl)-1H-indazole. A vial was charged with 4-bromo-6-chloro-1-(triisopropylsilyl)-1H-indazole (2.00 g, 5.16 mmol) and tetrahydrofuran (26 mL) under nitrogen. The reaction mixture was then cooled to −78° C. LDA (1.0 M in THF, 6.7 mL, 6.7 mmol) was added dropwise, and the reaction mixture was stirred at −78° C. for 1 h. A solution of iodine (1.70 g, 6.70 mmol) in THF (1.0 mL) was then added dropwise, and the reaction was warmed to rt with stirring while monitoring via LCMS. Upon completion, the reaction was quenched with 10% aqueous sodium thiosulfate solution. The aqueous layer was extracted with DCM, and the combined organic phases were washed with brine, dried over anhydrous sodium sulfate, and filtered. TFA (2.0 mL) was added dropwise to the DCM layer, and the mixture was stirred until TIPS deprotection was complete as indicated by TLC. The organic layer was concentrated under reduced pressure, and the crude material was purified by reverse phase chromatography to provide 4-bromo-6-chloro-5-iodo-1H-indazole (0.81 g, 2.27 mmol, 44% yield) as off-white solid. m/z (ESI): 356.8/358.8 (M+H)⁺.

Step 3. 4-Bromo-6-chloro-5-iodo-1-(tetrahydro-2H-pyran-2-yl)-1H-indazole. A vial was charged with 4-bromo-6-chloro-5-iodo-1H-indazole (1.00 g, 2.80 mmol), 4-methylbenzenesulfonic acid (24 mg, 0.14 mmol), 3,4-dihydro-2H-pyran (0.71 g, 0.77 mL, 8.39 mmol) and dichloromethane (14 mL).

The reaction mixture was stirred at rt while monitoring via LCMS. Upon completion, the reaction was concentrated under reduced pressure. The crude residue was dissolved in DMSO (2×4.0 mL) and purified by reverse phase chromatography to provide 4-bromo-6-chloro-5-iodo-1-(tetrahydro-2H-pyran-2-yl)-1H-indazole (1.10 g, 2.49 mmol, 89% yield) as off-white solid. m/z (ESI): 440.8/442.8 (M+H)⁺.

Step 4. 4-Bromo-6-chloro-5-cyclopropyl-1-(tetrahydro-2H-pyran-2-yl)-1H-indazole. A vial was charged with 4-bromo-6-chloro-5-iodo-1-(tetrahydro-2H-pyran-2-yl)-1H-indazole (0.50 g, 1.13 mmol), cyclopropylboronic acid (0.29 g, 3.40 mmol, potassium phosphate tribasic (0.87 g, 4.10 mmol), CombiBlocks), 1,1′-bis(diphenylphosphino)ferrocene-palladium dichloride (83 mg, 0.11 mmol), 1,4-dioxane (4.7 mL) and water (1.0 mL) under nitrogen. The mixture was heated to 100° C. and monitored via LCMS. Upon completion, the crude reaction was purified by reverse phase chromatography to provide 4-bromo-6-chloro-5-cyclopropyl-1-(tetrahydro-2H-pyran-2-yl)-1H-indazole (0.16 g, 0.46 mmol, 40% yield) as orange residue. LCMS m/z (ESI): 354.9/357.0 (M+H)⁺.

### (Z)-4-Bromo-6-chloro-5-(prop-1-en-1-yl)-1-(tetrahydro-2H-pyran-2-yl)-1H-indazole (Intermediate L)

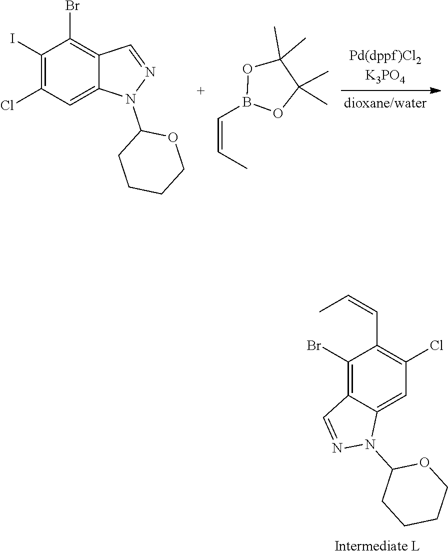

A vial was charged with (Z)-4,4,5,5-tetramethyl-2-(prop-1-en-1-yl)-1,3,2-dioxaborolane (0.25 g, 0.28 mL, 1.47 mmol, AstaTech), 4-bromo-6-chloro-5-iodo-1-(tetrahydro-2H-pyran-2-yl)-1H-indazole (0.50 g, 1.13 mmol, Intermediate K step 3), potassium phosphate tribasic (0.84 g, 3.96 mmol), 1,1′-bis(diphenylphosphino)ferrocene-palladium dichloride (83 mg, 0.11 mmol), water (0.8 mL) and 1,4-dioxane (3.8 mL). The reaction mixture was heated to 100° C. while monitoring via LCMS. Upon completion, the crude material was purified by reverse phase chromatography to provide (Z)-4-bromo-6-chloro-5-(prop-1-en-1-yl)-1-(tetrahydro-2H-pyran-2-yl)-1H-indazole (0.23 g, 0.65 mmol, 57% yield) as orange oil. m/z (ESI): 270.8/272.8 (M-THP+H)⁺.

### (Z)-6-Chloro-5-(prop-1-en-1-yl)-1-(tetrahydro-2H-pyran-2-yl)-4-(4,4,5,5-tetramethyl-1,3,2-dioxaborolan-2-yl)-1H-indazole (Intermediate M)

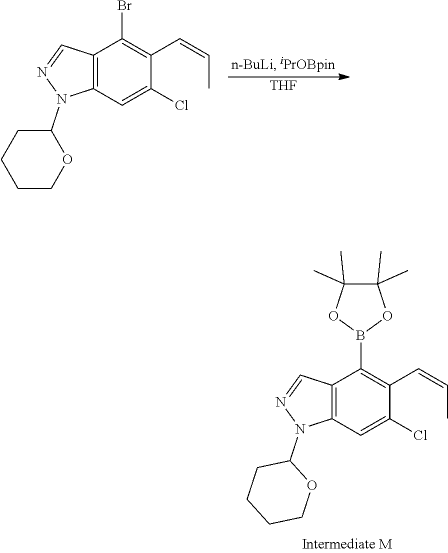 [SMILES: C/C=C\c1c(Cl)cc2c(cnn2C2CCCCO2)c1Br ; C/C=C\c1c(Cl)cc2c(cnn2C2CCCCO2)c1B1OC(C)(C)C(C)(C)O1 ; "THF"]

Intermediate L (1.00 g, 2.81 mmol) was dissolved in THF (14 mL) in a flask and the solution was cooled to −78° C. n-Butyl lithium (2.5 M in hexanes, 1.7 mL, 4.22 mmol) was added dropwise and the mixture was stirred at −78° C. for 10 min. 2-Isopropoxy-4,4,5,5-tetramethyl-1,3,2-dioxaborolane (0.79 g, 0.86 mL, 4.22 mmol) was then added dropwise and the mixture was stirred at −78° C. for 90 min. Saturated aqueous NH₄Cl solution (10 mL) was added, and the mixture was warmed to rt. The aqueous layer was extracted with EtOAc (3×15 mL) and the combined organic layers were dried over Na₂SO₄, filtered and volatiles were removed n vacuo. The residue was purified via reverse phase chromatography, eluting with a gradient of 5-100% MeCN/H₂O+0.1% TFA). To the product containing fractions was added saturated aqueous NaHCO₃ solution (20 mL) and the resulting aqueous phase was extracted with EtOAc (3×10 mL). The combined organic layers were dried over Na₂SO₄ and volatiles were removed in vacuo to give (Z)-6-chloro-5-(prop-1-en-1-yl)-1-(tetrahydro-2H-pyran-2-yl)-4-(4,4,5,5-tetramethyl-1,3,2-dioxaborolan-2-yl)-1H-indazole (0.55 g, 1.37 mmol, 49% yield). m/z (ESI): (M+H)⁺=403.0.

### rel-((1R,2S)-2-Methylcyclopropyl)boronic acid (Intermediate N)

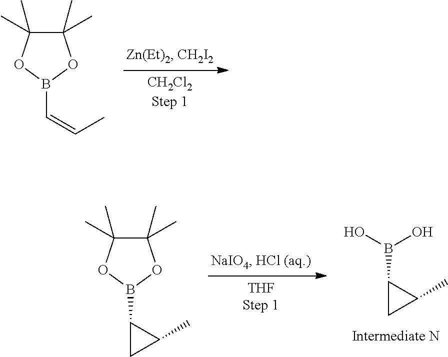

Step 1: rel-4,4,5,5-Tetramethyl-2-((1R,2S)-2-methylcyclopropyl)-1,3,2-dioxaborolane. To a cooled solution of dichloromethane (19.8 mL) in a 100 mL round-bottomed flask was added diethylzinc (1.0 M in hexane, 10.4 mL, 10.4 mmol). The mixture was allowed to stir at 0° C. for 10 min, and then diiodomethane (2.79 g, 0.84 mL, 10.41 mmol) was added dropwise. The reaction mixture was allowed to stir at this temperature for 25 min, then a pre-cooled solution of (Z)-4,4,5,5-tetramethyl-2-(prop-1-en-1-yl)-1,3,2-dioxaborolane (0.50 g, 3.00 mmol, Advanced ChemBlocks Inc.) in dichloromethane (10 mL) was added via cannula transfer. The reaction was then stirred at 0° C. for 40 min then allowed to stir at rt for 3.5 h. The reaction was quenched via the addition of cold 1 N HCl (50 mL) solution. The mixture was then transferred to a separatory funnel using dichloromethane (50 mL). The layers were separated, and the aqueous layer was extracted with dichloromethane (3-50 mL). The combined organic layers were dried with sodium sulfate, filtered, and concentrated under reduced pressure. The resultant orange oil was then filtered through a short silica gel plug (eluting with DCM) to provide 1-4,4,5,5-tetramethyl-2-((1R,2S)-2-methylcyclopropyl)-1,3,2-dioxaborolane (0.27 g, 1.46 mmol, 49% yield) as clear oil. ¹H NMR (400 MHz, CHLOROFORM-d) δ ppm 1.26 (s, 6H), 1.25 (s, 6H), 1.13-1.18 (m, 3H), 1.04-1.13 (m, 1H), 0.74-0.83 (m, 1H), 0.38 (dt, J=3.45, 1.62 Hz, 1H), −0.08 (d, J=6.90 Hz, 1H).

Step 2: rel-((1R,2S)-2-Methylcyclopropyl)boronic acid. Sodium (meta)periodate (0.91 g, 4.30 mmol, Sigma-Aldrich Corporation) was added to a rt solution of rel-4,4,5,5-tetramethyl-2-((1R,2S)-2-methylcyclopropyl)-1,3,2-dioxaborolane (0.26 g, 1.40 mmol) in tetrahydrofuran (10 mL) and water (2.6 mL). The reaction mixture was stirred for 30 min, and then 2 N HCl (0.47 mL, 0.94 mmol) was added. The mixture was stirred for 12 h, and then diluted with water (15 mL). The aqueous layer was extracted with EtOAc (3×15 mL). The combined organic layers were then dried with sodium sulfate, filtered, and concentrated under reduced pressure to provide rel-((1R,2S)-2-methylcyclopropyl)boronic acid (0.10 g, 1.04 mmol, 74% yield) as light pink oil. ¹H NMR (400 MHz, METHANOL-d₆) δ ppm 1.00-1.13 (m, 4H), 0.61-0.74 (m, 1H), 0.28-0.37 (m, 1H), −0.06-0.06 (m, 1H).

### 6-Chloro-5-((1S,2R)-2-methylcyclopropyl)-1-(tetrahydro-2H-pyran-2-yl)-4-(4,4,5,5-tetramethyl-1,3,2-dioxaborolan-2-yl)-1H-indazole (Intermediate O)

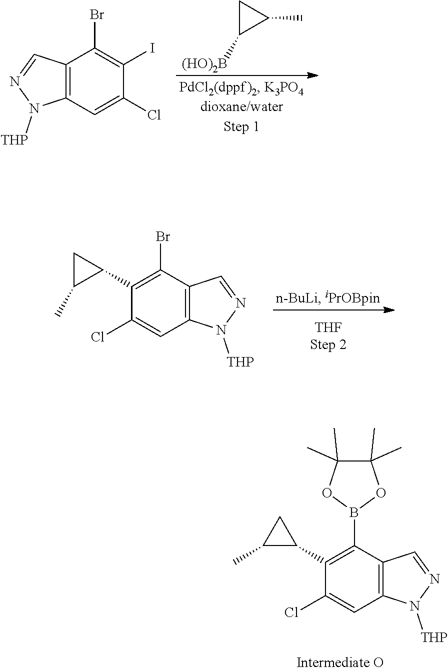

Step 1: rel-4-Bromo-6-chloro-5-((1S,2R)-2-methylcyclopropyl)-1-(tetrahydro-2H-pyran-2-yl)-1H-indazole. A vial was charged with 4-bromo-6-chloro-5-iodo-1-(tetrahydro-2H-pyran-2-yl)-1H-indazole (1.0 g, 2.3 mmol, Intermediate K, step 3), rel-((1R,2S)-2-methylcyclopropyl)boronic acid (0.41 g, 4.10 mmol, Intermediate N), potassium phosphate tribasic (1.70 g, 7.90 mmol), 1,1′-bis(diphenylphosphino)ferrocene-palladium dichloride (0.17 g, 0.23 mmol), water (1.5 mL) and 1,4-dioxane (7.5 mL). The reaction mixture was heated to 100° C. Upon completion of the reaction, the crude material was purified by reverse phase chromatography to provide rel-4-bromo-6-chloro-5-((1R,2S)-2-methylcyclopropyl)-1-(tetrahydro-2H-pyran-2-yl)-1H-indazole (0.40 g, 1.10 mmol, 48% yield) as orange oil. m/z (ESI): 369.0 (M+H)⁺.

Step 2: 6-Chloro-5-((1S,2R)-2-methylcyclopropyl)-1-(tetrahydro-2H-pyran-2-yl)-4-(4,4,5,5-tetramethyl-1,3,2-dioxaborolan-2-yl)-1H-indazole, rel-4-Bromo-6-chloro-5-((1R,2S)-2-methylcyclopropyl)-1-(tetrahydro-2H-pyran-2-yl)-1H-indazole (1.20 g, 3.30 mmol) was dissolved in THF (16 mL) and cooled to −78° C. n-Butyl lithium (2.0 mL, 4.90 mmol) was added dropwise, and the reaction mixture was stirred at −78° C. for 10 min. 2-Isopropoxy-4,4,5,5-tetramethyl-1,3,2-dioxaborolane (0.90 g, 1.00 mL, 4.90 mmol) was then added dropwise and the reaction mixture was stirred for 90 min. The reaction was quenched with saturated aqueous NH₄Cl solution (20 mL) and was allowed to warm to rt.

The aqueous phase was extracted with EtOAc (3-20 mL), and the combined organic layers were dried over Na₂SO₄, filtered and volatiles were removed in vacuo. The residue was purified via reverse phase column chromatography to yield rel-6-chloro-5-((1R,2S)-2-methylcyclopropyl)-1-(tetrahydro-2H-pyran-2-yl)-4-(4,4,5,5-tetramethyl-1,3,2-dioxaborolan-2-yl)-FH-indazole (0.74 g, 1.80 mmol, 55% yield). m/z (ESI): 417.2 (M+H)⁺.

The sample (9.5 g) was purified via SFC using a Chiralpak AD, 30×250 mm, 5 μm, column with a mobile phase of 20% 2-propanol using a flowrate of 150 mL/min to generate 3.58 g of peak 1 with an ee of >99%, and 5.02 g of peak 2 with an ee of >99%. Peak 2 yielded the desired isomer.

### (Z)-6-Methyl-5-(prop-1-en-1-yl)-1-(tetrahydro-2H-pyran-2-yl)-4-(4,4,5,5-tetramethyl-1,3,2-dioxaborolan-2-yl)-1H-indazole (Intermediate P)

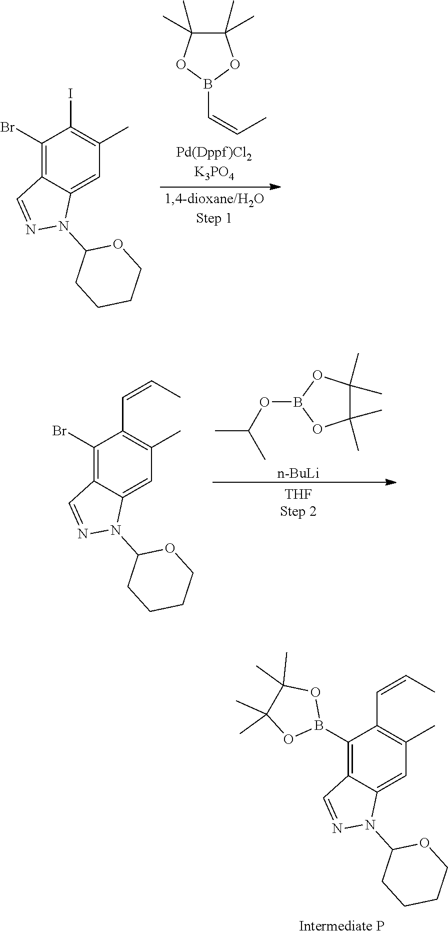

Step 1: (Z)-4-Bromo-6-methyl-5-(prop-1-en-1-yl)-1-(tetrahydro-2H-pyran-2-yl)-1H-indazole. A vial was charged with potassium phosphate tribasic (3.53 g, 16.6 mmol), 1,1′-bis(diphenylphosphino)ferrocene-palladium dichloride (0.35 g, 0.48 mmol), 4,4,5,5-tetramethyl-2-[(z)-prop-1-enyl]-1,3,2-dioxaborolane (1.1 mL, 5.7 mmol, PharmaBlock), 4-bromo-5-iodo-6-methyl-1-(tetrahydro-2H-pyran-2-yl)-1H-indazole (2.00 g, 4.75 mmol), water (3.2 mL) and 1,4-dioxane (16 mL). The reaction mixture was then heated to 100° C. After 1 h, the reaction was concentrated under reduced pressure. The crude material was purified by chromatography through a 80 g silica gel column, eluting with a gradient of 0-20% EtOAc in heptanes, to provide (Z)-4-bromo-6-methyl-5-(prop-1-en-1-yl)-1-(tetrahydro-2H-pyran-2-yl)-1H-indazole (1.20 g, 3.59 mmol, 76% yield) as clear oil. m/z (ESI): 335.0 (M+H)⁺.

Step 2: (Z)-6-Methyl-5-(prop-1-en-1-yl)-1-(tetrahydro-2H-pyran-2-yl)-4-(4,4,5,5-tetramethyl-1,3,2-dioxaborolan-2-yl)-1H-indazole. (Z)-4-bromo-6-methyl-5-(prop-1-en-1-yl)-1-(tetrahydro-2H-pyran-2-yl)-1H-indazole (1.03 g, 3.08 mmol) was dissolved in tetrahydrofuran (40 mL) and cooled to −78° C. n-Butyl lithium (2.5 M in hexane, 1.9 mL, 4.6 mmol) was added dropwise and the mixture was stirred at −78° C. for 12 min. 2-Isopropoxy-44,5,5-tetramethyl-1,3,2-dioxaborolane (1.1 mL, 5.6 mmol, Sigma-Aldrich Corporation) was then added dropwise and the mixture was stirred at −78° C. for 1.5 h at which point saturated aq. NH₄Cl was added. The mixture was warmed to rt and the aqueous layer was extracted with EtOAc. The combined organic layers were dried over Na₂SO₄, filtered and volatiles were removed in vacuo. The residue was purified via column chromatography (40 g) on silica gel using a gradient of 0-20% EtOAc in heptanes to provide (Z)-6-methyl-5-(prop-1-en-1-yl)-1-(tetrahydro-2H-pyran-2-yl)-4-(4,4,5,5-tetramethyl-1,3,2-dioxaborolan-2-yl)-1H-indazole (0.67 g, 1.75 mmol, 57% yield) as white solid. m/z (ESI): 383.2 (M+H)⁺.

### (S)-6-(7-Bromo-2-chloro-8-fluoroquinazolin-4-yl)-1-oxa-6-azaspiro[3.5]nonane (Intermediate Q)

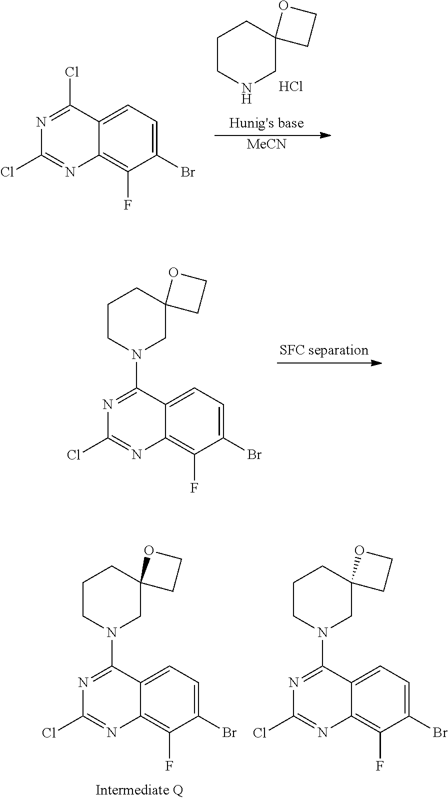

To a suspension solution of 7-bromo-2,4-dichloro-8-fluoroquinazoline (3.71 g, 12.5 mmol, PharmaBlock) and 1-oxa-6-azaspiro[3.5]nonane hydrochloride (2.05 g, 12.5 mmol, Enamine) in acetonitrile (50 mL) at 0° C. was added 1,1-dimethyltriethylamine (6.6 mL, 37.6 mmol). The reaction mixture was stirred at 0° C. for 15 min. The solid formed was collected by filtration, washed with acetonitrile and dried to give pure 6-(7-bromo-2-chloro-8-fluoroquinazolin-4-yl)-1-oxa-6-azaspiro[3.5]nonane (3.97 g, 10.3 mmol, 82% yield) as tan solid. ¹H NMR (400 MHz, DMSO-d₆) δ ppm 7.93 (dd. J=9.2, 1.5 Hz, 1H), 7.77 (dd, J=9.0, 6.7 Hz, 1H), 4.35-4.43 (m, 2H), 4.26 (d, J=13.4 Hz, 1H), 4.00-4.08 (m, 1H), 3.76 (d, J=13.2 Hz, 1H), 3.32-3.49 (m, 1H), 2.30-2.39 (m, 2H), 2.03-2.13 (m, 1H), 1.79-1.90 (m, 2H), 1.64-1.75 (m, 1H). m/z (ESI): 385.9/387.9 (M+H)⁺.

The sample was purified via SFC using a Chiralpak AS, 21×250 mm, 5 μm, column with a mobile phase of 25% methanol with 0.2% diethylamine using a flowrate of 150 mL/min to generate 1.82 g of peak 1 with an ee of >99% and 1.83 g of peak 2 with an ee of >96%. Peak assignment determined by SFC with a Chiralpak AS column with 20% methanol with 0.2% diethylamine. Peak 2 was desired isomer (S)-6-(7-bromo-2-chloro-8-fluoroquinazolin-4-yl)-1-oxa-6-azaspiro[3.5]nonane.

### (R)-1-(6,8-Difluoro-2-(((2R,7aS)-2-fluorotetrahydro-1H-pyrrolizin-7a(5H)-yl)methoxy)-7-(tributylstannyl)quinazolin-4-yl)-3-methylpiperidin-3-ol (Intermediate R)

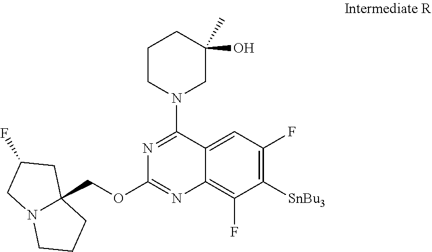

Step 1. (R)-1-(7-Bromo-2-chloro-6,8-difluoroquinazolin-4-yl)-3-methylpiperidin-3-ol. To a solution of 7-bromo-2,4-dichloro-6,8-difluoroquinazoline (5.00 g, 15.9 mmol) in acetonitrile (75 mL) was added (R)-3-methylpiperidin-3-ol hydrochloride (2.42 g, 15.9 mmol) and DIEA (8.3 mL g, 47.8 mmol) in sequence. Then the mixture was stirred at 15° C. for 3 h. The reaction mixture was quenched by addition of 80 mL water, then extracted with EtOAc. The combined organic layers were washed with brine, dried over Na₂SO₄, filtered and concentrated under reduced pressure. The residue was purified by column chromatography (SiO₂, petroleum ether/ethyl acetate 100:1-10:1) to give (R)-1-(7-bromo-2-chloro-6,8-difluoroquinazolin-4-yl)-3-methylpiperidin-3-ol (4.1 g, 10.4 mmol, 66% yield) as white solid. m/z (ESI): 394.0/392.0 (M+H)⁺.

Step 2. (R)-1-(7-Bromo-6,8-difluoro-2-(((2R,7aS)-2-fluorotetrahydro-1H-pyrrolizin-7a(5H)-yl)methoxy)quinazolin-4-yl)-3-methylpiperidin-3-ol. To a solution of (R)-1-(7-bromo-2-chloro-6,8-difluoroquinazolin-4-yl)-3-methylpiperidin-3-ol (50 g, 127 mmol) in tetrahydrofuran (750 mL) and N,N-dimethylformamide (750 mL) was added ((2R,7aS)-2-fluorotetrahydro-1H-pyrrolizin-7a(5H)-yl)methanol (22.3 g, 140 mmol), Cs₂CO₃ (49.8 g, 153 mmol) and DABCO (4.29 g, 38.2 mmol) in sequence. Then the mixture was stirred at 50° C. for 6 h. The reaction mixture was quenched by addition of 1.5 L water, and then extracted with EtOAc. The combined organic layers were washed with brine, dried over Na₂SO₄, filtered and concentrated under reduced pressure. The residue was purified by column chromatography (SiO₂, petroleum ether/ethyl acetate=10:1-0:1) to provide (R)-1-(7-bromo-6,8-difluoro-2-(((2R,7aS)-2-fluorotetrahydro-1H-pyrrolizin-7a(5H)-yl)methoxy)quinazolin-4-yl)-3-methylpiperidin-3-ol (26.0 g, 50.4 mmol, 40% yield) as white solid.

Step 3. (R)-1-(6,8-Difluoro-2-(((2R,7aS)-2-fluorotetrahydro-1H-pyrrolizin-7a(5H)-yl)methoxy)-7-(tributylstannyl)quinazolin-4-yl)-3-methylpiperidin-3-ol. To a 500 mL three-neck flask was added (R)-1-(7-bromo-6,8-difluoro-2-(((2R,7aS)-2-fluorotetrahydro-1H-pyrrolizin-7a(5H)-yl)methoxy)quinazolin-4-yl)-3-methylpiperidin-3-ol (10.0 g, 19.4 mmol) in 1,4-dioxane (100 mL). To the mixture was added PCy₃PdG₂ (1.15 g, 1.94 mmol) and lithium chloride (4.11 g, 97 mmol) in portions under N₂, then bis(tributyltin) (29.4 mL, 58.2 mmol) in 1,4-dioxane (50 mL) was added dropwise at 20° C. under N₂. The resulting mixture was stirred at 100° C. for 10 h. The reaction mixture was quenched by addition of 200 mL water, and then extracted with EtOAc. The combined organic layers were washed with brine, dried over Na₂SO₄, filtered and concentrated under reduced pressure. The residue was purified by column chromatography (SiO₂, petroleum ether/ethyl acetate=10:1-0:1) to give (R)-1-(6,8-difluoro-2-(((2R,7aS)-2-fluorotetrahydro-1H-pyrrolizin-7a(5H)-yl)methoxy)-7-(tributylstannyl)quinazolin-4-yl)-3-methylpiperidin-3-ol (5.00 g, 6.90 mmol, 36% yield) as yellow oil. m/z (ESI): 727.3/725.3 (M+H)⁺.

### 4-Bromo-6-methyl-5-((1S,2R)-2-methylcyclopropyl)-1-(tetrahydro-2H-pyran-2-yl)-1H-indazole (Intermediate S)

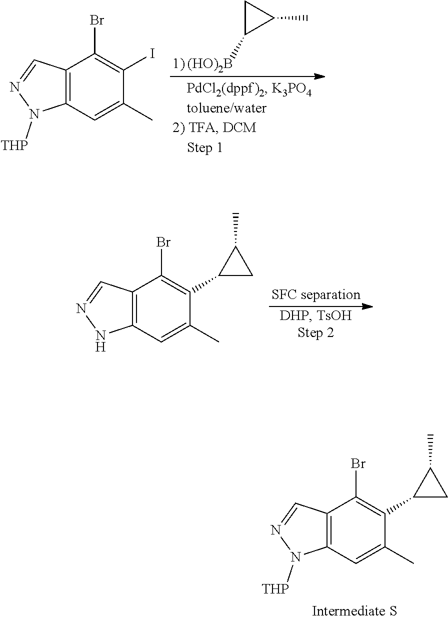

Step 1. 4-Bromo-6-methyl-5-((1S,2R)-2-methylcyclopropyl)-1H-indazole. A vial was charged with 4-bromo-5-iodo-6-methyl-1-(tetrahydro-2H-pyran-2-yl)-1H-indazole (10.0 g, 23.8 mmol, LabNetwork), rel-((1R,2S)-2-methylcyclopropyl)boronic acid (2.85 g, 28.5 mmol, Intermediate N), 1,1′-bis(diphenylphosphino)ferrocene-palladium dichloride (1.74 g, 2.38 mmol), potassium phosphate tribasic (17.6 g, 83 mmol), water (15.83 mL) and toluene (79 mL). The mixture was degassed then heated to 100° C. for 3 h. After cooling to rt, water was added and the aqueous layer was extracted with EtOAc. The combined organic layers were dried over Na₂SO₄, filtered and volatiles were removed in vacuo. The crude residue was purified via column chromatography, eluting with 0-100% EtOAc:EtOH 3:1+2% NEt₃/heptane, to yield rel-4-bromo-6-methyl-5-((1S,2R)-2-methylcyclopropyl)-1-(tetrahydro-2H-pyran-2-yl)-1H-indazole as colorless oil. The mixture was then dissolved in DCM (80 mL) and TFA (17.8 mL, 237 mmol) was added. The reaction mixture was stirred at rt for 6 h. The volatiles was removed under reduced pressure and the residue was neutralized with saturated NaHCO₃ solution. The aqueous layer was extracted with DCM and the combined organic layers were dried over Na₂SO₄, filtered and concentrated. The crude mixture was purified via column chromatography, eluting with 0-100% EtOAc:EtOH 3:1+2% NEt₃/heptane, to yield rel-4-bromo-6-methyl-5-((1S,2R)-2-methylcyclopropyl)-1H-indazole (5.5 g, 20.7 mmol, 87% yield). The sample was purified via SFC using a Chiralcel OD, 2×25 cm, 5 μm column with a mobile phase of 15% MeOH with 0.2% DEA using a flowrate of 100 mL/min. to generate 0.9 g of peak 1 with an ee of 99% and 2.88 g of peak 2 with an ee of 91.5%. Peak #1 is the desired product and was used in directly the subsequent step.

Step 2. 4-Bromo-6-methyl-5-((1S,2R)-2-methylcyclopropyl)-1-(tetrahydro-2H-pyran-2-yl)-1H-indazole. The mixture of 4-bromo-6-methyl-5-((1S,2R)-2-methylcyclopropyl)-1H-indazole-3-carbonitrile (0.28 g, 0.96 mmol), 3,4-dihydro-2H-pyran (0.24 mL, 0.29 mmol), and 4-methylbenzenesulfonic acid (8.3 mg, 0.048 mmol) in dichloromethane (5 mL) was stirred at rt overnight. The reaction mixture was purified by column chromatography on silical gel (4 g column), eluting with 0-20% EtOAc/heptane to afford 4-bromo-6-methyl-5-((1S,2R)-2-methylcyclopropyl)-1-(tetrahydro-2H-pyran-2-yl)-1H-indazole-3-carbonitrile (0.25 g, 0.67 mmol, 69% yield) as white solid. m/z (ESI): 395.8 (M+Na)⁺.

### (S)-4-((S)-7-(6-Chloro-5-((1S,2R)-2-methylcyclopropyl)-1H-indazol-4-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol (Example 98)

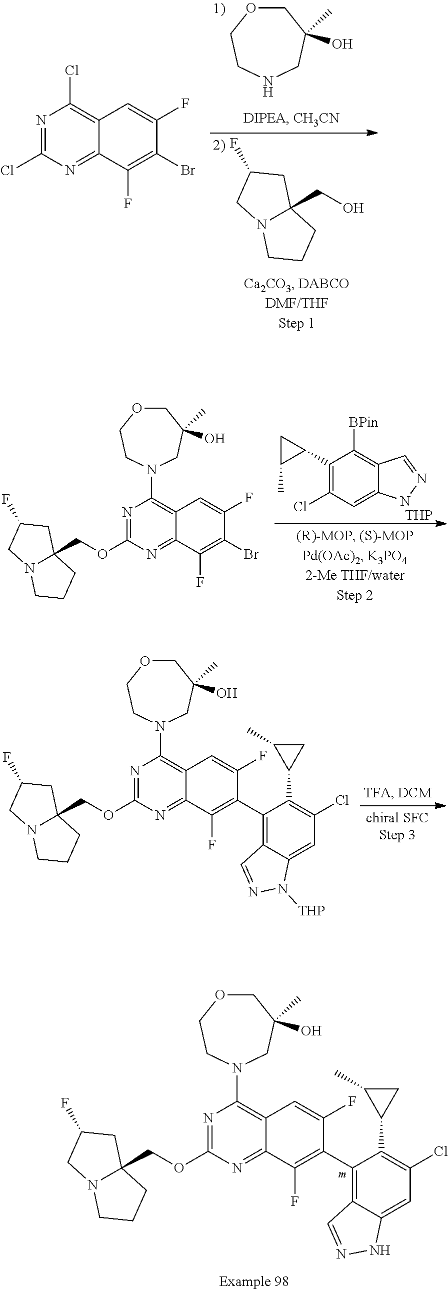

Step 1. (S)-4-(7-Bromo-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol. To a solution of 7-bromo-2,4-dichloro-6,8-difluoro-quinazoline (0.22 g, 0.69 mmol, PharmaBlock) in DCM (3 mL) was added (S)-6-methyl-1,4-oxazepan-6-ol hydrochloride (0.10 g, 0.60 mmol, Intermediate A1) followed by DIPEA (0.31 mL, 1.8 mmol). The reaction was stirred for 6 h. Then the reaction mixture was diluted with sat'd NH₄Cl and extracted with EtOAc. The organic extract was washed with sat'd NaCl solution and dried over MgSO₄. The filtrate was concentrated in vacuo to give the desired product. m/z (ESI): 409.9 (M+H)⁺.

To a vial was added ((2R,7aS)-2-fluorohexahydro-1H-pyro-7a-yl)methanol (0.15 g, 0.92 mmol, LabNetwork), cesium carbonate (0.60 g, 1.84 mmol), 1,4-diazabicyclo[2.2.2]octane (14 mg, 0.12 mmol). The solids were then suspended in N,N-dimethylformamide (1.0 mL) and tetrahydrofuran (2.0 mL), and (S)-4-(7-bromo-2-chloro-6,8-difluoroquinazolin-4-yl)-6-methyl-1,4-oxazepan-6-ol (0.25 g, 0.61 mmol) was added. The reaction mixture was then stirred at 35° C. for 9 h. The reaction was then diluted with water and extracted with EtOAc. The organic layers were combined, dried with sodium sulfate, filtered, and concentrated under reduced pressure. The crude product was purified via column chromatography (12 g) on silica gel using a gradient of 0-80% of a 3:1 EtOAc:EtOH with 2% triethylamine in heptane, to provide (S)-4-(7-bromo-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol (0.23 g, 0.43 mmol, 71% yield) as light yellow solid. m/z (ESI): 531.0 (M+H)⁺.

Step 2. (6S)-4-((7S)-7-(6-Chloro-5-((1S,2R)-2-methylcyclopropyl)-1-(tetrahydro-2H-pyran-2-yl)-1H-indazol-4-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol. A vial was charged with 6-chloro-5-((1S,2R)-2-methylcyclopropyl)-1-(tetrahydro-2H-pyran-2-yl)-4-(4,4,5,5-tetramethyl-1,3,2-dioxaborolan-2-yl)-1H-indazole (0.14 g, 0.34 mmol), Intermediate M (0.14 g, 0.34 mmol), potassium phosphate tribasic (0.15 g, 0.71 mmol), palladium (II) acetate (13 mg, 0.056 mmol), (S)-(−)-2-(diphenylphosphino)-2-methoxy-1-,1-binaphthyl (26 mg, 0.056 mmol), (R)-(+)-2-(diphenylphosphino)-2′-methoxy-1,1′-binaphthyl (26 mg, 0.056 mmol), 2-methyltetrahydrofuran (2.6 mL) and water (0.26 mL) under nitrogen. The mixture was heated to 80° C. for 3 h. After cooling to rt, the crude reaction was injected into a pre-packed C18 column (50 g), eluting with a gradient of 5-80% (0.1% formic acid MeCN)/(0.1% formic acid water) over 10 min. The desired fractions were basified with sat'd aqueous sodium bicarbonate and extracted with EtOAc. The combined organic phases were concentrated under reduced pressure to provide (6S)-4-((7S)-7-(6-chloro-5-((1S,2R)-2-methylcyclopropyl)-1-(tetrahydro-2H-pyran-2-yl)-1H-indazol-4-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol (0.16 g, 0.21 mmol, 75% yield) as tan solid. m/z (ESI): 741.2 (M+H)⁺.

Step 3. (S)-4-((S)-7-(6-Chloro-5-((1S,2R)-2-methylcyclopropyl)-1H-indazol-4-yl)-6,8-difluoro-2-(((2R,7aS)-2-fluorotetrahydro-1H-pyrrolizin-7a(H)-yl)methoxy)quinazolin-4-yl)-6-methyl-1,4-oxazepan-6-ol. A vial was charged with (6S)-4-((7S)-7-(6-chloro-5-((1S,2R)-2-methylcyclopropyl)-1-(tetrahydro-2H-pyran-2-yl)-1H-indazol-4-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol (0.16 g, 0.21 mmol) in DCM (2.0 mL), 2,2,2-Trifluoroacetic acid (0.7 mL, 25.0 equiv) was added, and the reaction mixture was stirred at rt while monitoring via LCMS. Upon completion, the mixture was cooled to 0° C. and carefully neutralized with sat'd aqueous sodium bicarbonate. The aqueous layer was extracted with DCM, and the combined organic phases were concentrated under reduced pressure. The crude material was dissolved in MeOH and injected into a pre-packed C18 column (30 g), eluting with a gradient of 5-100% (0.1% formic acid MeCN)/(0.1% formic acid water) over 10 min. The desired fractions were basified with saturated aqueous sodium bicarbonate and extracted with EtOAc. The combined organic phases were dried over anhydrous sodium sulfate, filtered and concentrated under reduced pressure to provide (S)-4-((S)-7-(6-chloro-5-((1S,2R)-2-methylcyclopropyl)-1H-indazol-4-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol (84 mg, 0.13 mmol, 45% yield) as off-white solid. ¹H NMR (400 MHz, METHANOL-d₄) δ ppm 8.10-8.18 (m, 1H) 7.80 (s, 1H) 7.65 (br s, 1H) 5.22-5.42 (m, 1H) 4.22-4.54 (m, 5H) 3.98-4.16 (m, 3H) 3.82-3.94 (m, 2H) 3.63-3.75 (m, 2H) 3.16-3.30 (m, 3H) 3.00-3.09 (m, 1H) 2.10-2.14 (m, 1H) 2.02 (s, 4H) 1.84-1.95 (m, 1H) 1.28 (d, J=1.46 Hz, 3H) 0.66-0.75 (m, 4H) −0.47-−0.13 (m, 1H) ¹⁹F NMR (376 MHz, METHANOL-d₄) δ ppm −119.06-−117.89 (m, 1F) −124.43-−123.12 (m, 1F) −174.43-−173.11 (m, 1F).

The sample (60 mg) was purified via SFC using a Chiralpak IE column (21×250 mm, 5 μm) with a mobile phase of 65% methanol with 0.2% triethylamine using a flowrate of 80 mL/min to generate 22 mg of peak 1 with an ee of >96%, and 23 mg of peak 2 (Example 98) with an cc of >90% ¹H NMR (600 MHz, DMSO-d₆) δ ppm 8.30-8.37 (m, 1H) 7.85 (s, 1H) 7.63-7.72 (m, 1H) 7.28-7.34 (m, 1H) 5.32 (s, 1H) 4.29-4.37 (m, 1H) 4.22-4.27 (m, 1H) 4.07-4.19 (m, 4H) 3.99-4.03 (m, 1H) 3.93-3.97 (m, 2H) 3.78-3.82 (m, 1H) 3.65-3.73 (m, 1H) 3.53-3.59 (m, 2H) 2.79-2.86 (m, 1H) 1.75-1.80 (m, 2 H) 1.63-1.72 (m, 1H) 1.56-1.62 (m, 1H) 1.46-1.52 (m, 1H) 1.39-1.45 (m, 1H) 1.18-1.22 (m, 1H) 1.15 (s, 3H) 0.64 (s, 4H) −0.44-−0.31 (m, 1H). m/z (ESI): 657.2 (M+H)⁺.

TABLE 17
Additional Examples 99-114. Prepared in an Analogous Manner to Examples 98.

| | | | Salt | | Method |
|---|---|---|---|---|---|
| Ex.# | Structure | Name | Form | Reagent | Change |
| 99 | 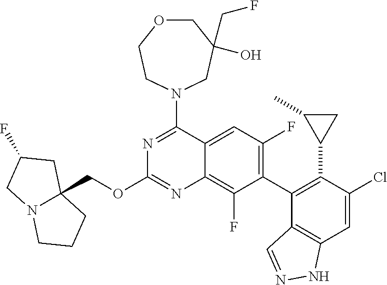 | 4-((R)-7-(6-Chloro-5- ((1S,2R)-2- methylcyclopropyl)- 1H-indazol-4-yl)-6,8- difluoro-2-(((2R,7aS)- 2-fluorotetrahydro-1H- pyrrolizin-7a(5H)- yl)methoxy)quinazolin- 4-yl)-6-(fluoromethyl)- 1,4-oxazepan-6-ol | Bis (2,2,2- tri- fluoro- aceate) | Step 1. 6- (Fluoro- methyl)- 1,4- oxazepan- 6-ol (CAS#: 1801455- 04-8, Enamine) | Chiral separation after Step 1. Details included below. |
| 100 | 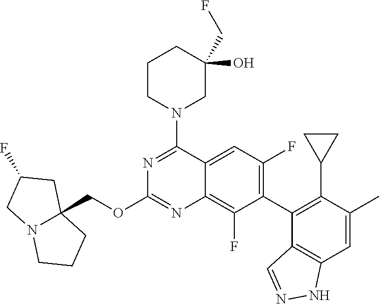 [SMILES: Cc1cc2[nH]ncc2c(-c2c(F)cc3c(N4CCC[C@](O)(CF)C4)nc(OC[C@@]45CCCN4C[C@H](F)C5)nc3c2F)c1C1CC1] | (3R)-1-(7-(5- Cyclopropyl-6-methyl- 1H-indazol-4-yl)-6,8- difluoro-2-(((2R,7aS)- 2-fluorotetrahydro-1H- pyrrolizin-7a(5H)- yl)methoxy)quinazolin- 4-yl)-3- (fluoromethyl)piperidin- 3-ol | Free base | Step 1: 3- (Fluoro- methyl) piperidin- 3-ol hydrochloride (CAS#: 2306262- 40-6, PharmaBlock) Step 2: 5-Chloro- 6-methyl- 1- tetrahydro- pyran-2-yl- 4-(4,4,5,5- tetramethyl- 1,3,2- | Additional Suzuki coupling performed (Step 2) with Cyclopro- paneboronic acid (CAS#: 411235-57- 9, Pharma- Block) prior to Step 3. Chiral separation after Step 1 |
| | | | | dioxaborolan- | and Step 3. |
| | | | | 2- | Details |
| | | | | yl)indazole | included |
| | | | | (CAS#: | below. |
| | | | | 2374152- | |
| | | | | 77-7, | |
| | | | | Pharmablock) | |
| 101 | 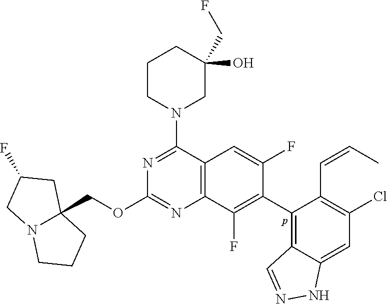 [SMILES: C/C=C\c1c(Cl)cc2[nH]ncc2c1-c1c(F)cc2c(N3CCC[C@](O)(CF)C3)nc(OC[C@@]34CCCN3C[C@H](F)C4)nc2c1F] | (3R)-1-(7-(6-Chloro-5- ((Z)-prop-1-en-1-yl)- 1H-indazol-4-yl)-6,8- difluoro-2-(((2R,7aS)- 2-fluorotetrahydro-1H- pyrrolizin-7a(5H)- yl)methoxy)quinazolin- 4-yl)-3- (fluoromethyl) piperidin-3-ol | Free base | Step 1: 3- (Fluoro- methyl) piperidin- 3-ol hydrochloride (CAS#: 2306262- 40-6, PharmaBlock) Step 2: (Z)-6- chloro-5- (prop-1-en- 1-yl)-1- (tetrahydro- 2H-pyran- 2-yl)-4- (4,4,5,5- | Chiral separation after Step 1 and Step 3. Details included below. |
| | | | | tetramethyl- | |
| | | | | 1,3,2- | |
| | | | | dioxaborolan- | |
| | | | | 2-yl)-1H- | |
| | | | | indazole | |
| | | | | (Intermediate | |
| | | | | M) | |
| 102 | 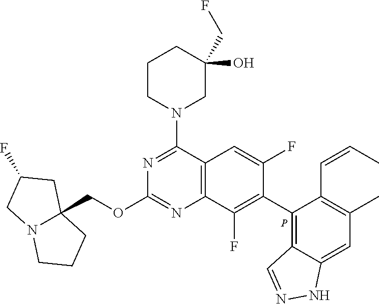 | (3R)-1-(6,8-Difluoro-2- (((2R,7aS)-2- fluorotetrahydro-1H- pyrrolizin-7a(5H)- yl)methoxy)-7-(6- methyl-5-((Z)-prop-1- en-1-yl)-1H-indazol-4- yl)quinazolin-4-yl)-3- (fluoromethyl) piperidin-3-ol | Free base | Step 1: 3- (Fluoro- methyl) piperidin- 3-ol hydrochloride (CAS#: 2306262- 40-6, PharmaBlock) Step 2: (Z)-6- Methyl-5- (prop-1-en- 1-yl)-1- (tetrahydro- 2H-pyran- 2-yl)-4- (4,4,5,5- | Chiral separation after Step 1 and Step 3. Details included below. |
| | | | | tetramethyl- | |
| | | | | 1,3,2- | |
| | | | | dioxaborolan- | |
| | | | | 2-yl)-1H- | |
| | | | | indazole | |
| | | | | (Intermediate | |
| | | | | P) | |
| 103 | 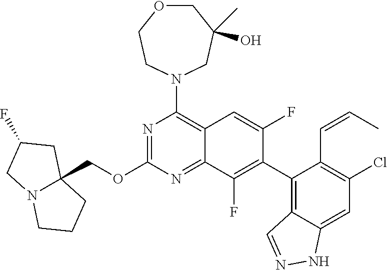 [SMILES: C/C=C\c1c(Cl)cc2[nH]ncc2c1-c1c(F)cc2c(N3CCOC[C@@](C)(O)C3)nc(OC[C@@]34CCCN3C[C@H](F)C4)nc2c1F] | (6S)-4-(7-(6-Chloro-5- ((Z)-prop-1-en-1-yl)- 1H-indazol-4-yl)-6,8- difluoro-2-(((2R,7aS)- 2-fluorotetrahydro-1H- pyrrolizin-7a(5H)- yl)methoxy)quinazolin- 4-yl)-6-methyl-1,4- oxazepan-6-ol | 2,2,2- tri- fluoro- aceate | Step 1: Intermediate A1 Step 2: (Z)-6- Chloro-5- (prop-1-en- 1-yl)-1- (tetrahydro- 2H-pyran- 2-yl)-4- (4,4,5,5- tetramethyl- 1,3,2- dioxaborolan- 2-yl)-1H- indazole | |
| | | | | (Intermediate | |
| | | | | M). | |
| 104 | 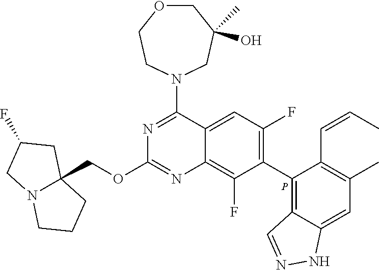 | (6S)-4-(6,8-Difluoro-2- (((2R,7aS)-2- fluorotetrahydro-1H- pyrrolizin-7a(5H)- yl)methoxy)-7-(6- methyl-5-((Z)-prop-1- en-1-yl)-1H-indazol-4- yl)quinazolin-4-yl)-6- methyl-1,4-oxazepan- 6-o1 | | Step 1: Intermediate A1 Step 2: (Z)-6- Methyl-5- (prop-1-en- 1-yl)-1- (tetrahydro- 2H-pyran- 2-yl)-4- (4,4,5,5- tetramethyl- 1,3,2- dioxaborolan- 2-yl)-1H- indazole | Step 3. Chiral separation performed on final compound, details included below. |
| | | | | (Intermediate | |
| | | | | P) | |
| 105 | 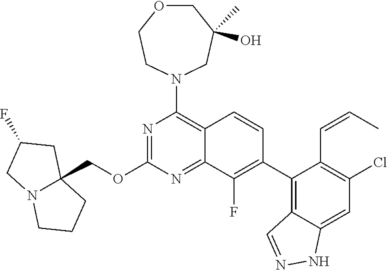 [SMILES: C/C=C\c1c(Cl)cc2[nH]ncc2c1-c1ccc2c(N3CCOC[C@@](C)(O)C3)nc(OC[C@@]34CCCN3C[C@H](F)C4)nc2c1F] | (6S)-4-(7-(6-Chloro-5- ((Z)-prop-1-en-1-yl)- 1H-indazol-4-yl)-8- fluoro-2-(((2R,7aS)-2- fluorotetrahydro-1H- pyrrolizin-7a(5H)- yl)methoxy)quinazolin- 4-yl)-6-methyl-1,4- oxazepan-6-ol | Bis (2,2,2- tri- fluoro- aceate) | Step 1: 7-Bromo- 2.4- dichloro-8- fluoroquina- zoline, Enamine (CAS#: 2168499- 15-6, Enamine) and Intermediate A1 Step 2: (Z)-6- Chloro-5- | CataCXium Pd A G3 was used as catalyst |
| | | | | (prop-1-en- | |
| | | | | 1-yl)-1- | |
| | | | | (tetrahydro- | |
| | | | | 2H-pyran- | |
| | | | | 2-yl)-4- | |
| | | | | (4,4,5,5- | |
| | | | | tetramethyl- | |
| | | | | 1,3,2- | |
| | | | | dioxaborolan- | |
| | | | | 2-yl)-1H- | |
| | | | | indazole | |
| | | | | (Intermediate | |
| | | | | M) | |
| 106 | 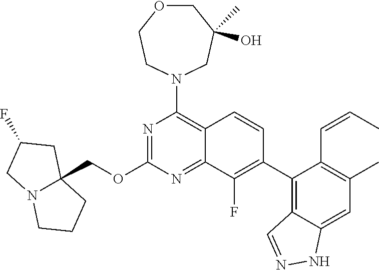 [SMILES: C/C=C\c1c(C)cc2[nH]ncc2c1-c1ccc2c(N3CCOC[C@@](C)(O)C3)nc(OC[C@@]34CCCN3C[C@H](F)C4)nc2c1F] | (6S)-4-(8-Fluoro-2- (((2R,7aS)-2- fluorotetrahydro-1H- pyrrolizin-7a(5H)- yl)methoxy)-7-(6- methyl-5-((Z)-prop-1- en-1-yl)-1H-indazol-4- yl)quinazolin-4-yl)-6- methyl-1,4-oxazepan- 6-ol | Bis (2,2,2- tri- fluoro- aceate) | Step 1: 7- Bromo-2,4- dichloro-8- fluoroquina- zoline, Enamine (CAS#: 2168499- 15-6, Enamine) and Intermediate A1 Step 1: (Z)-6- methyl-5- (prop-1-en- | CataCXium Pd A G3 was used as catalyst |
| | | | | 1-yl)-1- | |
| | | | | (tetrahydro- | |
| | | | | 2H-pyran- | |
| | | | | 2-yl)-4- | |
| | | | | (4,4,5,5- | |
| | | | | tetramethyl- | |
| | | | | 1,3,2- | |
| | | | | dioxaborolan- | |
| | | | | 2-yl)-1H- | |
| | | | | indazole | |
| | | | | (Intermediate | |
| | | | | P) | |
| 107 | 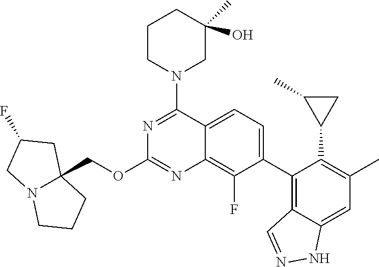 [SMILES: Cc1cc2[nH]ncc2c(-c2ccc3c(N4CCC[C@@](C)(O)C4)nc(OC[C@@]45CCCN4C[C@H](F)C5)nc3c2F)c1[C@H]1C[C@H]1C] | (R)-1-((S)-8-Fluoro-2- (((2R,7aS)-2- fluorotetrahydro-1H- pyrrolizin-7a(5H)- yl)methoxy)-7-(6- methyl-5-((1S,2R)-2- methylcyclopropyl)- 1H-indazol-4- yl)quinazolin-4-yl)-3- methylpiperidin-3-ol | | Step 1: 7- Bromo-2,4- dichloro-8- fluoroquina- zoline, Enamine (CAS#: 2168499- 15-6, Enamine) Step 2: 5-Chloro- 6-methyl- tetrahydro- pyran-2-yl- 4-(4,4,5,5- tetramethyl- | Additional Suzuki coupling performed (Step 1) with Intermediate N prior to Step 2. Chiral separation performed on final compound, detailed included below. |
| | | | | 1,3,2- | |
| | | | | dioxaborolan- | |
| | | | | 2- | |
| | | | | yl)indazole | |
| | | | | (CAS#: | |
| | | | | 2374152- | |
| | | | | 77-7, | |
| | | | | PharmaBlock) | |
| 108 | 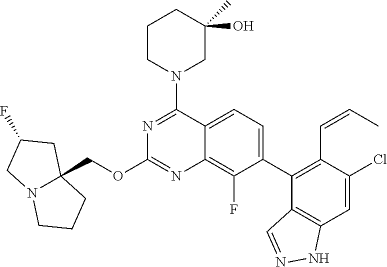 [SMILES: C/C=C\c1c(Cl)cc2[nH]ncc2c1-c1ccc2c(N3CCC[C@@](C)(O)C3)nc(OC[C@@]34CCCN3C[C@H](F)C4)nc2c1F] | (3R)-1-(7-(6-Chloro-5- ((Z)-prop-1-en-1-yl)- 1H-indazol-4-yl)-8- fluoro-2-(((2R,7aS)-2- fluorotetrahydro-1H- pyrrolizin-7a(5H)- yl)methoxy)quinazolin- 4-yl)-3- methylpiperidin-3-ol | Free base | Step 1: 7- Bromo-2,4- dichloro-8- fluoroquina- zoline, Enamine (CAS#: 2168499- 15-6, Enamine) Step 2: (Z)- 6-Chloro- 5-(prop-1- en-1-yl)-1- (tetrahydro- 2H-pyran- 2-yl)-4- | |
| | | | | (4,4,5,5- | |
| | | | | tetramethyl- | |
| | | | | 1,3,2- | |
| | | | | dioxaborolan- | |
| | | | | 2-yl)-1H- | |
| | | | | indazole | |
| | | | | (Intermediate | |
| | | | | M) | |
| 109 | 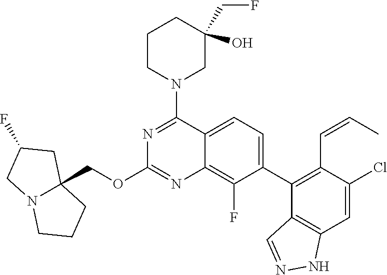 [SMILES: C/C=C\c1c(Cl)cc2[nH]ncc2c1-c1ccc2c(N3CCC[C@](O)(CF)C3)nc(OC[C@@]34CCCN3C[C@H](F)C4)nc2c1F] | (3R)-1-(7-(6-Chloro-5- ((Z)-prop-1-en-1-yl)- 1H-indazol-4-yl)-8- fluoro-2-(((2R,7aS)-2- fluorotetrahydro-1H- pyrrolizin-7a(5H)- yl)methoxy)quinazolin- 4-yl)-3- (fluoromethyl) piperidin-3-ol | Free base | Step 1: 7- Bromo-2,4- dichloro-8- fluoroquina- zoline, Enamine (CAS#: 2168499- 15-6, Enamine) and 3- (fluoro- methyl) piperidin-3-ol hydrochloride (CAS#: 2306262- 40-6, | Chiral separation after Step 1. Details included below. |
| | | | | PharmaBlock) | |
| | | | | Step 2: (Z)- | |
| | | | | 6-Chloro- | |
| | | | | 5-(prop-1- | |
| | | | | en-1-yl)-1- | |
| | | | | (tetrahydro- | |
| | | | | 2H-pyran- | |
| | | | | 2-yl)-4- | |
| | | | | (4,4,5,5- | |
| | | | | tetramethyl- | |
| | | | | 1,3,2- | |
| | | | | dioxaborolan- | |
| | | | | 2-yl)-1H- | |
| | | | | indazole | |
| | | | | (Intermediate | |
| | | | | M) | |
| 110 | 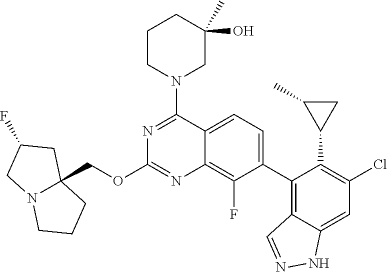 [SMILES: C[C@@H]1C[C@@H]1c1c(Cl)cc2[nH]ncc2c1-c1ccc2c(N3CCC[C@@](C)(O)C3)nc(OC[C@@]34CCCN3C[C@H](F)C4)nc2c1F] | (R)-1-((R)-7-(6-Chloro- 5-((1S,2R)-2- methylcyclopropyl)- 1H-indazol-4-yl)-8- fluoro-2-(((2R,7aS)-2- fluorotetrahydro-1H- pyrrolizin-7a(5H)- yl)methoxy)quinazolin- 4-yl)-3- methylpiperidin-3-ol | Free base | Step 1: 7- Bromo-2,4- dichloro-8- fluoroquina- zoline, Enamine (CAS#: 2168499- 15-6, Enamine) Step 3: 6- Chloro-5- ((1S,2R)-2- methylcyclo- propyl)-1- (tetrahydro- 2H-pyran- 2-yl)-4- | |
| | | | | (4,4,5,5- | |
| | | | | tetramethyl- | |
| | | | | 1,3,2- | |
| | | | | dioxaborolan- | |
| | | | | 2-yl)-1H- | |
| | | | | indazole | |
| | | | | (Intermediate | |
| | | | | O) | |
| 111 | 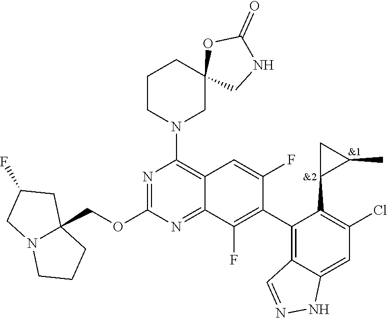 [SMILES: C[C@@H]1C[C@@H]1c1c(Cl)cc2[nH]ncc2c1-c1c(F)cc2c(N3CCC[C@]4(CNC(=O)O4)C3)nc(OC[C@@]34CCCN3C[C@H](F)C4)nc2c1F] | (5S)-7-(7-(6-Chloro-5~ (2-methylcyclopropyl)- 1H-indazol-4-yl)-6,8- difluoro-2-(((2R,7aS)- 2-fluorotetrahydro-1H- pyrrolizin-7a(5H)- yl)methoxy)quinazolin- 4-yl)-1-oxa-3,7- diazaspiro[4.5]decan-2- one 2,2,2- trifluoroacetate | 2,2,2- tri- fluoro- aceate | Step 1: Intermediate H Step 2: rel-6- Chloro-5- ((1R,2S)-2- methylcyclo- propyl)-1- (tetrahydro- 2H-pyran- 2-yl)-4- (4,4,5,5- tetramethyl- 1,3,2- dioxaborolan- 2-yl)-1H- indazole (Intermediate O) before | |
| | | | | chiral | |
| | | | | separation. | |
| 112 | 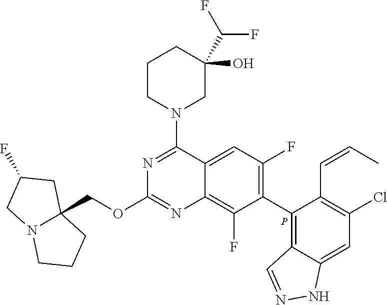 | (3R)-1-(7-(6-Chloro-5- ((Z)-prop-1-en-1-yl)- 1H-indazol-4-yl)-6,8- difluoro-2-(((2R,7aS)- 2-fluorotetrahydro-1H- pyrrolizin-7a(5H)- yl)methoxy)quinazolin- 4-yl)-3- (difluoromethyl) piperidin-3-ol | Bis (2,2,2- tri- fluoro- aceate) | Step 1: 7- Bromo-2,4- dichloro- 6.8- difluoro- quinazoline (CAS#: 2248318- 27-4, PharmaBlock) and 3- (difluoro- methyl)piperi- din-3-ol hydrochloride (CAS)#: 1909325- 03-6, Enamine) | Chiral separation after Step 1. Details included below. Separation of atroisomer performed after Step 3 by via RP HPLC. |
| | | | | Step 2: (Z)- | |
| | | | | 6-Chloro- | |
| | | | | 5-(prop-1- | |
| | | | | en-1-yl)-1- | |
| | | | | (tetrahydro- | |
| | | | | 2H-pyran- | |
| | | | | 2-yl)-4- | |
| | | | | (4,4,5,5- | |
| | | | | tetramethyl- | |
| | | | | 1,3,2- | |
| | | | | dioxaborolan- | |
| | | | | 2-yl)-1H- | |
| | | | | indazole | |
| | | | | (Intermediate | |
| | | | | M) | |
| 113 | 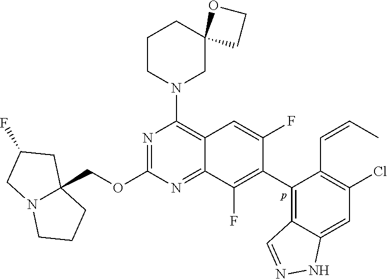 | (4S)-6-(7-(6-Chloro-5- ((Z)-prop-1-en-1-yl)- 1H-indazol-4-yl)-6,8- difluoro-2-(((2R,7aS)- 2-fluorotetrahydro-1H- pyrrolizin-7a(5H)- yl)methoxy)quinazolin- 4-yl)-1-oxa-6- azaspiro[3.5]nonane | | Step 1: 7- Bromo-2,4- dichloro- 6.8- difluoro- quinazoline (CAS#: 2248318- 27-4, PharmaBlock) and (4S)-1-oxa- 6- azaspiro[3.5] nonane (CAS#: 1527519- 18-1, | Chiral separation after Step 3. Details included below. |
| | | | | PharmaBlock) | |
| | | | | Step 2: (Z)- | |
| | | | | 6-Chloro- | |
| | | | | 5-(prop-1- | |
| | | | | en-1-yl)-1- | |
| | | | | (tetrahydro- | |
| | | | | 2H-pyran- | |
| | | | | 2-yl)-4- | |
| | | | | (4,4,5,5- | |
| | | | | tetramethyl- | |
| | | | | 1,3,2- | |
| | | | | dioxaborolan- | |
| | | | | 2-yl)-1H- | |
| | | | | indazole | |
| | | | | (Intermediate | |
| | | | | M) | |
| 114 | 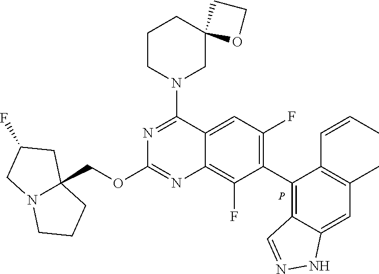 | (4S)-6-(6,8-Difluoro-2- (((2R,7aS)-2- fluorotetrahydro-1H- pyrrolizin-7a(5H)- yl)methoxy)-7-(6- methyl-5-((Z)-prop-1- en-1-yl)-1H-indazol-4- yl)quinazolin-4-yl)-1- oxa-6- azaspiro[3.5]nonane | | Step 1: 7- Bromo-2,4- dichloro- 6,8- difluoro- quinazoline (CAS#: 2248318- 27-4. PharmaBlock) and (45)-1-oxa- 6- azaspiro[3.5] nonane (CAS#: 1527519- 18-1, | Chiral separation after Step 3. Details included below. |
| | | | | PharmaBlock) | |
| | | | | Step 2: (Z)- | |
| | | | | 6-methyl- | |
| | | | | 5-(prop-1- | |
| | | | | en-1-yl)-1- | |
| | | | | (tetrahydro- | |
| | | | | 2H-pyran- | |
| | | | | 2-yl)-4- | |
| | | | | (4,4,5,5- | |
| | | | | tetramethyl- | |
| | | | | 1,3,2- | |
| | | | | dioxaborolan- | |
| | | | | 2-yl)-1H- | |
| | | | | indazole | |
| | | | | (Intermediate | |
| | | | | P) | |

TABLE 18
Conditions for chiral SFC separations.

| | | Peak to |
|---|---|---|
| Compound | SFC Conditions | Ex.# |
| 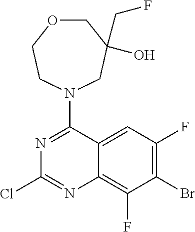 [SMILES: OC1(CF)COCCN(c2nc(Cl)nc3c(F)c(Br)c(F)cc23)C1] | Column: ChiralPak AD, 2 x 15 μm, 5 μm Mobile phase: 30% MeOH with 0.2% TEA Flowrate: 90 mL/min Yield: 700 mg sample was submitted to generate 323 mg of peak 1 with an ee of >99% and 346 mg of peak 2 with an ee of >99%. | Peak 1: Example 99 |
| 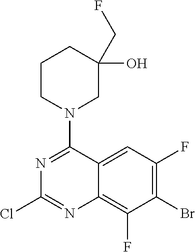 [SMILES: OC1(CF)CCCN(c2nc(Cl)nc3c(F)c(Br)c(F)cc23)C1] | Column: Chiralpak IC, 21 x 250 mm, 5 μm, Mobile phase: 25% 2-propanol with 0.2% triethylamine Flowrate: 80 mL/min Yield: 3.65 g sample was submitted to generate 1.72 g of peak 1 with an ee of >99% and 1.79 g of peak 2 with an ee of >95%. | Peak 1: Example 100 Example 101 Example 102 |
| 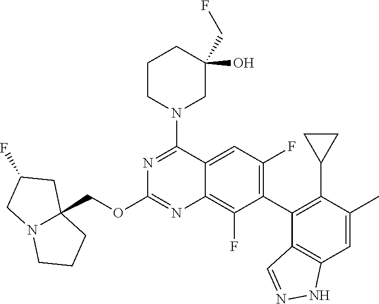 [SMILES: Cc1cc2[nH]ncc2c(-c2c(F)cc3c(N4CCC[C@](O)(CF)C4)nc(OC[C@@]45CCCN4C[C@H](F)C5)nc3c2F)c1C1CC1] | Column: Chiralcel OD, 21 x 250 mm, 5 μm Mobile phase: 30% MeOH with 0.2% TEA Flowrate: 120 mL/min Yield: 55 mg sample was submitted to generate 26 mg of peak I with an ee of >99% and 24 mg of peak 2 with an ee of >90% | Peak 2: Example 100 |
| 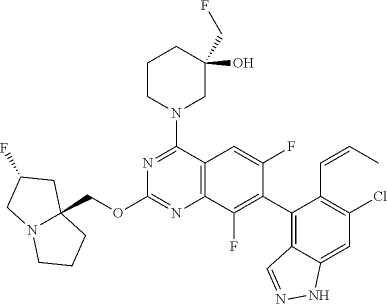 [SMILES: C/C=C\c1c(Cl)cc2[nH]ncc2c1-c1c(F)cc2c(N3CCC[C@](O)(CF)C3)nc(OC[C@@]34CCCN3C[C@H](F)C4)nc2c1F] | Column: Chiralcel OD, 2 x 25 cm, 5 μm Mobile phase: 30% 1:1 ACN:MeOH with 0.2% triethylamine Flowrate: 80 mL/min. Yield: 119 mg sample was submitted to generate 81 mg of peak 1 with an ee of 97% and 45 mg of peak 2 with an ee of 98% | Peak 2: Example 101 |
| 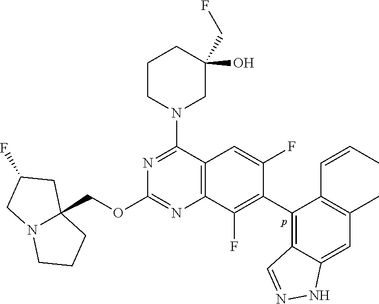 | Column: Chiralcel OD, 2 x 25 cm, 5 μm Mobile phase: 35% MeOH w/ 0.2% TEA Flowrate: 80 mL/min. Yield: 134 mg sample was submitted to generate 44.2 mg of peak 1 with an ee of 99% and 39.8 mg of peak 2 with an ee of 97% | Peak 2: Example 102 |
| 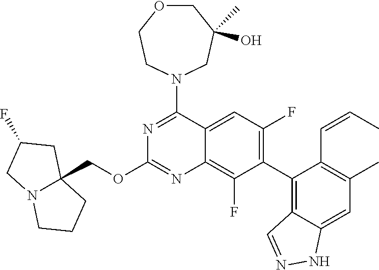 [SMILES: C/C=C\c1c(C)cc2[nH]ncc2c1-c1c(F)cc2c(N3CCOC[C@@](C)(O)C3)nc(OC[C@@]34CCCN3C[C@H](F)C4)nc2c1F] | Column: Chiralpak OD, 21 x 250 mm, 5 μm Mobile phase: 30% methanol with 0.2% tricthylamine Flowrate: 80 mL/min. Yield: 75 mg sample was submitted to generate 32 mg of peak 1 with an ee of >99% and 21.3 mg of peak 2 with an ee of >96% | Peak 2: Example 104 |
| 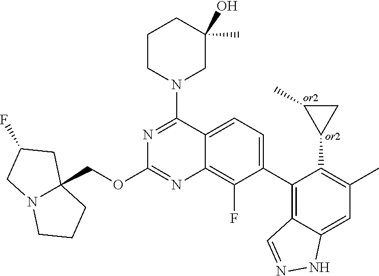 [SMILES: Cc1cc2[nH]ncc2c(-c2ccc3c(N4CCC[C@@](C)(O)C4)nc(OC[C@@]45CCCN4C[C@H](F)C5)nc3c2F)c1[C@H]1C[C@H]1C] | Column: Chiralcel OD, 21 x 250 mm, 5 μm column Mobile phase: 35% methanol with 0.2% triethylamine Flowrate: 100 mL/min. Yield: 70 mg sample was submitted to generate 8.6 mg of peaks 1/4 with an de of 99% and 20 mg of peak 2/3 with an ee of 85%. | Peak 2/3: Example 107 |
| 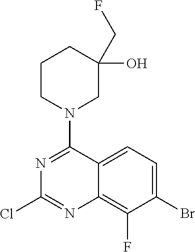 [SMILES: OC1(CF)CCCN(c2nc(Cl)nc3c(F)c(Br)ccc23)C1] | Column: Chiralpak IG, 21 x 250 mm, 5 μm Mobile phase: 25% MeOH Flowrate: 100 mL/min Yield: 887 mg sample was submitted to generate 346 mg of peak 1 with an ee of >99% and 380 mg of peak 2 with an ee of >96%. | Peak 1: Example 109 |
| 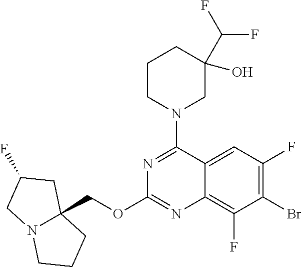 [SMILES: OC1(C(F)F)CCCN(c2nc(OC[C@@]34CCCN3C[C@H](F)C4)nc3c(F)c(Br)c(F)cc23)C1] | Column: ChiralPak IC, 20 x 25 um, 5 μm Mobile phase: 20% ethanol with 0.2% triethylamine Flowrate: 80 mL/min. Yield: 360 mg sample was submitted to generate 161 mg of peaks 1 with an ee of 99% and 153 mg of peak 2 with an ee of 99%. | Peak 1: Example 112 |
| 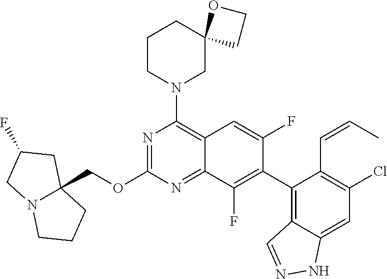 [SMILES: C/C=C\c1c(Cl)cc2[nH]ncc2c1-c1c(F)cc2c(N3CCC[C@]4(CCO4)C3)nc(OC[C@@]34CCCN3C[C@H](F)C4)nc2c1F] | Column: (S,S) Whelk-0, 20 x 25 μm, 5 μm Mobile phase: 30% ethanol with 0.2% tricthylamine Flowrate: 80 mL/min. Yield: 50 mg sample was submitted to generate 15 mg of peaks 1 with an ee of 99% and 14 mg of peak 2 with an ee of 93%. | Peak 2: Example 113 |
| 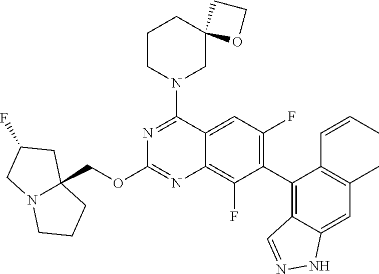 [SMILES: C/C=C\c1c(C)cc2[nH]ncc2c1-c1c(F)cc2c(N3CCC[C@]4(CCO4)C3)nc(OC[C@@]34CCCN3C[C@H](F)C4)nc2c1F] | Column: Chiralpak OD, 21 x 250 mm, 5 μm Mobile phase: 25% methanol with 0.2% triethylamine Flowrate: 80 mL/min. Yield: 70 mg sample was submitted to generate 20 mg of peaks 1 with an ee of 99% and 22 mg of peak 2 with an ee of 96%. | Peak 2: Example 114 |

TABLE 19
Analytical Data for Examples 99- 114.

| | MS | |
|---|---|---|
| Ex. | m/z (ESI): | |
| # | (M + H)⁺ | ¹H NMR |
| 99 | 675.0 | ¹H NMR (400 MHz, DMSO-d₄) δ ppm 8.10-8.24 (m, 1H), |
| | | 7.76-7.89 (m, 1 H), 7.55-7.69 (m, 1 H), 5.44-5.71 (m, 1 H), 4.62- |
| | | 4.75 (m, 2 H), 4.36-4.58 (m, 4 H), 3.84-4.20 (m, 8 H), 3.62-3.73 |
| | | (m, 1 H), 3.45-3.56 (m, 1 H), 2.51-2.79 (m, 2 H), 2.29-2.49 (m, 3 |
| | | H), 2.05-2.26 (m, 2 H), 1.22-1.37 (m, 1 H), 0.62-0.83 (m, 4 H), |
| | | −0.39-−0.16 (m, 1 H). |
| 100 | 625.0 | 1H NMR (600 MHz, DMSO-d₆) δ ppm 13.04 (s, 1H), 7.88 (br d, 1H, |
| | | J = 10.3 Hz), 7.57 (s, 1H), 7.48 (s, 1H), 5.33 (br s, 1H), 5.2-5.3 (m, 2H), |
| | | 4.4-4.4 (m, 1H), 4.3-4.4 (m, 1H), 4.3-4.3 (m, 1H), 4.11 (d, 1H, J = 10.3 |
| | | Hz), 4.0-4.0 (m, 3H), 3.89 (br d, 1H, J = 13.5 Hz), 3.53 (d, 1H, J = 13.2 |
| | | Hz), 3.0-3.1 (m, 2H), 3.03 (br s, 1H), 2.8-2.9 (m, 1H), 2.60 (s, 1H), |
| | | 2.1-2.2 (m, 1H), 2.07 (br s, 1H), 2.0-2.1 (m, 3H), 1.8-1.9 (m, 3H), 1.7- |
| | | 1.8 (m, 6H), 1.6-1.7 (m, 2H), 1.23 (br s, 1H), 0.6-0.7 (m, 3H), 0.1-0.1 |
| | | (m, 3H). |
| 101 | 645.2 | ¹H NMR (400 MHz, METHANOL-d₄) δ ppm 7.66-7.86 (m, 3 H) |
| | | 6.33-6.45 (m, 1 H) 5,63-5.76 (m, 1 H) 5.19-5.42 (m, 1 H) 4.37- |
| | | 4.54 (m, 1 H) 4.25-4.36 (m, 2 H) 4.13-4.24 (m, 1 H) 4.01-4.11 (m, |
| | | 1 H) 3.66-3.76 (m, 2 H) 3.45-3.62 (m, 2 H) 3.11-3.28 (m, 3 H) |
| | | 2.96-3.06 (m, 1 H) 2.29-2.41 (m, 1 H) 2.24-2.28 (m, 1 H) 2.09- |
| | | 2.22 (m, 3 H) 1.96-2.06 (m, 2 H) 1.76-1.95 (m, 5 H) 1.48-1.66 (m, |
| | | 1 H) 0.82-0.95 (m, 1 H) |
| 102 | 625.2 | ¹H NMR (500 MHz, METHANOL-d₄) δ ppm 7.74-7.82 (m, 1 H), |
| | | 7.60-7.66 (m, 1 H), 7.52-7.59 (m, 1 H), 6.40-6.47 (m, 1 H), 5.60- |
| | | 5.69 (m, 1 H), 5.22-5.41 (m, 1 H), 4.38-4.49 (m, 1 H), 4.30-4.36 |
| | | (m, 1 H), 4.23-4.28 (m, 1 H), 4.15-4.21 (m, 1 H), 4.04-4.11 (m, 1 |
| | | H), 3.68 (s, 3 H), 3.44-3.55 (m, 1 H), 3.21-3.29 (m, 2 H), 3.01-3.07 |
| | | (m, 1 H), 2.45 (s, 3 H), 2.22-2.41 (m, 2 H), 2.13-2.22 (m, 2 H), 1.98- |
| | | 2.06 (m, 2 H), 1.75-1.97 (m, 4 H), 1.23-1.30 (m, 3 H). |
| 103 | 643.2 | ¹H NMR (400 MHz, METHANOL-d₆) δ ppm 8.28 (br dd, J = 19.4, |
| | | 10.0 Hz, 1 H), 7.80-7.98 (m, 1 H), 7.62-7.80 (m, 1 H), 6.21-6.45 |
| | | (m, 1 H), 5.65-5.76 (m, 1 H), 5.49-5.65 (m, 1 H), 4.65-4.78 (m, 4 |
| | | H), 4.52-4.60 (m, 1 H), 4.44-4.51 (m, 1 H), 3,99-4.18 (m, 2 H), |
| | | 3.84-3.96 (m, 4 H), 3.59-3.77 (m, 2 H), 3.34-3.58 (m, 1 H), 2.51- |
| | | 2.78 (m, 2 H), 2.42-2.49 (m, 1 H), 2.31-2.41 (m, 2 H), 2.13-2.26 |
| | | (m, 1 H), 1.83 (s, 1 H), 1.24-1.34 (m, 5 H). |
| 104 | 623.2 | ¹H NMR (600 MHz, DMSO-d₆) δ ppm 8.23 (br d, J = 11.2 Hz, 1 H), |
| | | 7.64 (s, 1 H), 7.54 (s, 1 H), 6.32-6.42 (m, 1 H), 5.55-5.65 (m, 1 H), |
| | | 5.29 (s, 2 H), 4.24-4.34 (m, 1 H), 4.07-4.18 (m, 2 H), 3.91-4.04 (m, |
| | | 4 H), 3.67-3.76 (m, 1 H), 3.51-3.59 (m, 2 H), 3.25-3.39 (m, 1 H), |
| | | 2.99-3.12 (m, 2 H), 2.76-2.88 (m, 1 H), 2.37 (s, 2 H), 1.96-2.18 (m, |
| | | 3 H), 1.80-1.86 (m, 1 H), 1.73-1.80 (m, 2 H), 1.21-1.27 (m, 1 H), |
| | | 1.15 (s, 7 H). |
| 105 | 624.8 | ¹H NMR (500 MHz, METHANOL-d₄) δ ppm 8.39 (br t, J = 7.9 Hz, 1 |
| | | H), 7.82 (d, J = 1.0 Hz, 1 H), 7.75 (d, J = 7.8 Hz, 1 H), 7.42-7.62 (m, 1 |
| | | H), 6.41-6.51 (m, 1 H), 5.47-5.76 (m, 2 H), 4.74-4.80 (m, 2 H), |
| | | 4.51-4.66 (m, 2 H), 4.12-4.32 (m, 1 H), 3.89-4.10 (m, 6 H), 3.63- |
| | | 3.81 (m, 2 H), 3.46-3.56 (m, 1 H), 2.57-2.78 (m, 2 H), 2.32-2.53 |
| | | (m, 3 H), 2.14-2.29 (m, 1 H), 1.21-1.35 (m, 6 H). |
| 106 | 605.0 | ¹H NMR (400 MHz, METHANOL-d₄) δ ppm 8.29 (dd, J = 8.4, 5.2 Hz, |
| | | 1 H), 7.66 (d, J = 6.5 Hz, 1 H), 7.52-7.60 (m, 1 H), 7.39-7.46 (m, 1 |
| | | H), 6.48 (br d, J = 10.9 Hz, 1 H), 5.49-5.70 (m, 2 H), 4.69-4.79 (m, 3 |
| | | H), 4.48-4.57 (m, 2 H), 4.11-4.19 (m, 1 H), 3.87-4.09 (m, 6 H), |
| | | 3.64-3.76 (m, 2 H), 3.45-3.58 (m, 1 H), 2.57-2.81 (m, 2 H), 2.33- |
| | | 2.51 (m, 6 H), 2.12-2.26 (m, 1 H), 1.27-1.31 (m, 3 H), 1.21-1.25 |
| | | (m, 2 H). ¹⁹F NMR (376 MHz, METHANOL-d⁴) δ ppm −77.38-−77.27 |
| | | (m, 1 F), −129.32-−128.98 (m, 1 F), −174.32-−174.28 (m, 1 F) |
| 107 | 603.2 | ¹H NMR (400 MHz, METHANOL-d₄) δ ppm 7.88-8.09 (m, 1 H) |
| | | 7.49 (s, 3 H) 5.25-5.48 (m, 1 H) 3.99-4.49 (m, 4 H) 3.42-3.67 (m, 3 |
| | | H) 3.05-3.17 (m, 1 H) 2.63 (s, 3 H) 2.14-2.50 (m, 4 H) 2.03-2.13 |
| | | (m, 3 H) 1.71-2.00 (m, 4 H) 1.30 (br d, J = 2.51 Hz, 6 H) 0.72-0.97 |
| | | (m, 1 H) 0.57-0.65 (m, 3 H) −0.47-−0.19 (m, 1 H). |
| 108 | 608.8 | ¹H NMR (400 MHz, METHANOL-d₄) δ ppm 7.95 (dd, J = 13.23, 8.69 |
| | | Hz, 1 H), 7.78 (d, J = 0.91 Hz, 1 H), 7.74 (d, J = 7.79 Hz, 1 H), 7.27 (br |
| | | d, J = 5.97 Hz, 1 H), 6.42 (br d, J = 11.42 Hz, 1 H), 5.61-5.69 (m, 1 H). |
| | | 5.22-5.44 (m, 1 H), 4.32-4.36 (m, 1 H), 4.20-4.29 (m, 2 H), 4.03- |
| | | 4.11 (m, 1 H), 3.43-3.60 (m, 2 H), 3.19-3.31 (m, 3 H), 3.04 (td, |
| | | J = 9.50, 5.77 Hz, 1 H), 2.09-2.43 (m, 4 H), 1.97-2.03 (m, 2 H), 1.74- |
| | | 1.96 (m, 4 H), 1.29 (s, 3 H), 1.27 (s, 3 H) |
| 109 | 626.8 | ¹H NMR (500 MHz, METHANOL-d₄) δ ppm 7.96 (t, J = 9.28 Hz, 1 H), |
| | | 7.78 (s, 1 H), 7.74 (d, J = 12.07 Hz, 1 H), 7.29 (br s, 1 H), 6.42 (br d, |
| | | J = 11.03 Hz, 1 H), 5.62-5.68 (m, 1 H), 5.22-5.46 (m, 1 H), 4.29 (s, 4 |
| | | H), 4.10-4.16 (m, 1 H), 3.75 (s, 1 H), 3.51-3.59 (m, 1 H), 3.34-3.40 |
| | | (m, 1 H), 3.25-3.31 (m, 2 H), 3.06 (td, J = 9.47, 5.84 Hz, 1 H), 2.18 (br |
| | | d, J = 8.43 Hz, 4 H), 2.03 (s, 2 H), 1.79-1.97 (m, 4 H), 1.22-1.33 (m, |
| | | 6 H) |
| 110 | 622.8 | ¹H NMR (400 MHz, METHANOL-d₄) δ ppm 8.03 (br d, J = 8.15 Hz, 1 |
| | | H), 7.76 (s, 1 H), 7.38 (br d, J = 1.88 Hz, 1 H), 5.21-5.44 (m, 1 H), |
| | | 4.35 (br d, J = 5.43 Hz, 1 H), 4.29 (br d, J = 8.57 Hz, 2 H), 4.11 (s, 1 H), |
| | | 3.46-3.63 (m, 2 H), 3.20-3.31 (m, 3 H), 3.02-3.09 (m, 1 H), 2.33- |
| | | 2.47 (m, 1 H), 2.22-2.32 (m, 1 H), 2.08-2.22 (m, 3 H), 1.98-2.08 |
| | | (m, 2 H), 1.72-1.97 (m, 4 H), 1.19-1.34 (m, 5 H), 0.63-0.77 (m, 4 |
| | | H), −0.43-−0.23 (m, 1 H). |
| 111 | 682.3 | 1H NMR (400 MHz, METHANOL-d₄) δ ppm 7.59-7.86 (m, 3 H), |
| | | 5.47-5.72 (m, 1 H), 4.58-4.76 (m, 5 H), 4.36-4.53 (m, 1 H), 3.81- |
| | | 4.15 (m, 3 H), 3.57-3.78 (m, 2 H), 3.38-3.55 (m, 3 H), 2.54-2.87 |
| | | (m, 2 H), 2.31-2.50 (m, 3 H), 1.98-2.28 (m, 6 H), 1.83-1.94 (m, 1 |
| | | H), 1.23-1.36 (m, 1 H), 0.64-0.86 (m, 4 H). |
| 112 | 663.2 | ¹H NMR (400 MHz, DMSO-d₆) δ ppm 13.40 (s, 1 H), 7.89 (s, 1 H), |
| | | 7.85 (br d, J = 10.5 Hz, 1 H), 7.77 (s, 1 H), 6.35 (br d, J = 10.7 Hz, 1 H), |
| | | 5.79-6.16 (m, 1 H), 5.65-5.76 (m, 1 H), 5.63 (s, 1 H), 5.19-5.41 (m, |
| | | 1 H), 4.11-4.33 (m, 2 H), 3.95-4.09 (m, 2 H), 3.47 (s, 1 H), 3.18- |
| | | 3.26 (m, 1 H), 2.99-3.17 (m, 2 H), 2.79-2.93 (m, 1 H), 1.95-2.24 |
| | | (m, 4 H), 1.76-1.89 (m, 5 H), 1.17-1.30 (m, 5 H). |
| 113 | 639.2 | ¹H NMR (600 MHz, DMSO-d₆) δ 7.88 (s, 1H), 7.88 (d, 1H, J = 10.5 |
| | | Hz), 7.83 (br s, 1H), 6.35 (br d, 1H, J = 11.3 Hz), 5.67 (qd, 1H, J = 6.9, |
| | | 11.2 Hz), 5.32 (br s, 1H), 4.4-4.5 (m, 2H), 4.24 (br d, 1H, |
| | | J = 13.4 Hz), 4.10 (d, 1H, J = 10.3 Hz), 3.99 (d, 1H, J-10.4 Hz), 3.9-4.0 |
| | | (m, 1H), 3.58 (d, 1H, J = 13.4 Hz), 3.1-3.1 (m, 2H), 3.0-3.1 (m, 1H), |
| | | 2.8-2.9 (m, 1H), 2.3-2.4 (m, 1H), 2.1-2.2 (m, 3H), 2.0-2.0 (m, |
| | | 1H), 1.8-1.9 (m, 3H), 1.7-1.8 (m, 2H), 1.73 (br dd, 1H, J = 4.4, 9.5 Hz), |
| | | 1.1-1.3 (m, 4H). |
| 114 | 619.2 | ¹H NMR (400 MHz, METHANOL-d₄) δ ppm 7.82-7.90 (m, 1 H), |
| | | 7.66 (s, 1 H), 7.56 (s, 1 H), 6.40-6.46 (m, 1 H), 5.60-5.70 (m, 1 H), |
| | | 5.26-5.47 (m, 1 H), 4.54-4.66 (m, 2 H), 4.28-4.47 (m, 3 H), 4.07- |
| | | 4.18 (m, 1 H), 3.61-3.72 (m, 2 H), 3.06-3.23 (m, 2 H), 2.48-2.60 |
| | | (m, 2 H), 2.44 (s, 3 H), 2.19-2.40 (m, 3 H), 1.91-2.14 (m, 4 H), 1.74- |
| | | 1.85 (m, 1 H), 1.23-1.35 (m, 6 H). |

### (3R)-1-(7-(6-Chloro-5-((Z)-prop-1-en-1-yl)-III-indazol-4-yl)-2-((1-((dimethylamino)methyl)cyclopropyl)methoxy)-8-fluoroquinazolin-4-yl)-3-methylpiperidin-3-ol (Example 115)

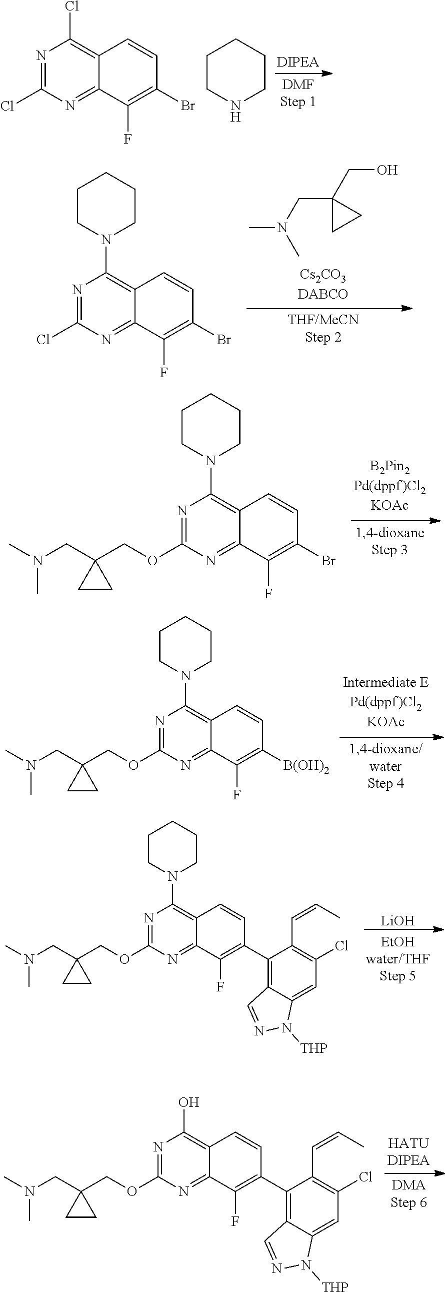

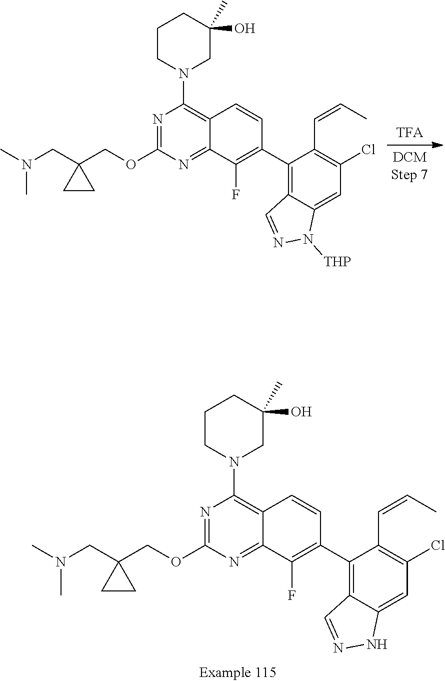

Step 1. 7-Bromo-2-chloro-8-fluoro-4-(piperidin-1-yl)quinazoline. A vial was charged with 7-bromo-2,4-dichloro-8-fluoroquinazoline (0.75 g, 2.5 mmol, LabNetwork), piperidine (0.25 mL, 2.5 mmol) and acetonitrile (7 mL). The contents was cooled to 0° C. then Hunigs base (1.3 mL, 7.6 mmol) was added dropwise. The reaction mixture was stirred at 0° C. and upon completion, diluted with water and extracted with DCM. The combined organic phases were dried over anhydrous sodium sulfate, filtered, and concentrated under reduced pressure. The crude residue was dissolved in DMSO and injected into a pre-packed C18 column (50 g), eluting with a gradient of 5-80% (0.1% formic acid MeCN)/(0.1% formic acid water) over 10 min. The desired fractions were concentrated under reduced pressure to provide to give 7-bromo-2-chloro-8-fluoro-4-(piperidin-1-yl)quinazoline as off-white solid. m/z (ESI): 343.9/345.9 (M+H)⁺.

Step 2. 1-(1-(((7-Bromo-8-fluoro-4-(piperidin-1-yl)quinazolin-2-yl)oxy)methyl)cyclopropyl)-N,N-dimethylmethanamine. A vial was charged with crude 7-bromo-2-chloro-8-fluoro-4-(piperidin-1-yl)quinazoline, (1-((dimethylamino)methyl)cyclopropyl)methanol (0.59 g, 4.6 mmol, Enamine), catalytic DABCO (0.1 equiv), cesium carbonate (2.48 g, 7.6 mmol) and THF/DMF (2:1, 0.2 M). The reaction mixture was stirred at it while monitoring via LCMS. Upon completion, the reaction was diluted with water and extracted with DCM. The combined organic phases were washed with brine, dried over anhydrous sodium sulfate, filtered, and concentrated under reduced pressure to provide 1-(1-(((7-bromo-8-fluoro-4-(piperidin-1-yl)quinazolin-2-yl)oxy)methyl)cyclopropyl)-N,N-dimethylmethanamine (0.77 g, 1.76 mmol, 69% yield) as tan solid. m/z (ESI): 436.8/438.8 (M+H)⁺.

Step 3. (2-((1-((Dimethylamino)methyl)cyclopropyl)methoxy)-8-fluoro-4-(piperidin-1-yl)quinazolin-7-yl)boronic acid. A vial was charged with 1-(1-(((7-bromo-8-fluoro-4-(piperidin-1-yl)quinazolin-2-yl)oxy)methyl)cyclopropyl)-N,N-dimethylmethanamine (0.75 g, 1.72 mmol), 1,1′-bis(diphenylphosphino)ferrocene-palladium dichloride (0.13 mg, 0.17 mmol), potassium acetate (0.34 g, 3.43 mmol), 4,4,4′,4′,5,5,5′,5′-octamethyl-2,2′-bi(1,3,2-dioxaborolane) (0.52 g, 2.06 mmol) and 1,4-dioxane (9 mL). The reaction mixture was heated to 100° C. and monitored via LCMS. Upon completion, the reaction mixture was cooled to rt, filtered, concentrated under reduced pressure, and the crude product was used directly in the next step. m/z (ESI): 403.0 (M+H)⁺.

Step 4. (3R)-1-(7-(6-Chloro-5-((Z)-prop-1-en-1-yl)-1-(tetrahydro-2H-pyran-2-yl)-1H-indazol-4-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol. A vial was charged with (2-((1-((dimethylamino)methyl)cyclopropyl)methoxy)-8-fluoro-4-(piperidin-1-yl)quinazolin-7-yl)boronic acid (0.68 g, 1.69 mmol), (Z)-4-bromo-6-chloro-5-(prop-1-en-1-yl)-1-(tetrahydro-2H-pyran-2-yl)-1H-indazole (0.60 g, 1.69 mmol), potassium carbonate (0.70 g, 5.1 mmol), 1,1′-bis(diphenylphosphino)ferrocene-palladium dichloride (0.12 g, 0.17 mmol), 1,4-dioxane (8.5 mL) and water (2.8 mL) under nitrogen. The mixture was heated to 100° C. for 1.5 h. After cooling to rt, the crude material was injected into a pre-packed C18 column (50 g), eluting with a gradient of 5-80% (0.1% formic acid MeCN)/(0.1% formic acid water) over 20 min. The desired fractions were concentrated under reduced pressure to provide (Z)-1-(1-(((7-(6-chloro-5-(prop-1-en-1-yl)-1-(tetrahydro-2H-pyran-2-yl)-1H-indazol-4-yl)-8-fluoro-4-(piperidin-1-yl)quinazolin-2-yl)oxy)methyl)cyclopropyl)-N,N-dimethylmethanamine (0.28 g, 0.43 mmol, 26% yield) as white solid. m/z (ESI): 633.0 (M+H)⁺.

Step 5. (Z)-7-(6-Chloro-5-(prop-1-en-1-yl)-1-(tetrahydro-2H-pyran-2-yl)-1H-indazol-4-yl)-2-((1-((dimethylamino)methyl)cyclopropyl)methoxy)-8-fluoroquinazolin-4-ol. A vial was charged with (Z)-1-(1-(((7-(6-chloro-5-(prop-1-en-1-yl)-1-(tetrahydro-2H-pyran-2-yl)-1H-indazol-4-yl)-8-fluoro-4-(piperidin-1-yl)quinazolin-2-yl)oxy)methyl)cyclopropyl)-N,N-dimethylmethanamine (0.28 g, 0.43 mmol), lithium hydroxide hydrate (0.18 g, 4.34 mmol), water (1.0 mL), ethanol (1.0 mL) and tetrahydrofuran (1.0 mL). The reaction mixture was heated to 70° C. while monitoring via LCMS. Upon completion, the reaction mixture was diluted with water and extracted with EtOAc. The combined organic phases were washed with brine, dried over anhydrous sodium sulfate, filtered and concentrated under reduced pressure to provide (Z)-7-(6-chloro-5-(prop-1-en-1-yl)-1-(tetrahydro-2H-pyran-2-yl)-1H-indazol-4-yl)-2-((1-((dimethylamino)methyl)cyclopropyl)methoxy)-8-fluoroquinazolin-4-ol (0.22 g, 0.39 mmol, 90% yield) as tan solid. m/z (ESI): 565.9 (M+H)⁺.

Step 6. (3R)-1-(7-(6-Chloro-5-((Z)-prop-1-en-1-yl)-1-(tetrahydro-2H-pyran-2-yl)-1H-indazol-4-yl)-2-((1-((dimethylamino)methyl)cyclopropyl)methoxy)-8-fluoroquinazolin-4-yl)-3-methylpiperidin-3-ol. A vial was charged with (Z)-7-(6-chloro-5-(prop-1-en-1-yl)-1-(tetrahydro-2H-pyran-2-yl)-1H-indazol-4-yl)-2-((1-((dimethylamino)methyl)cyclopropyl)methoxy)-8-fluoroquinazolin-4-ol (0.14 g, 0.25 mmol) and N,N-dimethylacetamide (2.5 mL). The solution was then charged with Hunig's base (0.22 mL, 1.27 mmol). HATU (0.39 g, 1.02 mmol), and (R)-3-methylpiperidin-3-ol hydrochloride (46 mg, 0.31 mmol, Synnovator). The resulting mixture was stirred at rt while monitoring via LCMS. Upon completion, the reaction mixture was injected into a pre-packed C18 column (50 g), eluting with a gradient of 5-80% (0.1% formic acid MeCN)/(0.1% formic acid water) over 10 min. The desired fractions were basified with saturated aqueous sodium bicarbonate and extracted with EtOAc. The combined organic phases were concentrated under reduced pressure to provide (3R)-1-(7-(6-chloro-5-((Z)-prop-1-en-1-yl)-1-(tetrahydro-2H-pyran-2-yl)-1H-indazol-4-yl)-2-((1-((dimethylamino)methyl)cyclopropyl)methoxy)-8-fluoroquinazolin-4-yl)-3-methylpiperidin-3-ol (53 mg, 0.08 mmol, 31% yield) as off-white solid. m/z (ESI): 662.8 (M+H)⁺.

Step 7. (3R)-1-(7-(6-Chloro-5-((Z)-prop-1-en-1-yl)-1H-indazol-4-yl)-2-((1-((dimethylamino)methyl)cyclopropyl)methoxy)-8-fluoroquinazolin-4-yl)-3-methylpiperidin-3-ol. A vial was charged with (3R)-1-(7-(6-chloro-5-((Z)-prop-1-en-1-yl)-1-(tetrahydro-2H-pyran-2-yl)-1H-indazol-4-yl)-2-((1-((dimethylamino)methyl)cyclopropyl)methoxy)-8-fluoroquinazolin-4-yl)-3-methylpiperidin-3-ol (53 mg, 0.08 mmol) and DCM (2 mL), 2,2,2-Trifluoroacetic acid (0.7 mL, −25.0 equiv) was then added and the reaction mixture was stirred at rt while monitoring via LCMS. Upon completion, the mixture was concentrated and redissolved in MeOH. The crude material was injected into a pre-packed C18 column (50 g), eluting with a gradient of 5-80% (0.1% formic acid MeCN)/(0.1% formic acid water) over 10 min. The desired fractions were basified with saturated aqueous sodium bicarbonate, and the aqueous layer was extracted with EtOAc. The combined organic phases were dried over anhydrous sodium sulfate, filtered and concentrated under reduced pressure. The impure material was further purified via SFC/MS purification with MeOH as co-solvent at 10-40% gradient to provide (3R)-1-(7-(6-chloro-5-((Z)-prop-1-en-1-yl)-1H-indazol-4-yl)-2-((1-((dimethylamino)methyl)cyclopropyl)methoxy)-8-fluoroquinazolin-4-yl)-3-methylpiperidin-3-ol as light-yellow solid (8.2 mg, 0.014 mmol, 5.6% yield). ¹H NMR (600 MHz, DMSO-d₆) δ ppm 7.92-8.01 (m, 1H) 7.80-7.88 (m, 1H) 7.66-7.74 (m, 1H) 7.25-7.35 (m, 1H) 6.36-6.43 (m, 1H) 5.58-5.67 (m, 1H) 4.30 (s, 2H) 4.03-4.11 (m, 1H) 3.82-3.92 (m, 1H) 3.38-3.50 (m, 1H) 3.13-3.25 (m, 1H) 2.86 (br s, 6H) 1.98-2.07 (m, 1H) 1.63-1.76 (m, 3H) 1.13-1.19 (m, 7H) 0.87 (br s, 2H) 0.77 (br s, 2H). m/z (ESI): 579.2 (M+H)⁺.

### (4S)-6-(7-(6-Chloro-5-((Z)-prop-1-en-1-yl)-1H-indazol-4-yl)-8-fluoro-2-(((2R,7aS)-2-fluorotetrahydro-1H-pyrrolizin-7a(5H)-yl)methoxy)quinazolin-4-yl)-1-oxa-6-azaspiro[3.5]nonane. (Example 116)

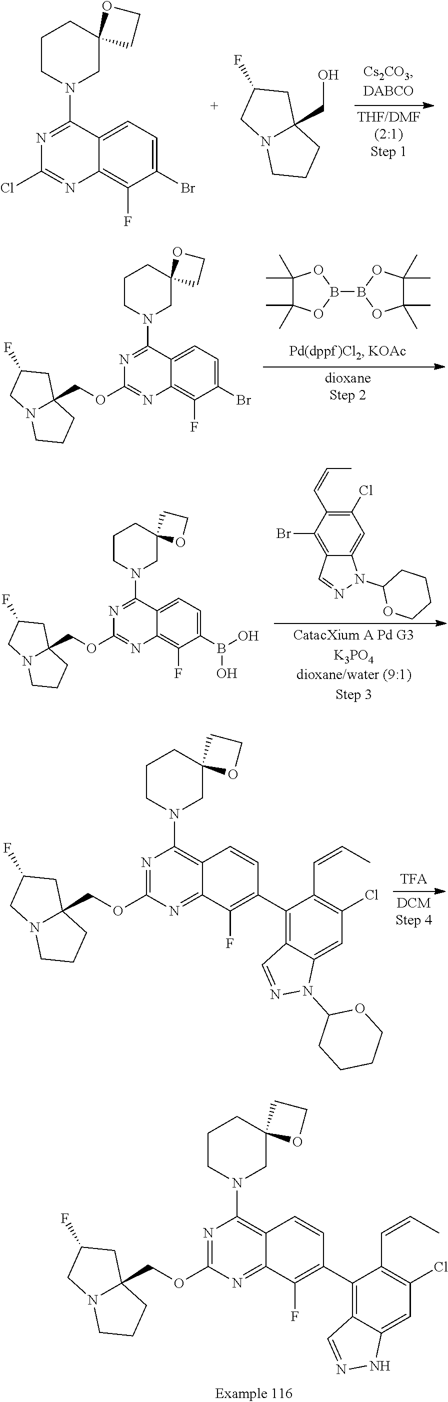

Step 1. (S)-6-(7-Bromo-8-fluoro-2-(((2R,7aS)-2-fluorotetrahydro-1H-pyrrolizin-7a(5H)-yl)methoxy)quinazolin-4-yl)-1-oxa-6-azaspiro[3.5]nonane. A mixture of (S)-6-(7-bromo-2-chloro-8-fluoroquinazolin-4-yl)-1-oxa-6-azaspiro[3.5]nonane (1.83 g, 4.73 mmol, Intermediate Q), ((2R,7aS)-2-fluorohexahydro-1H-pyro-7a-yl)methanol (1.01 g, 6.63 mmol, BLD Pharmatech), 1,4-diazabicyclo[2.2.2]octane (0.11 g, 0.95 mmol), and cesium carbonate (3.08 g, 9.47 mmol) in tetrahydrofuran (14 mL) and N,N-dimethylformamide (7 mL) was stirred at 60° C. for 16 h. The resulting reaction mixture was concentrated in vacuo to remove most of the THF. The above solution was added water and the precipitate was collected by filtration, washed with water and dried. The crude product was purified by chromatography through a silica gel column (40 g), eluting with a gradient of 0-100% EtOAc (with 10% 2 M NH₃ in MeOH) in heptane, to provide (S)-6-(7-bromo-8-fluoro-2-(((2R,7aS)-2-fluorotetrahydro-1H-pyrrolizin-7a(5H)-yl)methoxy)quinazolin-4-yl)-1-oxa-6-azaspiro[3.5]nonane (1.71 g, 3.35 mmol, 71% yield) as white foam. ¹H NMR (400 MHz, DMSO-d₆) δ ppm 7.82 (dd, J=9.0, 1.3 Hz, 1H), 7.54 (dd, J=9.0, 6.7 Hz, 1H), 5.13-5.44 (m, 1H), 4.39 (t, J=7.7 Hz, 2H), 4.07-4.16 (m, 2H), 4.00-4.07 (m, 1H), 3.86-3.95 (m, 1H), 3.68 (d, J=13.4 Hz, 1H), 3.32-3.41 (m, 1H), 3.05-3.20 (m, 2H), 3.03 (s, 1H), 2.79-2.90 (m, 1H), 2.30-2.40 (m, 2H), 2.10-2.20 (m, 1H), 1.98-2.09 (m, 3H), 1.73-1.92 (m, 5H), 1.62-1.73 (m, 1H). m/z (ESI): 508.9/510.9 (M+H)⁺.

Step 2. (8-Fluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)boronic acid. A vial was charged with (S)-6-(7-bromo-8-fluoro-2-(((2R,7aS)-2-fluorotetrahydro-1H-pyrrolizin-7a(5H)-yl)methoxy)quinazolin-4-yl)-1-oxa-6-azaspiro[3.5]nonane (0.11 g, 0.21 mmol), 4,4,4′,4′,5,5,5′,5′-octamethyl-2,2′-bi(1,3,2-dioxaborolane) (64 mg, 0.25 mmol), potassium acetate (41 mg, 0.42 mmol), and Pd(dppf)Cl₂ (12 mg, 0.017 mmol) in 1,4-dioxane (2 mL). The reaction mixture was then heated to 90° C. for 1 h. After cooling to rt, the reaction mixture was filtered through a 0.45 um PTFE Whatman filter and the filtrate was used as in the subsequent step.

Step 3. (4S)-6-(7-(6-Chloro-5-((Z)-prop-1-en-1-yl)-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)quinazolin-4-yl)-1-oxa-6-azaspiro[3.5]nonane. A vial was charged with (8-fluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)boronic acid (0.10 g, 0.21 mmol), CatacXium a Pd G3 (15 mg, 0.021 mmol), potassium phosphate (0.13 g, 0.63 mmol), (Z)-4-bromo-6-chloro-5-(prop-1-en-1-yl)-1-(tetrahydro-2H-pyran-2-yl)-1H-indazole (0.10 g, 0.29 mmol, Intermediate L) and degassed 1,4-dioxane (1 mL). The reaction mixture was heated to 80° C. for 45 min. After cooling to rt, the reaction mixture was subjected to reverse-phase column chromatography using a C18 column (50 g), 0.1% formic acid in MeCN/H₂O, gradient 10-100% over 10 min. The desired fractions were collected and neutralized with saturated NaHCO₃ solution. The aqueous mixture was extracted with EtOAc and the combined organic layers were washed with brine, dried over MgSO₄, filtered and concentrated under reduced pressure to provide (4S)-6-(7-(6-chloro-5-((Z)-prop-1-en-1-yl)-1-(tetrahydro-2H-pyran-2-yl)-1H-indazol-4-vi)-8-fluoro-2-(((2R,7aS)-2-fluorotetrahydro-1H-pyrrolizin-7a(5H)-yl)methoxy)quinazolin-4-yl)-1-oxa-6-azaspiro[3.5]nonane (32 mg, 0.045 mmol, 22% yield) as light yellow solid.

Step 4. (4S)-6-(7-(6-Chloro-5-((Z)-prop-1-en-1-yl)-1H-indazol-4-yl)-8-fluoro-2-(((2R,7aS)-2-fluorotetrahydro-1H-pyrrolizin-7a(5H)-yl)methoxy)quinazolin-4-yl)-1-oxa-6-azaspiro[3.5]nonane. To a 6-mL vial was charged with (4S)-6-(7-(6-chloro-5-((Z)-prop-1-en-1-yl)-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)quinazolin-4-yl)-1-oxa-6-azaspiro[3.5]nonane (32 mg, 0.045 mmol) in dichloromethane (1.5 mL). Trifluoroacetic acid (0.4 mL, 5.3 mmol) was added, and the reaction was stirred at rt for 1 h. The reaction mixture was slowly quenched with saturated NaHCO₃ solution (3 mL) and extracted with DCM. The combined organic layers were washed with brine, dried over MgSO₄, filtered and concentrated under reduced pressure. The crude material was purified by reverse-phase column chromatography using a C18 column (15 g), 0.1% formic acid in MeCN/H₂O, gradient 5-100% over 10 min. The desired fractions were neutralized with saturated NaHCO₃ solution and extracted with DCM. The combined organic layers were washed with brine, dried over MgSO₄, filtered and concentrated to give (4S)-6-(7-(6-chloro-5-((Z)-prop-1-en-1-yl)-1H-indazol-4-yl)-8-fluoro-2-(((2R,7aS)-2-fluorotetrahydro-1H-pyrrolizin-7a(5H)-yl)methoxy)quinazolin-4-yl)-1-oxa-6-azaspiro[3.5]nonane (9.5 mg, 0.015 mmol, 7.3% yield) as white solid. ¹H NMR (400 MHz, METHANOL-d₄) δ ppm 7.99 (t, J=8.8 Hz, 1H), 7.73-7.80 (m, 2H), 7.32 (br s, 1H), 6.43 (br d, J=11.1 Hz, 1H), 5.65 (dq, J=11.1, 6.9 Hz, 1H), 5.26-5.44 (m, 1H), 4.57 (td, J=7.8, 1.8 Hz, 2H), 4.38 (br d, J=10.9 Hz, 2H), 4.28-4.33 (m, 1H), 4.12 (br d. J=13.4 Hz, 1H), 3.80 (dd, J=13.4, 9.4 Hz, 1H), 3.38-3.55 (m, 2H), 3.21-3.30 (m, 1H), 3.08 (td, J=9.4, 6.1 Hz, 1H), 2.50 (t, J=7.7 Hz, 2H), 2.44 (br dd, J=15.3, 4.8 Hz, 1H), 2.32 (br d, J=4.0 Hz, 1H), 2.14-2.29 (m, 3H), 1.91-2.10 (m, 6H), 1.76-1.86 (m, 1H), 1.25 (d. J=6.9 Hz, 3H). ¹⁹F NMR (376 MHz. METHANOL-d₄) δ ppm −128.01 (s, 1F). −173.68 (s, 1F). m/z (ESI): 621.0 (M+H)⁺.

TABLE 20
Additional Examples 117 to 118. Prepared in an Analogous Manner to Example 116.

| | | | Salt | | |
|---|---|---|---|---|---|
| Ex.# | Structure | Name | Form | Reagent | |
| 117 | 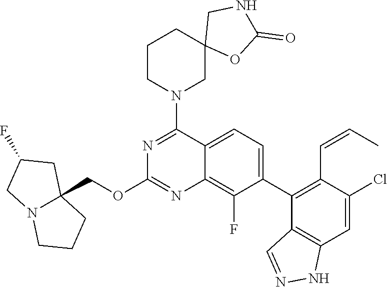 [SMILES: C/C=C\c1c(Cl)cc2[nH]ncc2c1-c1ccc2c(N3CCCC4(CNC(=O)O4)C3)nc(OC[C@@]34CCCN3C[C@H](F)C4)nc2c1F] | 7-(7-(6-Chloro-5-((Z)- prop-1-en-1-yl)-1H- indazol-4-yl)-8-fluoro- 2-(((2R,7aS)-2- fluorotetrahydro-1H- pyrrolizin-7a(5H)- yl)methoxy)quinazolin- 4-yl)-1-oxa-3,7- diazaspiro[4.5]decan-2- one | Bis (2,2,2- tri- fluoro- aceate) | Step 1: 1- Oxa-3,7- diazaspiro [4.5]decan- 2-one (CAS#: 1308384- 36-2, ChemSpace) Step 3: (Z)-4- Bromo-6- chloro-5- (prop-1-en- 1-yl)-1- (tetrahydro- 2H-pyran- 2-yl)-1H- | |
| | | | | indazole | |
| | | | | (Intermediate | |
| | | | | L) | |
| 118 | 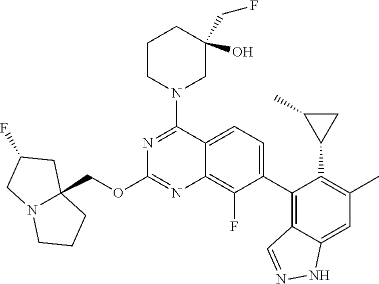 [SMILES: Cc1cc2[nH]ncc2c(-c2ccc3c(N4CCC[C@](O)(CF)C4)nc(OC[C@@]45CCCN4C[C@H](F)C5)nc3c2F)c1[C@H]1C[C@H]1C] | (R)-1-((S)-8-Fluoro-2- (((2R,7aS)-2- fluorotetrahydro-1H- pyrrolizin-7a(5H)- yl)methoxy)-7-(6- methyl-5-((1S,2R)-2- methylcyclopropyl)- 1H-indazol-4- yl)quinazolin-4-yl)-3- (fluoromethyl) piperidin-3-ol | Bis- trifluo roacet ate salt | Step 1: 3- (Fluoro- methyl) piperidin- 3-ol hydrochloride (CAS#: 2306262- 40-6, PharmaBlock) Step 2: 4-bromo-6- methyl-5- ((1S,2R)-2- methylcyclo- propyl)-1- (tetrahydro- 2H-pyran- | Chiral separation after Step 1. |
| | | | | 2-yl)-1H- | |
| | | | | indazole | |
| | | | | (Intermediate | |
| | | | | S) | |

TABLE 21
Conditions for chiral SFC separations.

| | | Peak to |
|---|---|---|
| Compound | SFC Conditions | Ex.# |
| 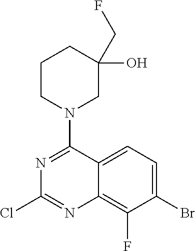 [SMILES: OC1(CF)CCCN(c2nc(Cl)nc3c(F)c(Br)ccc23)C1] | Column: Chiralpak IC, 21 x 250 mm, 5 μm, Mobile phase: 25% 2-propanol with 0.2% triethylamine Flowrate: 80 mL/min Yield: 3.65 g sample was submitted to generate 1.72 g of peak 1 with an ee of >99% and 1.79 g of peak 2 with an ee of >95%. | Peak 1: Example 118 |

TABLE 22
Analytical Data for Examples 117 to 118.

| | MS | |
|---|---|---|
| Ex. | m/z (ESI): | |
| # | (M + H)⁺ | ¹H NMR |
| 117 | 649.8 | ¹H NMR (400 MHz, METHANOL-d₄) δ ppm 7.98 (s, 1 H), 7.82 (s, 1 |
| | | H), 7.73-7.79 (m, 1 H), 7.37-7.53 (m, 1 H), 6.39-6.48 (m, 1 H), |
| | | 5.66 (br s, 2 H), 4.72 (s, 3 H), 4.51-4.62 (m, 1 H), 3.82-4.14 (m, 3 |
| | | H), 3.65-3.77 (m, 2 H), 3.41-3.57 (m, 3 H), 2.55-2.83 (m, 2 H), |
| | | 2.31-2.54 (m, 3 H), 1.99-2.29 (m, 4 H), 1.87-1.98 (m, 1 H), 1.19- |
| | | 1.31 (m, 3 H). |
| 118 | 621.0 | ¹H NMR (400 MHz, METHANOL-d₄) δ ppm 8.11-8.24 (m, 1 H), |
| | | 7.42-7.75 (m, 3 H), 5.47-5.71 (m, 1 H), 4.75-4.80 (m, 3 H), 4.53- |
| | | 4.63 (m, 1 H), 4.46 (br d, J = 9.41 Hz, 2 H), 4.29-4.38 (m, 1 H), 4.01- |
| | | 4.12 (m, 1 H), 3.77-4.00 (m, 3 H), 3.57-3.67 (m, 1 H), 3.45-3.55 |
| | | (m, 1 H), 2.68-2.81 (m, 1 H), 2.64 (s, 4 H), 2.33-2.52 (m, 3 H), 2.15- |
| | | 2.30 (m, 2 H), 2.04-2.15 (m, 1 H), 1.81-2.01 (m, 3 H), 1.14-1.28 |
| | | (m, 1 H), 0.65-0.82 (m, 1 H), 0.56-0.64 (m, 3 H), −0.43-−0.28 (m, 1 |
| | | H). |

### (3R)-1-(7-(6-Chloro-5-((Z)-prop-1-en-1-yl)-1H-indazol-4-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (Example 119)

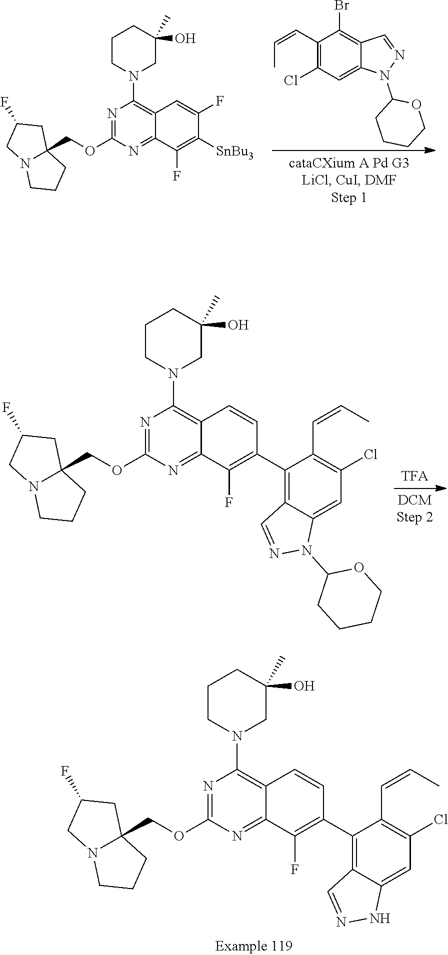

Step 1. (3R)-1-(7-(6-Chloro-5-((Z)-prop-1-en-1-yl)-1-(tetrahydro-2H-pyran-2-yl)-11H-indazol-4-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol. A vial was charged with (R)-1-(6,8-difluoro-2-(((2R,7aS)-2-fluorotetrahydro-1H-pyrrolizin-7a(5H)-yl)methoxy)-7-(tributylstannyl)quinazolin-4-yl)-3-methylpiperidin-3-ol (0.31 mg, 0.42 mmol, Intermediate R), (Z)-4-bromo-6-chloro-5-(prop-1-en-1-yl)-1-(tetrahydro-2H-pyran-2-yl)-1H-indazole (0.10 g, 0.28 mmol, Intermediate L), cataCXiumn a Pd G3 (21 mg, 0.028 mmol), lithium chloride (24 mg, 0.56 mmol), copper(I) iodide (27 mg, 0.14 mmol) and degassed N,N-dimethylformamide (2 mL). The reaction mixture was heated to 100° C. and monitored via LCMS. Upon completion, the cooled reaction mixture was injected into a pre-packed C18 column (50 g), eluting with a gradient of 5-80% (0.1% formic acid MeCN)/(0.1% formic acid water) over 10 min. The desired fractions were basified with saturated aqueous sodium bicarbonate and extracted with DCM. The combined organic phases were concentrated under reduced pressure to provide (3R)-1-(7-(6-chloro-5-((Z)-prop-1-en-1-yl)-1-(tetrahydro-2H-pyran-2-yl)-H-indazol-4-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (64 mg, 0.09 mmol, 32% yield) as tan solid. m/z (ESI): 711.0 (M+H)⁺.

Step 2. (3R)-1-(7-(6-Chloro-5-((Z)-prop-1-en-1-yl)-1H-indazol-4-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol. A vial was charged with (3R)-1-(7-(6-chloro-5-((Z)-prop-1-en-1-yl)-1-(tetrahydro-2H-pyran-2-yl)-1H-indazol-4-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (64 mg, 0.09 mmol) in DCM (2.0 mL), 2,2,2-Trifluoroacetic acid (0.7 mL, 25.0 equiv) was added, and the reaction mixture was stirred at rt for 1 h. The mixture was concentrated and redissolved in MeOH (1 mL). The crude material was injected into a pre-packed C18 column (50 g), eluting with a gradient of 5-100% (0.1% formic acid MeCN)/(0.1% formic acid water) over 10 min. The desired fractions were basified with saturated aqueous sodium bicarbonate, and the aqueous layer was extracted with EtOAc. The combined organic phases were dried over anhydrous sodium sulfate, filtered and concentrated under reduced pressure to provide (3R)-1-(7-(6-chloro-5-((Z)-prop-1-en-1-yl)-1H-indazol-4-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol (41 mg, 0.065 mmol, 23% yield) as white solid. ¹H NMR (400 MHz, METHANOL-d₄) δ ppm 7.83 (s, 3H) 6.34-6.44 (m, 1H) 5.62-5.77 (m, 1H) 5.22-5.44 (m, 1H) 4.18-4.37 (m, 3H) 4.00-4.09 (m, 1H) 3.36 (br d, J=3.14 Hz, 3H) 3.17-3.29 (m, 3H) 2.98-3.08 (m, 1H) 2.22-2.42 (m, 2H) 1.74-2.06 (m, 8H) 1.29 (br d, J=7.94 Hz, 7H). m/z (ESI): 627.0 (M+H)⁺.

### (3R)-1-(7-(6-Chloro-5-((Z)-prop-1-en-1-yl)-1H-indazol-4-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol

### Example 120

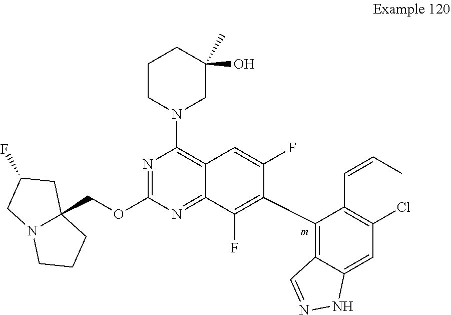 [SMILES: C/C=C\c1c(Cl)cc2[nH]ncc2c1-c1c(F)cc2c(N3CCC[C@@](C)(O)C3)nc(OC[C@@]34CCCN3C[C@H](F)C4)nc2c1F]

The sample (Example 119, 41 mg) was purified via SFC using a Chiralcel OD, 21×150 mm, 5 μm, column with a mobile phase of 35% methanol with 0.2% triethylamine using a flowrate of 125 mL/min to generate 11.5 mg of peak 1 with an ee of >99% and 9.5 mg of peak 2 with an ee of >90%. Peak 2 yielded Example 120.

### (R)-1-((S)-6,8-Difluoro-2-(((2R,7aS)-2-fluorotetrahydro-1H-pyrrolizin-7a(5H)-yl)methoxy)-7-(6-methyl-5-((1S,2R)-2-methylcyclopropyl)-1H-indazol-4-yl)quinazolin-4-yl)-3-methylpiperidin-3-ol bis(2,2,2-trifluoroacetate) (Example 121)

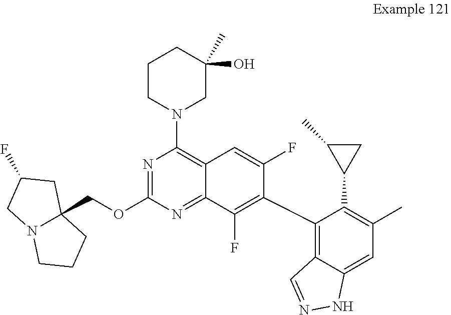 [SMILES: Cc1cc2[nH]ncc2c(-c2c(F)cc3c(N4CCC[C@@](C)(O)C4)nc(OC[C@@]45CCCN4C[C@H](F)C5)nc3c2F)c1[C@H]1C[C@H]1C]

Using an analogous method used for Example 119, (R)-1-(6,8-difluoro-2-(((2R,7aS)-2-fluorotetrahydro-1H-pyrrolizin-7a(5H)-yl)methoxy)-7-(tributylstannyl)quinazolin-4-yl)-3-methylpiperidin-3-ol (0.15 g, 0.21 mmol, Intermediate R), and 4-bromo-6-methyl-5-((1S,2R)-2-methylcyclopropyl)-1-(tetrahydro-2H-pyran-2-yl)-1H-indazole (60 mg, 0.17 mmol, Intermediate S) provided (R)-1-((S)-6,8-difluoro-2-(((2R,7aS)-2-fluorotetrahydro-1H-pyrrolizin-7a(5H)-yl)methoxy)-7-(6-methyl-5-((1S,2R)-2-methylcyclopropyl)-1H-indazol-4-yl)quinazolin-4-yl)-3-methylpiperidin-3-ol. ¹H NMR (400 MHz. METHANOL-d₄) δ ppm 7.97 (br d, J=10.03 Hz, 1H), 7.50-7.67 (m, 2H), 5.44-5.71 (m, 1H), 4.66-4.80 (m, 3H), 4.42-4.56 (m, 1H), 4.21-4.32 (m, 1H), 3.83-4.09 (m, 3H), 3.60 (d, J=13.38 Hz, 1H), 3.34-3.53 (m, 3H), 2.64 (d. J=2.51 Hz, 5H), 2.31-2.50 (m, 3H), 2.14-2.24 (m, 2H), 2.00-2.14 (m, 1 H), 1.73-1.92 (m, 3H), 1.32 (s, 3H), 1.17-1.29 (m, 1H), 0.68-0.80 (m, 1H), 0.65 (d, J=6.06 Hz, 3H), −0.29 (br dd, J=15.47, 5.64 Hz, 1H). m/z (ESI): 621.0 (M+H)⁺.

### Biological Evaluation

Provided in this section is the biological evaluation of the specific examples provided herein.

KRAS G12D TR-FRET Assay Compounds of interest were prepared in a dose-response titration in DMSO, and 80 nL were added via Labcyte Echo to each well of a 384-well plate (Perkin Elmer 6008280). The His-tagged KRAS G12D protein (Amgen) was diluted to 20 nM in Assay Buffer (20 mM HEPES, pH 7.4, 10 mM MgCl₂, 50 mM NaCl, 0.1% BSA, 0.01% Tween-20, 10 μM GDP) and 2 uL was added to the appropriate wells of the 384-well plate. The plate was incubated for 30 minutes at room temperature. Biotinylated KRPep-2d substrate (Amgen) was diluted to 20 nM in Assay Buffer and 2 μL was added to all wells and incubated for 1 hour at room temperature. Detection Reagent (0.4 nM LANCE Eu-W1024 Anti-6×-His (Perkin Elmer AD0401), 5 nM streptavidin-d2 (Cisbio 610SADLA)) was prepared in Assay Buffer, then 4 μL was added to the plate and incubated for 1 hour at room temperature. Plates were read using PerkinElmer EnVision (ex: 320 nm, em1: 665 nm, em2: 615 nm) and em1/em2 data was used to generate curve fits using a 4-parameter logistic model to calculate IC₅₀ values.

### KRAS G12D Coupled Nucleotide Exchange Assay

Purified GDP-bound KRAS protein (aa 1-169), containing both G12D and C118A amino acid substitutions and an N-terminal His-tag, was pre-incubated in assay buffer (25 mM HEPES pH 7.4, 10 mM MgCl₂, and 0.01% Triton X-100) with a compound dose-response titration for 2 h. Following compound pre-incubation, purified SOS protein (aa 564-1049) and GTP (Roche 10106399001) were added to the assay wells and incubated for an additional 30 min. To determine the extent of inhibition of SOS-mediated nucleotide exchange, purified GST-tagged cRAF (aa 1-149), nickel chelate AlphaLISA acceptor beads (PerkinElmer AL108R), and AlphaScreen glutathione donor beads (PerkinElmer 6765302) were added to the assay wells and incubated for 10 minutes. The assay plates were then read on a PerkinElmer EnVision Multilabel Reader, using AlphaScreen® technology, and data were analyzed using a 4-parameter logistic model to calculate IC₅₀ values.

### Phospho-ERK1/2 MSD Assay

AsPC-1 (ATCC® CRL-1682™) cells were cultured in RPMI 1640 Medium (ThermoFisher Scientific 11875093) containing 10% fetal bovine serum (ThermoFisher Scientific 16000044) and Ix penicillin-streptomycin-glutamine (ThermoFisher Scientific 10378016). Sixteen hours prior to compound treatment, AsPC-1 cells were seeded in 96-well cell culture plates at a density of 25,000 cells/well and incubated at 37° C., 5% CO₂. A compound dose-response titration was diluted in growth media, added to appropriate wells of a cell culture plate, and then incubated at 37° C., 5% CO₂ for 2 h. Following compound treatment, cells were washed with ice-cold Dulbecco's phosphate-buffered saline, no Ca²⁺ or Mg²⁺ (ThermoFisher Scientific 14190144), and then lysed in RIPA buffer (50 mM Tris-HCl pH 7.5, 1% Igepal, 0.5% sodium deoxycholate, 150 mM NaCl, and 0.5% sodium dodecyl sulfate) containing protease inhibitors (Roche 4693132001) and phosphatase inhibitors (Roche 4906837001). Phosphorylation of ERK1/2 in compound-treated lysates was assayed using Phospho-ERK1/2 Whole Cell Lysate kits (Meso Scale Discovery K151DWD) according to the manufacturer's protocol. Assay plates were read on a Meso Scale Discovery Sector Imager 6000, and data were analyzed using a 4-parameter logistic model to calculate IC₅₀ values.

TABLE 23
Biochemical and cellular activity of examples.

| | KRAS G12D | KRAS G12D | 2 h p-ERK |
|---|---|---|---|
| Ex. | TR-FRET | Coupled Exchange | (AsPC-1 cells), |
| # | IC₅₀ (μM) | IC₅₀ (μM) | IC₅₀ (μM) |
| 1 | 0.002 | 0.003 | 0.048 |
| 2 | 0.001 | 0.001 | 0.009 |
| 3 | 0.127 | 0.134 | NT |
| 4 | 0.0003 | 0.0005 | 0.426 |
| 5 | 0.003 | 0.003 | 0.303 |
| 6 | 0.005 | 0.004 | 0.123 |
| 7 | 0.012 | 0.011 | 0.812 |
| 8 | 0.022 | 0.011 | NT |
| 9 | 0.004 | 0.003 | 0.259 |
| 10 | 0.014 | 0.015 | 1.040 |
| 11 | 0.212 | 0.186 | NT |
| 12 | 0.001 | 0.001 | 0.005 |
| 13 | 0.968 | 1.254 | 0.205 |
| 14 | 0.001 | 0.002 | 0.003 |
| 15 | 0.004 | 0.004 | 0.297 |
| 16 | 0.002 | 0.002 | 0.132 |
| 17 | 0.011 | 0.009 | 0.744 |
| 18 | 0.046 | 0.037 | 2.890 |
| 19 | 0.111 | 0.037 | 7.740 |
| 20 | 0.002 | 0.002 | 0.058 |
| 21 | 0.040 | 0.040 | NT |
| 22 | 0.096 | 0.058 | NT |
| 23 | 0.002 | 0.002 | 0.157 |
| 24 | 0.070 | 0.080 | NT |
| 25 | 0.004 | 0.005 | 0.146 |
| 26 | 0.004 | 0.004 | 0.270 |
| 27 | 0.001 | 0.001 | 0.018 |
| 28 | 0.273 | 0.302 | NT |
| 29 | 0.001 | 0.001 | 0.015 |
| 30 | 0.003 | 0.003 | 0.012 |
| 31 | 0.003 | 0.002 | 0.027 |
| 32 | 0.005 | 0.003 | 0.025 |
| 33 | 0.005 | 0.003 | 0.004 |
| 34 | 0.002 | 0.002 | 0.002 |
| 35 | 0.122 | 0.043 | NT |
| 36 | 0.020 | 0.012 | 1.080 |
| 37 | 0.011 | 0.010 | 0.391 |
| 38 | 0.012 | 0.012 | 0.425 |
| 39 | 0.003 | 0.004 | 0.010 |
| 40 | 0.006 | 0.011 | 0.053 |
| 41 | 0.023 | 0.023 | 1.470 |
| 42 | 0.025 | 0.014 | 1.160 |
| 43 | 0.022 | 0.013 | 1.670 |
| 44 | 0.050 | 0.028 | 1.860 |
| 45 | 0.012 | 0.007 | 0.525 |
| 46 | 0.079 | 0.036 | 2.570 |
| 47 | 0.004 | 0.004 | 0.021 |
| 48 | 0.022 | 0.023 | NT |
| 49 | 0.010 | 0.010 | NT |
| 50 | 0.003 | 0.003 | 0.023 |
| 51 | 0.003 | 0.004 | 0.047 |
| 52 | 0.010 | 0.007 | 0.179 |
| 53 | 0.003 | 0.007 | 0.251 |
| 54 | 0.003 | 0.004 | 0.023 |
| 55 | 0.021 | 0.011 | 0.822 |
| 56 | 0.024 | 0.008 | 0.507 |
| 57 | 0.437 | 0.087 | NT |
| 58 | 0.001 | 0.000 | 0.003 |
| 59 | 0.021 | 0.008 | 0.039 |
| 60 | 0.015 | 0.012 | 1.170 |
| 61 | 0.729 | 0.272 | NT |
| 62 | 0.017 | 0.002 | 0.281 |
| 63 | 0.223 | 0.138 | NT |
| 64 | 0.000 | 0.000 | 0.074 |
| 65 | 0.011 | 0.006 | 0.260 |
| 66 | 0.657 | 0.589 | NT |
| 67 | 0.004 | 0.003 | 0.338 |
| 68 | 0.436 | 0.023 | 1.930 |
| 69 | 0.011 | 0.005 | 0.175 |
| 70 | 0.019 | 0.004 | 0.185 |
| 71 | 0.111 | 0.014 | 1.400 |
| 72 | 0.008 | 0.002 | 0.151 |
| 73 | 0.002 | 0.001 | 0.090 |
| 74 | 0.117 | 0.010 | 1.050 |
| 75 | 0.154 | 0.009 | 0.500 |
| 76 | 0.005 | 0.003 | 0.009 |
| 77 | 0.006 | 0.004 | 0.029 |
| 78 | 0.005 | 0.003 | 0.016 |
| 79 | 0.009 | 0.005 | 0.033 |
| 80 | 0.004 | 0.002 | 0.007 |
| 81 | 0.005 | 0.004 | 0.008 |
| 82 | 0.005 | 0.002 | 0.024 |
| 83 | 0.01 | 0.005 | 0.058 |
| 84 | 0.005 | 0.002 | 0.072 |
| 85 | 0.007 | 0.007 | 0.085 |
| 86 | 0.006 | 0.004 | 0.052 |
| 87 | 0.005 | 0.003 | 0.055 |
| 88 | 0.015 | 0.006 | 0.013 |
| 89 | 0.007 | 0.005 | 0.014 |
| 90 | 0.005 | 0.003 | 0.007 |
| 91 | 0.005 | 0.003 | 0.016 |
| 92 | 0.007 | 0.005 | 0.028 |
| 93 | 0.007 | 0.005 | 0.028 |
| 94 | 0.004 | 0.004 | 0.026 |
| 95 | 0.012 | 0.009 | 0.18 |
| 96 | 0.01 | 0.006 | 0.087 |
| 97 | 0.004 | 0.002 | 0.029 |
| 98 | 0.007 | 0.003 | 0.028 |
| 99 | 0.009 | 0.005 | 0.093 |
| 100 | 0.004 | 0.003 | 0.09 |
| 101 | 0.005 | 0.003 | 0.039 |
| 102 | 0.004 | 0.003 | 0.036 |
| 103 | 0.007 | 0.005 | 0.027 |
| 104 | 0.002 | 0.001 | 0.03 |
| 105 | 0.003 | 0.003 | 0.032 |
| 100 | 0.007 | 0.004 | 0.087 |
| 107 | 0.004 | 0.003 | 0.166 |
| 108 | 0.004 | 0.003 | 0.115 |
| 109 | 0.004 | 0.004 | 0.1 |
| 110 | 0.009 | 0.008 | 0.123 |
| 111 | 0.005 | 0.004 | 0.055 |
| 112 | 0.005 | 0.005 | 0.125 |
| 113 | 0.003 | 0.003 | 0.041 |
| 114 | 0.006 | 0.005 | 0.087 |
| 115 | 0.008 | 0.009 | 0.247 |
| 116 | 0.003 | 0.002 | 0.059 |
| 117 | 0.003 | 0.002 | 0.074 |
| 118 | 0.005 | 0.004 | 0.126 |
| 119 | 0.016 | 0.011 | 0.111 |
| 120 | 0.004 | 0.003 | 0.035 |
| 121 | 0.009 | 0.006 | 0.156 |

NT: not tested.

### REFERENCES

All references, for example, a scientific publication or patent application publication, cited herein are incorporated herein by reference in their entirety and for all purposes to the same extent as if each reference was specifically and individually indicated to be incorporated by reference in its entirety for all purposes.

1. A compound of Formula (I): 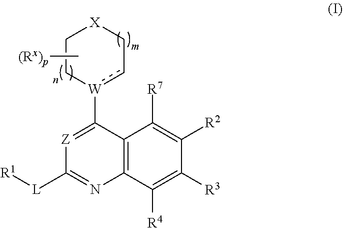
or a pharmaceutically acceptable salt of said compound, wherein; [symbol]
is a single bond or a double bond;
W is C, CH or N;
X is CH₂, O, S, S(O), S(O)(NR) or S(O)₂;
n is 0, 1, 2, or 3;
m is 0, 1, 2 or 3;
p is 0, 1, 2, 3 or 4;
each R^{x} is hydroxyl, halogen, oxo, cyano, —N(R)₂, C₁₋₄ alkyl, C₁₋₄ alkoxy, C₁₋₄ haloalkyl, C₁₋₄ haloalkoxy, 5-7 membered heteroaryl, -T-R^{y} or two R^{x} taken together with the same carbon or adjacent carbon atoms can form C₃₋₇ cycloalkyl, a 5-7 membered heterocycloalkyl, wherein each C₃₋₇ cycloalkyl or 5-7 membered heterocycloalkyl is further substituted with 0-3 occurrences of R^{y} or two R^{x} taken together can form a bridged ring where the bridge is selected from one of the following: —C₁₋₄ alkylene, —C₁₋₄ alkylene-O—C₁₋₄ alkylene-, —O—, —S— or —C₁₋₄ alkylene-S—C₁₋₄ alkylene- and wherein each C₁₋₄ alkylene is further substituted with 0-2 occurrences of R^{Y};
Z is CH, CR′ or N;
R′ is halogen, cyano or C₁₋₄ alkyl;
L is a bond, C₁₋₆ alkylene, C₁₋₆ alkenylene, —O—C₁₋₆ alkylene, —S—C₁₋₆ alkylene, NR, O or S, wherein each C₁₋₆ alkylene, —O—C₁₋₆ alkylene and —S—C₁₋₆ alkylene chain is substituted with 0-2 occurrences of R²;
R¹ is hydroxyl, aryl, heteroaryl, C₃₋₈ cycloalkyl or heterocycloalkyl optionally substituted with 0-3 occurrences of R⁵;
R² is hydrogen, hydroxyl, halogen, amino, C₁₋₄ alkyl, C₁₋₄ alkoxy, C₁₋₄ haloalkyl, C₂₋₄ alkenyl, C₂₋₄ alkynyl, C₃₋₈ cycloalkyl, cyano, or two R² on the same or adjacent carbon atoms can be taken together to form a C₃₋₇ cycloalkyl;
R³ is aryl or heteroaryl optionally substituted with 0-4 occurrences of R;
R⁴ is hydrogen, hydroxyl, halogen, C₁₋₄ alkyl, C₁₋₄ alkoxy, C₁₋₄ haloalkyl, C₂₋₄ alkenyl, C₂₋₄ alkynyl, C₃₋ₛ cycloalkyl or cyano;
each R⁵ is halogen, hydroxyl, oxo, amino, C₁₋₄ alkyl or -T-R^{y};
each R⁶ is halogen, hydroxyl, amino, cyano, C₁₋₄ alkyl, C₂₋₄ alkenyl, C₂₋₄ alkynyl, C₁₋₄ haloalkyl, C₃₋₇ cycloalkyl or two R⁶ taken on adjacent carbon atoms can be used to form a C₃₋₇ cycloalkyl, wherein each C₃₋₇ cycloalkyl is further substituted with 0-2 R⁷;
R⁷ is hydrogen, halogen or C₁₋₄ alkyl;
T is C₁₋₄ alkylene, —O—, —S—, —C₁₋₄ alkylene-C(O)— or —NR^{z}—C(O)—;
R^{Y} is halogen, hydroxyl, cyano, C₁₋₄ alkoxy or amino; and
R^{z} is hydrogen or C₁₋₄ alkyl.

2. The compound of claim 1, wherein Z is N.

3. The compound of claim 1, wherein L is —O-methylene substituted with 0 occurrences of R².

4. The compound of claim 1, wherein R¹ is 7-(hexahydro-1H-pyrrolizine) substituted with 0-3 occurrences of R⁵.

5. The compound of claim 4, wherein -L-R¹ is

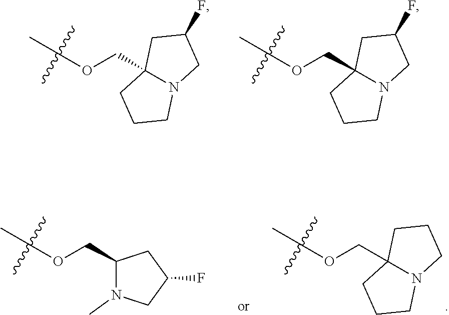

6. The compound of claim 1, wherein R³ is aryl substituted with 0-3 occurrences of R⁶.

7. The compound of claim 6, wherein R³ is phenyl or naphthyl substituted with 0-3 occurrences of R⁶.

8. The compound of claim 1, wherein R³ is heteroaryl substituted with 0-3 occurrences of R⁶.

9. The compound of claim 8, wherein R³ is 4-(1H-indazolyl) or 7-(1H-indazolyl) substituted with 0-3 occurrences of R⁶.

10. The compound of claim 1, wherein R³ is

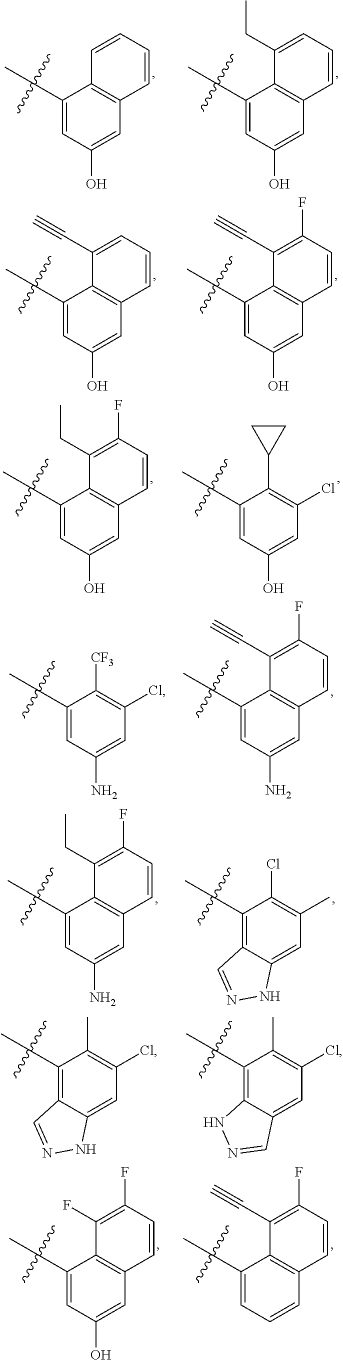

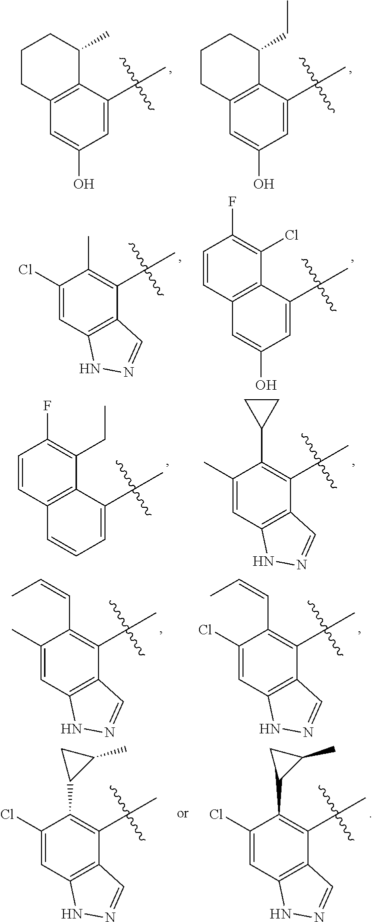

11. The compound of claim 1, wherein W is N.

12. The compound of claim 11, wherein X is CH₂.

13. The compound of claim 12, wherein 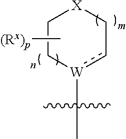
is

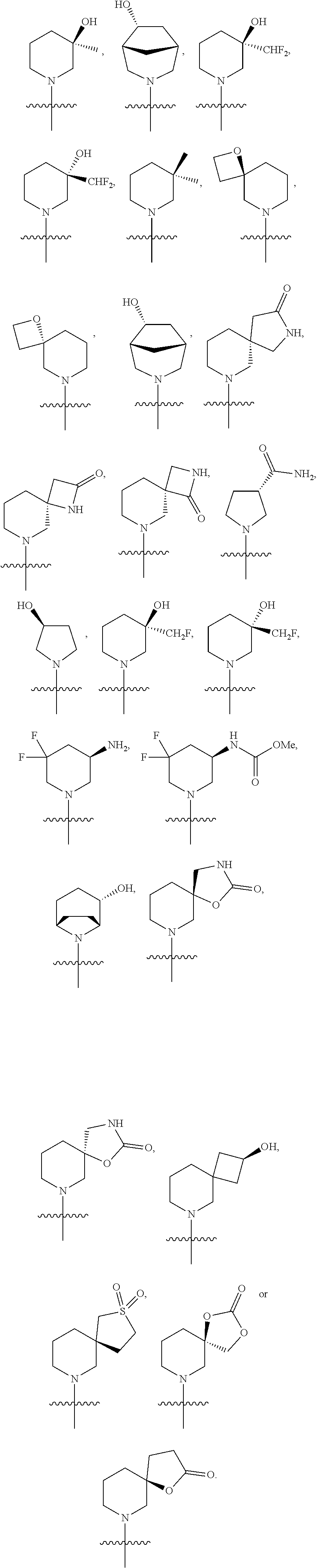

14. The compound of claim 11, wherein X is O.

15. The compound of claim 14, wherein 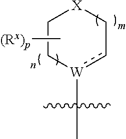
is

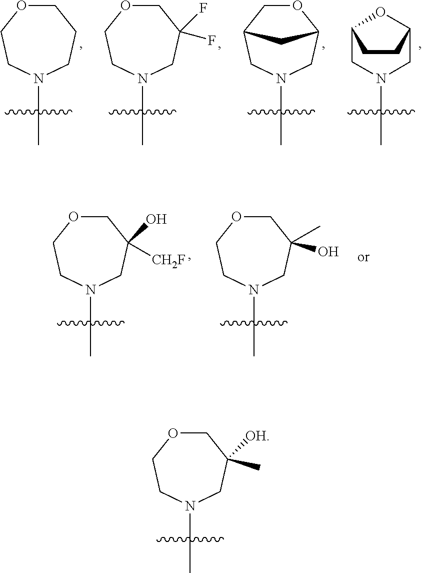

16. The compound of claim 1, wherein R² is hydrogen, methyl, fluorine, cyano, hydroxyl or amino.

17. The compound of claim 1, wherein R⁴ is fluorine or methyl.

18. The compound of claim 1, wherein R⁷ is hydrogen.

19. The compound of claim 1, wherein the compound is selected from one of the following compounds:
4-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol;
(3R)-1-(7-(8-Ethynyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol;
7-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
4-(7-(8-Ethyl-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol;
(3R)-1-(7-(8-Ethynyl-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol;
(5S)-7-(7-(8-Ethyl-7-fluoro-3-hydroxynaphthalen-1-yl)-6,8-difluoro-2-(((2S,4R)-4-fluoro-1-methylpyrrolidin-2-yl)methoxy)quinazolin-4-yl)-1-oxa-3,7-diazaspiro[4.5]decan-2-one;
4-(6,8-Difluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)-5-ethyl-6-fluoronaphthalen-2-ol;
4-(7-(8-Chloro-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol;
(3R)-1-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol;
5-Ethyl-6-fluoro-4-(8-fluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)naphthalen-2-ol;
4-(6,8-Difluoro-2-(((2R,7aS)-2-fluorotetrahydro-1H-pyrrolizin-7a(5H)-yl)methoxy)-4-(1-oxa-6-azaspiro[3.5]nonan-6-yl)quinazolin-7-yl)-5-ethyl-6-fluoronaphthalen-2-ol;
4-(7-(8-Ethyl-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol;
(S)-7-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
(R)-7-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
4-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol;
5-Chloro-4-(6,8-difluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)-6-fluoronaphthalen-2-ol;
(2S,4s)-6-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-azaspiro[3.5]nonan-2-ol;
7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-6-ol;
(R)-1-(7-(8-Ethynyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol;
(R)-1-(7-(8-Ethynyl-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol;
7-(7-(8-Ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
4-(7-(8-Chloro-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol;
4-(7-(7,8-Difluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol;
(5S)-7-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-2-thia-7-azaspiro[4.5]decane 2,2-dioxide; or
4-(7-(7,8-Difluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol.

20. The compound of claim 1, wherein the compound is selected from one of the following compounds:
4-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol;
(3R)-1-(7-(8-Ethynyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol;
7-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-1-oxa-3,7-diazaspiro[4.5]decan-2-one;
4-(7-(8-Ethyl-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol;
(3R)-1-(7-(8-Ethynyl-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol;
(5S)-7-(7-(8-Ethyl-7-fluoro-3-hydroxynaphthalen-1-yl)-6,8-difluoro-2-(((2S,4R)-4-fluoro-1-methylpyrrolidin-2-yl)methoxy)quinazolin-4-yl)-1-oxa-3,7-diazaspiro[4.5]decan-2-one;
4-(6,8-Difluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)-5-ethyl-6-fluoronaphthalen-2-ol;
4-(7-(8-Chloro-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-6-methyl-1,4-oxazepan-6-ol;
(3R)-1-(7-(8-Ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol; or
5-Ethyl-6-fluoro-4-(8-fluoro-2-(((2R,7aS)-2-fluorotetrahydro-1H-pyrrolizin-7a(5H)-yl)methoxy)-4-((S)-1-oxa-6-azaspiro[3.5]nonan-6-yl)quinazolin-7-yl)naphthalen-2-ol.

21. A pharmaceutical composition comprising the compound according to any one of claims 1-20 or a pharmaceutically acceptable salt of said compound, and a pharmaceutically acceptable excipient.

22. A compound according to any one of claims 1-20, or a tautomer thereof, or a pharmaceutically acceptable salt of said compound, or the pharmaceutical composition according to claim 21 for use as a medicament.

23-28. (canceled)

29. A method of treating cancer in a subject in need thereof, the method comprising administering to the subject a therapeutically effective amount of the compound according to any one of to any one of claims 1-20 or a pharmaceutically acceptable salt thereof or a pharmaceutical composition according to claim 21.

30. A method of treating cancer in a subject in need thereof, the method comprising administering to the subject a therapeutically effective amount of the compound according to any one of to any one of claims 1-20 or a pharmaceutically acceptable salt thereof or a pharmaceutical composition according to claim 21, wherein one or more cells express KRAS G12D mutant protein.

31. The method according to claim 29 or 30, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

32. The method according to claim 29 or 30, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

33. The method according to claim 32, wherein the cancer is non-small cell lung cancer.

34. The method according to claim 32, wherein the cancer is colorectal cancer.

35. The method according to claim 32, wherein the cancer is pancreatic cancer.

36. The method according to anyone of claims 29-35, wherein the subject has a cancer that was determined to have one or more cells expressing the KRAS G12D mutant protein prior to administration of the compound or a pharmaceutically acceptable salt thereof.
